(19) United States
(12) Patent Application Publication
(10) Pub. No.: US 2021/0139890 A1
(43) Pub. Date: May 13, 2021

(54) NOVEL CRISPR RNA TARGETING ENZYMES AND SYSTEMS AND USES THEREOF

(71) Applicant: Arbor Biotechnologies, Inc., Cambridge, MA (US)

(72) Inventors: David R. CHENG, Boston, MA (US); David A. SCOTT, Cambridge, MA (US); Winston X. YAN, Brookline, MA (US); Shaorong CHONG, Somerville, MA (US)

(21) Appl. No.: 16/626,932

(22) PCT Filed: Jul. 2, 2018
(86) PCT No.: PCT/US2018/040649

§ 371 Date: Dec. 27, 2019

Related U.S. Application Data
(63) Continuation-in-part of application No. 15/916,271, filed on Mar. 8, 2018, now Pat. No. 10,392,616.
Application No. 15/916,271 is a continuation-in-part of application No. 15/916,274, filed on Mar. 8, 2018.
(60) Provisional application No. 62/635,443, filed on Feb. 26, 2018.
(60) Provisional application No. 62/628,921, filed on Feb. 9, 2018.
(60) Provisional application No. 62/626,679, filed on Feb. 5, 2018.
(60) Provisional application No. 62/619,691, filed on Jan. 19, 2018.
(60) Provisional application No. 62/587,381, filed on Nov. 16, 2017.
(60) Provisional application No. 62/580,880, filed on Nov. 2, 2017.
(60) Provisional application No. 62/572,367, filed on Oct. 13, 2017.
(60) Provisional application No. 62/527,957, filed on Jun. 30, 2017.

(51) Int. Cl. C12N 15/11; C12N 15/113; C12N 9/22; C07K 14/195; C12N 15/62

(52) U.S. Cl. CPC C12N 15/11; C12N 15/111; C12N 15/113; C12N 2310/20; C07K 14/195; C12N 15/62; C12N 9/22

(57) ABSTRACT

The disclosure describes novel systems, methods, and compositions for the manipulation of nucleic acids in a targeted fashion. The disclosure describes non-naturally occurring, engineered CRISPR systems, components, and methods for targeted modification of a nucleic acid.

### CROSS REFERENCE TO RELATED APPLICATIONS

This application claims the benefit of priority of U.S. Application No. 62/527,957, filed Jun. 30, 2017; U.S. Application No. 62/572,367, filed Oct. 13, 2017; U.S. Application No. 62/580,880, filed Nov. 2, 2017; U.S. Application No. 62/587,381, filed Nov. 16, 2017; U.S. Application No. 62/619,691, filed Jan. 19, 2018; U.S. Application No. 62/626,679, filed Feb. 5, 2018; U.S. Application No. 62/628,921, filed Feb. 9, 2018; U.S. Application No. 62/635,443, filed Feb. 26, 2018; U.S. application Ser. No. 15/916,271, filed Mar. 8, 2018 and U.S. application Ser. No. 15/916,274, filed Mar. 8, 2018. The content of each of the foregoing applications is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 20, 2020, is named 51451-0020011_3.23.20_ST25 and is 1,925,607 bytes in size.

### FIELD OF THE INVENTION

The present disclosure relates to novel CRISPR systems and components, systems for detecting CRISPR systems, and methods and compositions for use of the CRISPR systems in, for example, nucleic acid targeting and manipulation.

### BACKGROUND

Recent advances in genome sequencing technologies and analysis have yielded significant insights into the genetic underpinning of biological activities in many diverse areas of nature, ranging from prokaryotic biosynthetic pathways to human pathologies. To fully understand and evaluate the vast quantity of information produced by genetic sequencing technologies, equivalent increases in the scale, efficacy, and ease of technologies for genome and epigenone manipulation are needed. These novel genone and epigenome engineering technologies will accelerate the development of novel applications in numerous areas, including biotechnology, agriculture, and human therapeutics.

Clustered Regularly Interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely diverse group of proteins effectors, non-coding elements, as well as loci architectures, some examples of which have been engineered and adapted to produce important biotechnologies.

The components of the systems involved in host defense include one or more effector proteins capable of modifying DNA or RNA and a RNA guide element that is responsible for targeting these protein activities to a specific sequence on the phage DNA or RNA. The RNA guide is composed of a CRISPR RNA (crRNA) and may require an additional trans-activating RNA (tracrRNA) to enable targeted nucleic acid manipulation by the effector protein(s). The crRNA consists of a direct repeat (DR) responsible for protein binding to the crRNA and a spacer sequence, which may be engineered to be complementary to a desired nucleic acid target sequence. In this way, CRISPR systems can be programmed to target DNA or RNA targets by modifying the spacer sequence of the crRNA.

CRISPR-Cas systems can be broadly classified into two classes: Class I systems are composed of multiple effector proteins that together form a complex around a crRNA, and Class 2 systems that consist of a single effector protein that complexes with the crRNA to target DNA or RNA substrates. The single-subunit effector compositions of the Class 2 systems provide a simpler component set for engineering and application translation, and has thus far bee important sources of programmable effectors. The discovery, engineering, and optimization of novel Class 2 systems may lead to widespread and powerful programmable technologies for genome engineering and beyond.

### SUMMARY

CRISPR-Cas systems are adaptive immune systems in archaea and bacteria that defend the species against foreign genetic elements. The characterization and engineering of Class 2 CRISPR-Cas systems, exemplified by CRISPR-Cas9, have paved the way for a diverse array of biotechnology applications in genome editing and beyond. Nevertheless, there remains a need for additional programmable effectors and systems for modifying nucleic acids and polynucleotides (i.e., DNA, RNA, or any hybrid, derivative, or modification) beyond the current CRISPR-Cas systems that enable novel applications through their unique properties.

The present disclosure provides methods for computational identification of new single-effector CRISPR Class 2 systems from genomic databases, together with the development of the natural loci into engineered systems, and experimental validation and application translation.

In on aspect, the disclosure provides engineered, non-naturally occurring Clustered Regularly Interspaced Short Palindromic Repeat (CRISPR)—associated (Cas) systems that include: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid; and ii) a Type VI-D CRISPR-Cas effector protein or a nucleic acid encoding the effector protein, wherein the effector protein includes or consists of an amino acid sequence having at least 85% sequence identity to an amino acid sequence provided in Table 2 (e.g., SEQ ID NOs. 1-31, and 200-350), wherein the effector protein is capable of binding (e.g., binds under appropriate conditions) to the RNA guide and of targeting the target nucleic acid sequence complementary to the RNA guide spacer sequence.

In some embodiments, the effector protein includes or consists of an amino acid sequence provided in Table 2 (e.g., SEQ ID NOs. 1-31, and 200-350). In some embodiments, the effector protein is RspCas13d (SEQ ID NO: 2) or EsCas13d (SEQ ID NO: 1).

In some embodiments, the effector protein includes at least two HEPN domains. In some embodiments, none, one, or two or more of the HEPN domains are catalytically deactivated.

In another aspect, the disclosure provides engineered, non-naturally occurring Clustered Regularly Interspaced Short Palindromic Repeat (CRISPR)—associated (Cas) systems that include: i) an RNA guide or a nucleic acid encoding the RNA guide, wherein the RNA guide includes a direct repeat sequence and a spacer sequence capable of hybridizing (e.g., hybridizes under appropriate conditions) to a target nucleic acid; ii) a CRISPR-associated protein or a nucleic acid encoding the CRISPR-associated protein; and iii) an accessory protein or a nucleic acid encoding the accessory protein, wherein the accessory protein includes at least one WYL domain, wherein the WYL domain includes an amino acid sequence PXXX₁XXXXXXXXXYL (SEQ ID NO: 198), wherein X₁ is C, V, I, L, P, F, Y, M, or W, and wherein X is any amino acid; and/or at least one ribbon-ribbon-helix (RHH) fold or at least one helix-turn-helix (HTH) domain; wherein the CRISPR-associated protein is capable of binding (e.g., binds under appropriate conditions) to the RNA guide and of targeting the target nucleic acid sequence complementary to the spacer sequence, and wherein the accessory protein modulates an activity of the CRISPR-associated protein.

In another aspect, the disclosure provides engineered, non-naturally occurring Clustered Regularly Interspaced Short Palindromic Repeat (CRISPR)—associated (Cas) systems that include: i) an RNA guide or a nucleic acid encoding the RNA guide, wherein the RNA guide includes a direct repeat sequence and a spacer sequence capable of hybridizing (e.g., hybridizes under appropriate conditions) to a target nucleic acid; ii) a CRISPR-associated protein or a nucleic acid encoding the CRISPR-associated protein; and an accessory protein or a nucleic acid encoding the accessory protein, wherein the accessory protein includes at least one WYL domain, and wherein the accessory protein includes an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in any one of Tables 4, 5, and 6 (e.g., SEQ ID NOs. 78-93, and 590-671); wherein the CRISPR-associated protein is capable of binding (e.g., binds under appropriate conditions) to the RNA guide and of targeting the target nucleic acid sequence complementary to the spacer sequence, and wherein the accessory protein modulates an activity of the CRISPR-associated protein.

In some embodiments, the activity is a nuclease activity (e.g., a DNAse activity, a targeted RNAse activity, or a collateral RNAse activity).

In some embodiments, the accessory protein increases the activity of the CRISPR-associated protein. In some embodiments, the accessory protein decreases the activity of the CRISPR-associated protein.

In some embodiments, the accessory protein includes or consists of an amino acid sequence provided in any one of Tables 4, 5, and 6 (e.g., SEQ ID NOs. 78-93, and 590-671).

In some embodiments, the accessory protein includes or is RspWYL1 (SEQ ID NO: 81).

In some embodiments, the targeting of the target nucleic acid results in a modification of the target nucleic acid.

In some embodiments, the CRISPR-associated protein is a Class 2 CRISPR-Cas system protein. In some embodiments, the CRISPR-associated protein includes a RuvC domain (e.g., at least one, two, three, or more RuvC domains). In some embodiments, the CRISPR-associated protein is selected from the group consisting of a Type VI Cas protein, a Type V Cas protein, and a Type II Cas protein. In some embodiments, the CRISPR-associated protein is a Cas13a protein, a Cas13b protein, a Cas13c protein, a Cas12a protein, or a Cas9 protein. In some embodiments, the CRISPR-associated protein is a Type VI-D CRISPR-Cas effector protein comprising at least two HEPN domains, wherein none, one, or two or more of the HEPN domains are catalytically deactivated.

In some embodiments, the effector protein includes an amino acid sequence having at least 85% sequence identity to an amino acid sequence provided in Table 2 (e.g., SEQ ID NOs. 1-31, and 200-350). In some embodiments, the effector protein includes or consists of an amino acid sequence provided in Table 2 (e.g., SEQ ID NOs. 1-31, and 200-350). In some embodiments, the effector protein includes or is RspCas13d (SEQ ID NO: 2) or EsCas13d (SEQ ID NO: 1).

In some embodiments, the target nucleic acid is an RNA. In some embodiments, the target nucleic acid is a DNA.

In some embodiments, the modification of the target nucleic acid is a cleavage event. In some embodiments, the modification results in: (a) decreased transcription; (b) decreased translation; or (c) both (a) and (b), of the target nucleic acid. In some embodiments, modification results in (a) increased transcription; (b) increased translation; or (c) both (a) and (b), of the target nucleic acid.

In some embodiments, the effector protein includes one or more amino acid substitutions within at least one of the HEPN domains. In some embodiments, the one or more one amino acid substitutions include an alanine substitution at an amino acid residue corresponding to R295, H300, R849, or H854 of SEQ ID NO: 1, or R288, H293, R820, or H825 of SEQ ID NO: 2. In some embodiments, the one or more amino acid substitutions result in a reduction of an nuclease activity of the Type VI-D CRISPR-Cas effector protein, as compared to the nuclease activity of the Type VI-D CRISPR-Cas effector protein without the one or more acid substitutions.

In some embodiments, the RNA guide includes a direct repeat sequence that includes or consists of a nucleotide sequence provided in Table 3 (e.g., SEQ ID NOs: 32-49, 52-77, 351-589). In some embodiments, the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G. In some embodiments, the direct repeat sequence includes or consists of either 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153).

In some embodiments, the spacer includes or consists of from about 15 to about 42 nucleotides.

In some embodiments, the RNA guide further includes a trans-activating CRISPR RNA (tracrRNA).

In some embodiments, the systems include a single-stranded donor template or a double-stranded donor template. In some embodiments, the donor template is a DNA or an RNA.

In some embodiments, the systems include a target RNA or a nucleic acid encoding the target RNA, wherein the target RNA includes a sequence that is capable of hybridizing (e.g., hybridizes under appropriate conditions) to the spacer sequence of the RNA guide.

In some embodiments, the systems are present in a delivery system (e.g., a nanoparticle, a liposome, an adeno-associated virus, an exosome, a microvesicle, and a gene-gun).

In another aspect, the disclosure provides a cell including any of the systems described herein. In some embodiments, the cell is a eukaryotic cell (e.g., a mammalian cell or a plant cell). In some embodiments, the cell is a prokaryotic cell (e.g., a bacterial cell).

In another aspect, the disclosure provides an animal model or a plant model including a cell that includes any of the systems described herein.

In another aspect, the disclosure provides methods of cleaving a target nucleic acid (and compositions for use in such methods), which include contacting a target nucleic acid with a system described herein, wherein the spacer sequence is complementary to at least 15 nucleotides of the target nucleic acid, wherein the CRISPR-associated protein or the Type VI-D CRISPR effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the CRISPR-associated protein or the Type VI-D CRISPR effector protein cleaves the target nucleic acid. In some embodiments, the target nucleic acid is within a cell.

In another aspect the disclosure provides methods of inducing dormancy or death of a cell which include contacting the cell with a system described herein (and compositions for use in such methods), wherein the spacer sequence is complementary to at least 15 nucleotides of the target nucleic acid, wherein the CRISPR-associated protein or the Type VI-D CRISPR effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the CRISPR-associated protein or the Type VI-D CRISPR-Cas effector protein cleaves a non-target nucleic acid within the cell, thereby inducing dormancy or death of the cell. In some embodiments, the death is via apoptosis, necrosis, necroptosis, or a combination thereof.

In some embodiments of any of the methods described herein (and compositions for use in such methods), the target nucleic acid is an RNA selected from the group consisting of an mRNA, a tRNA, a ribosomal RNA, a non-coding RNA, a lncRNA, or a nuclear RNA. In some embodiments of any of the methods described herein, the target nucleic acid is a DNA selected from the group consisting of chromosomal DNA, mitochondrial DNA, single-stranded DNA, or plasmid DNA.

In some embodiments of any of the methods described herein (and compositions for use in such methods), upon binding of the complex to the target nucleic acid, the CRISPR-associated protein or the Type VI-D CRISPR-Cas effector protein exhibits collateral RNAse activity.

In some embodiments of any of the methods described herein (and compositions for use in such methods), the cell is a cancer cell (e.g., a tumor cell). In some embodiments, the cell is an infectious agent cell or a cell infected with an infectious agent. In some embodiments, the cell is a bacterial cell, a cell infected with a virus, a cell infected with a prion, a fungal cell, a protozoan, or a parasite cell.

In another aspect, the disclosure provides methods of treating a condition or disease in a subject in need thereof and compositions for use in such methods. The methods include administering to the subject a system described herein, wherein the spacer sequence is complementary to at least 15 nucleotides of a target nucleic acid associated with the condition or disease, wherein the CRISPR-associated protein or the Type VI-D CRISPR-Cas effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the CRISPR-associated protein or the Type VI-D CRISPR-Cas effector protein cleaves the target nucleic acid, thereby treating the condition or disease in the subject.

In some embodiments of the methods described herein (and compositions for use in such methods), the condition or disease is a cancer or an infectious disease. In some embodiments, the condition or disease is cancer, and wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In another aspect, the disclosure provides the use of a system described herein in a method selected from the group consisting of RNA sequence specific interference; RNA sequence-specific gene regulation; screening of RNA, RNA products, ncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

In some embodiments of any of the systems described herein, the effector protein is fused to a base-editing domain, an RNA methyltransferase, an RNA demethylase, a splicing modifier, a localization factor, or a translation modification factor. In some embodiments of any of the systems described herein, the CRISPR-associated protein is fused to a base-editing domain (e.g., Adenosine Deaminase Acting on RNA (ADAR) 1 (ADAR1), ADAR2, apolipoprotein B mRNA editing enzyme, catalytic polypeptide-like (APOBEC)), and activation-induced cytidine deaminase (AID)), an RNA methyltransferase, an RNA demethylase, a splicing modifier, a localization factor, or a translation modification factor.

In some embodiments, the systems described herein include an RNA-binding fusion polypeptide that includes an RNA-binding domain (e.g., MS2) and a base-editing domain (e.g., ADAR1, ADAR2, APOBEC, or AID).

In another aspect, the disclosure provides method of modifying an RNA molecule, comprising contacting the RNA molecule with a system described herein.

In yet another aspect, the disclosure provides methods of detecting a target RNA in a sample (and compositions for use in such methods). The methods include: a) contacting the sample with: (i) an RNA guide or a nucleic acid encoding the RNA guide, wherein the RNA guide includes a direct repeat sequence and a spacer sequence capable of hybridizing (e.g., hybridizes under appropriate conditions) to the target RNA; (ii) a Type VI-D CRISPR-Cas effector protein or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNAse activity and cleaves the labeled detector RNA; and b) measuring a detectable signal produced by cleavage of the labeled detector RNA, wherein said measuring provides for detection of the single-stranded target RNA in the sample. In some embodiments, the methods further include comparing the detectable signal with a reference signal and determining the amount of target RNA in the sample. In some embodiments, the target RNA is single-stranded. In some embodiments, the target RNA is double-stranded. In some embodiments, the methods further include transcribing (e.g., using a T7 polymerase) a DNA molecule (e.g., a DNA molecule present in the sample) to produce the target RNA. In some embodiments, the target RNA was transcribed from a DNA molecule. In some embodiments, the methods further include pre-amplifying a nucleic acid in the sample (e.g., via isothermal amplification, recombinase polymerase amplification (RPA), or immunoprecipitation) prior to the contacting step.

In some embodiments, the methods further include contacting the sample with an accessory protein comprising at least one WYL domain. In some embodiments, the accessory protein includes an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in any one of Tables 4, 5, and 6. In some embodiments, the accessory protein includes or is RspWYL1 (SEQ ID NO: 81).

In some embodiments, the effector protein includes an amino acid sequence having at least 85% sequence identity to an amino acid sequence provided in Table 2 (e.g., SEQ ID NOs. 1-31, and 200-350).

In some embodiments, the measuring is performed using gold nanoparticle detection, fluorescence polarization, colloid phase transition/dispersion, electrochemical detection, and semiconductor based-sensing.

In some embodiments, the labeled detector RNA includes a fluorescence-emitting dye pair, a fluorescence resonance energy transfer (FRET) pair, or a quencher/fluor pair. In some embodiments, the labeled detector RNA produces a first detectable signal prior to cleavage by the effector protein and a second detectable signal after cleavage by the effector protein.

In some embodiments, a detectable signal is produced when the labeled detector RNA is cleaved by the effector protein.

In some embodiments, upon cleavage of the labeled detector RNA by the effector protein, an amount of detectable signal produced by the labeled detector RNA is decreased. In some embodiments, upon cleavage of the labeled detector RNA by the effector protein, an amount of detectable signal produced by the labeled detector RNA is increased.

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes a direct repeat sequence and a spacer sequence capable of hybridizing (e.g., hybridizes under appropriate conditions) to a target nucleic acid; ii) a CRISPR-associated protein or a nucleic acid encoding the CRISPR-associated protein; and an accessory protein or a nucleic acid encoding the accessory protein, wherein the accessory protein includes at least one WYL domain, and wherein the accessory protein includes an amino acid sequence having at least 85% sequence identity to an amino acid sequence provided in any one of Tables 4, 5, and 6 (e.g., SEQ ID NOs. 78-93, and 590-671); wherein the CRISPR-associated protein is capable of binding (e.g., binds under appropriate conditions) to the RNA guide and of targeting the target nucleic acid sequence complementary to the spacer sequence, and wherein the accessory protein modulates an activity of the CRISPR-associated protein.

In some embodiments, the activity is a nuclease activity (e.g., a DNAse activity or an RNAse activity). In some embodiments, the RNAse activity is targeted RNAse activity or a collateral RNAse activity.

In some embodiments, the accessory protein increases the activity of the CRISPR-associated protein. In some embodiments, the accessory protein decreases the activity of the CRISPR-associated protein.

In some embodiments, the accessory protein includes one WYL domain. In some embodiments, the accessory protein includes two WYL domains. In some embodiments, the accessory protein further includes a helix-turn-helix (HTH) fold. In some embodiments, the accessory protein further includes a ribbon-helix-helix (RHH) fold.

In some embodiments, the accessory protein includes or consists of an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in any one of Tables 4, 5, and 6 (e.g., SEQ ID NOs. 78-93, and 590-671). In some embodiments, the accessory protein includes or consists of an amino acid sequence provided in any one of Tables 4, 5, and 6 (e.g., SEQ ID NOs. 78-93, and 590-671). In some embodiments, the accessory protein is RspWYL1 (SEQ ID NO: 81).

In some embodiments, the target nucleic acid includes or is an RNA. In some embodiments, the target nucleic acid includes or is a DNA.

In some embodiments, the targeting of the target nucleic acid results in a modification (e.g., a cleavage event) of the target nucleic acid. In some embodiments, the modification results in cell toxicity. In some embodiments, the modification results in decreased transcription and/or decreased translation of the target nucleic acid. In some embodiments, the modification results in increased transcription and/or increased translation of the target nucleic acid.

In some embodiments, the CRISPR-associated protein is a Class 2 CRISPR-Cas system protein. In some embodiments, the CRISPR-associated protein includes a RuvC domain. In some embodiments, the CRISPR-associated protein is selected from the group consisting of a Type VI Cas protein, a Type V Cas protein, and a Type II Cas protein. In some embodiments, the CRISPR-associated protein is a Cas13a protein, a Cas13b protein, a Cas13c protein, a Cas12a protein, or a Cas9 protein.

In some embodiments, the CRISPR-associated protein is a Type VI-D CRISPR-Cas effector protein comprising at least two HEPN domains (e.g., two, three, four, or more HEPN domains). In some embodiments, the Type VI-D CRISPR-Cas effector protein includes two HEPN domains. In some embodiments, at least one (e.g., one, two, three, four, or more) of the HEPN domains is catalytically inactivated.

In some embodiments, the Type VI-D CRISPR-Cas effector protein includes or consists of an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in Table 2 (e.g., SEQ ID NOs. 1-31, and 200-350). In some embodiments, the Type VI-D CRISPR-Cas effector protein includes or consists of an amino acid sequence provided in Table 2 (e.g., SEQ ID NOs. 1-31, and 200-350). In some embodiments, the Type VI-D CRISPR-Cas effector protein is RspCas13d (SEQ ID NO: 2) or EsCas13d (SEQ ID NO: 1).

In some embodiments, the Type VI-D CRISPR-Cas effector protein includes or consists of one or more (e.g., two, three, four, five or six) amino acid substitutions within at least one of the HEPN domains. In some embodiments, the Type VI-D CRISPR-Cas effector protein includes six or less (e.g., five, four, three, two or one) amino acid substitutions within at least one of the HEPN domains. In some embodiments, the one or more one amino acid substitutions include or consist of an alanine substitution at an amino acid residue corresponding to R295, H300, R849, or H854 of SEQ ID NO: 1, or R288, H293, R820, or H825 of SEQ ID NO: 2. In some embodiments, the one or more amino acid substitutions result in a reduction of an RNAse activity of the Type VI-D CRISPR-Cas effector protein, as compared to the RNAse activity of the Type VI-D CRISPR-Cas effector protein without the one or more acid substitutions.

In some embodiments, the CRISPR-associated proteins include or consist of at least one (e.g., two, three, four, five, six, or more) nuclear localization signal (NLS). In some embodiments, the CRISPR-associated protein include or consist of at least one (e.g., two, three, four, five, six, or more) nuclear export signal (NES). In some embodiments, the CRISPR-associated protein includes at least one (e.g., two, three, four, five, six, or more) NLS and at least one (e.g., two, three, four, five, six, or more) NES.

In some embodiments, the direct repeat sequence includes 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G. In some embodiments, the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G. In some embodiments, the direct repeat sequence includes or consists of a nucleotide sequence provided in Table 3 (e.g., SEQ ID NOs 32-49, 52-77, 351-589). In some embodiments, the direct repeat sequence includes or consists of either 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153).

In some embodiments, the spacer includes from about 15 to about 42 nucleotides. In some embodiments, the RNA guide includes a trans-activating CRISPR RNA (tracrRNA).

In some embodiments of the systems described herein, the systems include a single-stranded donor template or a double-stranded donor template (e.g., a single-stranded DNA, a double stranded DNA, a single-stranded RNA, or a double stranded RNA).

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G; and ii) a Type VI-D CRISPR-Cas effector protein or a nucleic acid encoding the effector protein, wherein the effector protein is capable of binding (e.g., binds under appropriate conditions) to and of targeting the target nucleic acid sequence complementary to the RNA guide spacer sequence, and wherein the target nucleic acid is an RNA.

In one aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G; and ii) a Type VI-D CRISPR-Cas effector protein or a nucleic acid encoding the effector protein, wherein the effector protein is capable of binding (e.g., binds under appropriate conditions) to and of targeting the target nucleic acid sequence complementary to the RNA guide spacer sequence, and wherein the target nucleic acid is an RNA.

In some embodiments, the Type VI-D CRISPR-Cas effector protein includes at least two HEPN domains. In some embodiments, the protein is about 1200 amino acids or less (e.g., 1100, 1000, 1050, 900, 950, 800 amino acids) in length.

In other embodiments, the targeting of the target nucleic acid results in a modification of the target nucleic acid. In some embodiments, the modification of the target nucleic acid is a cleavage event. In some embodiments, the modification results in cell toxicity.

In some embodiments, the modification results in decreased transcription and/or decreased translation of the target nucleic acid. In some embodiments, the modification results in increased transcription and/or increased translation of the target nucleic acid.

In various embodiments, the systems further include a donor template nucleic acid. In some embodiments, the donor template nucleic acid is a DNA or an RNA.

In some embodiments, the Type VI-D CRISPR-Cas effector protein includes one or more (e.g., two, three, four, five or six) amino acid substitutions within at least one of the HEPN domains. In some embodiments, the one or more amino acid substitutions include an alanine substitution at an amino acid residue corresponding to R295, H300, R849, or H854 of SEQ ID NO: 1, or R288, H293, R820, or H825 of SEQ ID NO: 2. In some embodiments, the one or more amino acid substitutions result in a reduction of an RNAse activity of the Type VI-D CRISPR-Cas effector protein, as compared to the RNAse activity of the Type VI-D CRISPR-Cas effector protein without the one or more amino acid substitutions.

In some embodiments, the Type VI-D CRISPR-Cas effector protein includes or consists of an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in Table 2 (e.g., SEQ ID NOs. 1-31, and 200-350). In some embodiments, the Type VI-D CRISPR-Cas effector protein includes or consists of an amino acid sequence provided in Table 2 (e.g., SEQ ID NOs. 1-31, and 200-350). In some embodiments, the Type VI-D CRISPR-Cas effector protein is RspCas13d (SEQ ID NO: 2) or EsCas13d (SEQ ID NO: 1).

In some embodiments, the systems include an accessory protein or a nucleic acid encoding the accessory protein, wherein the accessory protein includes at least one WYL domain, and wherein the accessory protein includes or consists of an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in any one of Tables 4, 5, and 6 (e.g., SEQ ID NOs. 78-93, and 590-671).

In some embodiments, the accessory protein includes two WYL domains. In some embodiments, the accessory protein further includes a helix-turn-helix (HTH) fold and/or a ribbon-helix-helix (RHH) fold. In some embodiments, the accessory protein is RspWYL1 (SEQ ID NO: 81).

In some embodiments, the accessory protein modulates (e.g., increases or decreases) an activity of the Type VI-D CRISPR-Cas effector protein. In some embodiments, the activity is an RNAse activity, an RNA-binding activity, or both. In some embodiments, the RNAse activity is a targeted RNAse activity or a collateral RNAse activity.

In some embodiments, the CRISPR-associated protein includes at least one (e.g., two, three, four, five, six, or more) nuclear localization signal (NLS). In some embodiments, the CRISPR-associated protein includes at least one (e.g., two, three, four, five, six, or more) nuclear export signal (NES). In some embodiments, the CRISPR-associated protein includes at least one (e.g., two, three, four, five, six, or more) NLS and at least one (e.g., two, three, four, five, six, or more) NES.

In some embodiments, the direct repeat sequence includes or consists of a nucleotide sequence provided in Table 3 (e.g., SEQ ID NOs 32-49, 52-77, 351-589). In some embodiments, the direct repeat sequence includes or consists of either 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153).

In some embodiments, the spacer sequence includes or consists of from about 15 to about 42 nucleotides.

In some embodiments, the systems provided herein include a single-stranded donor template or a double-stranded donor template (e.g., an RNA or a DNA molecule).

In some embodiments, the systems provided herein include a target RNA or a nucleic acid encoding the target RNA, wherein the target RNA includes a sequence that is capable of hybridizing (e.g., hybridizes under appropriate conditions) to the spacer sequence of the RNA guide.

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G; and ii) a Type VI-D CRISPR-Cas effector protein and/or a nucleic acid encoding the effector protein, wherein the effector protein is about 1200 or fewer amino acids, and wherein the effector protein is capable of binding (e.g., binds under appropriate conditions) to the RNA guide and of targeting the target nucleic acid sequence complementary to the spacer sequence.

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G; and ii) a Type VI-D CRISPR-Cas effector protein and/or a nucleic acid encoding the effector protein, wherein the effector protein is about 1200 or fewer amino acids, and wherein the effector protein is capable of binding (e.g., binds under appropriate conditions) to the RNA guide and of targeting the target nucleic acid sequence complementary to the spacer sequence.

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G; and ii) a Type VI-D CRISPR-Cas effector protein or a nucleic acid encoding the effector protein, wherein the effector protein is about 950 or fewer amino acids in length, and wherein the effector protein is capable of binding (e.g., binds under appropriate conditions), to the RNA guide and of targeting the target nucleic acid sequence complementary to the spacer sequence.

In another aspect, the disclosure provides engineered, non-naturally occurring Clustered Regularly Interspaced Short Palindromic Repeat (CRISPR)—associated (Cas) systems that include or consist of: i) an RNA guide or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G; and ii) a Type VI-D CRISPR-Cas effector protein or a nucleic acid encoding the effector protein, wherein the effector protein is about 950 or fewer amino acids in length, and wherein the effector protein is capable of binding (e.g., binds under appropriate conditions) to the RNA guide and of targeting the target nucleic acid sequence complementary to the spacer sequence.

In another aspect, the disclosure provides engineered, non-naturally occurring Clustered Regularly Interspaced Short Palindromic Repeat (CRISPR)—associated (Cas) systems that include or consist of: i) an RNA guide (e.g., a crRNA) or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G; ii) a Type VI-D CRISPR-Cas effector protein or a nucleic acid encoding the effector protein, wherein the effector protein is capable of binding (e.g., binds under appropriate conditions) to the RNA guide and of targeting the target nucleic acid sequence complementary to the spacer sequence; and iii) an accessory protein, wherein the accessory protein includes at least one WYL domain, wherein the accessory protein includes or consists of an amino acid sequence having at least 85% sequence identity to an amino acid sequence provided in any one of Tables 4, 5, and 6 (e.g., SEQ ID NOs. 78-93, and 590-671), and wherein the accessory protein is capable of regulating (e.g., regulates under appropriate conditions) an activity of the effector protein.

In another aspect, the disclosure provides engineered, non-naturally occurring Clustered Regularly Interspaced Short Palindromic Repeat (CRISPR)—associated (Cas) systems that include or consist of: i) an RNA guide (e.g., a crRNA) or a nucleic acid encoding the RNA guide, wherein the RNA guide includes or consists of a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G; ii) a Type VI-D CRISPR-Cas effector protein or a nucleic acid encoding the effector protein, wherein the effector protein is capable of binding (e.g., binds under appropriate conditions) to the RNA guide and of targeting the target nucleic acid sequence complementary to the spacer sequence; and iii) an accessory protein, wherein the accessory protein includes at least one WYL domain, wherein the accessory protein includes or consists of an amino acid sequence having at least 85% sequence identity to an amino acid sequence provided in any one of Tables 4, 5, and 6 (e.g., SEQ ID NOs. 78-93, and 590-671), and wherein the accessory protein is capable of regulating (e.g., regulates under appropriate conditions) an activity of the effector protein.

In some embodiments, the accessory protein is RspWYL1 (SEQ ID NO: 81).

In some embodiments, the effector protein includes at least two HEPN domains. In some embodiments, the effector protein includes or consists of an amino acid sequence having at least 85% sequence identity to an amino acid sequence provided in Table 2 (e.g., SEQ ID NOs. 1-31, and 200-350). In some embodiments, the effector protein is RspCas13d (SEQ ID NO: 2) or EsCas13d (SEQ ID NO: 1).

In some embodiments, the CRISPR-associated protein (e.g., Type VI-D CRISPR-Cas effector protein) is fused to a base-editing domain (e.g., Adenosine Deaminase Acting on RNA (ADAR) 1; ADAR2; apolipoprotein B mRNA editing enzyme, catalytic polypeptide-like (APOBEC); and activation-induced cytidine deaminase (AID)). In some embodiments, the base-editing domain is further fused to an RNA-binding domain.

In some embodiments, the CRISPR associated protein (e.g., a Type VI-D CRISPR-Cas effector protein) is fused to a RNA methyltransferase, a RNA demethylase, a splicing modifier, a localization factor, or a translation modification factor.

In some embodiments, the CRISPR-associated (e.g., a Type VI-D CRISPR-Cas effector protein) further includes a linker sequence. In some embodiments, the CRISPR-associated protein (e.g., a Type VI-D CRISPR-Cas effector protein) includes one or more mutations or amino acid substitutions that render the CRISPR-associated protein unable to cleave RNA.

In some embodiments, the systems described herein also include an RNA-binding fusion polypeptide that includes an RNA-binding domain and a base-editing domain (e.g., ADAR1, ADAR2, APOBEC, and AID). In some embodiments, the RNA-binding domain is MS2, PP7, or Qbeta.

In some embodiments, the systems described herein include a nucleic acid encoding the CRISPR-associated protein (e.g., a Type VI-D CRISPR-Cas effector protein). In some embodiments, the nucleic acid encoding the CRISPR-associated protein is operably linked to a promoter (e.g., a constitutive promoter or an inducible promoter). In some embodiments, the nucleic acid encoding the CRISPR-associated protein is codon-optimized for expression in a cell (e.g., a mammalian cell or a bacterial cell).

In some embodiments, the systems described herein include a nucleic acid encoding the accessory protein. In some embodiments, the nucleic acid encoding the accessory protein is operably linked to a promoter (e.g., a constitutive promoter or an inducible promoter). In some embodiments, the nucleic acid encoding the accessory protein is codon-optimized for expression in a cell.

In some embodiments, the systems described herein include a nucleic acid encoding one or more RNA guides (e.g., crRNAs). In some embodiments, the nucleic acid encoding the one or more RNA guides is operably linked to a promoter (e.g., a constitutive promoter or an inducible promoter).

In some embodiments, the systems described herein include a nucleic acid encoding a target nucleic acid (e.g., a target RNA). In some embodiments, the nucleic acid encoding the target nucleic acid is operably linked to a promoter (e.g., a constitutive promoter or an inducible promoter).

In some embodiments, the systems described herein include a nucleic acid encoding a CRISPR-associated protein and a nucleic acid encoding an accessory protein in a vector. In some embodiments, the system further includes one or more nucleic acids encoding an RNA guide present in the vector.

In some embodiments, the systems provided herein include a nucleic acid encoding a Type VI-D CRISPR-Cas effector protein in a vector.

In some embodiments, the systems provided herein include a nucleic acid encoding the Type VI-D CRISPR-Cas effector protein and a nucleic acid encoding the accessory protein in a vector. In some embodiments, the system further includes one or more nucleic acids encoding one or more RNA guides (e.g., crRNAs) in the vector.

In some embodiments, the vectors included in the systems are viral vectors (e.g., retroviral vectors, lentiviral vectors, adenoviral vectors, adeno-associated vectors, and herpes simplex vectors. In some embodiments, the vectors included in the system are phage vectors.

In some embodiments, the systems provided herein are in a delivery system. In some embodiments, the delivery system is a nanoparticle, a liposome, an exosome, a microvesicle, and a gene-gun.

The disclosure also provides a cell (e.g., a eukaryotic cell or a prokaryotice cell (e.g., a bacterial cell)) comprising a system described herein. In some embodiments, the eukaryotic cell is a mammalian cell (e.g., a human cell) or a plant cell. The disclosure also provides animal models (e.g., rodent, rabbit, dog, monkey, or ape models) and plant model that include the cells.

In another aspect, the disclosure provides methods of cleaving a target nucleic acid (and compositions for use in such methods), wherein the methods include contacting the target nucleic acid with a system described herein, wherein the spacer sequence is complementary to at least 15 nucleotides of the target nucleic acid, wherein the CRISPR-associated protein or the Type VI-D CRISPR effector protein associates with the RNA guide to form a complex, wherein the complex binds to a target nucleic acid sequence that is complementary to the at least 15 nucleotides of the spacer sequence; and wherein upon binding of the complex to the target nucleic acid sequence the CRISPR-associated protein or the Type VI-D CRISPR effector protein cleaves the target nucleic acid. In some embodiments of the methods, the target nucleic acid is within a cell.

In another aspect, the disclosure also provides methods of inducing dormancy or death of a cell (and compositions for use in such methods), wherein the methods include contacting the cell with a system described herein, wherein the spacer sequence is complementary to at least 15 nucleotides of the target nucleic acid, wherein the Type VI-D CRISPR effector protein associates with the RNA guide to form a complex, wherein the complex binds to a target nucleic acid sequence that is complementary to the at least 15 nucleotides of the spacer sequence, and wherein upon binding of the complex to the target nucleic acid sequence the Type VI-D CRISPR-Cas effector protein cleaves a non-target nucleic acid within the cell, thereby inducing dormancy or death of the cell. In some embodiments of the methods described herein, the death of the cell is via apoptosis, necrosis, necroptosis, or a combination thereof.

In some embodiments, the target nucleic acid is an RNA molecule (e.g., an mRNA, a tRNA, a ribosomal RNA, a non-coding RNA, a lncRNA, or a nuclear RNA). In some embodiments, the target nucleic acid is a DNA molecule (e.g., chromosomal DNA, mitochondrial DNA, single-stranded DNA, or plasmid DNA).

In some embodiments of the methods described herein, upon binding of the complex to the target nucleic acid, the CRISPR-associated protein or the Type VI-D CRISPR-Cas effector protein exhibits collateral RNAse activity.

In some embodiments, the cell is a cancer cell (e.g., a tumor cell). In some embodiments, the cell is an infectious agent cell or a cell infected with an infectious agent. In some embodiments, the cell is a bacterial cell, a cell infected with a virus, a cell infected with a prion, a fungal cell, a protozoan, or a parasite cell.

In another aspect, the disclosure provides methods of treating a condition or disease in a subject in need thereof (and compositions for use in such methods, the methods include administering to the subject a system described herein, wherein the spacer sequence is complementary to at least 15 nucleotides of a target nucleic acid associated with the condition or disease, wherein the CRISPR-associated protein or the Type VI-D CRISPR-Cas effector protein associates with the RNA guide to form a complex, wherein the complex binds to a target nucleic acid sequence that is complementary to the at least 15 nucleotides of the spacer sequence; and wherein upon binding of the complex to the target nucleic acid sequence the CRISPR-associated protein or the Type VI-D CRISPR-Cas effector protein cleaves the target nucleic acid, thereby treating the condition or disease in the subject.

In some embodiments, the condition or disease is a cancer or an infectious disease. In some embodiments, the condition or disease is cancer, and wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

In another aspect, the disclosure provides the use of a system described herein in a method selected from the group consisting of RNA sequence specific interference; RNA sequence-specific gene regulation; screening of RNA, RNA products, ncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

In some embodiments, the methods described herein are performed either in vitro, in vivo, or ex vivo.

The disclosure also provides methods of modifying an RNA molecule (and compostions for use in such methods), including contacting the RNA molecule with a system described herein. In some embodiments, the spacer sequence is complementary to at least 15 nucleotides of the RNA molecule.

The disclosure also provides methods of detecting a target RNA (e.g., a single-stranded RNA or a double-stranded RNA) in a sample, the methods including: a) contacting the sample with: (i) an RNA guide or a nucleic acid encoding the RNA guide, wherein the RNA guide includes a direct repeat sequence and a spacer sequence capable of hybridizing (e.g., hybridizes under appropriate conditions) to the target RNA; (ii) a Type VI-D CRISPR-Cas effector protein or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNAse activity and cleaves the labeled detector RNA; and b) measuring a detectable signal produced by cleavage of the labeled detector RNA, wherein said measuring provides for detection of the single-stranded target RNA in the sample.

In some embodiments, the Type VI-D CRISPR-Cas effector protein includes at least two HEPN domains. In some embodiments, the Type VI-D CRISPR-Cas effector protein is about 1200 amino acids or less in length.

In some embodiments, the Type VI-D CRISPR-Cas effector protein includes or consists of an amino acid sequence having at least 80% (e.g., 81%, 82%, 83%, 84%, 85%, 86%, 87% 88%, 89%, 90%, 91%, 92%, 93%, 94%, 95%, 96%, 97%, 98%, 99%, or 100%) sequence identity to an amino acid sequence provided in Table 2 (e.g., SEQ ID NOs. 1-31, and 200-350). In some embodiments, the Type VI-D CRISPR-Cas effector protein includes or consists of an amino acid sequence provided in Table 2 (e.g., SEQ ID NOs. 1-31, and 200-350). In some embodiments, the Type VI-D CRISPR-Cas effector protein is RspCas13d (SEQ ID NO: 2) or EsCas13d (SEQ ID NO: 1).

In some embodiments, the effector protein includes one or more amino acid substitutions within at least one of the HEPN domains. In some embodiments, the one or more amino acid substitutions include an alanine substitution at an amino acid residue corresponding to R295, H300, R849, or H854 of SEQ ID NO: 1, or R288, H293, R820, or H825 of SEQ ID NO: 2.

In some embodiments, the methods further include comparing the detectable signal with a reference signal and determining the amount of target RNA in the sample.

In some embodiments, the measuring is performed using gold nanoparticle detection, fluorescence polarization, colloid phase transition/dispersion, electrochemical detection, and semiconductor based-sensing.

In some embodiments, the labeled detector RNA includes a fluorescence-emitting dye pair. In some embodiments, the labeled detector RNA includes a fluorescence resonance energy transfer (FRET) pair. In some embodiments, the labeled detector RNA includes a quencher/fluor pair.

In some embodiments, upon cleavage of the labeled detector RNA by the effector protein, an amount of detectable signal produced by the labeled detector RNA is decreased.

In some embodiments, upon cleavage of the labeled detector RNA by the effector protein, an amount of detectable signal produced by the labeled detector RNA is increased. In some embodiments, the labeled detector RNA produces a first detectable signal prior to cleavage by the effector protein and a second detectable signal after cleavage by the effector protein.

In some embodiments, a detectable signal is produced when the labeled detector RNA is cleaved by the effector protein.

In some embodiments, the labeled detector RNA includes a modified nucleobase, a modified sugar moiety, a modified nucleic acid linkage, or a combination thereof.

In one aspect, the disclosure relates to engineered, non-naturally occurring Clustered Regularly Interspaced Short Palindromic Repeat (CRISPR)—associated (Cas) systems that include: an RNA guide or a nucleic acid encoding the RNA guide, wherein the RNA guide includes a direct repeat sequence and a spacer sequence capable of hybridizing (e.g., hybridizes under appropriate conditions) to a target nucleic acid, wherein the direct repeat sequence includes 5′-X1X2X3X4TX5TX6AAAC-3′ (SEQ ID NO: 151) at the 3′ terminal end of the RNA guide, and wherein X1 is A or C or G, X2 is G or T, X3 is A or G, X4 is C or G or T, X5 is C or T, and X6 is A or G; and a Type VI-D CRISPR-Cas effector protein or a nucleic acid encoding the effector protein, wherein the effector protein is capable of binding (e.g., binds under appropriate conditions) to the RNA guide and of targeting the target nucleic acid sequence complementary to the spacer sequence, and wherein the target nucleic acid is an RNA.

In one aspect, the disclosure relates to engineered, non-naturally occurring Clustered Regularly Interspaced Short Palindromic Repeat (CRISPR)—associated (Cas) systems that include or consist of: an RNA guide or a nucleic acid encoding the RNA guide, wherein the RNA guide includes a direct repeat sequence and a spacer sequence capable of hybridizing (e.g., hybridizes under appropriate conditions) to a target nucleic acid, wherein the direct repeat sequence includes 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G; and a Type VI-D CRISPR-Cas effector protein or a nucleic acid encoding the effector protein, wherein the effector protein is capable of binding (e.g., binds under appropriate conditions) to the RNA guide and of targeting the target nucleic acid sequence complementary to the spacer sequence, and wherein the target nucleic acid is an RNA.

In some embodiments of these systems, the Type VI-D CRISPR-Cas effector proteins include at least two HEPN domains. In some embodiments, the Type VI-D CRISPR-Cas effector proteins include an amino acid sequence having at least 90% identity to an amino acid sequence selected from the group consisting of SEQ ID NO: 12, SEQ ID NO: 1, and SEQ ID NO: 10. In other embodiments, the Type VI-D CRISPR-Cas effector proteins include an amino acid sequence having at least 95% sequence identity to an amino acid sequence provided in Table 2 (e.g., SEQ ID NOs. 1-31, and 200-350), or they can include an amino acid sequence provided in Table 2.

In various embodiments, the direct repeat sequence can include a nucleotide sequence provided in Table 3 (e.g., SEQ ID NOs 32-49, 52-77, 351-589).

In some embodiments, the targeting of the target nucleic acid results in a modification of the target nucleic acid. For example, the modification of the target nucleic acid can be a cleavage event.

In the new systems, the Type VI-D CRISPR-Cas effector proteins can include one or more amino acid substitutions within at least one of the HEPN domains resulting in a reduction of an RNAse activity of the Type VI-D CRISPR-Cas effector protein, as compared to the RNAse activity of the Type VI-D CRISPR-Cas effector protein without the one or more amino acid substitutions, e.g., 2, 3, 4, 5, 6, 7, or 8 amino acid substitutions. In some embodiments, the one or more amino acid substitutions include an alanine substitution at an amino acid residue corresponding to R295, H300, R849, or H854 of SEQ ID NO: 1, or R288, H293, R820, or H825 of SEQ ID NO: 2.

In some embodiments, the Type VI-D CRISPR-Cas effector protein is fused to a base-editing domain, e.g., to an RNA methyltransferase, a RNA demethylase, a splicing modifier, a localization factor, or a translation modification factor.

In various embodiments, the Type VI-D CRISPR-Cas effector protein includes at least one nuclear localization signal (NLS), at least one nuclear export signal (NES), or both.

In some embodiments, the direct repeat sequence includes either 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153). In some embodiments, the spacer consists of from about 15 to about 42 nucleotides.

In another aspect of the disclosure, the systems include the nucleic acid encoding the Type VI-D CRISPR-Cas effector protein, operably linked to a promoter. For example, the promoter can be a constitutive promoter.

In some embodiments, the nucleic acid encoding the Type VI-D CRISPR-Cas effector protein is codon-optimized for expression in a cell. In various embodiments, the nucleic acids encoding the Type VI-D CRISPR-Cas effector protein are operably linked to a promoter within in a vector, e.g., selected from the group consisting of a retroviral vector, a lentiviral vector, a phage vector, an adenoviral vector, an adeno-associated vector, and a herpes simplex vector.

In another aspect, the system is present in a delivery system selected from the group consisting of a nanoparticle, a liposome, an exosome, a microvesicle, and a gene-gun.

In some embodiments, the systems can further include a target RNA or a nucleic acid encoding the target RNA, wherein the target RNA includes a sequence that is capable of hybridizing (e.g., hybridizes under appropriate conditions) to the spacer sequence of the RNA guide.

In another aspect, the disclosure includes one or more cells that include the systems described herein.

In another aspect, the disclosure provides methods of cleaving a target nucleic acid.

The methods include contacting the target nucleic acid with a system as described herein; wherein the spacer sequence is complementary to at least 15 nucleotides of the target nucleic acid; wherein the Type VI-D CRISPR-Cas effector protein associates with the RNA guide to form a complex;

wherein the complex binds to a target nucleic acid sequence that is complementary to the at least 15 nucleotides of the spacer sequence; and wherein upon binding of the complex to the target nucleic acid sequence, the Type VI-D CRISPR-Cas effector protein cleaves the target nucleic acid.

In another aspect, the disclosure provides methods of inducing dormancy or death of a cell, e.g., in vitro or in vivo (and compositions for use in such methods), the method including contacting the cell with a system as described herein; wherein the spacer sequence is complementary to at least 15 nucleotides of the target nucleic acid within the cell; wherein the Type VI-D CRISPR-Cas effector protein associates with the RNA guide to form a complex; wherein the complex binds to the target nucleic acid sequence that is complementary to the at least 15 nucleotides of the spacer sequence; and wherein after binding of the complex to the target nucleic acid sequence, the Type VI-D CRISPR-Cas effector protein cleaves a non-target nucleic acid within the cell, thereby inducing dormancy or death of the cell.

In these methods, the cell can be a bacterial cell, a cell infected with a virus, a cell infected with a prion, a fungal cell, a protozoan, or a parasite cell.

In other embodiments, the disclosure provides methods of modifying a target nucleic acid in a sample, in which the methods include contacting the sample with a system as described herein, e.g., with fusion proteins; wherein the spacer sequence is complementary to at least 15 nucleotides of the target nucleic acid within the sample; wherein the Type VI-D CRISPR-Cas effector protein fused to the base editing domain associates with the RNA guide to form a complex; wherein the complex binds to the target nucleic acid sequence that is complementary to the at least 15 nucleotides of the spacer sequence; and wherein after binding of the complex to the target nucleic acid sequence, the Type VI-D CRISPR-Cas effector protein fused to the base-editing domain modifies at least one nucleobase of the target nucleic acid.

In another aspect, the disclosure provides methods of detecting a single-stranded target RNA in a sample. These methods include: a) contacting the sample with: (i) a RNA guide or a nucleic acid encoding the RNA guide, wherein the RNA guide includes a direct repeat sequence and a spacer sequence capable of hybridizing (e.g., hybridizes under appropriate conditions) to the target RNA; (ii) a Type VI-D CRISPR-Cas effector protein or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the Type VI-D CRISPR-Cas effector protein exhibits collateral RNAse activity and cleaves the labeled detector RNA; and b) measuring a detectable signal produced by cleavage of the labeled detector RNA, wherein said measuring provides for detection of the single-stranded target RNA in the sample.

In these methods, the effector protein includes an amino acid sequence having at least 90% sequence identity to an amino acid sequence provided in Table 2 (e.g., SEQ ID NOs. 1-31, and 200-350). These methods can further include comparing the detectable signal with a reference signal and determining the amount of target RNA in the sample.

The term “cleavage event,” as used herein, refers to a break in a target nucleic acid created by a nuclease (e.g., a Type VI-D CRISPR-Cas effector protein) of a CRISPR system described herein. In some embodiments, the cleavage event is a single-stranded RNA break. In some embodiments, the cleavage event is a double-stranded RNA break. In some embodiments, the cleavage event is a double-stranded DNA break. In some embodiments, the cleavage event is a single-stranded DNA break.

The terms “CRISPR system” or “Clustered Interspaced Short Palindromic Repeat (CRISPR)-associated (Cas) system” as used herein refer to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

The term “CRISPR array” as used herein refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat,” or “CRISPR direct repeat,” or “direct repeat,” as used herein, refer to multiple short direct repeating sequences, which show very little or no sequence variation within a CRISPR array.

The term “CRISPR RNA” or “crRNA” as used herein refers to a RNA molecule including a guide sequence used by a CRISPR effector to target a specific nucleic acid sequence. Typically, crRNAs contains a sequence that mediates target recognition and a sequence that forms a duplex with a tracrRNA. In some embodiments, the crRNA: tracrRNA duplex binds to a CRISPR effector.

The terms “donor template” or “donor template nucleic acid,” as used herein refers to a nucleic acid molecule that can be used by one or more cellular proteins to modify the sequence of a target nucleic acid after a CRISPR-associated protein described herein has altered the target nucleic acid. In some embodiments, the donor template nucleic acid is a double-stranded nucleic acid. In some embodiments, the donor template nucleic acid is a single-stranded nucleic acid. In some embodiments, the donor template nucleic acid is linear.

In some embodiments, the donor template nucleic acid is circular (e.g., a plasmid). In some embodiments, the donor template nucleic acid is an exogenous nucleic acid molecule. In some embodiments, the donor template nucleic acid is an endogenous nucleic acid molecule (e.g., a chromosome). In some embodiments, the donor template is a DNA molecule. In some embodiments, the donor template is an RNA molecule.

The term “CRISPR effector,” “effector,” “CRISPR-associated protein,” or “CRISPR enzyme” as used herein refers to a protein that carries out an enzymatic activity or that binds to a target site on a nucleic acid specified by a RNA guide. Indifferent embodiments, a CRISPR effector has endonuclease activity, nickase activity, exonuclease activity, transposase activity, and/or excision activity. In some embodiments, the CRISPR-associated protein is a Type VI Cas protein, a Type V Cas protein, or a Type II Cas protein. I some embodiments, the CRISPR-associated protein is a Cas13a protein, a Cas13b protein, a Cas13c protein, a Cas13d protein, a Cas12a protein, or a Cas9 protein. In some embodiments, the CRISPR-associated protein is a Type VI-D CRISPR-Cas effector protein described herein.

The term “RNA guide” as used herein refers to any RNA molecule that facilitates the targeting of a protein described herein to a target nucleic acid. Exemplary “RNA guides” include, but are not limited to, crRNAs or crRNAs in combination with cognate trans-activating RNAs (tracrRNAs). The latter may be independent RNAs or fused as a single RNA using a linker. In some embodiments, the RNA guide is engineered to include a chemical or biochemical modification. In some embodiments, an RNA guide may include one or more nucleotides.

The term “origin of replication,” as used herein, refers to a nucleic acid sequence in a replicating nucleic acid molecule (e.g., a plasmid or a chromosome) that is recognized by a replication initiation factor or a DNA replicase.

As used herein, the term “targeting” refers to the ability of a complex including a CRISPR-associated protein and a RNA guide, such as a crRNA, to bind to a specific target nucleic acid and not to other nucleic acids that do not have the same sequence as the target nucleic acid.

As used herein, the term “target nucleic acid” refers to a specific nucleic acid sequence that specifically binds to a complex including a CRISPR-associated protein and a RNA guide described herein. In some embodiments, the target nucleic acid is or includes a gene. In some embodiments, the target nucleic acid is or includes a non-coding region (e.g., a promoter). In some embodiments, the target nucleic acid is single-stranded. In some embodiments, the target nucleic acid is double-stranded.

The terms “trans-activating crRNA” or “tracrRNA” as used herein refer to an RNA including a sequence that forms a structure required for a CRISPR-associated protein to bind to a specified target nucleic acid.

The term “collateral RNAse activity,” as used herein in reference to A CRISPR-associated protein, refers to non-specific RNAse activity of a CRISPR-associated protein after the enzyme has bound to and/or modified a specifically-targeted nucleic acid. In some embodiments, a CRISPR-associated protein (e.g., a Type VT-D CRISPR-Cas effector protein) exhibits collateral RNAse activity after binding to a target nucleic acid (e.g., a target RNA).

A nucleic acid that is cleaved or degraded by a CRISPR-associated protein in a non-specific manner (i.e., when the protein exhibits collateral RNAse activity) is referred to herein as a “non-target nucleic acid.”

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting. Citation or identification of any document in this application is not an admission that such document is available as prior art to the present invention.

Other features and advantages of the invention will be apparent from the following detailed description, and from the claims.

### BRIEF FIGURE DESCRIPTION

FIG. 1 depicts a schematic representation of a maximum likelihood tree topology for an exemplary subset of Cas13d, with the genomic arrangement of the genes encoding predicted protein components of Type VI-D system components shown to the right. Each locus sequence is identified by a protein accession or gene number, with the species name provided where available. Key proteins and CRISPR arrays are depicted as follows: white—Cas13d, horizontal stripes—WYL1 accessory protein, light gray—WYL domain containing protein, vertical stripes—Cas1, dark gray—Cas2.

FIG. 2A depicts a schematic tree comparing the different type VI subtype locus structures. Gene arrows are shown roughly proportional to size. Labels denote the following: WYL—WYL domain, HEPN—HEPN nuclease domain.

FIG. 2B depicts a size comparison for Cas13 proteins from the 4 type VI subtypes; error bars specify the mean and standard deviation.

FIG. 3 depicts a phylogenetic tree of Cas1 proteins from type II and type VI CRISPR-Cas systems. The tree was constructed for a non-redundant set of Cas1 proteins associated with Cas13d and type II and type VI CRISPR-Cas systems as described previously (see (Peters et al., 2017)). Several Cas1 proteins associated with subtype I-E systems were selected for an outgroup. Each sequence is denoted by a local numeric identifier, CRISPR-Cas type and species name (if available). Cas1 proteins associated with Cas13d are denoted by “CAS-VI-D”, and those associated with Cas13a by “CAS-VI-A”. Several branches were collapsed and are shown by triangles with CRISPR-Cas system indicated on the right. Support values are indicated for selected branches.

FIGS. 4A and 4B depict a phylogenetic tree constructed for a combined set of Cas13d sequences described (light gray) and previously described Cas13a sequences. Each sequence is denoted by a protein locus tag and species name (if available). Cas13d proteins form a clade with a 100% bootstrap support value (shown on branch).

FIGS. 5A, 5B and 5C depict a multiple sequence alignment of Cas13d protein sequences (RspCas13d (SEQ ID NO: 2) and EsCas13d (SEQ ID NO: 1) and Cas13a protein sequences (LbaCas13a (SEQ ID NO: 156), LbuCas13a (SEQ ID NO: 157), LshCas13a (SEQ ID NO: 158)). Previously identified domains of Cas13a are highlighted with varying background shading as indicated in the figure (NTD, N-terminal domain). Note the nearly complete absence of a counterpart to the Helical-1 domain of Cas13a in Cas13d (the alignment in this region cannot be considered reliable).

FIG. 6 depicts a phylogenetic tree of the WYL1 protein family. Exemplary WYL1 proteins associated with Cas13d are denoted by gray. In cases when a CRISPR array and/or other cas genes are present in the vicinity of the respective WYL1 gene (within 10 kb up- and downstream), the description includes “CRISPR”. Several branches were collapsed and are indicated by triangles. Domain organization is schematically shown next to each branch. Abbreviation: WYL—WYL domain (usually fused to a characteristic C-terminal subdomain); RHH—ribbon helix helix superfamily DNA binding domain.

FIG. 7 depicts a multiple sequence alignment of exemplary WYL1 protein sequences. The RHH domain is denoted by ‘r’ and the WYL domain fused to the characteristic C-terminal subdomain is denoted by ‘y’ underneath the alignment. The predicted secondary structure elements are shown (E, extended conformation (β-strand), H, α-helix).

FIG. 8 depicts a design of minimal engineered CRISPR-Cas systems for the Rsp and Es type VI-D CRISPR loci (referred to as RspCas13d and EsCas13d systems), with a spacer library tiling pACYC184 (both top strand and bottom strand).

FIG. 9 depicts a schematic of the bacterial negative selection screen used to evaluate functional parameters of RspCas13d and EsCas13d systems.

FIGS. 10A and 10B depict a negative control condition from bacterial screens for EsCas13d and RspCas13d systems, respectively. Solid and dashed lines represent both possible direct repeat (DR) orientations cloned into the screening library. Non-targeting CRISPR arrays (with spacers matching a GFP open reading frame) inserted into EsCas13d and RspCas13d screening systems showed minimal levels of depletion in bacterial negative selection screens (no GFP open reading frame was included in our screen system).

FIGS. 11A and 11B depict a negative control condition from bacterial screens for EsCas13d and RspCas13d systems, respectively. Solid and dashed lines represent both possible direct repeat (DR) orientations cloned into the screening library. Deletion of EsCas13d and RspCas13d-RspWYL1 open reading frames from the EsCas13d and RspCas13d screening systems resulted in minimal depletion of library CRISPR array elements in bacterial negative selection screens.

FIGS. 12A and 12B depict the distribution and magnitude of crRNA depletion from bacterial screens for EsCas13d and RspCas13d, respectively. Depletion value was calculated by normalized sequencing reads from the screen output divided by normalized reads from the pre-transformation screen input library for each crRNA spacer and orientation. Solid and dashed lines represent both possible direct repeat (DR) orientations cloned into the screening library. cloned into the screening library. The vertical dashed lines demarcate the intersection of the ranked screen hits with the depletion fraction of 0.1, below which we define as strongly depleted.

FIGS. 13A and 13B depict the location of strongly depleted targets of the active DR orientation over the strands and genetic features of the pACYC184 plasmid for EsCas13d and RspCas13d systems, respectively. Light gray outlines represent the total number of spacers (y-axis) targeting a location, while short bars depict the locations of strongly depleted spacers with heatmap color proportional to magnitude of depletion. Directional expression data for pACYC184 is plotted as a heatmap between the x-axes.

FIGS. 14A and 14B depict web logos for the 5′ and 3′ 30 nt regions flanking strongly depleted targets for EsCas13d and RspCas13d systems, and show no evidence of PFS or PAM requirements.

FIG. 14C depicts violin plots of bit scores of all possible PFS targeting rules of up to length 3 involving the target site and +/−15 nt flanking region, for BzCas13b, RspCas13d, and EsCas13d systems. Dots represent data points outside of the discernable density of the violin plot. These dots accurately recapitulate the known PFS positions of BzCas13b, as shown above the dots.

FIG. 15 depicts bar charts showing the fraction of hits for RspCas13d and EsCas13d systems according to features of the plasmid for all targets.

FIGS. 16A and 16B depict heatmaps of the fraction (# strongly depleted spacers)/(#strongly depleted spacers+# non-depleted spacers) for all target regions (CRISPR arrays with active direct repeat orientation only) with no predicted secondary structure between specific start (x-axis) and end (y-axis) locations. White boxes indicate specific target regions (bounded by start (x-axis) and end (y-axis) locations), where selection of spacers with no predicted secondary structure maximized targeting efficacy, while minimizing the number of screen spacers eliminated due to the presence of predicted secondary structure. Targets these spacer populations are referred to as “low secondary structure targets” for RspCas13d and EsCas13d respectively.

FIG. 16C depicts bar charts showing the fraction of hits for RspCas13d and EsCas13d systems according to features of the plasmid for low secondary structure targets.

FIG. 17 depicts a schematic of the RNA extraction from bacterial screen, next-generation sequencing (NGS), and alignment to determine the mature crRNA for EsCas13d.

Distribution of read counts by crRNA sequence location is depicted on the right, and the predicted EsCas13d mature crRNA secondary structure is shown.

FIG. 18 depicts a coomassie blue stained polyacrylamide gel of purified recombinant proteins EsCas13d, RspCas13d, and RspWYL1 respectively.

FIG. 19 depicts schematic representions of the major products identified from next-generation sequencing of in vitro cleaved RNA fragments from the pre-crRNA processing with EsCas13d and RspCas13d. The black line represents the direct repeats and associated secondary structure, the box represents the full-length spacer, and the filled triangle represents the cleavage sites. The lengths described are for processed EsCas13d crRNAs, with RspCas13d having one extra nucleotide due to the 31 nt natural length spacer used for instead of 30. Not depicted are the 3-4 nt at the 5′ end of the pre-crRNA from T7 in vitro transcription.

FIGS. 20A, 20B, 20C, and 20D depict denaturing gels displaying Cas13d mediated cleavage of their cognate pre-crRNAs over a dose titration of effector concentration. The dependence of Cas13d crRNA biogenesis on divalent metal cations was evaluated with the introduction of 100 mM EDTA to the standard reaction conditions.

FIG. 21 depicts a denaturing gel displaying LwaCas13a at a final concentration of 100 nM processing of pre-crRNA (200 nM) without the presence of EDTA, and under reaction conditions supplemented with increasing concentrations of EDTA (3.3-100 mM).

FIGS. 22A and 22B depict a titration of Apo EsCas13d and RspCas13d (100-0.4 nM) over a non-targeted ssDNA substrate (100 nM).

FIGS. 23A and 23B depict a titration of EsCas13d and RspCas13d in complex with crRNA (100-0.4 nM) over non-targeted ssDNA substrates (100 nM).

FIGS. 24A and 24B depict a titration of EsCas13d and RspCas13d in complex with crRNA (100-0.4 nM) over targeted ssDNA substrates (100 nM). Saturation of target cleavage activity was observed at approx. 50 nM RspCas13d-crRNA complex and 100 nM EsCas13d-crRNA complex.

FIGS. 25A and 25B depict representative denaturing gels displaying the targeted RNase activity of EsCas13d and RspCas13d effector proteins, with substrate RNA cleavage occurring when the crRNA matches its complementary target ssRNA. RNA substrates are 5′ labeled with IRDye 800.

FIGS. 26A and 26B depict representative denaturing gels displaying non-specific RNase activity of the Cas13d effectors upon targeted substrate recognition, demonstrated by the cleavage of fluorescein dUTP body-labeled collateral RNA upon activation of the target nuclease activity. For all reactions, EsCas13d-crRNA and RspCas13d-crRNA complexes were formed by pre-incubating Cas13d and cognate crRNA for 5 minutes at 37° C., prior to adding target and/or collateral ssRNA and incubating the reaction for 30 minutes.

FIGS. 26C and 26D depict denaturing gels displaying cleavage reactions of the Cas13d-crRNA complex over two distinct ssRNA substrates, short 150 nt target RNAs (top) and longer 800 nt fluorescent body-labeled ssRNA substrates (bottom) for EsCas13d and RspCas13d. The labels A and B correspond to matching crRNA/substrate pairs.

FIG. 27A depicts a comparative depletion plot of bacterial screens performed on RspCas13d only (solid line, long dashes) versus RspCas13d with RspWYL1 (short and medium dashes). The dashed vertical lines demarcate the intersection of the ranked screen hits with the depletion fraction of 0.1, below which we define as strongly depleted.

FIG. 27B depicts spacer depletion ratios for RspCas13d with and without RspWYL1.

FIG. 28 depicts a depletion plot of bacterial screens using only RspWYL1 and the repeat-spacer-repeat library associated with RspCas13d.

FIGS. 29A and 29B depict representative activity of titrating different molar ratios of purified RspWYL1 to a fixed dose of RspCas13d. FIG. 29A is an ssRNA substrate cleavage assay, and FIG. 29B evaluate the effect of RspWYL1 on collateral activity.

FIG. 29C depicts the effect on RNA cleavage of titrating RspWYL1 (800 to 0.4 nM) while holding fixed the concentration of Apo RspCas13d (200 nM) for target ssRNA.

FIG. 29D depicts the effect on RNA cleavage of titrating RspWYL1 (800 to 0.4 nM) while holding fixed the concentration of Apo RspCas13d (200 nM) for collateral ssRNA activity.

FIG. 29E depicts the effect on RNA cleavage of titrating RspWYL1 (800 to 0.4 nM) while holding fixed the concentration of RspCas13d-crRNA complex (50 nM) for target ssRNA.

FIG. 29F depicts the effect on RNA cleavage of titrating RspWYL1 (800 to 0.4 nM) while holding fixed the concentration of RspCas13d-crRNA complex (50 nM) for collateral ssRNA activity.

FIGS. 30A and 30B depict representative activity of titrating different molar ratios of purified RspWYL1 to a fixed dose of EsCas13d. FIG. 30A is an ssRNA substrate cleavage assay, and FIG. 30B evaluate the effect of RspWYL1 on collateral activity of EsCas13d. In both of these reactions, RspWYL1 was pre-incubated along with the pre-crRNA and Cas13d effector for 5 minutes at 37° C. before incubation with substrate RNA. The final concentration of Cas13d in the reaction is 33 nM with a 2:1 ratio of Cas13d to pre-crRNA.

FIG. 31 shows that RspWYL1 enhances the activity of type VI-B effector BzCas13b. Representative gel displaying the ability of RspWYL1 to enhance target cleavage and collateral activity for Cas13 enzymes of subtype VI-B, demonstrating modularity beyond Type VI-D. In this reaction RspWYL1 was pre-incubated along with the pre-crRNA and BzCas13b effector for 5 minutes at 37 C before incubation with substrate RNA.

FIGS. 32A and 32B show that EsCas13d and RspCas13d, respectively, are capable of specific detection of RNA species using the collateral effect of the enzymes, and additionally, demonstrate differential activity over short ribonucleotide oligomer substrates. The poly-G and poly-U labels refer to substrates containing 5 identical ribonucleotide bases, with the 5′ end modified with a FAM labeled fluorescent ribonucleotide and the 3′ end modified with an Iowa Black FQ fluorescent quencher. These data were collected 60 minutes after incubation at 37° C. The error bars represent S.E.M. of four technical replicates.

FIGS. 33A and 33B depict the distribution and magnitude of crRNA depletion for primary screening of EsCas13d and RspCas13d (effector only), respectively, in the absence of tetracycline. The value of crRNA depletion was calculated by normalized sequencing reads from the screen output divided by normalized reads from the pre-transformation screen input library for each crRNA spacer and orientation. The vertical dashed lines demarcate the intersection of the ranked screen hits with the depletion fraction of 0.1, below which we define as strongly depleted.

FIGS. 34A and 34B depict the location of strongly depleted targets of the active DR orientation over the strands and genetic features of the pACYC184 plasmid for EsCas13d and RspCas13d (effector only), respectively. Light gray outlines represent the total number of spacers (y-axis) targeting a location, while short horizontal bars depict the locations of strongly depleted spacers with heatmap color proportional to magnitude of depletion.

### DETAILED DESCRIPTION

### CRISPR Class 2 RNA-Guided RNases

In one aspect, provided herein is a novel family of CRISPR Class 2 effectors having two strictly conserved RX4-6H motifs, characteristic of Higher Eukaryotes and Prokaryotes Nucleotide-binding (HEPN) domains. CRISPR Class 2 effectors that contain two HEPN domains have been previously characterized and include, for example, CRISPR Cas13a (C2c2), Cas13b, and Cas13c.

HEPN domains have been shown to be RNAse domains and confer the ability bind to and cleave any target RNA molecule. In some embodiments, a HEPN domain comprises the amino acid sequence RXXXXH, wherein X is any amino acid (SEQ ID NO: 94). The target RNA may be any suitable form of RNA, including but not limited to mRNA, tRNA, ribosomal RNA, non-coding RNA, lincRNA, and nuclear RNA. For example, in some embodiments, the CRISPR-associated protein recognizes and cleaves targets located on the coding strand of open reading frames (ORFs).

In one embodiment, the disclosure provides a family of CRISPR Class 2 effectors, referred to herein generally as Type VI-D CRISPR-Cas effector proteins, Cas13d or Cas3. Direct comparison of the Type VI-D CRISPR-Cas effector proteins with the effector of these other systems shows that Type VI-D CRISPR-Cas effector proteins are significantly smaller (e.g., 20% fewer amino acids), and have less than 10% sequence similarity in multiple sequence alignments to other previously described effector proteins. This newly-identified family of CRISPR Class 2 effectors can be used in a variety of applications, and are particularly suitable for therapeutic applications since they are significantly smaller than other effectors (e.g., CRISPR Cas13a, Cas13b, or Cas13c effectors) which allows for the packaging of the effectors and/or nucleic acids encoding the effectors into delivery systems having size limitations.

In bacteria, the Type VI-D CRISPR-Cas systems include a single effector (approximately 920 amino acids in length), and one or none accessory proteins (approximately 380 amino acids in length) within close proximity to a CRISPR array. The CRISPR array includes direct repeat sequences typically 36 nucleotides in length, which are generally well conserved, especially on the 3′ end which ends with TNTNAAAC (SEQ ID NO: 154). Reduced consensus of the nucleotide sequence in the 5′ end of the direct repeats suggests that the crRNA is processed from the 5′ end. With few exceptions, the 21 nucleotide sequence immediately upstream of the 3′ end TNTNAAAC (SEQ ID NO: 154) starts with a highly conserved A and exhibits sequence complementarity that suggests strong base pairing for an RNA loop structure. The spacers contained in the Cas13d CRISPR arrays are most commonly 30 nucleotides in length, with the majority of variation in length contained in the range of 28 to 36 nucleotides.

Exemplary Type VI-D CRISPR-Cas effector proteinsare provided below in Table 2 (e.g., SEQ ID NOs. 1-31, and 200-350). In some embodiments, a Type VI-D CRISPR-Cas effector proteins include an amino acid sequence having at least about 80% (e.g., 81%, 82%, 83%, 84%, 85%, 86%, 87% 88%, 89%, 90%, 91%, 92%, 93%, 94%, 95%, 96%, 97%, 98%, 99%, or 100%) identity to the amino acid sequence of any one of Table 2 (e.g., SEQ ID NOs. 1-31, and 200-350). In some embodiments, a Type VI-D CRISPR-Cas effector proteins includes the amino acid sequence of any one of Table 2 (e.g., SEQ ID NOs. 1-31, and 200-350). In some embodiments, the Type VI-D CRISPR-Cas effector proteins is DS499551 (SEQ ID NO: 1; also referred to herein as EsCas13d) or LARF01000048 (SEQ ID NO: 2; also referred to herein as RspCas13d), the amino acid sequences of each are provided below:

>NP_005358205.1 (EsCas13d)
[Eubacterium siraeum DSM 15702]
(SEQ ID NO: 1)
MGKKIHARDLREQRKTDRTEKFADQNKKREAERAVPKKDAAVSVKSVSSV
SSKKDNVTKSMAKAAGVKSVFAVGNTVYMTSFGRGNDAVLEQKIVDTSHE
PLNIDDPAYQLNVVTMNGYSVTGHRGETVSAVTDNPLRRFNGRKKDEPEQ
SVPTDMLCLKPTLEKKFFGKEFDDNIHIQLIYNILDIEKILAVYSTNAIY
ALNNMSADENIENSDFFMKRTTDETFDDFEKKKESTNSREKADFDAFEKF
IGNYRLAYFADAFYVNKKNPKGKAKNVLREDKELYSVLTLIGKLRHWCVH
SEEGRAEFWLYKLDELKDDFKNVLDVVYNRPVEEINNRFIENNKVNIQIL
GSVYKNTDIAELVRSYYEFLITKKYKNMGFSIKKLRESMLEGKGYADKEY
DSVRNKLYQMTDFILYTGYINEDSDRADDLVNTLRSSLKEDDKTTVYCKE
ADYLWKKYRESIREVADALDGDNIKKLSKSNIEIQEDKLRKTFISYADSV
SEFTKLIYLLTRFLSGKEINDLVTTLINKFDNIRSFLEIMDELGLDRTFT
AEYSFFEGSTKYLAELVELNSFVKSCSFDINAKRTMYRDALDILGIESDK
TEEDIEKMIDNILQIDANGDKKLKKNNGLRNFIASNVIDSNRFKYLVRYG
NPKKIRETAKCKPAVRFVLNEIPDAQIERYYEACCPKNTALCSANKRREK
LADMIAEIKFENFSDAGNYQKANVTSRTSEAEIKRKNQAIIRLYLTVMYI
MLKNLVNVNARYVIAFHCVERDTKLYAESGLEVGNIEKNKTNLTMAVMGV
KLENGIIKTEFDKSFAENAANRYLRNARWYKLILDNLKKSERAVVNEFRN
TVCHLNAIRNININIKEIKEVENYFALYHYLIQKHLENRFADKKVERDTG
DFISKLEEHKTYCKDFVKAYCTPFGYNLVRYKNLTIDGLFDKNYPGKDDS
DEQK
>WP_046441786.1 (RspCas13d)
[Ruminococcus sp. N15.MGS-57]
(SEQ ID NO: 2)
MAKKNKMKPRELREAQKKARQLKAAEINNNAAPAIAAMPAAEVIAPVAEK
KKSSVKAAGMKSILVSKNKMYITSFGKGNSAVLEYEVDNNDYNQTQLSSK
GSSNIELRGVNEVNITFSSKHGFESGVEINTSNPTHRSGESSPVRGDMLG
LKSELEKRFFGKTFDDNIHIQLIYNILDIEKILAVYVTNIVYALNNMLSI
KDSESYDDFMGYLSARNTYEVFTHPDKSNLSDKAKGNIKKSFSTENDLLK
TKRLGYFGLEEPKTKDTRVSQAYKKRVYHMLAIVGQIRQSVFHDKSSKLD
EDLYSFIDIIDSEYRETLDYLVDERFDSINKGFIQGNKVNISLLIDMMKG
YEADDIIRLYYDFIVLKSQKNLGFSIKKLREKMLDEYGFRFKDKQYDSVR
SKMYKLMDFLLFCNYYRNDVVAGEALVRKLRFSMTDDEKEGIYADEASKL
WGKERNDFENIADHMNGDVIKELGKADMDFDEKILDSEKKNASDLLYFSK
MIYMLTYFLDGKEINDLLTTLISKFDNIKEFLKIMKSSAVDVECELTAGY
KLENDSQRITNELFIVKNIASMRKPASSAKLTMERDALTILGIDDNITDD
RISEILKLKEKGKGIHGLRNFITNNVIESSREVYLIKYANAQKIRKVAKN
EKVVMFVLGGIPDTQIERYYKSCVEFPDMNSSLEVKRSELARMIKNISFD
DFKNVKQQAKGRENVAKERAKAVIGLYLTVMYLLVKNLVNVNARYVIAIH
CLERDFGLYKEIIPELASKNLKNDYRILSQTLCELCDKSPNLFLKKNERL
RKCVEVDINNADSSMTRKYRNCIAHLTVVRELKEYIGDIRTVDSYFSIYH
YVMQRCITKRENDTKQEEKIKYEDDLLKNHGYTKDFVKALNSPFGYNIPR
FKNLSIEQLFDRNEYLTEK

In some embodiments, the CRISPR-associated proteins described herein (e.g., Type VI-D CRISPR-Cas effector proteins) are from about 800 to about 1150 amino acids long, such as about 850 to about 1100 amino acids in length, e.g., about 850 to about 1050, about 850 to about 1000 amino acids long, or about 850 to about 950 amino acids long.

In some embodiments, the CRISPR-associated proteins (e.g., Type VI-D CRISPR-Cas effector proteins) have RNAse activity (e.g., collateral RNAse activity). In some embodiments, the CRISPR-associated proteins have DNAse activity. In some embodiments, the DNAse and/or RNAse activity is mediated by a single or both HEPN domains present in the CRISPR-associated proteins.

In some embodiments, a CRISPR-associated protein (e.g., Type VI-D CRISPR-Cas effector protein) is derived from a Ruminococcus or Eubacterium bacterium. In some embodiments, the CRISPR associated protein is derived from a human stool sample bacterial source.

Collateral RNase Activity

In some embodiments, a complex comprised of (but not limited to) a CRISPR-associated protein and a crRNA is activated upon binding to a target nucleic acid (e.g., a target RNA). Activation induces a conformational change, which results in the complex acting as a non-specific RNase, cleaving and/or degrading nearby RNA molecules (e.g., ssRNA or dsRNA molecules) (i.e., “collateral” effects).

Collateral-Free RNA Cleavage

In other embodiments, a complex comprised of (but not limited to) the CRISPR-associated protein and a crRNA does not exhibit collateral RNase activity subsequent to target recognition. This “collateral-free” embodiment may comprise wild-type or engineered effector proteins.

PAM/PFS-Independent Targeting

In some embodiments, a CRISPR-associated protein (e.g., a Type VI-D CRISPR-Cas effector protein described herein) recognizes and cleaves the target nucleic acid without any additional requirements adjacent to or flanking the protospacer (i.e., protospacer adjacent motif “PAM” or protospacer flanking sequence “PFS” requirements).

Deactivated/Inactivated CRISPR-Associated Proteins

Where the CRISPR-associated proteins described herein have nuclease activity, the CRISPR-associated proteins can be modified to have diminished nuclease activity, e.g., nuclease inactivation of at least 50%, at least 60%, at least 70%, at least 80%, at least 90%, at least 95%, at least 97%, or 100% as compared with the wild type CRISPR-associated proteins. The nuclease activity can be diminished by several methods known in the art, e.g., introducing mutations into the nuclease domains of the proteins. In some embodiments, catalytic residues for the nuclease activities are identified, and these amino acid residues can be substituted by different amino acid residues (e.g., glycine or alanine) to diminish the nuclease activity. In some embodiments, the amino acid substitution is a conservative amino acid substitution. In some embodiments, the amino acid substitution is a non-conservative amino acid substitution.

In some embodiments, the CRISPR-associated proteins described herein (e.g., a Type VI-D CRISPR-Cas effector protein) are modified to comprise one or more mutations (e.g., amino acid deletions, insertions, or substitutions) in at least one HEPN domain. In some embodiments, the CRISPR associate protein includes one, two, three, four, five, six, seven, eight, nine, or more amino acid substitutions in at least one HEPN domain. For example, in some embodiments, the one or more mutations comprise a substitution (e.g., an alanine substitution) at an amino acid residue corresponding to R295, H300, R849, H854 of SEQ ID NO: 1, or R288, H293, R820, or H825 of SEQ ID NO: 2. The presence of at least one of these mutations results in a CRISPR-associated protein having reduced nuclease activity (e.g., RNAse activity) as compared to the nuclease activity of the CRISPR-associated protein from which the protein was derived (i.e., lacking the mutation).

The inactivated CRISPR-associated proteins can be fused or associated with one or more functional domains (e.g., via fusion protein, linker peptides, “GS” linkers, etc.). These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Kruppel associated box (KRAB), VP64, VP16, Fok1, P65, HSF1, MyoD1, Adenosine Deaminase Acting on RNA (ADAR) 1, ADAR2, APOBEC, cytidine deaminase (AID), mini-SOG, APEX, and biotin-APEX. In some embodiments, the functional domain is a base editing domain (e.g., ADAR1, ADAR2, APOBEC, or AID). In some embodiments, the CRISPR-associated protein is fused to one functional domain. In some embodiments, the CRISPR-associated protein is fused to multiple (e.g., two, three, four, five, six, seven, eight, or more) functional domains. In some embodiments, the functional domain (e.g., a base editing domain) is further fused to an RNA-binding domain (e.g., MS2). In some embodiments, the CRISPR-associated protein is associated to or fused to a functional domain via a linker sequence (e.g., a flexible linker sequence or a rigid linker sequence). Exemplary linker sequences and functional domain sequences are provided in Table 10.

The positioning of the one or more functional domains on the inactivated CRISPR-associated proteins is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the CRISPR-associated protein. In some embodiments, the functional domain is positioned at the C-terminus of the CRISPR-associated protein. In some embodiments, the inactivated CRISPR-associated protein is modified to comprise a first functional domain at the N-terminus and a second functional domain at the C-terminus.

Various examples of inactivated CRISPR-associated proteins fused with one or more functional domains and methods of using the same are described, e.g., in International Publication No. WO 2017/219027, which is incorporated herein by reference in its entirety, and in particular with respect to the features described herein.

Split Enzymes

The present disclosure also provides a split version of the CRISPR-associated proteins described herein (e.g., a Type VI-D CRISPR-Cas effector protein). The split version of the CRISPR-associated protein may be advantageous for delivery. In some embodiments, the CRISPR-associated proteins are split into two parts of the enzyme, which together substantially comprise a functioning CRISPR-associated protein.

The split can be done in a way that the catalytic domain(s) are unaffected. The CRISPR-associated protein may function as a nuclease or may be an inactivated enzyme, which is essentially a RNA-binding protein with very little or no catalytic activity (e.g., due to mutation(s) in its catalytic domains). Split enzymes are described, e.g., in Wright, Addison V., et al. “Rational design of a split-Cas9 enzyme complex,” Proc. Nat'l. Acad. Sci., 112.10 (2015): 2984-2989, which is incorporated herein by reference in its entirety.

In some embodiments, the nuclease lobe and α-helical lobe are expressed as separate polypeptides. Although the lobes do not interact on their own, the crRNA recruits them into a ternary complex that recapitulates the activity of full-length CRISPR-associated proteins and catalyzes site-specific DNA cleavage. The use of a modified crRNA abrogates split-enzyme activity by preventing dimerization, allowing for the development of an inducible dimerization system.

In some embodiments, the split CRISPR-associated protein can be fused to a dimerization partner, e.g., by employing rapamycin sensitive dimerization domains. This allows the generation of a chemically inducible CRISPR-associated protein for temporal control of the activity of the protein. The CRISPR-associated protein can thus be rendered chemically inducible by being split into two fragments and rapamycin-sensitive dimerization domains can be used for controlled re-assembly of the protein.

The split point is typically designed in silico and cloned into the constructs. During this process, mutations can be introduced to the split CRISPR-associated protein and non-functional domains can be removed. In some embodiments, the two parts or fragments of the split CRISPR-associated protein (i.e., the N-terminal and C-terminal fragments), can form a full CRISPR-associated protein, comprising, e.g., at least 70%, at least 80%, at least 90%, at least 95%, or at least 99% of the sequence of the wild-type CRISPR-associated protein.

Self-Activating or Inactivating Enzymes

The CRISPR-associated proteins described herein (e.g., a Type VI-D CRISPR-Cas effector protein) can be designed to be self-activating or self-inactivating. For example, the target sequence can be introduced into the coding construct of the CRISPR-associated protein. Thus, the CRISPR-associated protein can cleave the target sequence, as well as the construct encoding the protein thereby self-inactivating their expression. Methods of constructing a self-inactivating CRISPR system are described, e.g., in Epstein, and Schaffer, Mol. Ther. 24 (2016): S50, which is incorporated herein by reference in its entirety.

In some other embodiments, an additional crRNA, expressed under the control of a weak promoter (e.g., 7SK promoter), can target the nucleic acid sequence encoding the CRISPR-associated protein to prevent and/or block its expression (e.g., by preventing the transcription and/or translation of the nucleic acid). The transfection of cells with vectors expressing the CRISPR-associated protein, the crRNAs, and crRNAs that target the nucleic acid encoding the CRISPR-associated protein can lead to efficient disruption of the nucleic acid encoding the CRISPR-associated protein and decrease the levels of CRISPR-associated protein, thereby limiting the genome editing activity.

In some embodiments, the genome editing activity of the CRISPR-associated protein can be modulated through endogenous RNA signatures (e.g., miRNA) in mammalian cells. A CRISPR-associated protein switch can be made by using a miRNA-complementary sequence in the 5′-UTR of mRNA encoding the CRISPR-associated protein. The switches selectively and efficiently respond to miRNA in the target cells. Thus, the switches can differentially control the genome editing by sensing endogenous miRNA activities within a heterogeneous cell population. Therefore, the switch systems can provide a framework for cell-type selective genome editing and cell engineering based on intracellular miRNA information (see, e.g., Hirosawa et al. Nucl. Acids Res., 2017, 45(13): e118).

Inducible CRISPR-Associated Proteins

The CRISPR-associated proteins (e.g., Type VI-D CRISPR-Cas effector proteins) can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al. “Optical control of mammalian endogenous transcription and epigenetic states,” Nature, 500.7463 (2013): 472). Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche, Volz, and Zhang, “A split-Cas9 architecture for inducible genome editing and transcription modulation,” Nature Biotech., 33.2 (2015): 139-142).

Furthermore, expression of the CRISPR-associated proteins can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless, Stephen J. et al. “Direct and specific chemical control of eukaryotic translation with a synthetic RNA-protein interaction,” Nucl Acids Res., 40.9 (2012): e64-e64).

Various embodiments of inducible CRISPR-associated proteins and inducible CRISPR systems are described, e.g., in U.S. Pat. No. 8,871,445, US Publication No. 2016/0208243, and International Publication No. WO 2016/205764, each of which is incorporated herein by reference in its entirety.

### Functional Mutations

In some embodiments, the CRISPR-associated proteins include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence PKKKRKV (SEQ ID NO: 135); the NLS from nucleoplasmin (e.g., the nucleoplasmin bipartite NLS with the sequence KRPAATKKAGQAKKKK (SEQ ID NO: 136)); the c-myc NLS having the amino acid sequence PAAKRVKLD (SEQ ID NO: 137) or RQRRNELKRSP (SEQ ID NO: 138); the hRNPA1 M9 NLS having the sequence NQSSNFGPMKGGNFGGRSSGPYGGGGQYFAKPRNQGGY (SEQ ID NO: 139); the sequence RMRIZFKNKGKDTAELRRRRVEVSVELRKAKKDEQILKRRNV (SEQ ID NO: 140) of the IBB domain from importin-alpha; the sequences VSRKRPRP (SEQ ID NO: 141) and PPKKARED (SEQ ID NO: 142) of the myoma T protein; the sequence PQPKKKPL (SEQ ID NO: 143) of human p53; the sequence SALIKKKKKMAP (SEQ ID NO: 144) of mouse c-abl IV; the sequences DRLRR (SEQ ID NO: 145) and PKQKKRK (SEQ ID NO: 146) of the influenza virus NS1; the sequence RKLKKKIKKL (SEQ ID NO: 147) of the Hepatitis virus delta antigen; the sequence REKKKFLKRR (SEQ ID NO: 148) of the mouse Mx1 protein; the sequence KRKGDEVDGVDEVAKKKSKK (SEQ ID NO: 149) of the human poly(ADP-ribose) polymerase; and the sequence RKCLQAGMNLEARKTKK (SEQ ID NO: 150) of the human glucocorticoid receptor. In some embodiments, the CRISPR-associated protein includes at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the CRISPR-associated proteins described herein are mutated at one or more amino acid residues to alter one or more functional activities. For example, in some embodiments, the CRISPR-associated protein is mutated at one or more amino acid residues to alter its helicase activity. In some embodiments, the CRISPR-associated protein is mutated at one or more amino acid residues to alter its nuclease activity (e.g., endonuclease activity or exonuclease activity). In some embodiments, the CRISPR-associated protein is mutated at one or more amino acid residues to alter its ability to functionally associate with an RNA guide. In some embodiments, the CRISPR-associated protein is mutated at one or more amino acid residues to alter its ability to functionally associate with a target nucleic acid.

In some embodiments, the CRISPR-associated proteins described herein are capable of cleaving a target nucleic acid molecule. In some embodiments, the CRISPR-associated protein cleaves both strands of the target nucleic acid molecule. However, in some embodiments, the CRISPR-associated protein is mutated at one or more amino acid residues to alter its cleaving activity. For example, in some embodiments, the CRISPR-associated protein may comprise one or more mutations that render the enzyme incapable of cleaving a target nucleic acid. In other embodiments, the CRISPR-associated protein comprise one or more mutations such that the enzyme is capable of cleaving a single strand of the target nucleic acid (i.e., nickase activity). In some embodiments, the CRISPR-associated protein is capable of cleaving the strand of the target nucleic acid that is complementary to the strand to which the RNA guide hybridizes. In some embodiments, the CRISPR-associated protein is capable of cleaving the strand of the target nucleic acid to which the guide RNA hybridizes.

In some embodiments, a CRISPR-associated protein described herein can be engineered to have a deletion in one or more amino acid residues to reduce the size of the enzyme while retaining one or more desired functional activities (e.g., nuclease activity and the ability to interact functionally with a RNA guide). The truncated CRISPR-associated protein can be advantageously used in combination with delivery systems having load limitations.

Nucleic acids encoding the proteins (e.g., a CRISPR-associated protein or an accessory protein) and RNA guides (e.g., a crRNA) described herein are also provided. In some embodiments, the nucleic acid is a synthetic nucleic acid. In some embodiments, the nucleic acid is a DNA molecule. In some embodiments, the nucleic acid is an RNA molecule (e.g., an mRNA molecule). In some embodiments, the nucleic acid is an mRNA. In some embodiments, the mRNA is capped, polyadenylated, substituted with 5-methylcytidine, substituted with pseudouridine, or a combination thereof. In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter. Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a β-actin promoter. For example, a U6 promoter can be used to regulate the expression of an RNA guide molecule described herein.

In some embodiments, the nucleic acid(s) are present in a vector (e.g., a viral vector or a phage). The vectors can include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein. In another aspect, the present disclosure also provides amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences described herein.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

In some embodiments, the CRISPR-associated proteins and accessory proteins described herein can be fused to one or more peptide tags, including a His-tag, GST-tag, or myc-tag. In some embodiments, the CRISPR-associated proteins or accessory proteins described herein can be fused to a detectable moiety such as a fluorescent protein (e.g., green fluorescent protein or yellow fluorescent protein).

The proteins described herein (e.g., CRISPR-associated proteins or accessory proteins) can be delivered or used as either nucleic acid molecules or polypeptides. When nucleic acid molecules are used, the nucleic acid molecule encoding the CRISPR-associated proteins can be codon-optimized. The nucleic acid can be codon optimized for use in any organism of interest, in particular human cells or bacteria. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/ and these tables can be adapted in a number of ways. See Nakamura et al. Nucl Acids Res. 28:292 (2000), which is incorporated herein by reference in its entirety. Computer algorithms for codon optimizing a particular sequence for expression in a particular host cell are also available, such as Gene Forge (Aptagen; Jacobus, Pa.).

### RNA Guides

In some embodiments, the CRISPR systems described herein include at least RNA guide (e.g., a crRNA). The architecture of multiple RNA guides is known in the art (see, e.g., International Publication Nos. WO 2014/093622 and WO 2015/070083, the entire contents of each of which are incorporated herein by reference). In some embodiments, the CRISPR systems described herein include multiple RNA guides (e.g., one, two, three, four, five, six, seven, eight, or more RNA guides). In some embodiments, the RNA guide includes a crRNA. In some embodiments, the RNA guide includes a crRNA and a tracrRNA. In some embodiments, the RNA guide is an engineered construct that includes a tracrRNA and a crRNA (in a single RNA guide). Sequences for RNA guides from multiple CRISPR systems are known in the art and can be searched using public databases (see, e.g., Grissa et al. (2007) Nucleic Acids Res. 35 (web server issue): W52-7; Grissa et al. (2007) BMC Bioinformatics 8: 172; Grissa et al. (2008) Nucleic Acids Res. 36 (web server issue): W145-8; and Moller and Liang (2017) PeerJ 5: e3788; see also the CRISPR database available at: crispr.i2bc.paris-saclay.fr/crispr/BLAST/CRISPRsBlast.php; and MetaCRAST available at: github.com/molleraj/MetaCRAST).

In some embodiments, the CRISPR systems described herein include at least one crRNA or a nucleic acid encoding at least one crRNA. In some embodiments, the crRNA includes a direct repeat sequence, a spacer sequence, and a direct repeat sequence, which is typical of precursor crRNA (pre-crRNA) configurations in other CRISPR systems. In some embodiments, the crRNA includes a truncated direct repeat sequence and a spacer sequence, which is typical of processed or mature crRNA. The CRISPR-associated protein is capable of cleaving pre-crRNA to form processed or mature crRNA. The CRISPR-associated protein forms a complex with the mature crRNA, and the spacer sequence directs the complex to a sequence-specific binding with the target nucleic acid that is complementary to the spacer sequence. The resulting complex comprises the CRISPR-associated protein and the mature crRNA bound to the target RNA.

In some embodiments, the CRISPR systems described herein include a mature crRNA. In some embodiments, the CRISPR systems described herein include a pre-crRNA.

In some embodiments, the CRISPR systems described herein include a plurality of crRNAs (e.g., 2, 3, 4, 5, 10, 15, or more) or a plurality of nucleic acids encoding a plurality of crRNAs. Generally, the crRNAs described herein include a direct repeat sequence and a spacer sequence. In certain embodiments, the crRNA includes, consists essentially of, or consists of a direct repeat sequence linked to a guide sequence or spacer sequence.

In some embodiments, the CRISPR system described herein includes an RNA guide (e.g., a crRNA) or a nucleic acid encoding the RNA guide. In some embodiments, the RNA guide comprises or consists of a direct repeat sequence and a spacer sequence capable of hybridizing (e.g., hybridizes under appropriate conditions) to a target nucleic acid, wherein the direct repeat sequence comprises 5′-X₁X₂X₃X₄TX₅TX₆AAAC-3′ (SEQ ID NO: 151) at the 3′ terminal end of the RNA guide, and wherein X₁ is A or C or G, X₂ is G or T, X₃ is A or G, X₄ is C or G or T, X₅ is C or T, and X₆ is A or G. In some embodiments, the RNA guide comprises or consists of a direct repeat sequence and a spacer sequence capable of hybridizing (e.g., hybridizes under appropriate conditions) to a target nucleic acid, wherein the direct repeat sequence comprises 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G.

Exemplary RNA guide direct repeat sequences and effector protein pairs are provided in Table 3. In some embodiments, the direct repeat sequence comprises or consists of a nucleic acid sequence listed in Table 3 (e.g., SEQ ID NOs 32-49, 52-77, 351-589). In some embodiments, the direct repeat sequence comprises or consists of a nucleic acid having a nucleic acid sequence listed in Table 3 with a truncation of the initial three 5′ nucleotides. In some embodiments, the direct repeat sequence comprises or consists of a nucleic acid having a nucleic acid sequence listed in Table 3 with a truncation of the initial four 5′ nucleotides. In some embodiments, the direct repeat sequence comprises or consists of a nucleic acid having a nucleic acid sequence listed in Table 3 with a truncation of the initial five 5′ nucleotides. In some embodiments, the direct repeat sequence comprises or consists of a nucleic acid having a nucleic acid sequence listed in Table 3 with a truncation of the initial six 5′ nucleotides. In some embodiments, the direct repeat sequence comprises or consists of a nucleic acid having a nucleic acid sequence listed in Table 3 with a truncation of the initial seven 5′ nucleotides. In some embodiments, the direct repeat sequence comprises or consists of a nucleic acid having a nucleic acid sequence listed in Table 3 with a truncation of the initial eight 5′ nucleotides.

In some embodiments, the direct repeat sequence comprises or consists of the nucleic acid sequence 5′-GAACTACACCCGTGCAAAATTGCAGGGGTCTAAAAC-3′ (SEQ ID NO: 34) or 5′-CTACTACACTGGTGCAAATTTGCACTAGTCTAAAAC-3′ (SEQ ID NO: 72). In some embodiments, the direct repeat sequence comprises or consists of the nucleic acid sequence 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153).

In some embodiments, the CRISPR-associated protein comprises the amino acid sequence of SEQ ID NO: 1 and the crRNA comprises a direct repeat sequence, wherein the direct repeat sequence comprises or consists of the nucleic acid sequence 5′-GAACTACACCCGTGCAAAATTGCAGGGGTCTAAAAC-3′ (SEQ ID NO: 34) or 5′-CACCCGTGCAAAATTGCAGGGGTCTAAAAC-3′ (SEQ ID NO: 152). In some embodiments, the CRISPR-associated protein comprises the amino acid sequence of SEQ ID NO: 2 and the crRNA comprises a direct repeat sequence, wherein the direct repeat sequence comprises or consists of the nucleic acid sequence 5′-CTACTACACTGGTGCAAATTTGCACTAGTCTAAAAC-3′ (SEQ ID NO: 72) or 5′-CACTGGTGCAAATTTGCACTAGTCTAAAAC-3′ (SEQ ID NO: 153).

Multiplexing RNA Guides

Type VI CRISPR-Cas effectors have been demonstrated to employ more than one RNA guide, thus enabling the ability of these effectors, and systems and complexes that include them, to target multiple nucleic acids. In some embodiments, the CRISPR systems described herein include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Type VI-D CRISPR-Cas effector proteins described herein enables these effectors to be able to target multiple target nucleic acids (e.g., target RNAs) without a loss of activity. In some embodiments, the Type VI-D CRISPR-Cas effector proteins may be delivered in complex with multiple RNA guides directed to different target nucleic acids. In some embodiments, the Type VI-D CRISPR-Cas effector proteins may be co-delivered with multiple RNA guides, each specific for a different target nucleic acid. Methods of multiplexing using CRISPR-associated proteins are described, for example, in U.S. Pat. No. 9,790,490 B2, and EP 3009511 B1, the entire contents of each of which are expressly incorporated herein by reference.

Spacer Lengths

The spacer length of crRNAs can range from about 15 to 50 nucleotides. In some embodiments, the spacer length of an RNA guide is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the direct repeat length of the RNA guide is at least 16 nucleotides, or is from 16 to 20 nucleotides (e.g., 16, 17, 18, 19, or 20 nucleotides). In some embodiments, the spacer length is from about 15 to about 42 nucleotides. In some embodiments, the direct repeat length of the RNA guide is 19 nucleotides.

The crRNA sequences can be modified in a manner that allows for formation of a complex between the crRNA and CRISPR-associated protein and successful binding to the target, while at the same time not allowing for successful nuclease activity (i.e., without nuclease activity/without causing indels). These modified guide sequences are referred to as “dead crRNAs,” “dead guides,” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective RNA guides that have nuclease activity. Dead guide sequences of RNA guides can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional CRISPR-associated protein as described herein, and a crRNA, wherein the crRNA comprises a dead crRNA sequence whereby the crRNA is capable of hybridizing to a target sequence such that the CRISPR system is directed to a genomic locus of interest in a cell without detectable nuclease activity (e.g., RNAse activity).

A detailed description of dead guides is described, e.g., in International Publication No. WO 2016/094872, which is incorporated herein by reference in its entirety.

Inducible Guides

RNA guides (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of RNA guides (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

Chemical Modifications

Chemical modifications can be applied to the crRNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther., 24 (2014), pp. 374-387); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem., 48.4 (2005): 901-904). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized crRNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the crRNA includes one or more phosphorothioate modifications. In some embodiments, the crRNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 2016 Sep. 10; 233:74-83; WO 2016205764; and U.S. Pat. No. 8,795,965 B2; each which is incorporated by reference in its entirety.

Sequence Modifications

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effectors, gene activators, or gene repressors. In some embodiments, the aptamers can be specific to a protein, which in turn is specific to and recruits and/or binds to specific gene effectors, gene activators, or gene repressors. The effectors, activators, or repressors can be present in the form of fusion proteins. In some embodiments, the RNA guide has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, and PRR1.

Accordingly, in some embodiments, the aptamer is selected from binding proteins specifically binding any one of the adaptor proteins as described herein. In some embodiments, the aptamer sequence is a MS2 binding loop (5′-ggcccAACAUGAGGAUCACCCAUGUCUGCAGgggcc-3′ (SEQ ID NO: 169)). In some embodiments, the apatamer sequence is a QBeta binding loop (5′-ggcccAUGCUGUCUAAGACAGCAUgggcc-3′ (SEQ ID NO: 170)). In some embodiments, the aptamer sequence is a PP7 binding loop (5′-ggcccUAAGGGUUUAUAUGGAAACCCUUAgggcc-3′ (SEQ ID NO: 173)). A detailed description of aptamers can be found, e.g., in Nowak et al., “Guide RNA engineering for versatile Cas9 functionality,” Nucl. Acid. Res., 2016 Nov. 16; 44(20):9555-9564; and WO 2016205764, which are incorporated herein by reference in their entirety.

### Target Nucleic Acids

The target nucleic acids can be a DNA molecule or a RNA molecule. As described above, in some embodiments, the CRISPR-associated proteins described herein have RNAse activity. Thus, the target nucleic acids can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease (e.g., an infectious disease or a cancer). Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

Guide: Target Sequence Matching Requirements

In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 100%. The RNA guides can be about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′ ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Target Nucleic Acids to Regulate Collateral RNAse Activity Activation

In some embodiments, the CRISPR systems described herein further comprise a target nucleic acid (e.g., a linear or circular nucleic acid) which may advantageously be used to activate the collateral RNAse activity of a Type VI-D CRISPR-Cas effector protein in a controlled manner. By regulating the expression and/or delivery of the target nucleic acid, the activation of the collateral RNAse activity of the effector protein may be controlled. For example, exogenous target nucleic acid may be included in the system to increase the activation rate of the collateral RNAse activity of a Type VI-D CRISPR-Cas effector protein. In some embodiments, the target nucleic acid is a DNA molecule. In some embodiments, the target nucleic acid is an RNA molecule (e.g., a mRNA molecule). In some embodiments, when the target nucleic acid is an RNA, the system includes a DNA molecule (e.g., a plasmid DNA) that codes for the target nucleic acid that is specifically targeted by the Type VI-D CRISPR-Cas effector protein and crRNA complex, operably linked to a promoter. In some embodiments, the promoter is an inducible promoter. In some embodiments, the promoter is a constitutive promoter.

Accessory Proteins

In one aspect, the CRISPR systems described herein includes at least one accessory protein. As shown in Example 4, the inventors have surprisingly discovered that the accessory proteins described herein enhance the nuclease activity of CRISPR-associated proteins (e.g., Type VI-D CRISPR-Cas effector proteins) as compared to the nuclease activity of the CRISPR associated protein in the absence of the accessory protein. The ability of the accessory proteins described herein to enhance the nuclease activity of CRISPR-associated proteins is particularly desireable in clinical and therapeutic applications. Therefore, CRISPR systems including at least one accessory protein are provided herein. For example, an accessory protein described herein may be used in combination with CRISPR-associated proteins known in the art in order to enhance their nuclease activity. Alternatively, an accessory protein may be used in combination with a Type VI-D CRISPR-Cas effector protein described herein to enhance its nuclease activity (e.g., collateral RNAse activity or targeted RNAse activity).

In some embodiments, the accessory protein includes a WYL domain (PFAM: PF13280), which has been predicted to be a ligand-sensing domain, which can regulate CRISPR-Cas systems. WYL domains are SH3 beta-barrel fold containing domains named for three conserved amino acids found in some domains belonging to the WYL-like superfamily. One WYL domain protein, s117009, has been found to be a negative regulator of the Synechocystis sp. I-D CRISPR-Cas system (see, e.g., Hein et al. (2013) RNA Biol. 10: 852-64).

In some embodiments, the accessory protein includes at least one WYL domain. In some embodiments, the accessory protein includes two WYL domains. In some embodiments, the accessory protein includes a helix-turn-helix (HTH) fold. In some embodiments, the accessory protein includes a ribbon-helix-helix (RHH) fold. In some embodiments, the accessory protein includes at least one WYL domain, wherein the WYL domain comprises the amino acid sequence PXXX₁XXXXXXXXXYL (SEQ ID NO: 198), wherein X₁ is C, V, I, L, P, F, Y, M, or W, and wherein X is any amino acid. In some embodiments, the accessory protein includes at least one WYL domain, wherein the WYL domain comprises the amino acid sequence PXXX₁XXXXXXXXXYL (SEQ ID NO: 198), wherein X₁ is C, V, I, L, P, F, Y, M, or W, and wherein X is any amino acid; and at least one ribbon-ribbon-helix (RHH) fold or at least one helix-turn-helix (HTH) domain. In some embodiments, the amino acid sequence of the WYL domain is separate from (i.e., does not overlap with) an RHH fold or an HTH fold.

In some embodiments, the accessory proteins describe herein modulate the RNAse activity of a CRISPR-associated protein. In some embodiments, the accessory protein modulates (e.g., increases or decreases) the collateral RNAse activity of a CRISPR-associated protein. In some embodiments, the accessory protein modulates (e.g., increases or decreases) the RNA-binding activity of a CRISPR-associated protein. In some embodiments, the accessory protein modulates (e.g., increases or decreases) the crRNA processing activity of a CRISPR-associated protein. In some embodiments, the accessory protein modulates (e.g., increases or decreases) the targeted RNAse activity of a CRISPR-associated protein.

In some embodiments, the accessory proteins described herein enhances the RNAse activity of a CRISPR-associated protein (e.g., a Cas13a protein, a Cas13b protein, a Cas13c protein, a Cas12a protein, a Cas9 protein). In some embodiments, the accessory protein enhances the collateral RNAse activity of a CRISPR-associated protein. In some embodiments, the accessory protein enhances the crRNA processing activity of a CRISPR-associated protein. In some embodiments, the accessory protein enhances the RNA-binding activity of a CRISPR-associated protein. In some embodiments, the accessory protein enhances the targeted RNAse activity of a CRISPR-associated protein. CRISPR systems comprising an accessory protein described herein are particularly useful in applications where increased sequence-specific or collateral RNA degradation is desireable. For example, in diagnostic applications, enhanced RNAse activity provides a greater degree of sensitivity, allowing the detection of lower concentrations of a target RNA. In some embodiments, an accessory protein described herein enhances the RNAse activity of the ternary complex of multiple CRISPR Type VI effectors. The ability of the accessory protein to enhance the RNAse of multiple effectors is particularly useful in applications where combinations of Type VI effectors of different sub-types are used together, for example in multi-channel diagnostic applications. In some embodiments, the accessory protein can enhance the RNAse activity of Type VI effectors outside the Cas13d family thereby providing a valuable tool for screening the activity of uncharacterized Type VI effectors.

Exemplary accessory proteins are provided below in Tables 4, 5 and 6 (e.g., SEQ ID NOs. 78-93, and 590-671). In some embodiments, the accessory proteins include an amino acid sequence having at least about 80% identity (e.g., 81%, 82%, 83%, 84%, 85%, 86%, 87%, 88%, 89%, 90%, 91%, 92%, 93%, 94%, 95%, 96%, 97%, 98%, 99%, or 100% identity) to the amino acid sequence of any one of Tables 4, 5 and 6 (e.g., SEQ ID NOs. 78-93, and 590-671). In some embodiments, the accessory protein includes the amino acid sequence of any one of the proteins in Tables 4, 5 and 6 (e.g., SEQ ID NOs. 78-93, and 590-671). In some embodiments, the accessory protein is RspWYL1 (SEQ ID NO: 81).

### Methods of Using CRISPR Systems

The CRISPR systems described herein have a wide variety of utilities including modifying (e.g., deleting, inserting, translocating, inactivating, or activating) a target polynucleotide or nucleic acid in a multiplicity of cell types. The CRISPR systems have a broad spectrum of applications in, e.g., DNA/RNA detection (e.g., specific high sensitivity enzymatic reporter unlocking (SHERLOCK)), tracking and labeling of nucleic acids, enrichment assays (extracting desired sequence from background), controlling interfering RNA or miRNA, detecting circulating tumor DNA, preparing next generation library, drug screening, disease diagnosis and prognosis, and treating various genetic disorders.

### DNA/RNA Detection

In one aspect, the CRISPR systems described herein can be used in DNA or RNA detection. CRISPR-associated proteins can be reprogrammed with CRISPR RNAs (crRNAs) to provide a platform for specific RNA sensing. Upon recognition of its RNA target, activated CRISPR-associated proteins engage in “collateral” cleavage of nearby non-targeted RNAs. This crRNA-programmed collateral cleavage activity allows the CRISPR systems to detect the presence of a specific RNA by triggering programmed cell death or by nonspecific degradation of labeled RNA.

The SHERLOCK method (Specific High Sensitivity Enzymatic Reporter UnLOCKing) provides an in vitro nucleic acid detection platform with attomolar sensitivity based on nucleic acid amplification and collateral cleavage of a reporter RNA, allowing for real-time detection of the target. To achieve signal detection, the detection can be combined with different isothermal amplification steps. For example, recombinase polymerase amplification (RPA) can be coupled with T7 transcription to convert amplified DNA to RNA for subsequent detection. The combination of amplification by RPA, T7 RNA polymerase transcription of amplified DNA to RNA, and detection of target RNA by collateral RNA cleavage-mediated release of reporter signal is referred as SHERLOCK. Methods of using CRISPR in SHERLOCK are described in detail, e.g., in Gootenberg, et al. “Nucleic acid detection with CRISPR-Cas13a/C2c2,” Science, 2017 Apr. 28; 356(6336):438-442, which is incorporated herein by reference in its entirety.

The CRISPR-associated proteins can further be used in Northern blot assays, which use electrophoresis to separate RNA samples by size. The CRISPR-associated proteins can be used to specifically bind and detect the target RNA sequence. The CRISPR-associated proteins can also be fused to a fluorescent protein (e.g., GFP) and used to track RNA localization in living cells. More particularly, the CRISPR-associated proteins can be inactivated in that they no longer cleave RNAs as described above. Thus, CRISPR-associated proteins can be used to determine the localization of the RNA or specific splice variants, the level of mRNA transcripts, up- or down-regulation of transcripts and disease-specific diagnosis. The CRISPR-associated proteins can be used for visualization of RNA in (living) cells using, for example, fluorescent microscopy or flow cytometry, such as fluorescence-activated cell sorting (FACS), which allows for high-throughput screening of cells and recovery of living cells following cell sorting. A detailed description regarding how to detect DNA and RNA can be found, e.g., in International Publication No. WO 2017/070605, which is incorporated herein by reference in its entirety.

In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference herein in its entirety.

In some embodiments, the CRISPR systems described herein can be used to detect a target RNA in a sample (e.g., a clinical sample, a cell, or a cell lysate). The collateral RNAse activity of the Type VI-D CRISPR-Cas effector proteins described herein is activated when the effector proteins bind to a target nucleic acid. Upon binding to the target RNA of interest, the effector protein cleaves a labeled detector RNA to generate a signal (e.g., an increased signal or a decreased signal) thereby allowing for the qualitative and quantitative detection of the target RNA in the sample. The specific detection and quantification of RNA in the sample allows for a multitude of applications including diagnostics. In some embodiments, the methods include contacting a sample with: i) an RNA guide (e.g., crRNA) and/or a nucleic acid encoding the RNA guide, wherein the RNA guide consists of a direct repeat sequence and a spacer sequence capable of hybridizing to the target RNA; (ii) a Type VI-D CRISPR-Cas effector protein and/or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNAse activity and cleaves the labeled detector RNA; and b) measuring a detectable signal produced by cleavage of the labeled detector RNA, wherein said measuring provides for detection of the single-stranded target RNA in the sample. In some embodiments, the methods further comprise comparing the detectable signal with a reference signal and determining the amount of target RNA in the sample. In some embodiments, the measuring is performed using gold nanoparticle detection, fluorescence polarization, colloid phase transition/dispersion, electrochemical detection, and semiconductor based-sensing. In some embodiments, the labeled detector RNA includes a fluorescence-emitting dye pair, a fluorescence resonance energy transfer (FRET) pair, or a quencher/fluor pair. In some embodiments, upon cleavage of the labeled detector RNA by the effector protein, an amount of detectable signal produced by the labeled detector RNA is decreased or increased. In some embodiments, the labeled detector RNA produces a first detectable signal prior to cleavage by the effector protein and a second detectable signal after cleavage by the effector protein. In some embodiments, a detectable signal is produced when the labeled detector RNA is cleaved by the effector protein. In some embodiments, the labeled detector RNA comprises a modified nucleobase, a modified sugar moiety, a modified nucleic acid linkage, or a combination thereof. In some embodiments, the methods include the multi-channel detection of multiple independent target RNAs in a sample (e.g., two, three, four, five, six, seven, eight, nine, ten, fifteen, twenty, thirty, forty, or more target RNAs) by using multiple Type VI-D CRISPR-Cas systems, each including a distinct orthologous effector protein and corresponding RNA guides, allowing for the differentiation of multiple target RNAs in the sample. In some embodiments, the methods include the multi-channel detection of multiple independent target RNAs in a sample, with the use of multiple instances of Type VI-D CRISPR-Cas systems, each containing an orthologous effector protein with differentiable collateral RNAse substrates. Methods of detecting an RNA in a sample using CRISPR-associated proteins are described, for example, in U.S. Patent Publication No. 2017/0362644, the entire contents of which are incorporated herein by reference.

Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The CRISPR-associated proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965, WO 2016205764, and WO 2017070605; each of which is incorporated herein by reference herein in its entirety.

RNA Isolation, Purification, Enrichment, and/or Depletion

The CRISPR systems (e.g., CRISPR-associated proteins) described herein can be used to isolate and/or purify the RNA. The CRISPR-associated proteins can be fused to an affinity tag that can be used to isolate and/or purify the RNA-CRISPR-associated protein complex. These applications are useful, e.g., for the analysis of gene expression profiles in cells.

In some embodiments, the CRISPR-associated proteins can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice variants, isoforms, etc.).

These methods are described, e.g., in U.S. Pat. No. 8,795,965, WO 2016205764, and WO 2017070605; each of which is incorporated herein by reference herein in its entirety.

High-Throughput Screening

The CRISPR systems described herein can be used for preparing next generation sequencing (NGS) libraries. For example, to create a cost-effective NGS library, the CRISPR systems can be used to disrupt the coding sequence of a target gene, and the CRISPR-associated protein transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with e.g. fusion complexes with the appropriate effectors such as kinases or enzymes.

In some embodiments, crRNAs that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast, 2017 Sep. 8. doi: 10.1002/yea.3278; and Hlavova et al., “Improving microalgae for biotechnology—from genetics to synthetic biology,” Biotechnol. Adv., 2015 Nov. 1; 33:1194-203, both of which are incorporated herein by reference in the entirety.

In some embodiments, the CRISPR systems provided herein can be used to induce death or dormancy of a cell (e.g., a microorganism such as an engineered microorganism). These methods can be used to induce dormancy or death of a multitude of cell types including prokaryotic and eukaryotic cells, including, but not limited to mammalian cells (e.g., cancer cells, or tissue culture cells), protozoans, fungal cells, cells infected with a virus, cells infected with an intracellular bacteria, cells infected with an intracellular protozoan, cells infected with a prion, bacteria (e.g., pathogenic and non-pathogenic bacteria), protozoans, and unicellular and multicellular parasites. For instance, in the field of synthetic biology it is highly desireable to have mechanisms of controlling engineered microorganisms (e.g., bacteria) in order to prevent their propagation or dissemination. The systems described herein can be used as “kill-switches” to regulate and/or prevent the propagation or dissemination of an engineered microorganism. Further, there is a need in the art for alternatives to current antibiotic treatments. The systems described herein can also be used in applications where it is desirable to kill or control a specific microbial population (e.g., a bacterial population). For example, the systems described herein may include an RNA guide (e.g., a crRNA) that targets a nucleic acid (e.g., an RNA) that is genus-, species-, or strain-specific, and can be delivered to the cell. Upon complexing and binding to the target nucleic acid, the collateral RNAse activity of the Type VI-D CRISPR-Cas effector proteins is activated leading to the cleavage of non-target RNA within the microorganisms, ultimately resulting in dormancy or death.

In some embodiments, the methods comprise contacting the cell with a system described herein including a Type VI-D CRISPR-Cas effector proteins or a nucleic acid encoding the effector protein, and a RNA guide (e.g., a crRNA) or a nucleic acid encoding the RNA guide, wherein the spacer sequence is complementary to at least 15 nucleotides (e.g., 16, 17, 18, 19, 20, 21, 22, 23, 24, 25, 26, 27, 28, 29, 30, 35, 40, 45, 50 or more nucleotides) of a target nucleic acid (e.g., a genus-, strain-, or species-specific RNA guide). Without wishing to be bound by any particular theory, the cleavage of non-target RNA by the Type VI-D CRISPR-Cas effector proteins may induce programmed cell death, cell toxicity, apoptosis, necrosis, necroptosis, cell death, cell cycle arrest, cell anergy, a reduction of cell growth, or a reduction in cell proliferation. For example, in bacteria, the cleavage of non-target RNA by the Type VI-D CRISPR-Cas effector proteins may be bacteriostatic or bacteriocidal.

Applications in Plants

The CRISPR systems described herein have a wide variety of utility in plants. In some embodiments, the CRISPR systems can be used to engineer genomes of plants (e.g., improving production, making products with desired post-translational modifications, or introducing genes for producing industrial products). In some embodiments, the CRISPR systems can be used to introduce a desired trait to a plant (e.g., with or without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr. Opin. Allergy Clin. Immunol., 2011 June; 11(3):222-8, and WO 2016205764 A1; both of which are incorporated herein by reference in the entirety.

Gene Drives

Gene drive is the phenomenon in which the inheritance of a particular gene or set of genes is favorably biased. The CRISPR systems described herein can be used to build gene drives. For example, the CRISPR systems can be designed to target and disrupt a particular allele of a gene, causing the cell to copy the second allele to fix the sequence. Because of the copying, the first allele will be converted to the second allele, increasing the chance of the second allele being transmitted to the offspring. A detailed method regarding how to use the CRISPR systems described herein to build gene drives is described, e.g., in Hammond et al., “A CRISPR-Cas9 gene drive system targeting female reproduction in the malaria mosquito vector Anopheles gambiae,” Nat. Biotechnol., 2016 January; 34(1):78-83, which is incorporated herein by reference in its entirety.

Pooled-Screening

As described herein, pooled CRISPR screening is a powerful tool for identifying genes involved in biological mechanisms such as cell proliferation, drug resistance, and viral infection. Cells are transduced in bulk with a library of RNA guide-encoding vectors described herein, and the distribution of RNA guides is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods., 2017 March; 14(3):297-301, which is incorporated herein by reference in its entirety.

Saturation Mutagenesis (Bashing)

The CRISPR systems described herein can be used for in situ saturating mutagenesis. In some embodiments, a pooled RNA guide library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature, 2015 Nov. 12; 527(7577):192-7, which is incorporated herein by reference in its entirety.

RNA-Related Applications

The CRISPR systems described herein can have various RNA-related applications, e.g., modulating gene expression, degrading a RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016/205764 A1, which is incorporated herein by reference in its entirety. In different embodiments, the methods described herein can be performed in vitro, in vivo, or ex vivo.

For example, the CRISPR systems described herein can be administered to a subject having a disease or disorder to target and induce cell death in a cell in a diseased state (e.g., cancer cells or cells infected with an infectious agent). For instance, in some embodiments, the CRISPR systems described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

Modulating Gene Expression

The CRISPR systems described herein can be used to modulate gene expression. The CRISPR systems can be used, together with suitable RNA guides, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable RNA guides can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPR as substitutes for e.g., interfering ribonucleic acids (such as siRNAs, shRNAs, or dsRNAs). The methods of modulating gene expression are described, e.g., in WO 2016205764, which is incorporated herein by reference in its entirety.

Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable RNA guides are selectively expressed (for example spatially or temporally under the control of a regulated promoter, for example a tissue- or cell cycle-specific promoter and/or enhancer), this can be used to protect the cells or systems (in vivo or in vitro) from RNA interference (RNAi) in those cells. This may be useful in neighboring tissues or cells where RNAi is not required or for the purposes of comparison of the cells or tissues where the CRISPR-associated proteins and suitable crRNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the RNA guides can recruit the RNA targeting proteins to these molecules so that the RNA targeting proteins are able to bind to them. These methods are described, e.g., in WO 2016205764 and WO 2017070605, both of which are incorporated herein by reference in the entirety.

Modifying Riboswitches and Controlling Metabolic Regulations

Riboswitches are regulatory segments of messenger RNAs that bind small molecules and in turn regulate gene expression. This mechanism allows the cell to sense the intracellular concentration of these small molecules. A specific riboswitch typically regulates its adjacent gene by altering the transcription, the translation or the splicing of this gene. Thus, in some embodiments, the riboswitch activity can be controlled by the use of the RNA targeting proteins in combination with suitable RNA guides to target the riboswitches. This may be achieved through cleavage of, or binding to, the riboswitch. Methods of using CRISPR systems to control riboswitches are described, e.g., in WO 2016205764 and WO 2017070605, both of which are incorporated herein by reference in their entireties.

RNA Modification

In some embodiments, the CRISPR-associated proteins described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-associated protein includes one or more mutations (e.g., in a catalytic domain), which renders the CRISPR-associated protein incapable of cleaving RNA.

In some embodiments, the CRISPR-associated proteins can be used with an RNA-binding fusion polypeptide comprising a base-editing domain (e.g., ADAR1, ADAR2, APOBEC, or AID) fused to an RNA-binding domain, such as MS2 (also known as MS2 coat protein), Qbeta (also known as Qbeta coat protein), or PP7 (also known as PP7 coat protein). The amino acid sequences of the RNA-binding domains MS2, Qbeta, and PP7 are provided below:

MS2 (MS2 coat protein)
(SEQ ID NO: 171)
MASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATNS
DCELIVKAMQGLLKDGNPIPSAIAANSGIY
Qbeta (Qbeta coat protein)
(SEQ ID NO: 172)
MAKLETVTLGNIGKDGKQTLVLNPRGVNPTNGVASLSQAGAVPALEKRVT
VSVSQPSRNRKNYKVQVKIQNPTACTANGSCDPSVTRQAYADVTFSFTQY
STDEERAFVRTELAALLASPLLIDAIDQLNPAY
PP7 (PP7 coat protein)
(SEQ ID NO: 155)
MSKTIVLSVGEATRTLTEIQSTADRQIFEEKVGPLVGRLRLTASLRQNGA
KTAYRVNLKLDQADVVDCSTSVCGELPKVRYTQVWSHDVTIVANSTEASR
KSLYDLTKSLVVQATSEDLVVNLVPLGR

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR systems for base editing are described, e.g., in International Publication No. WO 2017/219027, which is incorporated herein by reference in its entirety, and in particular with respect to its discussion of RNA modification.

RNA Splicing

In some embodiments, an inactivated CRISPR-associated protein described herein (e.g., a CRISPR associated protein having one or more mutations in a catalytic domain) can be used to target and bind to specific splicing sites on RNA transcripts. Binding of the inactivated CRISPR-associated protein to the RNA may sterically inhibit interaction of the spliceosome with the transcript, enabling alteration in the frequency of generation of specific transcript isoforms. Methods of using CRISPR systems to alter splicing are described, e.g., in International Publication No. WO 2017/219027, which is incorporated herein by reference in its entirety, and in particular with respect to its discussion of RNA splicing.

Therapeutic Applications

The CRISPR systems described herein can have various therapeutic applications. In some embodiments, the new CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). Methods of designing exogenous donor template nucleic acids are described, for example, in International Publication No. WO 2016/094874 A1, the entire contents of which are expressly incorporated herein by reference.

In one aspect, the CRISPR systems described herein can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016/205764 A1, both of which are incorporated herein by reference in the entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular against RNA viruses. The CRISPR-associated proteins can target the viral RNAs using suitable RNA guides selected to target viral RNA sequences.

The CRISPR systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

Further, the CRISPR systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

### Delivery

Through this disclosure and the knowledge in the art, the CRISPR systems described herein, or components thereof, nucleic acid molecules thereof, and/or nucleic acid molecules encoding or providing components thereof, can be delivered by various delivery systems such as vectors, e.g., plasmids and viral delivery vectors (e.g., adeno-associated virus AAV vectors). The CRISPR-associated proteins and/or any of the RNAs (e.g., RNA guides) and/or accessory proteins can be delivered using suitable vectors, e.g., plasmids or viral vectors, such as adeno-associated viruses (AAV), lentiviruses, adenoviruses, and other viral vectors, or combinations thereof. The proteins and one or more crRNAs can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. Exemplary delivery methods and the doses are described, e.g., in WO 2016205764 A1 and U.S. Pat. No. 8,454,972 B2, both of which are incorporated herein by reference in the entirety.

In some embodiments, the delivery is via a recombinant adeno-associated virus (rAAV) vector. For example, in some embodiments, a modified AAV vector may be used for delivery. Modified AAV vectors can be based on one or more of several capside types, including AAV1, AV2, AAV5, AAV6, AAV8, AAV 8.2. AAV9, AAV rhlO, modified AAV vectors (e.g., modified AAV2, modified AAV3, modified AAV6) and pseudotyped AAV (e.g., AAV2/8, AAV2/5 and AAV2/6). Exemplary AAV vectors and techniques that may be used to produce rAAV particles are known in the art (see, e.g., Aponte-Ubillus et al. (2018) Appl. Microbiol. Biotechnol. 102(3): 1045-54; Zhong et al. (2012) J. Genet. Syndr. Gene Ther. S1: 008; West et al. (1987) Virology 160: 38-47 (1987); Tratschin et al. (1985) Mol. Cell. Biol. 5: 3251-60); U.S. Pat. Nos. 4,797,368 and 5,173,414; and International Publication Nos. WO 2015/054653 and WO 93/24641, each of which are incorporated by reference).

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-associated proteins and/or an accessory protein, each operably linked to a promoter (e.g., the same promoter or a different promoter); (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR-associated proteins. In some embodiments, the CRISPR-associated proteins and/or RNA guides are coupled to one or more CPPs to effectively transport them inside cells (e.g., plant protoplasts). In some embodiments, the CRISPR-associated proteins and/or RNA guides are encoded by one or more circular or non-circular DNA molecules that are coupled to one or more CPPs for cell delivery.

CPPs are short peptides of fewer than 35 amino acids derived either from proteins or from chimeric sequences capable of transporting biomolecules across cell membrane in a receptor independent manner. CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin 3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

### Methods of Identifying CRISPR-Associated Protein Families

In one aspect, the disclosure relates to the use of computational methods and algorithms to search for and identify novel protein families that exhibit a strong co-occurrence pattern with certain other features within naturally occurring genone sequences.

In certain embodiments, these computational methods are directed to identifying protein families that co-occur in close proximity to CRISPR arrays. However, the methods disclosed herein are useful in identifying proteins that naturally occur within close proximity to other features, both non-coding and protein-coding (for example, CRISPR Cas1 proteins). It should be understood that the methods and calculations described herein may be performed on one or more computing devices.

In some embodiments, a set of genomic sequences are obtained from genomic or metagenomic databases. The databases comprise short reads, contig level data, assembled scaffolds, or complete organisms. Likewise, the database may comprise genomic sequence data from prokaryotic organisms, or eukarVotic organisms, or may include data from mretagenomic environmtental samples. Exemplary database repositories include NCBI RefSeq, NCBI GenBank, NCBI Whole Genome Shotgun (WGS), and JGI Integrated Microbial Genomes (IMG).

In some embodiments, a minimum size requirement is imposed to select genome sequence data of a specified minimum length. In certain exemplary embodiments, the minimum contig length may be 100 nucleotides, 500 nt, 1 kb, 2 kb, 3 kb, 4 kb, 5 kb, 10 kb, 20 kb, 40 kb, or 50 kb.

In some embodiments, known or predicted proteins are extracted from the complete or a selected set of genome sequence data. In some embodiments, known or predicted proteins are taken from extracting coding sequence (CDS) annotations provided by the source database. In some embodiments, predicted proteins are determined by applying a computational method to identify proteins from nucleotide sequences. In some embodiments, the GeneMark Suite is used to predict proteins from genome sequences. In some embodiments, Prodigal is used to predict proteins from genome sequences. In some embodiments, multiple protein prediction algorithms may be used over the same set of sequence data with the resulting set of proteins de-duplicated.

In some embodiments, CRISPR arrays are identified from the genome sequence data. In some embodiments, PILER-CR is used to identify CRISPR arrays. In some embodiments, CRISPR Recognition Tool (CRT) is used to identify CRISPR arrays. In some embodiments, multiple CRISPR array identification tools may be used over the same set of sequence data with the resulting set of CRISPR arrays de-duplicated.

In some embodiments, proteins in close proximity to CRISPR arrays are identified. In some embodiments, proximity is defined as a nucleotide distance, and may be within 20 kb, 15 kb, or 5 kb. In some embodiments, proximity is defined as the number of open reading frames (ORFs) between a protein and a CRISPR array, and certain exemplary distances may be 10, 5, 4, 3, 2, 1, or 0 ORFs. The proteins identified as being within close proximity to a CRISPR array are then grouped into clusters of homologous proteins. In some embodiments, blastclust is used to form protein clusters. In certain other embodiments, mmseqs2 is used to form protein clusters.

To establish a pattern of strong co-occurrence between the members of a protein cluster with CRISPR arrays, a BLAST search of each member of the protein family may be performed over the complete set of known and predicted proteins previously compiled. In some embodiments, UBLAST or mmseqs2 may be used to search for similar proteins. In some embodiments, a search may be performed only for a representative subset of proteins in the family.

In some embodiments, the clusters of proteins within close proximity to CRISPR arrays are ranked or filtered by a metric to determine co-occurrence. One exemplary metric is the ratio of the size of the protein cluster against the number of BLAST matches up to a certain E value threshold. In some embodiments, a constant E value threshold may be used. In other embodiments, the E value threshold may be determined by the most distant members of the protein cluster. In some embodiments, the global set of proteins is clustered and the co-occurrence metric is the ratio of the size of the CRISPR associated cluster against the size(s) of the containing global cluster(s).

In some embodiments, a manual review process is used to evaluate the potential functionality and the minimal set of components of an engineered system based on the naturally occurring locus structure of the proteins in the cluster. In some embodiments, a graphical representation of the protein cluster may assist in the manual review, and may contain information including pairwise sequence similarity, phylogenetic tree, source organisms/environments, and a graphical depiction of locus structures. In some embodiments, the graphical depiction of locus structures may filter for nearby protein families that have a high representation. In some embodiments, representation may be calculated by the ratio of the number of related nearby proteins against the size(s) of the containing global cluster(s). In certain exemplary embodiments, the graphical representation of the protein cluster may contain a depiction of the CRISPR array structures of the naturally occurring loci. In some embodiments, the graphical representation of the protein cluster may contain a depiction of the number of conserved direct repeats versus the length of the putative CRISPR array, or the number of unique spacer sequences versus the length of the putative CRISPR array. In some embodiments, the graphical representation of the protein cluster may contain a depiction of various metrics of co-occurrence of the putative effector with CRISPR arrays predict new CRISPR-Cas systems and identify their components.

The broad natural diversity of CRISPR-Cas defense systems contains a wide range of activity mechanisms and functional elements that can be harnessed for programmable biotechnologies. In a natural system, these mechanisms and parameters enable efficient defense against foreign DNA and viruses while providing self vs. non-self-discrimination to avoid self-targeting. In an engineered system, the same mechanisms and parameters also provide a diverse toolbox of molecular technologies and define the boundaries of the targeting space. For instance, systems Cas9 and Cas13a have canonical DNA and RNA endonuclease activity and their targeting spaces are defined by the protospacer adjacent motif (PAM) on targeted DNA and protospacer flanking sites (PFS) on targeted RNA, respectively.

The methods described herein can be used to discover additional mechanisms and parameters within single subunit Class 2 effector systems that can be more effectively harnessed for programmable biotechnologies.

### Pooled-Screening

To efficiently validate the activity of the engineered novel CRISPR-Cas systems and simultaneously evaluate in an unbiased manner different activity mechanisms and functional parameters, a new pooled-screening approach was developed in E. coli. First, from the computational identification of the conserved protein and noncoding elements of the novel CRISPR-Cas system, these separate components were assembled into an engineered locus, which in one embodiment is on a singe artificial expression vector based on the pET-28+a backbone; in another embodiment, multiple compatible expression plasmids were used to recapitulate the engineered locus. To construct the vector, in one embodiment, DNA synthesis was used to assemble the components together; in another embodiment, molecular cloning was used for assembly. In another embodiment, the proteins and noncoding elements are transcribed on a single mRNA transcript, and different ribosomal binding sites are used to translate individual proteins.

Second, a library of unprocessed crRNAs consisting of the direct repeat::spacer::direct repeat sequence was cloned into the engineered locus. In one embodiment, the spacers were targeting a second plasmid, pACYC184, and the spacers were of the length found in the natural CRISPR array. This crRNA library was cloned into the vector backbone containing the proteins and noncoding elements (e.g. pET-28a+), and then subsequently transformed the library into E. coli along with the second target plasmid (e.g., pACYC184). It is important to have the plasmid(s) containing the engineered loci be on compatible origin(s) of replication with respect to the target plasmid to enable bacterial co-transformation. Consequently, each resulting E. coli cell contains no more than one targeting spacer.

Third, the E. coli were grown under antibiotic selection. In one embodiment, triple antibiotic selection is used: kanamycin for ensuring successful transformation of the pET-28a+ vector containing the engineered CRISPR-Cas effector system, and chloramphenicol and tetracycline for ensuring successful co-transformation of the pACYC184 target vector. Since pACYC184 normally confers resistance to chloramphenicol and tetracycline, under antibiotic selection, positive activity of the novel CRISPR-Cas system targeting the plasmid will eliminate cells that actively express the proteins, noncoding elements, and specific active elements of the crRNA library. Using deep sequencing (e.g., next-generation sequencing), examining the population of surviving cells at a later time point compared to an earlier time point results in a depleted signal specifically for the active elements compared to the inactive crRNAs.

Since the pACYC184 plasmid contains a diverse set of features and sequences that may affect the activity of a CRISPR-Cas system, mapping the active crRNAs from the pooled screen onto pACYC184 provides patterns of activity that can be suggestive of different activity mechanisms and functional parameters in a broad, hypothesis-agnostic manner. In this way, the features required for reconstituting the novel CRISPR-Cas system in a heterologous prokaryotic species can be more comprehensively tested and studied.

The key advantages of the in vivo pooled-screen described herein include:

(1) Versatility—engineered locus design allows multiple proteins and/or noncoding elements to be expressed; the library cloning strategy enables both transcriptional directions of the computationally predicted crRNA to be expressed;

(2) Comprehensive tests of activity mechanisms & functional parameters—Evaluates diverse interference mechanisms, including DNA or RNA cleavage; examines co-occurrence of features such as transcription, plasmid DNA replication; and flanking sequences for crRNA library can be used to reliably determine PAMs with complexity equivalence of 4N's;

(3) Sensitivity—pACYC184 is a low copy plasmid, enabling high sensitivity for CRISPR-Cas activity since even modest interference rates can eliminate the antibiotic resistance encoded by the plasmid; and

(4) Efficiency—Optimized molecular biology steps to enable greater speed and throughput RNA-sequencing and protein expression samples can be directly harvested from the surviving cells in the screen.

The novel CRISPR-Cas families described herein were evaluated using this in vivo pooled-screen to evaluate their operational elements, mechanisms and parameters, as well as their ability to be active and reprogrammed in an engineered system outside of their natural cellular environment.

### EXAMPLES

The invention is further described in the following examples, which do not limit the scope of the invention described in the claims.

### Example 1—Building an Expanded Database of CRISPR-Cas Systems, and Searching for Type VI-D RNA-Targeting Systems

We developed a computational pipeline to produce an expanded database of class 2 CRISPR-Cas systems from genomic and metagenomic sources. Genome and metagenome sequences were downloaded from NCBI (Benson et al., 2013; Pruitt et al., 2012), NCBI whole genome sequencing (WGS), and DOE JGI Integrated Microbial Genomes (Markowitz et al., 2012). Proteins were predicted (Meta-GeneMark (Zhu et al., 2010) using the standard model MetaGeneMark_v1.mod, and Prodigal (Hyatt et al., 2010) in anon mode) on all contigs at least 5 kb in length, and de-duplicated in favor of pre-existing annotations to construct a complete protein database. CRISPR arrays were identified and protein sequences for ORFs located within +/−10 kb from CRISPR arrays were grouped into CRISPR-proximal protein clusters. Clusters of fewer than 4 proteins, or comprising proteins from fewer than 3 contigs were discarded. Each of these remaining protein clusters were considered to be a putative effector of a CRISPR-Cas system. In addition to the CRISPR array and putative effector protein, many CRISPR-Cas systems also include additional proteins that enable adaptation, crRNA processing, and defense. Potential additional CRISPR-Cas system components associated with each of the predicted effectors were identified as clusters of protein-coding genes with high effector co-occurrence, and CRISPR enrichment or CRISPR representation of at least 15%.

Effector co-occurrence was calculated as the percentage of loci containing the effector that also contain the potential co-occurring protein. The high co-occurrence threshold was a function of the cohesiveness of the effector cluster (more homogenous clusters requiring a higher threshold). The CRISPR enrichment was calculated as follows: 1) Up to 20 unique proteins were sampled from each protein cluster, and UBLAST (Edgar, 2010) was used to generate a rank ordered list of proteins by E-value from the complete protein database, 2) An E-value threshold was imposed to recover at least 50% of the members of the cluster, and 3) CRISPR enrichment was calculated by dividing the number of CRISPR-proximal proteins below the E-value threshold by the total number of proteins below the threshold. CRISPR representation was calculated as the percentage of effector-proximal proteins in a CRISPR-proximal protein cluster. All clustering operations were performed using mmseqs2 (Steinegger and Söding, 2017).

This information was incorporated into a database of (predicted) CRISPR-Cas systems, each composed of: 1) a CRISPR array, 2) a putative effector, and optionally, 3) clusters of potential co-acting proteins. Aggregating and processing a collection of more than 10 Tb of prokaryotic genomic and metagenomic sequence data from multiple sources, our pipeline produced a database of 293,985 putative CRISPR-Cas systems. One important difference from previously reported computational pipelines (Shmakov et al., 2015, 2017a; Smargon et al., 2017) is that we perform minimal filtering (e.g., imposing a minimum size on putative effector) in the intermediate stages of the search in order to expand the range for potential discovery of novel CRISPR-Cas systems. As such, the resulting database of putative CRISPR-Cas loci includes all previously characterized class 2 CRISPR-Cas systems, but also contains a considerable amount of noise, such as degraded, non-functional CRISPR-Cas loci.

For functional characterization of this database of candidate CRISPR-Cas systems, we constructed multiple sequence alignment for each family of putative effectors using MAFFT (Katoh and Standley, 2013) and conducted an HMM search using HMMer (Eddy, 2011) against protein family databases Pfam (Finn et al., 2014) and Uniprot (Bateman et al., 2017), as well as a BLASTN search of CRISPR spacer sequences against a reference set of phages. This analysis led to the detection of protein families corresponding to all previously identified class 2 CRISPR-Cas systems, indicating a minimal false negative rate. To identify novel class 2 CRISPR-Cas systems, features included above for the prediction of the functions of putative CRISPR-Cas systems were used to rank candidate families for follow-up functional evaluation.

### Genomic Survey of Type VI-D RNA-Targeting CRISPR-Cas Systems

To expand the repertoire of Cas nucleases for RNA manipulation and sensing, we searched our database for type VI CRISPR-Cas systems with effector proteins containing two HEPN-domains each (2-HEPN proteins). In addition to the previously identified 2-HEPN proteins, Cas13a, Cas13b, and Cas13c, we detected a group of 2-HEPN proteins distantly related to Cas13a (effectors of type VI-A), primarily in Eubacterium and Ruminococcus, which we denote Cas13d. The amino acid sequences of Cas13d proteins show less than 8% identity to the most similar Cas13a sequences; nevertheless, statistically significant sequence similarity between Cas13d and Cas13a can be demonstrated using PSI-BLAST initiated with a profile made from the multiple alignment of Cas13a (E-value=0.002). This significant similarity is primarily due to the conservation of the HEPN domain sequences between Cas13a and Cas13d, whereas the remaining portions of the protein sequences in the two families are highly divergent; in particular, Cas13d proteins lack a counterpart to the Helical-1 domain of Cas13a (FIGS. 5A-C). Phylogenetic analysis of the Cas13 proteins clearly shows that Cas13a and Cas13d form strongly supported clades (FIGS. 4A-B).

Additionally, Cas13d effectors are notably smaller than previously characterized class 2 CRISPR effectors, with a median size of 928 aa. For comparison, this median size is 190 aa (17%) less than that of Cas13c, more than 200 aa (18%) less than that of Cas13b, and more than 300 aa (26%) less than that of Cas13a (FIG. 2B). Taken together, these lines of evidence suggest that this distinct group of class 2 CRISPR-Cas systems are best classified as Type VI-D, with the effector denoted Cas13d (FIG. 2A).

We found that 77% of Cas13d genes occur adjacent to CRISPR arrays, and for 19%, the adaptation module (Cas1 and Cas2 genes) is present in the vicinity (FIG. 1), suggesting that many Type VI-D loci encode CRISPR-Cas systems that are active in both adaptation and interference. Phylogenetic analysis indicates that Cas1 proteins associated with Type VI-D are monophyletic and, in accord with previous observations on other type VI systems, are affiliated with the type II-A clade (FIG. 3). Thus, in the case of type VI, the adaptation module seems to have co-evolved with the effector module.

Spacer sequences from CRISPR arrays within 3 kb of Cas13d effectors were extracted. In the case of multiple contigs containing the same Cas13d sequence (e.g., duplicated locus), only the contig containing the longest CRISPR array was used. Subsequent spacer analysis closely follows the method described previously (Shmakov et al., 2017b). Briefly, the resulting 198 spacers were de-duplicated by comparison of direct and reverse complement sequences, to produce a set of 182 unique spacers. A BLASTN (Camacho et al., 2009) search with the command line parameters -word_size 7 -gapopen 5 -gapextend 2 -reward 1 -penalty -3 was performed for the unique spacer set against a database comprising the virus and prokaryotic sequences in NCBI. To identify prophage regions, (i) all ORFs within 3 kb of prokaryotic matches were collected; (ii) a PSI-BLAST search was conducted against the proteins extracted from the virus part of NCBI, using the command line parameters -seg no -evalue 0.000001 -dbsize 20000000; and (iii) a spacer hit was classified as prophage if it overlapped with an ORF with a viral match, or if two or more ORFs with viral matches were identified within the neighborhood of the spacer hit.

The CRISPR arrays adjacent to Cas13d genes contain 198 spacers total, of which 182 are unique. A BLASTN search of the unique spacer sequences against a database comprising known phages and NCBI prokaryotic sequences revealed 7 spacers with significant hits (defined as E-value<0.0001, alignment length at least 24, 0 gaps, and no more than one mismatch). One spacer, from Ruminococcus flavefaciens FD-1, showed significant matches against the Arthrobacter dsDNA phage Gordon (alignment length=28, 1 mismatch) and against a putative prophage region in an uncultured Flavonifractor sequence (alignment length=24, 0 mismatches). A different spacer, from a gut metagenome sequence, resulted in a significant match against a putative prophage region in Bacillus soli (alignment length=24, 0 mismatches). The remaining five spacer matches targeted ORFs in prokaryotic sequences, but were not classified as being in prophage regions. The presence of spacers homologous to DNA phage genomic sequences in an RNA-targeting CRISPR-Cas system might appear unexpected but is in line with similar observations on type VI-A and type VI-B systems (Smargon et al., 2017). Presumably, type VI systems abrogate the reproduction of DNA phages by cleaving phage mRNAs, but the mechanistic details of the antivirus activity of these systems remain to be characterized experimentally.

Examination of the additional genes in the vicinity of Cas13d led to the identification in most of the VI-D loci of potential accessory proteins containing WYL domains (so denoted after three amino acids that were conserved in the originally identified group of these domains) and additionally, ribbon-helix-helix (RHH) DNA-binding domains (FIG. 6).

For phylogenetic analysis of these Cas13d-associated WYL-domain containing proteins, we compiled a data set of WYL proteins. In addition to automatically identified WYL proteins, we used PSI-BLAST (Altschul et al., 1997) to search over a local set of NCBI sourced proteins using RspWYL1 as a query. The results with E-value 0.01 or lower were added to the set of WYL proteins. Proteins smaller than 150 aa were discarded from the data set, and UCLUST (Edgar, 2010) with identity threshold 0.90 was used to obtain a non-redundant set. We then added all WYL proteins identified in the vicinity of Cas13d genes to form a set of 3908 WYL sequences for phylogenetic analysis. Multiple alignment and phylogeny of protein sequences were constructed as described previously (Peters et al., 2017).

Briefly, the sequences were clustered by similarity, and for each cluster, a multiple alignment was built using MUSCLE (Edgar, 2004). Alignments were combined into larger aligned clusters by HHalign (Yu et al., 2015) if the resulting score between the two alignments was higher than the threshold; otherwise, the scores were recorded in a similarity matrix. The matrix was used to reconstruct a UPGMA tree. For each cluster, the alignment was filtered as follows: the alignment positions with the gap character fraction values of 0.5 and homogeneity values of 0.1 or less were removed. The remaining positions were used for tree reconstruction using FastTree with the WAG evolutionary model and the discrete gamma model with 20 rate categories. The same program was used to compute SH (Shimodaira-Hasegawa)-like node support values

The WYL-domain proteins contained in Type VI-D loci fall into six strongly supported branches of the broader phylogenetic tree of WYL-domain proteins. The branch we denote WYL1 is a single WYL-domain protein associated primarily with Ruminococcus. Multiple sequence alignment of WYL1 shows an N-terminal RHH domain, as well as a pattern of primarily hydrophobic conserved residues, including an invariant tyrosine-leucine doublet corresponding to the original WYL motif (FIG. 7). Other VI-D loci contain duplicated genes encoding WYL-domain proteins, as in Ruminococcus flavefaciens, or a fusion of two WYL-domain proteins, as in Eubacterium sp. Although a substantial majority of the VI-D loci encode WYL-domain proteins, phylogenetic analysis indicates that these CRISPR-associated WYL proteins are scattered among different branches of the WYL family tree, i.e., are polyphyletic. Thus, the VI-D CRISPR-Cas systems appear to have acquired WYL-domain proteins on several independent occasions, suggesting a role for this protein in modulating the CRISPR-Cas function.

Exemplary Type VI-D CRISPR-Cas effector proteins are provided in TABLES 1 and 2 (e.g., SEQ ID NOs. 1-31, and 200-350). Exemplary Type VI-D CRISPR-Cas direct repeat sequences are provided in TABLE 3 (e.g., SEQ ID NOs 32-49, 52-77, 351-589). Exemplary Type VI-D CRISPR-Cas associated WYL accessory proteins are provided in TABLES 1, 4, 5, and 6. In some embodiments, a Type VI-D CRISPR-Cas effector protein comprises an exemplary motif provided in TABLE 7 (e.g., SEQ ID NOs: 94-98, 672 and 673).

TABLE 1
Representative Cas13d Effector and WYL1 Accessory Proteins

| | | | # | | | Effector |
|---|---|---|---|---|---|---|
| Species | Cas13d Accession | WYL1 Accession | spacers | cas1 | cas2 | size |
| Eubacterium sp. Ann11 (NZ_NFLV01000009) | NZ_NFLV01000009_111 | N/A | 9 | Y | Y | 1006 |
| Eubacterium sp. An3 (NFIR01000008) | NFIR01000008_78 | N/A | 2 | Y | Y | 1001 |
| Ruminococcus albus (NZ_FOAT01000009) | WP_074833651.1 | N/A | 6 | N | N | 944 |
| Ruminococcus bicirculans (NZ_HF545617) | WP_041337480.1 | WP_041337479.1 | 6 | N | N | 918 |
| Ruminococcus flavefaciens (DBYI01000091) | DBYI01000091_43 | N/A | 11 | Y | Y | 958 |
| Ruminococcus flavefaciens (NZ_FPJT01000005) | WP_075424065.1 | N/A | 4 | N | N | 967 |
| Ruminococcus flavefaciens FD-1 (NZ_ACOK01000100) | WP_009985792.1 | N/A | 5 | N | N | 933 |
| Ruminococcus flavefaciens FD-1 (NZ_ACOK01000100) | NZ_ACOK01000100_5 | N/A | 5 | N | N | 949 |
| Ruminococcus sp. CAG:57 (CBFS010000062) | CDC65743.1 | SCH71532.1 | 2 | N | N | 922 |
| Ruminococcus sp. N15.MGS-57 (LARF01000048) | LARF01000048_8 | LARF01000048_7 | 3 | N | N | 919 |
| Ruminococcus sp. UBA7013 (DJXD01000002) | DJXD01000002_3 | N/A | 9 | Y | Y | 877 |
| Eubacterium siraeum DSM 15702 (DS499551) | WP_005358205.1 | N/A | 18 | N | N | 954 |
| Eubacterium siraeum DSM 15702 (NZ_KB907524) | WP_005358205.1 | N/A | 7 | N | N | 954 |
| animal-digestive system-orangutan individual fecal | 3300010266 \| Ga0129314_1001134_19 | N/A | 6 | N | N | 981 |
| (3300010266 \| Ga0129314_1001134) | | | | | | |
| arthropoda-digestive system-cubitermes and | 3300006226 \| Ga0099364_10024192_5 | N/A | 13 | Y | Y | 1054 |
| nasutitermes termite gut | | | | | | |
| (3300006226 \| Ga0099364_10024192) | | | | | | |
| arthropoda-digestive system-cubitermes and | 3300006226 \| Ga0099364_10024192_5 | N/A | 13 | Y | Y | 1043 |
| nasutitermes termite gut | | | | | | |
| (3300006226 \| Ga0099364_10024192) | | | | | | |
| gut metagenome (CDTW01032418) | CDTW01032418_55 | CDTW01032418_59 | 4 | N | N | 906 |
| gut metagenome (CDYS01033339) | CDYS01033339_14 | CDYS01033339_20 | 5 | N | N | 906 |
| gut metagenome (CDYU01004315) | CDYU01004315_2 | CDYU01004315_3 | 2 | N | N | 925 |
| gut metagenome (CDYU01023067) | CDYU01023067_140 | N/A | 5 | N | N | 906 |
| gut metagenome (CDYX01024884) | CDYX01024884_4 | CDYX01024884_5 | 8 | N | N | 923 |
| gut metagenome (CDZD01043528) | CDZD01043528_308 | N/A | 4 | N | N | 906 |
| gut metagenome (CDZE01002059) | CDZE01002059_22 | CDZE01002059_21 | 8 | N | N | 923 |
| gut metagenome (CDZF01024873) | CDZF01024873_75 | N/A | 4 | N | N | 906 |
| gut metagenome (CDZF01043927) | CDZF01043927_109 | N/A | 4 | N | N | 906 |
| gut metagenome (CDZK01015063) | CDZK01015063_14 | N/A | 3 | N | N | 923 |
| gut metagenome (CDZK01015063) | CDZK01015063_14 | N/A | 3 | N | N | 921 |
| gut metagenome (CDZR01037537) | SCH71549.1 | SCH71532.1 | 2 | N | N | 922 |
| gut metagenome (CDZT01047721) | CDZT01047721_3 | WP_041337479.1 | 4 | N | N | 929 |
| gut metagenome (CDZU01022944) | CDZU01022944_3 | WP_041337479.1 | 4 | N | N | 929 |
| gut metagenome (CDZV01031905) | CDZV01031905_3 | WP_041337479.1 | 4 | N | N | 929 |
| gut metagenome (CEAA01017658) | CEAA01017658_2 | N/A | 3 | N | N | 922 |
| gut metagenome (OCTW011587266) | OCTW011587266_5 | N/A | 2 | N | N | 911 |
| gut metagenome (OCVV011003687) | OCVV011003687_3 | N/A | 7 | N | N | 947 |
| gut metagenome (OCVV011003687) | OCVV011003687_3 | N/A | 7 | N | N | 955 |
| gut metagenome (ODAI010069496) | ODAI010069496_4 | N/A | 2 | N | N | 824 |
| gut metagenome (ODAI011611274) | ODAI011611274_2 | N/A | 4 | Y | N | 1009 |
| human gut metagenome (OATA01000148) | OATA01000148_47 | OATA01000148_62 | 13 | N | N | 918 |
| human gut metagenome (OAVJ01001264) | OAVJ01001264_7 | OAVJ01001264_6 | 3 | N | N | 921 |
| human gut metagenome (OBAE01000973) | OBAE01000973_3 | OBAE01000973_4 | 5 | N | N | 923 |
| human gut metagenome (OBAI01000753) | OBAI01000753_39 | N/A | 9 | N | N | 918 |
| human gut metagenome (OBAQ01000162) | OBAQ01000162_41 | OBAQ01000162_28 | 13 | N | N | 918 |
| human gut metagenome (OBAR01000289) | OBAR01000289_55 | N/A | 9 | N | N | 922 |
| human gut metagenome (OBAS01000138) | OBAS01000138_55 | OBAS01000138_57 | 11 | N | N | 922 |
| human gut metagenome (OBCV01000332) | OBCV01000332_2 | OBCV01000332_3 | 2 | N | N | 922 |
| human gut metagenome (OBDE01000870) | OBDE01000870_1 | N/A | 5 | N | N | 796 |
| human gut metagenome (OBHU01001207) | SCJ27598.1 | SCJ27525.1 | 9 | N | N | 919 |
| human gut metagenome (OBII01002626) | OBII01002626_5 | N/A | 5 | N | N | 860 |
| human gut metagenome (OBII01002626) | OBII01002626_3 | N/A | 5 | N | N | 850 |
| human gut metagenome (OBJF01000033) | OBJF01000033_8 | N/A | 6 | N | N | 955 |
| human gut metagenome (OBJF01000033) | OBJF01000033_8 | N/A | 6 | N | N | 939 |
| human gut metagenome (OBKG01000025) | OBKG01000025_26 | OBKG01000025_25 | 8 | N | N | 922 |
| human gut metagenome (OBKR01000858) | OBKR01000858_3 | OBKR01000858_4 | 5 | N | N | 922 |
| human gut metagenome (OBVH01003037) | OBVH01003037_1 | N/A | 6 | N | N | 955 |
| human gut metagenome (OBVH01003037) | OBVH01003037_2 | N/A | 6 | N | N | 939 |
| human gut metagenome (OBVY01000267) | OBVY01000267_8 | 0BVY01000267_8 | 5 | N | N | 924 |
| human gut metagenome (OBXZ01000094) | OBXZ01000094_20 | N/A | 2 | N | N | 943 |
| human gut metagenome (OBXZ01000094) | OBXZ01000094_20 | N/A | 2 | N | N | 939 |
| human gut metagenome (OCHB01002119) | OCHB01002119_1 | OCHB01002119_2 | 2 | N | N | 925 |
| human gut metagenome (OCHC01000012) | OCHC01000012_250 | OCHC01000012_251 | 7 | N | N | 919 |
| human gut metagenome (OCHD01001741) | OCHD01001741_1 | N/A | 9 | N | N | 922 |
| human gut metagenome (OCHE01000387) | OCHE01000387_10 | OCHE01000387_8 | 5 | N | N | 922 |
| human gut metagenome (OCHK01000325) | OCHK01000325_37 | OCHK01000325_38 | 11 | N | N | 922 |
| human gut metagenome (OCHN01000290) | OCHN01000290_35 | N/A | 22 | N | N | 803 |
| human gut metagenome (OCHS01000450) | OCHS01000450_6 | N/A | 9 | N | N | 922 |
| human gut metagenome (OCHU01001749) | OCHU01001749_1 | N/A | 11 | N | N | 918 |
| human gut metagenome (OCPQ01000020) | OCPQ01000020_138 | OCPQ01000020_137 | 8 | N | N | 919 |
| human gut metagenome (OCPS01000464) | OCPS01000464_4 | OCPS01000464_5 | 4 | N | N | 919 |
| human gut metagenome (OCPU01001206) | OCPU01001206_17 | OCPU01001206_15 | 4 | N | N | 808 |
| human gut metagenome (OCPV01000148) | OCPV01000148_47 | OCPV01000148_62 | 16 | N | N | 918 |
| human gut metagenome (OCQA01000142) | OCQA01000142_55 | OCQA01000142_56 | 11 | N | N | 922 |
| human gut metagenome (OFMN01000509) | OFMN01000509_2 | N/A | 12 | N | N | 918 |
| human gut metagenome (OFMU01000310) | OFMU01000310_31 | OFMU01000310_30 | 5 | N | N | 922 |
| human gut metagenome (OFMV01000268) | OFMV01000268_25 | OFMV01000268_23 | 5 | N | N | 924 |
| human gut metagenome (OFRY01000077) | OFRY01000077_43 | OFRY01000077_29 | 11 | N | N | 918 |
| human gut metagenome (OGCM01002738) | OGCM01002738_3 | OGCM01002738_4 | 4 | N | N | 919 |
| human gut metagenome (OGCO01000353) | OGCO01000353_15 | OGCO01000353_16 | 2 | N | N | 922 |
| human gut metagenome (OGCQ01002817) | SCJ27598.1 | N/A | 7 | N | N | 919 |
| human gut metagenome (OGOC01002653) | OGOC01002653_3 | OGOC01002653_4 | 5 | N | N | 924 |
| human gut metagenome (OGOI01001249) | OGOI01001249_5 | OGOI01001249_4 | 5 | N | N | 922 |
| human gut metagenome (OGOK01000323) | OGOK01000323_15 | N/A | 10 | N | N | 921 |
| human gut metagenome (OGOL01000786) | OGOL01000786_27 | OGOL01000786_26 | 6 | N | N | 922 |
| human gut metagenome (OGOO01001137) | OGOO01001137_18 | OGOO01001137_17 | 5 | N | N | 920 |
| human gut metagenome (OGOP01001824) | OGOP01001824_10 | OGOP01001824_8 | 5 | N | N | 921 |
| human gut metagenome (OGOY01000326) | SCH71549.1 | SCH71532.1 | 2 | N | N | 922 |
| human gut metagenome (OGPA01000243) | OGPA01000243_2 | WP_041337479.1 | 4 | N | N | 929 |
| human gut metagenome (OGPB01000314) | OGPB01000314_7 | OGPB01000314_5 | 5 | N | N | 922 |
| human gut metagenome (OGPJ01000449) | OGPJ01000449_26 | OGPJ01000449_25 | 3 | N | N | 919 |
| human gut metagenome (OGPK01001709) | OGPK01001709_2 | OGPK01001709_3 | 3 | N | N | 919 |
| human gut metagenome (OGPQ01001037) | OGPQ01001037_3 | OGPQ01001037_4 | 3 | N | N | 922 |
| human gut metagenome (OGPS01000624) | OGPS01000624_23 | N/A | 12 | N | N | 954 |
| human gut metagenome (OGPS01000672) | OGPS01000672_3 | OGPS01000672_4 | 6 | N | N | 919 |
| human gut metagenome (OGPU01000173) | OGPU01000173_30 | OGPU01000173_31 | 5 | N | N | 922 |
| human gut metagenome (OGPY01000296) | SCH71549.1 | OGPY01000296_5 | 2 | N | N | 922 |
| human gut metagenome (OGQH01000331) | OGQH01000331_48 | OGQH01000331_47 | 2 | N | N | 919 |
| human gut metagenome (OGQO01007270) | OGQO01007270_2 | OGQO01007270_1 | 2 | N | N | 922 |
| human gut metagenome (OGQU01002289) | OGQU01002289_9 | OGQU01002289_8 | 5 | N | N | 924 |
| human gut metagenome (OGQV01000794) | OGQV01000794_21 | OGQV01000794_21 | 3 | N | N | 922 |
| human gut metagenome (OGQW01001429) | OGQW01001429_6 | OGQW01001429_5 | 5 | N | N | 915 |
| human gut metagenome (OGQX01000605) | OGQX01000605_8 | OGQX01000605_9 | 6 | N | N | 919 |
| human gut metagenome (OGQZ01000194) | OGQZ01000194_33 | OGQZ01000194_32 | 4 | N | N | 922 |
| human gut metagenome (OGRA01000610) | OGRA01000610_24 | OGRA01000610_25 | 5 | N | N | 922 |
| human gut metagenome (OGRE01001635) | OGRE01001635_6 | OGRE01001635_5 | 5 | N | N | 926 |
| human gut metagenome (OGRF01000967) | OGRF01000967_2 | OGRF01000967_4 | 5 | N | N | 922 |
| human gut metagenome (OGRG01000028) | OGRG01000028_3 | OGRG01000028_5 | 3 | N | N | 919 |
| human gut metagenome (OGRH01000378) | OGRH01000378_2 | N/A | 11 | N | N | 918 |
| human gut metagenome (OGRN01001989) | OGRN01001989_2 | N/A | 8 | N | N | 925 |
| human gut metagenome (OGRQ01003333) | OGRQ01003333_5 | OGRQ01003333_4 | 7 | N | N | 923 |
| human gut metagenome (OGRT01000617) | OGRT01000617_3 | OGRT01000617_5 | 5 | N | N | 921 |
| human gut metagenome (OGRU01000829) | OGRU01000829_2 | OGRU01000829_3 | 5 | N | N | 915 |
| human gut metagenome (OGSD01001176) | OGSD01001176_18 | OGSD01001176_17 | 3 | N | N | 922 |
| human gut metagenome (OGUL01000592) | OGUL01000592_19 | OGUL01000592_6 | 7 | N | N | 918 |
| human gut metagenome (OGWY01002732) | OGWY01002732_3 | N/A | 10 | N | N | 952 |
| human gut metagenome (OGXI01000433) | OGXI01000433_6 | OGXI01000433_8 | 5 | N | N | 922 |
| human gut metagenome (OGXJ01002463) | OGXJ01002463_5 | OGXJ01002463_4 | 2 | N | N | 922 |
| human gut metagenome (OGXL01002096) | OGXL01002096_10 | OGXL01002096_9 | 4 | N | N | 923 |
| human gut metagenome (OGYD01000683) | OGYD01000683_23 | OGYD01000683_21 | 2 | N | N | 919 |
| human gut metagenome (OGYL01002810) | OGYL01002810_3 | WP_041337479.1 | 3 | N | N | 925 |
| human gut metagenome (OGYU01002161) | OGYU01002161_4 | OGYU01002161_2 | 5 | N | N | 922 |
| human gut metagenome (OGYY01000371) | OGYY01000371_37 | OGYY01000371_36 | 4 | N | N | 922 |
| human gut metagenome (OGZC01000639) | OGZC01000639_10 | N/A | 12 | N | N | 984 |
| human gut metagenome (OHAI01000724) | OHAI01000724_7 | OHAI01000724_6 | 5 | N | N | 922 |
| human gut metagenome (OHAJ01000052) | OHAJ01000052_20 | N/A | 3 | N | N | 956 |
| human gut metagenome (OHAN01001071) | OHAN01001071_11 | OHAN01001071_10 | 4 | N | N | 922 |
| human gut metagenome (OHAR01000226) | OHAR01000226_9 | OHAR01000226_10 | 3 | N | N | 926 |
| human gut metagenome (OHBL01000590) | OHBL01000590_7 | OHBL01000590_6 | 5 | N | N | 919 |
| human gut metagenome (OHBM01000552) | OHBM01000552_13 | OHBM01000552_14 | 2 | N | N | 922 |
| human gut metagenome (OHBP01000023) | OHBP01000023_129 | SCH71532.1 | 3 | N | N | 922 |
| human gut metagenome (OHBQ01000429) | OHBQ01000429_2 | N/A | 3 | N | N | 928 |
| human gut metagenome (OHBW01001448) | OHBW01001448_1 | OHBW01001448_2 | 5 | N | N | 924 |
| human gut metagenome (OHCE01000125) | OHCE01000125_17 | OHCE01000125_19 | 6 | N | N | 918 |
| human gut metagenome (OHCH01000211) | OHCH01000211_3 | OHCH01000211_4 | 4 | N | N | 922 |
| human gut metagenome (OHCP01000044) | OHCP01000044_27 | N/A | 6 | Y | N | 1023 |
| human gut metagenome (OHCW01000317) | OHCW01000317_3 | OHCW01000317_6 | 8 | N | N | 921 |
| human gut metagenome (OHDC01002972) | OHDC01002972_3 | N/A | 6 | N | N | 921 |
| human gut metagenome (OHDP01000241) | OHDP01000241_4 | N/A | 19 | N | N | 954 |
| human gut metagenome (OHDS01000019) | OHDS01000019_133 | SCH71532.1 | 3 | N | N | 922 |
| human gut metagenome (OHDT01000502) | OHDT01000502_2 | N/A | 2 | N | N | 925 |
| human gut metagenome (OHEG01001211) | OHEG01001211_2 | OHEG01001211_3 | 4 | N | N | 924 |
| human gut metagenome (OHEL01001488) | OHEL01001488_6 | OHEL01001488_5 | 3 | N | N | 928 |
| human gut metagenome (OHFA01000290) | OHFA01000290_5 | N/A | 21 | N | N | 954 |
| human gut metagenome (OHFV01000201) | OHFV01000201_5 | N/A | 19 | N | N | 954 |
| human gut metagenome (OHFX01001477) | OHFX01001477_3 | OHFX01001477_2 | 3 | N | N | 922 |
| human gut metagenome (OHGN01001355) | OHGN01001355_3 | N/A | 3 | N | N | 926 |
| human gut metagenome (OHGX01000264) | OHGX01000264_3 | OHGX01000264_3 | 4 | N | N | 925 |
| human gut metagenome (OHHD01000480) | OHHD01000480_3 | OHHD01000480_4 | 3 | N | N | 926 |
| human gut metagenome (OHHR01000227) | OHHR01000227_3 | OHHR01000227_4 | 5 | N | N | 922 |
| human gut metagenome (OHIB01002708) | OHIB01002708_3 | N/A | 3 | N | N | 818 |
| human gut metagenome (OHIJ101000315) | OHIJ01000315_7 | OHIJ01000315_5 | 5 | N | N | 922 |
| human gut metagenome (OHJG01000198) | OHJG01000198_33 | OHJG01000198_31 | 4 | N | N | 918 |
| human gut metagenome (OHJJ101000127) | OHJJ01000127_35 | OHJJ01000127_33 | 6 | N | N | 918 |
| human gut metagenome (OHJK01001285) | OHJK01001285_9 | N/A | 10 | N | N | 1001 |
| human gut metagenome (OHJS01001864) | OHJS01001864_3 | OHJS01001864_5 | 5 | N | N | 921 |
| human gut metagenome (OHJT01001977) | OHJT01001977_4 | N/A | 4 | N | N | 954 |
| human gut metagenome (OHJZ01000157) | OHJZ01000157_5 | N/A | 21 | N | N | 954 |
| human gut metagenome (OHKC01000402) | OHKC01000402_5 | OHKC01000402_6 | 3 | N | N | 926 |
| human gut metagenome (OHKH01000861) | OHKH01000861_3 | OHKH01000861_2 | 3 | N | N | 928 |
| human gut metagenome (OHKW01000215) | OHKW01000215_41 | OHKW01000215_38 | 8 | N | N | 921 |
| human gut metagenome (OHLH01003112) | OHLH01003112_3 | N/A | 5 | N | N | 921 |
| human gut metagenome (OHLO01000586) | OHLO01000586_3 | OHLO01000586_4 | 5 | N | N | 919 |
| human gut metagenome (OHLY01001101) | OHLY01001101_3 | N/A | 10 | N | N | 954 |
| human gut metagenome (OHME01000303) | OHME01000303_3 | OHME01000303_4 | 4 | N | N | 925 |
| human gut metagenome (OHMF01000395) | OHMF01000395_24 | OHMF01000395_25 | 3 | N | N | 923 |
| human gut metagenome (OHMH01000024) | OHMH01000024_3 | SCH71532.1 | 3 | N | N | 922 |
| human gut metagenome (OHMQ01000465) | OHMQ01000465_4 | OHMQ01000465_2 | 5 | N | N | 922 |
| human gut metagenome (OHMW01000451) | OHMW01000451_18 | OHMW01000451_20 | 3 | N | N | 922 |
| human gut metagenome (OHNF01001864) | OHNF01001864_4 | OHNF01001864_6 | 3 | N | N | 922 |
| human gut metagenome (OHNP01000278) | OHNP01000278_34 | OHNP01000278_33 | 4 | N | N | 925 |
| human gut metagenome (OHOI01000307) | OHOI01000307_2 | OHOI01000307_3 | 4 | N | N | 925 |
| human gut metagenome (OHOK01001322) | OHOK01001322_2 | OHOK01001322_3 | 5 | N | N | 923 |
| human gut metagenome (OHPC01000165) | OHPC01000165_40 | OHPC01000165_39 | 5 | N | N | 922 |
| human gut metagenome (OHPD01001131) | OHPD01001131_4 | N/A | 8 | N | N | 954 |
| human gut metagenome (OHPE01000834) | OHPE01000834_1 | N/A | 5 | N | N | 922 |
| human gut metagenome (OHPP01000240) | OHPP01000240_36 | OHPP01000240_35 | 8 | N | N | 921 |
| human gut metagenome (OHPW01002065) | OHPW01002065_2 | N/A | 10 | N | N | 954 |
| human gut metagenome (OHQE01002584) | OHQE01002584_3 | N/A | 3 | N | N | 922 |
| human gut metagenome (OHRD01000126) | OHRD01000126_17 | OHRD01000126_19 | 7 | N | N | 918 |
| human gut metagenome (OHRM01001189) | OHRM01001189_3 | OHRM01001189_5 | 8 | N | N | 921 |
| human gut metagenome (OHSG01000119) | OHSG01000119_6 | OHSG01000119_5 | 2 | N | N | 924 |
| human gut metagenome (OHSI01000544) | OHSI01000544_10 | N/A | 15 | N | N | 1001 |
| human gut metagenome (OHSM01000196) | OHSM01000196_10 | N/A | 6 | Y | N | 1023 |
| human gut metagenome (OHSQ01001407) | OHSQ01001407_1 | OHSQ01001407_2 | 5 | N | N | 924 |
| human gut metagenome (OHST01000977) | OHST01000977_4 | N/A | 13 | N | N | 954 |
| human gut metagenome (OHSZ01000559) | OHSZ01000559_4 | OHSZ01000559_5 | 5 | N | N | 919 |
| human gut metagenome (OHTG01000221) | OHTG01000221_40 | OHTG01000221_38 | 8 | N | N | 921 |
| human gut metagenome (OHTH01000201) | OHTH01000201_42 | OHTH01000201_39 | 8 | N | N | 921 |
| human gut metagenome (OHUA01000395) | OHUA01000395_26 | OHUA01000395_24 | 5 | N | N | 923 |
| human gut metagenome (OHUN01000170) | OHUN01000170_40 | OHUN01000170_39 | 5 | N | N | 922 |
| human gut metagenome (OHUP01000072) | SCJ27598.1 | SCJ27525.1 | 7 | N | N | 919 |
| human gut metagenome (OHUY01000263) | OHUY01000263_2 | OHUY01000263_5 | 7 | N | N | 919 |
| human gut metagenome (OHVU01001109) | OHVU01001109_1 | N/A | 5 | N | N | 919 |
| human gut metagenome (OHWI01000399) | SCJ27598.1 | SCJ27525.1 | 4 | N | N | 919 |
| human gut metagenome (OHXU01000245) | SCJ27598.1 | SCJ27525.1 | 6 | N | N | 919 |
| human gut metagenome (OHXZ01000057) | OHXZ01000057_25 | OHXZ01000057_26 | 7 | N | N | 919 |
| human gut metagenome (OHYD01000532) | SCJ27598.1 | N/A | 4 | N | N | 919 |
| human gut metagenome (OHYU01000376) | OHYU01000376_4 | OHYU01000376_6 | 7 | N | N | 919 |
| human gut metagenome (OIBL01000128) | SCH71549.1 | N/A | 2 | N | N | 922 |
| human gut metagenome (OIBN01003740) | OIBN01003740_1 | N/A | 7 | N | N | 919 |
| human gut metagenome (OICI01000194) | OICI01000194_18 | OICI01000194_16 | 7 | N | N | 919 |
| human gut metagenome (OIDC01000397) | OIDC01000397_3 | OIDC01000397_5 | 5 | N | N | 919 |
| human gut metagenome (OIDU01000174) | OIDU01000174_25 | N/A | 5 | N | N | 919 |
| human gut metagenome (OIEE01000042) | OIEE01000042_11 | OIEE01000042_12 | 5 | N | N | 922 |
| human gut metagenome (OIEL01000292) | OIEL01000292_3 | WP_041337479.1 | 4 | N | N | 925 |
| human gut metagenome (OIEN01002196) | OIEN01002196_3 | N/A | 8 | Y | Y | 933 |
| human gut metagenome (OIGD01000177) | OIGD01000177_59 | OIGD01000177_43 | 14 | N | N | 918 |
| human gut metagenome (OIXA01002812) | OIXA01002812_3 | OIXA01002812_2 | 3 | N | N | 929 |
| human gut metagenome (OIXU01000818) | OIXU01000818 _5 | N/A | 2 | N | N | 955 |
| human gut metagenome (OIXU01000818) | OIXU01000818 _6 | N/A | 2 | N | N | 939 |
| human gut metagenome (OIXV01006344) | OIXV01006344_7 | N/A | 11 | N | N | 918 |
| human gut metagenome (OIYU01000175) | OIYU01000175_4 | OIYU01000175_5 | 4 | N | N | 921 |
| human gut metagenome (OIZA01000315) | OIZA01000315_9 | N/A | 3 | N | N | 945 |
| human gut metagenome (OIZB01000622) | OIZB01000622_13 | N/A | 3 | N | N | 923 |
| human gut metagenome (OIZB01000622) | OIZB01000622_13 | N/A | 3 | N | N | 921 |
| human gut metagenome (OIZI01000180) | OIZI01000180_12 | N/A | 3 | N | N | 963 |
| human gut metagenome (OIZI01000180) | OIZI01000180_12 | N/A | 3 | N | N | 947 |
| human gut metagenome (OIZU01000200) | OIZU01000200_48 | WP_041337479.1 | 6 | N | N | 929 |
| human gut metagenome (OIZW01000344) | OIZW01000344_20 | OIZW01000344_21 | 4 | N | N | 922 |
| human gut metagenome (OIZX01000427) | OIZX01000427 _25 | N/A | 4 | N | N | 961 |
| human gut metagenome (OIZX01000427) | OIZX01000427 _26 | N/A | 4 | N | N | 977 |
| human gut metagenome (OJMG01000332) | OJMG01000332_24 | WP_041337479.1 | 6 | N | N | 925 |
| human gut metagenome (OJMI01000733) | OJMI01000733_4 | OJMI01000733_5 | 5 | N | N | 922 |
| human gut metagenome (OJMJ01002228) | OJMJ01002228_5 | OJMJ01002228_2 | 5 | N | N | 919 |
| human gut metagenome (OJMK01000275) | OJMK01000275_31 | N/A | 6 | N | N | 939 |
| human gut metagenome (OJMM01002900) | OJMM01002900_7 | N/A | 6 | Y | N | 980 |
| human gut metagenome (OJMM01002900) | OJMM01002900_7 | N/A | 6 | Y | N | 979 |
| human gut metagenome (OJMN01000417) | OJMN01000417_22 | OJMN01000417_21 | 3 | N | N | 920 |
| human gut metagenome (OJNI01000536) | OJNI01000536_4 | OJNI01000536_5 | 3 | N | N | 920 |
| human gut metagenome (OJNR01001167) | OJNR01001167_9 | N/A | 5 | N | N | 954 |
| human gut metagenome (OJNS01001527) | OJNS01001527_9 | N/A | 2 | N | N | 954 |
| human gut metagenome (OJNT01000812) | OJNT01000812_6 | OJNT01000812_5 | 5 | N | N | 922 |
| human gut metagenome (OJOE01000269) | OJOE01000269_30 | OJOF01000269_29 | 5 | N | N | 922 |
| human gut metagenome (OJOH01001697) | SCH71549.1 | OJOH01001697_5 | 2 | N | N | 922 |
| human gut metagenome (OJOL01000697) | OJOL01000697_12 | OJOL01000697_13 | 5 | N | N | 922 |
| human gut metagenome (OJOP01001093) | OJOP01001093_3 | N/A | 5 | N | N | 954 |
| human gut metagenome (OJPG01000139) | OJPG01000139_73 | OJPG01000139_77 | 3 | N | N | 918 |
| human gut metagenome (OJPS01000131) | OJPS01000131_3 | OJPS01000131_4 | 3 | N | N | 918 |
| human gut metagenome (OJPX01000614) | OJPX01000614_4 | OJPX01000614_6 | 3 | N | N | 920 |
| human gut metagenome (OJQH01000635) | OJQH01000635_3 | OJQH01000635_4 | 3 | N | N | 918 |
| human gut metagenome (OJRG01001951) | OJRG01001951_4 | N/A | 3 | N | N | 920 |
| human gut metagenome (OJRP01000045) | OJRP01000045_31 | OJRP01000045_30 | 5 | N | N | 918 |
| human gut metagenome (OKRZ01002949) | OKRZ01002949_5 | OKRZ01002949_4 | 3 | N | N | 922 |
| human gut metagenome (OKSB01002689) | OKSB01002689_10 | OKSB01002689_10 | 4 | N | N | 922 |
| human gut metagenome (OKSC01004083) | OKSC01004083_2 | N/A | 2 | N | N | 906 |
| human gut metagenome (OKSD01002505) | OKSD01002505_11 | OKSD01002505_10 | 2 | N | N | 922 |
| human gut metagenome (OKSK01000361) | OKSK01000361_17 | OKSK01000361_20 | 3 | N | N | 922 |
| human gut metagenome (OKSNO1001169) | OKSNO1001169_3 | N/A | 13 | N | N | 1001 |
| human gut metagenome (OKSP01001453) | OKSP01001453_2 | N/A | 13 | N | N | 954 |
| human gut metagenome (OKSV01000264) | OKSV01000264_32 | OKSV01000264_31 | 5 | N | N | 922 |
| human gut metagenome (OKTJ01001834) | OKTJ01001834_4 | N/A | 6 | N | N | 921 |
| human gut metagenome (OKTR01000164) | OKTR01000164_10 | N/A | 6 | Y | N | 1023 |
| human gut metagenome (OKTU01000352) | OKTU01000352_17 | OKTU01000352_19 | 3 | N | N | 922 |
| human gut metagenome (OKUL01000400) | OKUL01000400_17 | OKUL01000400_16 | 7 | N | N | 919 |
| human gut metagenome (OKUR01000327) | OKUR01000327_17 | OKUR01000327_16 | 5 | N | N | 919 |
| human gut metagenome (OKVB01000375) | OKVB01000375_17 | OKVB01000375_16 | 7 | N | N | 919 |
| human gut metagenome (OKVC01000355) | OKVC01000355_17 | OKVC01000355_16 | 4 | N | N | 919 |
| human gut metagenome (OKVF01000105) | OKVF01000105_32 | OKVF01000105_31 | 5 | N | N | 922 |
| human gut metagenome (OKVK01000317) | SCH71549.1 | OKVK01000317_4 | 2 | N | N | 922 |
| human gut metagenome (OLFT01003273) | OLFT01003273_1 | OLFT01003273_2 | 3 | N | N | 925 |
| human gut metagenome (OLGH01000826) | OLGH01000826_1 | OLGH01000826_4 | 5 | N | N | 924 |
| human gut metagenome (OLGN01000304) | OLGN01000304_32 | OLGN01000304_31 | 9 | N | N | 920 |
| human gut metagenome (OLHE01000257) | OLHE01000257_41 | OLHE01000257_40 | 2 | N | N | 923 |
| human gut metagenome (PPYE01106492) | PPYE01106492_34 | PPYE01106492_32 | 2 | N | N | 922 |
| human gut metagenome (PPYE01385196) | PPYE01385196_3 | PPYE01385196_4 | 3 | N | N | 925 |
| human gut metagenome (PPYE01512733) | PPYE01512733_3 | PPYE01512733_2 | 4 | N | N | 919 |
| human gut metagenome (PPYF01129432) | PPYF01129432_15 | N/A | 9 | N | N | 918 |
| human gut metagenome (PPYF01670242) | PPYF01670242_39 | PPYF01670242_38 | 10 | N | N | 919 |
| human metagenome (ODEE01001565) | ODEE01001565_1 | N/A | 6 | N | N | 919 |
| human metagenome (ODFV01004017) | ODFV01004017_1 | N/A | 6 | N | N | 921 |
| human metagenome (ODFW01000112) | ODFW01000112_43 | ODFW01000112_41 | 5 | N | N | 924 |
| human metagenome (ODGN01000188) | ODGN01000188_50 | ODGN01000188_49 | 2 | N | N | 919 |
| human metagenome (ODHH01000275) | ODHH01000275_14 | ODHH01000275_15 | 4 | N | N | 919 |
| human metagenome (ODHP01001712) | ODHP01001712_3 | ODHP01001712_4 | 4 | N | N | 918 |
| human metagenome (ODHV01000466) | ODHV01000466_16 | ODHV01000466_16 | 5 | N | N | 925 |
| human metagenome (ODHZ01001211) | ODHZ01001211_7 | ODHZ01001211_6 | 5 | N | N | 921 |
| human metagenome (ODIH01000145) | ODIH01000145_73 | N/A | 2 | N | N | 919 |
| human metagenome (ODJZ01000182) | ODJZ01000182_13 | ODJZ01000182_15 | 2 | N | N | 921 |
| human metagenome (ODKA01005851) | ODKA01005851_3 | N/A | 6 | N | N | 924 |
| human metagenome (ODLN01002572) | ODLN01002572_7 | N/A | 8 | N | N | 924 |
| human metagenome (ODQJ01000729) | ODQJ01000729_25 | N/A | 9 | N | N | 919 |
| human metagenome (ODTU01003882) | ODTU01003882_3 | ODTU01003882_4 | 5 | N | N | 924 |
| human metagenome (ODUN01000242) | ODUN01000242_23 | ODUN01000242_22 | 3 | N | N | 922 |
| human metagenome (ODVQ01003982) | ODVQ01003982_3 | ODVQ01003982_4 | 5 | N | N | 919 |
| human metagenome (ODVR01002077) | ODVR01002077_3 | ODVR01002077_4 | 4 | N | N | 922 |
| human metagenome (ODVS01001471) | ODVS01001471_9 | ODVS01001471_8 | 5 | N | N | 924 |
| human metagenome (ODWX01000843) | ODWX01000843_3 | ODWX01000843_2 | 3 | N | N | 922 |
| human metagenome (ODXC01000747) | ODXC01000747_3 | ODXC01000747_4 | 2 | N | N | 922 |
| human metagenome (ODXE01000717) | ODXE01000717_15 | ODXE01000717_17 | 5 | N | N | 925 |
| human metagenome (ODXO01005124) | ODXO01005124_2 | ODXO01005124_1 | 3 | N | N | 922 |
| human metagenome (ODXP01000624) | ODXP01000624_4 | ODXP01000624_4 | 5 | N | N | 919 |
| human metagenome (ODYC01000377) | ODYC01000377_16 | ODYC01000377_17 | 5 | N | N | 924 |
| human metagenome (ODYJ01000298) | ODYJ01000298_33 | ODYJ01000298_33 | 4 | N | N | 919 |
| human metagenome (OEBA01002798) | OEBA01002798_7 | OEBA01002798_6 | 5 | N | N | 922 |
| human metagenome (OEEK01000163) | OEEK01000163_43 | OEEK01000163_44 | 5 | N | N | 922 |
| human metagenome (OEFH01000394) | OEFH01000394_40 | OEFH01000394_36 | 2 | N | N | 922 |
| human metagenome (OEFW01000634) | OEFW01000634_7 | OEFW01000634_8 | 5 | N | N | 922 |
| human metagenome (OEHT01000244) | OEHT01000244_15 | OEHT01000244_17 | 5 | N | N | 922 |
| human metagenome (OEJW01000623) | OEJW01000623_11 | OEJW01000623_13 | 6 | N | N | 922 |
| human-digestive system-homo sapiens | 3300007296 \| Ga0104830_100502_31 | 3300007296 \| Ga0104830_100502_30 | 5 | N | N | 919 |
| (3300007296 \| Ga0104830_100502) | | | | | | |
| human-digestive system-homo sapiens | 3300007299 \| Ga0104319_1000623_29 | 3300007299 \| Ga0104319_1000623_28 | 8 | N | N | 924 |
| (3300007299 \| Ga0104319_1000623) | | | | | | |
| human-digestive system-homo sapiens | 3300007361 \| Ga0104787_100954_14 | N/A | 3 | N | N | 923 |
| (3300007361 \| Ga0104787_100954) | | | | | | |
| human-digestive system-homo sapiens | 3300007361 \| Ga0104787_100954_14 | N/A | 3 | N | N | 921 |
| (3300007361 \| Ga0104787_100954) | | | | | | |
| human-digestive system-homo sapiens | 3300008272 \| Ga0111092_1001379_1 | N/A | 3 | N | N | 921 |
| (3300008272 \| Ga0111092_1001379) | | | | | | |
| human-digestive system-homo sapiens | 3300008496 \| Ga0115078_100057_51 | 3300008496 \| Ga0115078_100057_50 | 3 | N | N | 922 |
| (3300008496 \| Ga0115078_100057) | | | | | | |
| mammals-digestive system-asian elephant fecal- | 3300001598 \| EMG_10000232_1 | N/A | 2 | N | N | 963 |
| elephas maximus (3300001598 \| EMG_10000232) | | | | | | |
| mammals-digestive system-asian elephant fecal- | 3300001598 \| EMG_10003641_1 | N/A | 11 | Y | N | 1057 |
| elephas maximus (3300001598 \| EMG_10003641) | | | | | | |
| mammals-digestive system-feces | 3300018475 \| Ga0187907_10006632_17 | N/A | 18 | Y | Y | 977 |
| (3300018475 \| Ga0187907_10006632) | | | | | | |
| mammals-digestive system-feces | 3300018475 \| Ga0187907_10006632_17 | N/A | 18 | Y | Y | 971 |
| (3300018475 \| Ga0187907_10006632) | | | | | | |
| mammals-digestive system-feces | 3300018493 \| Ga0187909_10005433_18 | N/A | 18 | Y | Y | 977 |
| (3300018493 \| Ga0187909_10005433) | | | | | | |
| mammals-digestive system-feces | 3300018493 \| Ga0187909_10005433_18 | N/A | 18 | Y | Y | 971 |
| (3300018493 \| Ga0187909_10005433) | | | | | | |
| mammals-digestive system-feces | 3300018493 \| Ga0187909_10024847_5 | N/A | 4 | N | N | 1141 |
| (3300018493 \| Ga0187909_10024847) | | | | | | |
| mammals-digestive system-feces | 3300018493 \| Ga0187909_10030832_9 | N/A | 10 | N | N | 927 |
| (3300018493 \| Ga0187909_10030832) | | | | | | |
| mammals-digestive system-feces | 3300018494 \| Ga0187911_10005861_19 | N/A | 18 | Y | Y | 977 |
| (3300018494 \| Ga0187911_10005861) | | | | | | |
| mammals-digestive system-feces | 3300018494 \| Ga0187911_10005861_18 | N/A | 18 | Y | Y | 971 |
| (3300018494 \| Ga0187911_10005861) | | | | | | |
| mammals-digestive system-feces | 3300018494 \| Ga0187911_10019634_9 | N/A | 11 | N | N | 927 |
| (3300018494 \| Ga0187911_10019634) | | | | | | |
| mammals-digestive system-feces | 3300018494 \| Ga0187911_10037073_4 | N/A | 4 | N | N | 1141 |
| (3300018494 \| Ga0187911_10037073) | | | | | | |
| mammals-digestive system-feces | 3300018494 \| Ga0187911_10069260_3 | N/A | 2 | N | N | 900 |
| (3300018494 \| Ga0187911_10069260) | | | | | | |
| mammals-digestive system-feces | 3300018495 \| Ga0187908_10006038_18 | N/A | 18 | Y | Y | 977 |
| (3300018495 \| Ga0187908_10006038) | | | | | | |
| mammals-digestive system-feces | 3300018495 \| Ga0187908_10006038_19 | N/A | 18 | Y | Y | 971 |
| (3300018495 \| Ga0187908_10006038) | | | | | | |
| mammals-digestive system-feces | 3300018495 \| Ga0187908_10013323_2 | N/A | 4 | N | N | 1141 |
| (3300018495 \| Ga0187908_10013323) | | | | | | |
| mammals-digestive system-feces | 3300018878 \| Ga0187910_10006931_17 | N/A | 18 | Y | Y | 977 |
| (3300018878 \| Ga0187910_10006931) | | | | | | |
| mammals-digestive system-feces | 3300018878 \| Ga0187910_10006931_17 | N/A | 18 | Y | Y | 971 |
| (3300018878 \| Ga0187910_10006931) | | | | | | |
| mammals-digestive system-feces | 3300018878 \| Ga0187910_10015336_15 | N/A | 4 | N | N | 1141 |
| (3300018878 \| Ga0187910_10015336) | | | | | | |
| mammals-digestive system-feces | 3300018878 \| Ga0187910_10040531_1 | N/A | 3 | N | N | 927 |
| (3300018878 \| Ga0187910_10040531) | | | | | | |
| mammals-digestive system-feces | 3300019376 \| Ga0187899_10021543_4 | N/A | 4 | N | N | 880 |
| (3300019376 \| Ga0187899_10021543) | | | | | | |
| metagenome (OGCZ01001955) | OGCZ01001955_1 | N/A | 4 | N | N | 926 |
| metagenome (OGDS01000069) | OGDS01000069_10 | N/A | 3 | N | N | 956 |
| metagenome (OGDY01002059) | OGDY01002059_17 | N/A | 10 | N | N | 952 |
| metagenome (OGEU01000713) | OGEU01000713_24 | OGEU01000713_23 | 6 | N | N | 923 |
| metagenome (OGFM01002125) | OGFM01002125_3 | OGFM01002125_4 | 6 | N | N | 928 |
| metagenome (OGGS01001705) | OGGS01001705_3 | OGGS01001705_5 | 5 | N | N | 922 |
| metagenome (OGGV01005531) | OGGV01005531_2 | N/A | 2 | N | N | 922 |
| metagenome (OGHW01002048) | OGHW01002048_1 | OGHW01002048_2 | 4 | N | N | 922 |
| metagenome (OGIE01002059) | OGIE01002059_21 | OGIE01002059_22 | 4 | N | N | 922 |
| metagenome (OGII01000819) | OGII01000819_21 | OGII01000819_22 | 4 | N | N | 922 |
| metagenome (OGJI01000038) | OGJI01000038 151 | OGJI01000038_150 | 2 | N | N | 926 |
| metagenome (OGJK01007642) | OGJK01007642_2 | N/A | 2 | N | N | 925 |
| metagenome (OGJY01000516) | OGJY01000516_18 | OGJY01000516_19 | 6 | N | N | 925 |
| metagenome (OGKA01000617) | OGKA01000617_2 | OGKA01000617_3 | 3 | N | N | 919 |
| metagenome (OGKE01000029) | OGKE01000029_151 | OGKE01000029_150 | 2 | N | N | 926 |
| metagenome (OGKG01000020) | OGKG01000020_152 | OGKG01000020_150 | 2 | N | N | 926 |
| metagenome (UGKG01002483) | OGKG01002483_14 | N/A | 7 | N | N | 954 |
| metagenome (OGKW01000585) | OGKW01000585_4 | OGKW01000585_4 | 4 | N | N | 918 |
| metagenome (OGLJ01000192) | OGLJ01000192_54 | OGLJ01000192_55 | 3 | N | N | 925 |
| metagenome (OGLM01001314) | OGLM01001314_21 | N/A | 20 | N | N | 954 |
| metagenome (OGMO01000062) | OGMO01000062_69 | OGMO01000062_68 | 6 | N | N | 925 |
| metagenome (OGMP01001167) | OGMP01001167_15 | OGMP01001167_14 | 6 | N | N | 921 |
| metagenome (OGNV01000836) | OGNV01000836_4 | OGNV01000836_6 | 3 | N | N | 922 |
| metagenome (OGUJ01000114) | OGUJ01000114_43 | N/A | 9 | N | N | 941 |
| metagenome (OGUJ01000114) | OGUJ01000114_45 | N/A | 9 | N | N | 937 |
| metagenome (OJKY01000879) | OJKY01000879_3 | N/A | 12 | Y | N | 1023 |
| metagenome (OLJF01000187) | OLJF01000187_58 | N/A | 5 | N | N | 922 |
| uncultured Clostridiales bacterium (OMWO01000091) | OMWO01000091_3 | N/A | 4 | N | N | 880 |
| uncultured Ruminococcus sp. (FMFL01000053) | SCJ27598.1 | SCJ27525.1 | 10 | N | N | 919 |

TABLE 2
Amino Acid Sequences of Cas13d Effector Proteins

>LARF01000048_8
[Ruminococcus sp. N15.MGS-57]
MAKKNKMKPRELREAQKKARQLKAAEINNNAAPAIAAMPAAEVIAPVAEKKKSSVKAAGMKSILVSKNKMYITSFGKGNSAVLE
YEVDNNDYNQTQLSSKGSSNIELRGVNEVNITFSSKHGFESGVEINTSNPTHRSGESSPVRGDMLGLKSELEKRFFGKTFDDNI
HIQLIYNILDIEKILAVYVTNIVYALNNMLSIKDSESYDDFMGYLSARNTYEVFTHPDKSNLSDKAKGNIKKSFSTFNDLLKTK
RLGYFGLEEPKTKDTRVSQAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEASKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKRENDTKQEEKIKYEDDLLKNHGYTKDFVKALNSPFGYNIPRFKNLSIEQLFDRNEYLTEK (SEQ
ID NO: 2)
>WP_005358205.1
[[Eubacterium] siraeum DSM 15702]
MGKKIHARDLREQRKTDRTEKFADQNKKREAERAVPKKDAAVSVKSVSSVSSKKDNVTKSMAKAAGVKSVFAVGNTVYMTSFGR
GNDAVLEQKIVDTSHEPLNIDDPAYQLNVVTMNGYSVTGHRGETVSAVTDNPLRRFNGRKKDEPEQSVPTDMLCLKPTLEKKFF
GKEFDDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKAKNVLREDKELYSVLTLIGKLRHWCVHSEEGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKKLSKSNIEIQEDKLRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIESDKTEEDIEKMIDNILQIDANGDKKLKKNNGLRNFIASNVIDSNRFKYLVRYGNPKKIRETAKCKPAVRFVLNEI
PDAQIERYYEACCPKNTALCSANKRREKLADMIAEIKFENFSDAGNYQKANVTSRTSEAEIKRKNQAIIRLYLTVMYIMLKNLV
NVNARYVIAFHCVERDTKLYAESGLEVGNIEKNKTNLTMAVMGVKLENGIIKTEFDKSFAENAANRYLRNARWYKLILDNLKKS
ERAVVNEFRNTVCHLNAIRNININIKEIKEVENYFALYHYLIQKHLENRFADKKVERDTGDFISKLEEHKTYCKDFVKAYCTPF
GYNLVRYKNLTIDGLFDKNYPGKDDSDEQK (SEQ ID NO: 1)
>33000102661Ga0129314_1001134_19
[animal-digestive system-orangutan individual fecal]
MGKKIHARDLREQRKNDRTTKFAEQNKKREAQMAVQKKDAAVSAKSVSSVSSKKGNVTKSMAKAAGVKSVFAVGKNTVYMTSFG
RGNDAVLEQKIVDTSHEPLNIDDPAYQLNVVTMNGYSVTGHRGETVSAITDNPLRRFNGGKKDKPEQSVPADMLCLKPTLEKKF
FGKEFDDNIHIQLIYNILDIEKILAVYSTNAVYALNNTIADENNENWDLFANFSTDNTYGELINAATYKESTDDVSTDDEKRRE
AEKKKREAKIAEKILADYEKFRKNNRLAYFADAFYIEKNKSKSKSQNKAEGIKRGKKEIYSILALIAKLRHWCVHSEDGRAEFW
LYKLDELEDDFKNVLDVVYNRPVEEINDDFVERNKVNIQILHSKCENSDIAELTRSYYEFLITKKYKNMGFSIKKLREIILEGT
EYNDNKYDTVRNKLYQMVDFILYRGYINENSERAEALVNALRSTLNEDDKTKLYSSEAAFLKRKYMKIIREVTDSLDVKKLKEL
KKNAFTIPDNELRKCFISYADSVSEFTKLIYLLTRFLSGKEINDLVTTLINKFDNIRSFLEIMDELGLERTFTDEYSFFEGSTK
YLAELIELNSFVKSCSFDMSAKRPMYRDALDILGIESDKSEDDIKRMIDNILQVDANGKKLPNKNHGLRNFIASNVVESNRFEY
LVRYGNPKKIRETAKCKPAVRFVLNEIPDAQIERYYKAYYLDEKSLCLANMQRDKLAGVIADIKFDDFSDAGSYQKANATSTKI
TSEAEIKRKNQAIIRLYLTVMYIMLKNLVNVNARYVIAFHCLERDTKLYAESGLEVGNIEKNKTNLTMAVMGVKLENGIIKTEF
DKSLAENAANRYLRNARWYKLILDNLKMSERAVVNEFRNTVCHLNAIRNININIDGIKEVENYFALYHYLIQKHLENRFADNGG
STGDYIGKLEEHKTYCKDFVKAYCTPFGYNLVRYKNLTIDGLFDKNYPGKDDSDKQK (SEQ ID NO: 3)
>33000062261Ga0099364_10024192_5
[arthropoda-digestive system-cubitermes and nasutitermes termite gut]
MSQSTKTKAKRMGVKSVLAHGKDEKGHIKLAITAFGKGNKAELAIQTDEKGSNLAKTYKERNITANKIVSEGIQTSGTIAGEGH
ATFLNNPAEHVGTDYLKLKETLEMEFFGKSFPGDSVRIQIIHQILDIQKLLGIYITDIIYCINNLRDETHLDHESDIVGLSMSN
TNVNLALNQMRPYFGFFGEAFRPVGDDKVKEITLSDEVRKNIEKIIALEEQKRNPSTPRFKQENINLEIENAMGKFKSKDAFET
AKKKYNRIVADETNAKTLRILGAMRQITAHFKDQATLFMSDVELPKILKKEFSKADWQTVEDYYAKLVDRINEGFCKNAATNVH
FLTELLPEESKKQLTEDYFRFAILKEGKNLGVNMKRLREVMFALFVPELTAPETKKRYDSYRAKIYGLTDFLLFKHIHNTKQLE
EWVAVLRETSNEDAKENLYDEFARTAWNTVGDSAKQLIENMQSYFTKKEKEITKTAQPVLSTSSIAHTSKKITQFSSFAKLLAF
LCNFWEGKEINELLSAYIHKFENIQEFINLLEKLEGKKPQFTENYALFNEAAGQRAGEIAQNLRILASIGKMKPDLGDAKRQLY
KAAIEMLGIDTEEYISDEWLEPNMLLAQPPKEPKKDNEKYRKEPHKYSYEKDMETYRKKLREYEETWRSLIDYEYLMPETNPFR
NFVAKQVIESRRFMYLVRYTKPKTVRALMSNRAIVHYVLSRIADIQDHHMTESQIDRYYQNLPQYNEQQHKNVSLETKIDALAD
YLCKYTFEKNVLKQKNGIVLNTKSATKNVEIEHLKALTGLYLTVAYIAVKNLVKANARYYIAFSIFERDYALFEKKLGKDTLEK
YVKPFKYIDKGEEKEGKNNFFALTEYLLDKDNSLRYQWNNDLSDEENKQALRKHLDKKEIRSQRHFSQYWLDIFARQIENAKKT
SESGYLLTAARNCALHLNVLTALPEFVGEFRKTGDKMTSYFELYHFLLQKLMLAEAGLNLDEYRERIDTYQTACKDLINITYVS
LGYNLPRYKNLTCEPLFDEESATGKERQTRLDEKSKEKKQRKGGQK (SEQ ID NO: 4)
>NZ_NFLV01000009_111
[Eubacterium sp. Ann]
MSKKQRPKDIRKRQEEEKREKYKKQEELRKKQEELRKEQEQRREDQKELEKIKKEVGEEGEKKKSRAKALGLKSTFILDRDEQK
VLMTSFGQGNKAVRDKYIIGDKVSDINDDRKNKKAALLVEVCGKSFNISKKENDDCDPVKVNNPVVSRNKKDDDLIHCRKKLEE
LYFGEQFKDNIHIQLIYNILDIEKILAVQVNNIVFALNNLLSWSGEEKFDLIGYLGVNDTYEKFRDAKGKRKGLYEKFSTLIEK
KRMRYFGSTFYPLNEKGEEITSNDKKEWEQFEKKCYHLLAVLGMMRQATAHGDSKRRAEIYKLGKEFDKSEARGCRQEARKELD
DLYRKKIHEMNQSFLKNSKRDILMLFRIYDAESKEAKRKLAQEYYEFIMLKSYKNTGFSIKHLRETVIDKMDEDIKEKIKDDKY
NPIRRKLYRIMDFVIYQYYQESEQQEEAMELVRKLRNAETKVEKELTYRKEAEKLKEELEKIIRNSILSVCDRILAEMNEKRHK
KVNQESSDTDSEEPLDPEISEGITFIKETAHSFSEMIYLLTVFLDGKEINILLTQLIHCFDNISSFMDTMKEENLLTKLKEDYE
IFEESKEISKELRIINSFARMTEPVPKTEKTMFIDAAQILGYSNDEKELEGYVDALLDTKNKTKDKERKGFEKYIWNNVIKSTR
FRYLVRYADPKKVRAFAANKKVVAFVLKDIPDEQIKAYYNSCFSQNSDSSSNMSIAFQDGDSNKKGTSVHDMMRKALTEKITGL
NFGDFEEESKKGIRREESDKNIIRLYLTVLYLVQKNLIYVNSRYFLAFHCAERDEVLYNGETIDNNKEKGSEKDWKKFAKEFII
EHPPKKKVKDYLAKNFEYSNKWSLRVFRNSVQHLNVIRDAYKYIKCIDDNKDVQSYFALYHYLVQRYISEMAENLTDKGELSEG
RLQYYLSQVENYRTYCKDFVKALNVPFAYNLPRYKNLSIDELFDRNNYLPNKAKKWISEKKENGEYVMEDCGNKGAGQVENA
(SEQ ID NO: 5)
>NFIR01000008_78
[Eubacterium sp. An3]
MAKKLRPKELREKRRMAEKEEHKKQEKLRKEQEELRKKQEKQREDQKELEKIKKEEGGEGEKKKSGAKALGLKSTFILDRDEQK
MLMTSFGRGNKAVRDKYIIGDKVSDIDDSWENKKAALSVEVCGKSFNISKKENDDCEPVKVNNPVLSGNKKDDDLIHCRKNLEE
MYFGQQFKDNIHIQLIYNILDIEKILAVQINNIVFILNNLLRWSGEEEFDLIGSLGVNHTYEEFRGRNKNYGKFSELIKQSQMR
YFGSTFCLFNENEERITSENKKEWKRFEKKCYHLLAVLGMMRQATAHGDSKRRAEIYKLGKEFDRLEARGCRPEARKELDELYK
KKIHEMNQGFLKNSKSDILMLFRIYNAESKEAKRKLAQEYYEFIMLKSYKNTGFSIKHLRETMIDKMDEDKKEKLKDDKYNPIR
RKIYRIMDFMIYQYYQEPEHQEEAEELVRKLRNAEIEAKKELAYRKEAEKLKKELEKIIFNSVLPSCDRILSEMDERRNKKVNQ
ESSDTDKEEPLDSEIAEGITFIKETAHSFSEMIYLLTVFLDGKEINILLTQLIHCFDNISSFMDTMEEENLLTKLKEDYEIFEE
SKEISRELRIINSFARMTEPVPKTERIMFIEAAQILGYSNGEKELEGYVDALLDTKNKTNDKKKKGFVRYIWNNVIKSTRFRYL
VRYADPKKVRAFAANKKVVAFVLKDIPDDQIRAYYNSCFRQNSDSSSNNSNASWDADSNKRDISVSDMRKALTEKITGLNFGDF
EEESKKGIRKEESDKNIIRLYLTVLYLVQKNLIYVNSRYFLAFHCAERDEMLYNGETIDNNKEKGSEKDWRKFAKQFIMEHSPK
KKVKDYLAKNFEYSNKWSLKEFRNSVQHLNVIRDAHKYIKYINDNKDVQSYFALYHYLVQRYISERAANRTDKESLSEGRLQYY
LSQVKEYRTYCKDFVKALNVPFAYNLPRYKNLSIDELFDRNNYLPNKAKKWIPEKKENGEYVMEDCGNKDAGQVENA (SEQ
ID NO: 6)
>CDY501033339_14
[gut metagenome]
MEREVKKPPKKSLAKAAGLKSTFVISPQEKELAMTAFGRGNDALLQKRIVDGVVRDVAGEKQQFQVQRQDESRFRLQNSRLADR
TVTADDPLHRAETPRRQPLGAGMDQLRRKAILEQKYFGRTFDDNIHIQLIYNILDIHKMLAVPANHIVHTLNLLGGYGETDFVG
MLPAGLPYDKLRVVKKKNGDTVDIKADIAAYAKRPQLAYLGAAFYDVTPGKSKRDAARGRVKREQDVYTILSLMSLLRQFCAHD
SVRIWGQNTPAALYGLQALPQDMKDLLDDGWRRALGGVNDHFLDTNKVNLLTLFEYYGAETKQERVALTQDFYRFVVLKEQKNM
GFSLRRLREELLKLPDAAYLTGQEYDSVRQKLYMLLDFLLCRLYAQERADRCEELVSALRCALSDEEKDAVYQAEAAALWQALG
DTLRRELLPLLKGKKLQDKDKKKLDELGLSRDVLDGVLFRPAQQGSRANADYFCRLMHLSTWFMDGKEINTLLTTLISKLENID
SLRSVLESMGLAYSFVPAYAMFDHSRYIAGQLRVVNNIARMRKPAIGAKREMYRAAVVLLGVDSPEAAAAITDDLLQIDPETGK
VRPRGDSARDTGLRNFVANNVVESRRFTYLLRYMTPEQARVLAQNEKLIAFVLSTVPSAQLERYCRTCGREDITGRPAQIRYLT
AQIMGVRYESFTDVEQRGRGDNPKKERYKALIGLYLTVLYLAVKNMVNCNARYVIAFYCRDRDTALYQKEVCWYDLEEDKKSGK
QRQVEDYTALTRYFVSQGYLNRHACGYLRSNMNGISNGLLAAYRNAVDHLNVIPPLGSLCRDIGRVDSYFALYHYAVQQYLNGR
YYRKTPREQELFAAMAQHRTWCSDLVKALNTPFGYNLARYKNLSIDGLFDREGDHVVREDGEKPAE (SEQ ID NO: 7)
>CDYU01004315_2
[gut metagenome]
MAKKNKMKPRELREAQKKARQLKAAEIKNNAVPAIAAMPAAEAAAPAVEKKKSSVKAAGMKSILVSENKMYITSFGKGNSAVLE
YEVDKVDNNNYNKTQLSSKDNSNIELGDVNEVNITFSSKRGNESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQCVFHDKSGAKRFDLYSFINNIDPEYRDTLDYLVEERLKSINKDF
IEGNKVNISLLIDMMKGYEADDIIRLYYDFIVLKSQKNLGFSIKKLREKMLEEYGYRFKDKQYDSVRSKMYKLMDFLLFCNYYR
NDVVAGEALVRKLRFSMTDDEKEGIYADEASKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDTKQEEKIKYEDDLLKNHGYTKDFVKALNSPFGYNIPRFKNLSIEQLFDRNEYLTE
K (SEQ ID NO: 8)
>CDYX01024884_4
[gut metagenome]
MAKKNKMKPRELREAQKKARQLKAAEINNNAVPAIAAMPAAEVIAPAAEKKKSSVKAAGMKSILVSENKMYITSFGKGNSAVLE
YEVDKVDDNDYNKTQLSSKDNSNIELGNVNEVNITFSSRRGFESGVEINTSNPTHRSGESSSVRGDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDKRVSEAYKKRVYHMLAIVGQIRQSVFHDKSNELDEYLYSFIDIIDSEYRDTLDYLVDERFDSINKGF
IQGNKVNISLLIDMMKDDYEADDIIRLYYDFIVLKSQKNLGFSIKKLREKMLEEYGFRFKDKQYDSVRSKMYKIMDFLLFCNYY
RNDVVAGEALVRKLRFSMTDDEKEGIYADEAAKLWGKFRNDFENIADHMNGDVIKELGKADMDFDEKILDSEKKNASDLLYFSK
MIYMLTYFLDGKEINDLLTTLISKFDNIKEFLKIMKSSAVDVECELTAGYKLFNDSQRITNELFIVKNIASMRKPAASAKLTMF
RDALTILGIDDNITDDRISEILKLKEKGKGIHGLRNFITNNVIESSRFVYLIKYANAQKIREVAKNEKVVMFVLGGIPDTQIER
YYKSCVEFPDMNSSLEVKRSELARMIKNICFDDFKNVKQQAKGRENVAKERAKAVIGLYLTVMYLLVKNLVNVNARYVIAIHCL
ERDFGLYKEIVSELASKNLKNDYRILSQTLCELCDKSPNLFLKKNERLRKCVEVDINNADSSMTRKYRNCIAHLTVVRELKEYI
GDIRAVDSYFSIYHYVMQRCITKRGNDTKQEDKIKYEDDLLKNHGYTKDFVKALNSPFGYNIPRFKNLSIEQLFDRNEYLTEK
(SEQ ID NO: 9)
>CDTW01032418_55
[gut metagenome]
MEREVKKPPKKSLAKAAGLKSTFVISPQEKELAMTAFGRGNDALLQKRIVDGVVRDVAGEKQQFQVQRQDESRFRLQNSRLADR
TVTADDPLHRAETPRRQPLGAGMDQLRRKAILEQKYFGRTFDDNIHIQLIYNILDIHKMLAVPANHIVHTLNLLGGYGETDFVG
MLPAGLPYDKLRVVKKKNGDTVDIKADIAAYAKRPQLAYLGAAFYDVTPGKSKRDAARGRVKREQDVYAILSLMSLLRQFCAHD
SVRIWGQNTTAALYHLQALPQDMKDLLDDGWRRALGGVNDHFLDTNKVNLLTLFEYYGAETKQARVALTQDFYRFVVLKEQKNM
GFSLRRLREELLKLPDAAYLTGQEYDSVRQKLYMLLDFLLCRLYAQERADRCEELVSALRCALSDEEKDTVYQAEAAALWQALG
DTLRRKLLPLLKGKKLQDKDKKKSDELGLSRDVLDGVLFRPAQQGSRANADYFCRLMHLSTWFMDGKEINTLLTTLISKLENID
SLRSVLESMGLAYSFVPAYAMFDHSRYIAGQLRVVNNIARMRKPAIGAKREMYRAAVVLLGVDSPEAAAAITDDLLQIDPETGK
VRPRSDSARDTGLRNFIANNVVESRRFTYLLRYMTPEQARVLAQNEKLIAFVLSTVPDTQLERYCRTCGREDITGRPAQIRYLT
AQIMGVRYESFTDVEQRGRGDNPKKERYKALIGLYLTVLYLAVKNMVNCNARYVIAFYCRDRDTALYQKEVCWYDLEEDKKSGK
QRQVEDYTALTRYFVSQGYLNRHACGYLRSNMNGISNSLLTAYRNAVDHLNAIPPLGSLCRDIGRVDSYFALYHYAVQQYLNGR
YYRKTPREQELFAAMAQHRTWCSDLVKALNTPFGYNLARYKNLSIDGLFDREGDHVVREDGEKPAE (SEQ ID NO: 10)
>CDZT01047721_3
[gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSFSTFNDLL
KTKRLGYFGLEEPKTKDTRVSEAYKKRVYHMLAIVGQIRQCVFHDLSEHSEYDLYSFIDNSKKVYRECRETLNYLVDERFDSIN
KGFIQGNKVNISLLIDMMKDDYEADDIIHLYYDFIVLKSQKNLGFSIKKLREKMLDEYGFRFKDKQYDSVRSKMYKLMDFLLFC
NYYRNDVVAGEALVRKLRFSMTDDEKEGIYADEAEKLWGKFRNDFENIADHMNGDVIKELGKADMDFDEKILDSEKKNASDLLY
FSKMIYMLTYFLDGKEINDLLTTLISKFDNIKEFLKIMKSSAVDVECELTAGYKLFNDSQRITNELFIVKNIASMRKPAASAKL
TMFRDALTILGIDDKITDDRISEILKLKEKGKGIHGLRNFITNNVIESSRFVYLIKYANAQKIREVAKNEKVVMFVLGGIPDTQ
IERYYKSCVEFPDMNSSLKVKRSELARMIKNIRFDDFKNVKQQAKGRENVAKERAKAVIGLYLTVMYLLVKNLVNVNARYVIAI
HCLERDFGLYKEIIPELASKNLKNDYRILSQTLCELCDDRDESPNLFLKKNRRLRKCVEVDINNADSSMTRKYRNCIAHLTVVR
ELKEYIGDIRTVDSYFSIYHYVMQRCITKREDDTKQEEKIKYEDDLLKNHGYTKDFVKALNSPFGYNIPRFKNLSIEQLFDRNE
YLTEK (SEQ ID NO: 11)
>ODXP01000624_4
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDSSNIELRGVNEVNITFSSKHGFGSGVEINTSNPTHRSGESSPVRWDMLGLKSELEKRFFGKTFDDNI
HIQLIYNILDIEKILAVYVTNIVYALNNMLGIKKSESHDDFMGYLSAKNTYDVFTNPNGSTLSDDKKKNIRKSLRKFNDLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKIIDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNRRLRKCVEVDINNADSSMTRKYRNCIAHLTVVRELKEYIGDIR
TVDSYFSIYHYVMQRCITKRENDTKQEEKIKYEDDLLKNHGYTKDFVKALNSPFGYNIPRFKNLSIEQLFDRNEYLTEK (SEQ
ID NO: 12)
>ODKA01005851_3
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSEDSSNIELCGVNKVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTNALEAYKKRVYYMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKMLDEYGFRFKDKQYDPVRSKMYKLMDFLLFCNYYRN
DVVAGEALVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVNVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNISFDDFKNVKQQAKGRENVAKERAKAVIGLYLTVMYLLVENLVNVNARYVIAIHCLER
DFGLYKEIISELASKNLKNDYRILSQTLCELCDNCDESPNLFLKKNERLRKCVEVDINNADSNMTRKYRNCIAHLTVVRELNKY
IKDIRTVDSYFSIYHYVMQRCITKREDDKKQEEKIKYEDDLLKNHGYTKDFVKALNSPFGYNIPRFKNLSIEQLFDRNEYLTEK
(SEQ ID NO: 13)
>OGPQ01001037_3
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNKTQLSSKDNSNIELGDVDEVNITFSSKHGFGSGVEINTSNPTHRSGESSPVRWDMLGLKSELEKRFFGKTFDDNI
HIQLIYNILDIEKILAVYVTNIVYALNNMLGEGGDESHDDIMGYLSAKNTYDVFTDPDESDLSKNIKGNIKKSLSKFNDLLKTK
RLGYFGLEEPKTKDKRASEAYKKRVYHMLAIVGQIRQSVFHDKSNELDEYLYSFIDIIDSEYRDTLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
IAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSMGAKRRELAKMIKSISFEDFKDVKQQAKGRENVAKERAKAVIGLYLTVMYLLVKNLVNVNARYVIAIHCLERDF
GLYKEIIPELASKNLKNDYRILSQTLCELCDNGDESPNLFLKKNKRLRKCVEVDINNADSNMTRKYRNCIAHLTVVRELNKYIK
DIRTVDSYFSIYHYVMQRCITKRENDTKQEEKINYEDDLLKNHGYTKDFVKALNSPFGYNIPRFKNLSIEQLFDRNEYLTEK
(SEQ ID NO: 14)
>CDZK01015063_14
[gut metagenome]
MFMAKKNKMKPRERREAQKKARQLKAAEINNNAVPAIAAMHAAEVIAPAAEKKKSSVKAAGMKSILVSENKMYITSFGKGNSAV
LEYEVDNNDYNQTQLSSKDNSNIELCGVTKVNITFSSKHGFESGVEINTSNPTHRSGESSPVRGDMLGLKSELEKRFFGKTFDD
NIHIQLIYNILDIEKILAVYVTNIVYALNNMLGEGDESNYDFMGYLSTFNTYKVFTNPNGSTLSDDKKENIRKSLSKFNALLKT
KRLGYFGLEEPKTKDTRASEAYKKRVYHMLAIVGQIRQCVFHDKSGAKRFDLYSFINNIYPEYRDTLDYLVEERLKSINKDFIQ
GNKVNISLLIDMMKGYEADDIIRLYYDFIVLKSQKNLGFSIKKLREKMLDEYGFRFKDKQYDSVRSKMYKLMDFLLFCNYYRND
VVAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDKITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDDRDESPNLFLKKNKRLRKCVEVDINNADSSMTRKYRNCIAHLTVVRELKKYI
GDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 15)
>33000015981EMG_10000232_1
[mammals-digestive system-asian elephant fecal-elephas maximus]
MYNIDKLWLTHILFVSLTAGKKNETILEQEINKDSNKKNILVNPTKFDANIKEVRMVSIKPEKYNETVVNNPYYVKDGQVVGQD
YLGIKDKLEDTFFGKTYDDNIHIQIAYKLLDIRKIMGMSVGSAVFSLNNLQQRPVGENPNDIVGQIKTDTSFDEIPDNYAKADK
DFIDILLDYTRYFDNVFEKQSISVDDKTKDILNNLKECETVSVKTVGTIDRINKNDPNNNNYTIFKIGGLKIKLKGILSNVDVG
TKLNIEGQIRRNNDYRDKKGKLCRSYSLLTGAKYSISHEVYNPDTYTFNYDILRLVSYLRQAVVHNNNDDYIDWLYSIDNKKET
KDILNAANKVFESQLEAFNKDFNANAQKNVYMIASVLNDSPKTMFKEEIKDIYEKYYNFVLFKENRNVGINLRNIRNNIFYEDI
KPNYDEKELSRERAKINTLLDYFIYQDFNNNEKLAEDVIARLQPTKQEVDKVQVYADVTKEFKVRNPKLVDRILSTVKNTIEAK
IENFIPDNCVPSSSIKVSSLAKYVYVLAKFLDTKEVNNLLTSLINSFENIGSLVKVLKDEKGYSIYKDRFALLNQKNPFDLAND
FILVKNLATMKTKLAKANVKDVKNKVGKRLYCSAINLFKDKNDEVILDNQEFEDIMSEFSSNVGNKKNRRGTAGSKIRNFLINN
VIDSRRFYFIIKYYDTRRCHEIIQNENLVRFILGREDMPTDQLIRYYKTITGNECNNRNQIIDTLVKKLKEVSFRKLLLKGERL
KEIGNDQDNQEVESLKSLIGLYLTICYLIVKGIVNVNSVYLLAWSAYERDMYYLYNEDMEDKNTNHDYLKAATDFYNNKSCYQK
RHKYLIKDIEEARQNSNNLNYKDYRNKVCHYNICTSFMDYANNIGKVSCYFDIYNYCFQRYFAKKNDNLSTLLDTYNCYNKDYL
KLLNMPFAYNMARYKNLTIADLFNDKYPSENKEATASND (SEQ ID NO: 16)
>33000015981EMG_10003641_1
[mammals-digestive system-asian elephant fecal-elephas maximus]
MEETKVTKETTIEKQSTKRHKQKSKKTATKMSGLKSALVINNHEMLLTSFGKGNNAIAEKRYILDGDIETINNKNKKFDANNDS
KVVVIKGISNPNGQLTNPLFDQSPTAIQPNRTSGNDMIGIRRMLERKYFVHNEENKEFQDNIRIQIAYCILDIEKILMPHINNI
CFEINNMLRLEGYQEDSFMGSFNLYKPYDAFIATTDDKESSRRDNFAKLMTSKQVRYLGNALYSDSLSNLTKDEILDGKRSKEL
KKYYQELCLLGMVRQSMIHSNQFNSSIYTLDSSYDSTMNTAELLGKGDDSSLVALATDARVEARAILDEIYKKGVDSINNSFLS
NSINDLENLFKIYKCDSSEKKTELIKQYYDFCIRKPQMNMGFSITTIREGMFTRCSEANTLLLCDEGSTVKLNVHDTMKSKFYK
NLDFMIYKYYKYENPEKGEKLIEDLRSKIKGKKKEDEDKKQRYAEESACILKAKRDIIKKDLTEAANKDLFADLVKSNKNEKQK
FKNEYEELLKPFMIPVKVDYFSELIYLVTRFLSGKEINDLLTQLINKFENIAAFIRMYQNDQGKLEFTANYKMFEIDPQKDIPK
DGKRVLSGSAKIAYYLRTINYIARMESFEIKSDKTAINDAISLLGYNSNEHRDEFITYTMAKHVVDKYQNTDYQKIVKDFLSAN
KTLDCKSKNMQAFVSELKNAHLSENYEQREKEIYELADTNLPAYFSEEDKEKLARYIVHSDGTYKKFLKESFYAIEELPNEGFR
NFISNNVINSRRFNYIMRFCNPEKIANIGKNKVLISFALSSLAEKTDMIAKYYRVFCDRIDDQKTMEDYLVNKLTKISYTEFLN
VNQKANAEKNKEKDRSQKLIGLYITLLYEIVKNLVNINSRYNIAFQRCDNDSIMILQGQYDERAVQESKLTKKFISNQKLNSYS
CRYLTHNISQLDRCNDFIRQYRNKVAHLEVVSNIDEYLSGIKHIESYYALYHYLMQKCLLKNYRIEDHSQNEYKNLNDFSSKLD
KHGTYVKDFVKALNVPFGYNLPRYKNLSIDELFDRNKLKTGGTIEMKGE (SEQ ID NO: 17)
>33000184751Ga0187907_10006632_17
[mammals-digestive system-feces]
MKERIDMIEKKKSYAKGMGLKSTLVSDSKVYMTSFGNGNDARLEKVVENNAISCLVDKKEAFVAEITDKNAGYKIINKKFGHPK
GYDVVANNPLYTGPVQQDMLGLKETLEKRYFGSSVSGNDNICIQVIHNILDIEKILAEYITNAAYAVNNIAGLDKDIIGFGKFS
TVYTFDEFAEPDRHKERFIKDGKLDTKLINQLKNQYDEFDAFLDDTRFGYFGKAFFCKEGDKYLNKQDNERYHILALLSGLRNW
VVHNNEVESKIDRKWLYNLDKNLDKEYITTLDYMYSDIADELTKSFSKNSAANVNYIAEILNIDSKTFAEQYFRFSIMKEQKNL
GFTLTKLRECMLDREELSDIRDNHKVFDSIRSKLYTMMDFVIYRYYIEEAKKIENENKTLSDDKKKLSEKDIFIISLRGSFSEE
QKDKLYSDEAERLWAKLGKLMLEIKKFRGQMTRDYKKSDTPTLNRILPESEDVSTFSKLMYALTMFLDGKEINELLTTLINKFD
NIQSMLKIMPLIGVNAKFSSDYAFFNNSEKIADELKLIKSFARMGEPVANAKRDMMIDAIKILGTDLDDNEIKKLADSFFKDSN
GKLLHKGKHGMRNFIINNVVNNKRFHYIIRYGDPAHLHEIAKNEVVVRFVLGRIADIQKKQGKGGKNQIDRYYEICIGNGYGKS
VSEKIDALTKVIINMNYDQFEAKRKVIENTGRDNAEREKYKKIISLYLTVIYQILKNLVNVNSRYVIGFHCVERDAQLYKEKGY
DINTNNLESKGFTSVTKLCVGIADDDPVKYKNVEIELKERALASFDALEKENPELYEKYNMYSEKQKEAELEKQINREKAKTAL
NAHLRNTKWNVIIRENIRNTEKDACKQFRNKADHLEVARYAYKYINDISEVNSYFQLYHYIMQRIIIDSSGNNANGMIKKYYES
VISDKKYNDRLLKLLCVPFGYCIPRFKNLSIEALFDKNEAAKYDKIKKKVAVR (SEQ ID NO: 18)
>33000184751Ga0187907_10006632_17
[mammals-digestive system-feces]
MIEKKKSYAKGMGLKSTLVSDSKVYMTSFGNGNDARLEKVVENNAISCLVDKKEAFVAEITDKNAGYKIINKKFGHPKGYDVVA
NNPLYTGPVQQDMLGLKETLEKRYFGSSVSGNDNICIQVIHNILDIEKILAEYITNAAYAVNNIAGLDKDIIGFGKFSTVYTFD
EFAEPDRHKERFIKDGKLDTKLINQLKNQYDEFDAFLDDTRFGYFGKAFFCKEGDKYLNKQDNERYHILALLSGLRNWVVHNNE
VESKIDRKWLYNLDKNLDKEYITTLDYMYSDIADELTKSFSKNSAANVNYIAEILNIDSKTFAEQYFRFSIMKEQKNLGFTLTK
LRECMLDREELSDIRDNHKVFDSIRSKLYTMMDFVIYRYYIEEAKKIENENKTLSDDKKKLSEKDIFIISLRGSFSEEQKDKLY
SDEAERLWAKLGKLMLEIKKFRGQMTRDYKKSDTPTLNRILPESEDVSTFSKLMYALTMFLDGKEINELLTTLINKFDNIQSML
KIMPLIGVNAKFSSDYAFFNNSEKIADELKLIKSFARMGEPVANAKRDMMIDAIKILGTDLDDNEIKKLADSFFKDSNGKLLHK
GKHGMRNFIINNVVNNKRFHYIIRYGDPAHLHEIAKNEVVVRFVLGRIADIQKKQGKGGKNQIDRYYEICIGNGYGKSVSEKID
ALTKVIINMNYDQFEAKRKVIENTGRDNAEREKYKKIISLYLTVIYQILKNLVNVNSRYVIGFHCVERDAQLYKEKGYDINTNN
LESKGFTSVTKLCVGIADDDPVKYKNVEIELKERALASFDALEKENPELYEKYNMYSEKQKEAELEKQINREKAKTALNAHLRN
TKWNVIIRENIRNTEKDACKQFRNKADHLEVARYAYKYINDISEVNSYFQLYHYIMQRIIIDSSGNNANGMIKKYYESVISDKK
YNDRLLKLLCVPFGYCIPRFKNLSIEALFDKNEAAKYDKIKKKVAVR (SEQ ID NO: 19)
>33000184941Ga0187911_10069260_3
[mammals-digestive system-feces]
MSTKKRFRYSVAAKAAGLKSSLAVDTDRTVMTSFGHGNAAILEKEIVDGEISVLNIENPAFDAVINDKKYALTGHHAGVHALVD
QPQNRSDAVHIRGALEKKYFGDTFADNIHVQIAYNILDITKILTVYANNVVYALNNLVHADDDTQADELDSLGNFSAGTSYAKS
KSKSKSKQQDFVELFIKKKEIHGYFGDTFAFLDKRIADADKEKQVYAMLACLGSLRQACSHYRIRYSVNGKNVDADADTWLFSS
AQLDQTDPLFSEMLNRIYSHKIKTVNQNFFENNRKANFPILKKMYPETTLKVLMNEYYDFSIRKGYKNFGFSIKSLREALLSPQ
YESLIGVQIKDNKEYDTVRSKLYQLFDFALTRYFNQHPDMVDAFVVELRSLAKDEDAKNAVYEKYAKAVWNDVKQPIAVMLSYM
NGSAIKNIKAFELKPDQKELNGIMNSNALDVPHFCKLVYFLTRFLDGKEINDLLTTLVNKFDNIHSFNQVLTALGLSASYEADY
KIFEDSGRVVEYLREINSFARMTVDMEKIKRSAYKKALLILGSSKYSDEDLDARVDEMLGVDYNQNGEKIKVRVDTGFRNFIAN
NVVESSRFHYLIRYCHPRKIRNLAGNAALIEYQLRRLPELQILRYYEACTEPIKRTARTMDEKIGTLIDLIVKMDFSQFEDVQQ
NDRVRVFSDAEKKEKIRKMREKQRYQSIISLYLTMLYLIVKNLVNINARYVMAFQAWERDNYLLLQLSGKEAEAEYLNLTRHFI
EPLDGAKPYLKKRPVEYLKKDMAMVGNSSIRHFRNATVHLNVIMEAHRYTKDIKYIGSYYALYHYILQRHLLDKIEEDSYAEKT
VSEKLWESQISQYGTYSKDFVKALCCPFGYNLPRFKNLSIEQLFDRNESKEITDATAPRQ (SEQ ID NO: 20)
>33000184931Ga0187909_10030832_9
[mammals-digestive system-feces]
MAKKKKAKQRREEQEAARMNKIQSAVKAKAETAPAVSSAFVEKRKDKQSKKTFAKASGLKSTLAVDNSAVMTVFGRGNEAKLDH
RINADLQSESLHPQAALKNVHAPNKQKIHFIGRMQDMNLTADHPLHSHDGERAVGADLLCAKDKLEQLYFGRTFNDNIHIQLIY
QILDIQKILALHANNIIFALDNLLHKKNDELSDDFVGMGRMRATIGYDAFRNSTNQKVQETYREFQEFVRRKELLYFGSAFYNG
DTRRDEKVIYHILSLAASVRQFCFHNDYTSDDGKGFIKADWMYRLEEALPAEYKDTLDALYLEGVEGLDQSFLKNNTVNIQILC
SIFNHDDPNKIAEEYYGFLMTKEYKNMGFSIKKLRECMLELPELSGYKEDQYNSVRSKLYKLFDFIIAHYFRKHPEKGEEMVDC
LRLCMTEDEKDSHYEGTAKKLVRELAYDMQEAAEQANGSNITQMQKNEQQGKTKGMFAIRDEIRVSRKPVSYFSKVIYVMTLLL
DGKEINDLLTTLINKFENIVSFEDVLRQLNVDCTFKPEFAFFGYDRCRNISGELRLINSFARMQKPSAKAKHVMYRDALRILGL
DNGMSEEALDQEVRRILQIGADGKPIKNANKGFRNFIASNVIESSRFRYLVRYNNPHKTRMIAQNEAIVRFVLSEIPDEQIRRY
YDVCRDPKLPRSSSREAQVDILTGIITDVNYRIFEDVPQSKKINKDRPDANDRMTLKKQRYQAIVSLYLTVMYLVTKNLVYVNS
RYVMAFHALERDAYLYGITNIKGDYRKLTDNLLADENYKKFGHFKNKKWRGIAEQNLRNSDVPVIKSFRNMAAHISVIRNIDLY
IGDIQKVDSYFALYHFLMQKLIQRVVPENTKGLSDQTKKYYDALEQYNTYCKDFVKAYCTPFAYVTPRYKNLTIDGLFDRNRPG
EDK (SEQ ID NO: 21)
>33000184931Ga0187909_10024847_5
[mammals-digestive system-feces]
MGVEKNKVFESVIMNFDQERKYGFIEYKETNNLFFHMENVKNPKEIVKGAKVRFEIYENPKPKKQNQRFSAINVEVITDETHKE
AKIQKNEFKTFDQFTKELQETQKVNGETKKEHITKNKHTNVKAAGVKSVFAVDDGNVLITSFGRGNAADIETLKSDDDKTINLT
ETENQKKYVVTNKRSNVKGLADNPTKVESIIPGETQIGFKSILEKHFFGRTFNDNIHIQIIHNILDIKKILAVHTNNIVYALDN
IHERGRENSAEKPIDMIGAGGISTSKEYEQYCSEKSDYEDNFLKQLINNERIAYFGNAFFKDEGNKKVMRTEKEIYYILGMLNE
VRNVSTHFTEEDNRDWAKANLYNLSNRLKVGSKEVLNQLYKEKIDKIDANGFVNKGCKRDFSILFKSLNLTTDKDKGELVVGFY
DFSIRKNYKNIGFSIKTLREYMLKISNSTLCADTISNNAIRPKAYKLYDFIIWHYYMNKPDKINDFVEKLRTQNKNDEKIKLYY
DEAVCLLSELGREIHTMTSCVHNIENTSYEITDKKQKEYYKMQINSLNSADKVSDFSKVIYLVTLFLDGKEINDLLTTLINKFD
NIASLLSVLEKQSGKKVEFVENYSFFNSSNLLKEKTLNKSENYTCKIVEELREINSFARMTGDCKIRKSAFEDASQLLGYHDKT
VNNLFEVLRLKELESKDWKKRTDDEQQEYDRLLNKHHYFKSGKKLPDTGLRNFIINNVIESRRFNYIVRYADPKKIRKCTENNE
LLKFAFKDVPDSQVDRYYNICVTNKITNATREEKIERLVDIIKSMNLSKVATVKQRDKQDNVEKQKQLAIMSLYLNILYQIAKN
LVYVNSRYVMAFHSLERDSQMLFDAYYDVKRGYCDLSTVLLFGVDDLQNRNRGSYKYLRDNRRSNKDVIETFGDFKGKVSKVVE
KKNQGLTNEIYDSLCNVAGTTKTEVQNEIKSILKSNGLDESASSYLSHKLVNKVHSYKYLKQNLDCADNTMINQFRNNVAHLNT
IRNMDGIENVTGITSYFQIYHYLMQKALYKEFKKCRENAVRKWIPYITENAEPKYVYWNKKEQQEVEVSFNPKIFGYMENIKNH
SNTYCKDFVKALCAPFAYNLPRFKNLSIEELFDMHELSEEPKESMKLTD (SEQ ID NO: 22)
>WP_074833651.1
[Ruminococcus albus]
MAKKSKGMSLREKRELEKQKRIQKAAVNSVNDTPEKTEEANVVSVNVRTSAENKHSKKSAAKALGLKSGLVIGDELYLTSFGRG
NEAKLEKKISGDTVEKLGIGAFEVAERDESTLTLESGRIKDKTARPKDPRHITVDTQGKFKEDMLGIRSVLEKKIFGKTFDDNI
HVQLAYNILDVEKIMAQYVSDIVYMLHNTDKTERNDNLMGYMSIRNTYKTFCDTSNLPDDTKQKVENQKREFDKIIKSGRLGYF
GEAFMVNSGNSTKLRPEKEIYHIFALMASLRQSYFHGYVKDTDYQGTTWAYTLEDKLKGPSHEFRETIDKIFDEGFSKISKDFG
KMNKVNLQILEQMIGELYGSIERQNLTCDYYDFIQLKKHKYLGFSIKRLRETMLETTPAECYKAECYNSERQKLYKLIDFLIYD
LYYNRKPARIEEIVDKLRESVNDEEKESIYSVEAKYVYESLSKVLDKSLKNSVSGETIKDLQKRYDDETANRIWDISQHSISGN
VNCFCKLIYIMTLMLDGKEINDLLTTLVNKFDNIASFIDVMDELGLEHSFTDNYKMFADSKAICLDLQFINSFARMSKIDDEKS
KRQLFRDALVILDIGNKDETWINNYLDSDIFKLDKEGNKLKGARHDFRNFIANNVIKSSRFKYLVKYSSADGMIKLKTNEKLIG
FVLDKLPETQIDRYYESCGLDNAVVDKKVRIEKLSGLIRDMKFDDFSGVKTSNKAGDNDKQDKAKYQAIISLYLMVLYQIVKNM
IYVNSRYVIAFHCLERDFGMYGKDFGKYYQGCRKLTDHFIEEKYMKEGKLGCNKKVGRYLKNNISCCTDGLINTYRNQVDHFAV
VRKIGNYAAYIKSIGSWFELYHYVIQRIVFDEYRFALNNTESNYKNSIIKHHTYCKDMVKALNTPFGYDLPRYKNLSIGDLFDR
NNYLNKTKESIDANSSIDSQ (SEQ ID NO: 23)
>WP_041337480.1
[Ruminococcus bicirculans]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGFRFKDKQYDSVRSKMYKLMDFLLFCNYYRNDIA
AGESLVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLGVKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDKSPNLFLKKNERLRKCVEVDINNADSSMTRKYRNCIAHLTVVRELKEYIGDICT
VDSYFSIYHYVMQRCITKRENDTKQEEKIKYEDDLLKNHGYTKDFVKALNSPFGYNIPRFKNLSIEQLFDRNEYLTEK (SEQ
ID NO: 24)
>DBYI01000091_43
[Ruminococcus flavefaciens]
MKKKIKARDLREAKKQEKLAAFSAKANTVYENEDKNVEAFPEALNLRSIKKSMNKAAGLKSTLIDGKSLYLTAFGKGNNAVVEH
MIATDDSYSLKTLENEPSLKVKAADELKVTFMSRRPFVQESELSAVNPLHSGKDKPNKSAGQDMLGLKSELEKRYFGKIFDDNL
HIQIIHNILDIEKIIAVYATNITAAIDHMVDDDNEQYLQGDFIGYMNTLNTYEVFMEPSKNPRLDSNARKNIENSREKFEYLLD
TQRLGYLSLEYDKRSKDKRKSEEIKKRLYHLVAFAGQLRQWSFHSVEGLPRTWIYQLDNPKLAQEYRDTLDYFFNERFDAINKD
FIETNNINLHILKEVFPAEDFQKLAALYYDFIVKKTFKNIGFSIKNLREQMLECDEAEKIRSKDMNSVRSKLYKLFDFCIFYQY
FIDEERSRENVNYLRSTLNDEQKDAFYEEEGKRLWSENRKKFIYFCDNINKWVKNDYSDEVAKCIDLNEFRVNSNVSYFSKLLY
AMSFFLDGKEINDLLTTLINKFDNIRSFIDTANFLNIDVKFTKDYDFFNIICDYAGELNIIKNIARMKKPSPSAKKNMYRDALT
ILGIPTEMSDEQLDAEIDKILEKKINPVTGKTEKGKNPFRNFIANNVIENKRFIYVIKFCNPKNVRKLVNNTKVTEFVLKRMPE
TQIDRYFESCIEGNLNPTTEKKIEKLAEMIKNIKFEEFRNVKQKVRDNSQEAVEKERFKAIIGLYLTVIYLLVKNLVNVNSRYV
MAFHCLERDAKLYGVQNIGGDYLALTAKLCAEGDDYGKKLSEAKQNINQDKVQMPKNYFLARNKRWREAIEQDIDNAKKWFIGE
KFNNVKNYRNNVAHLTAIRNCAEFIGEITKIDSYFALYHYLIQRQLAGRLDPNHPGFEKNYPQYAPLFKWNTYVKDMVKALNSP
FGYNIPRFKDLSIDALFDRNEMKEETDDEKKIQT (SEQ ID NO: 25)
>WP_075424065.1
[Ruminococcus flavefaciens]
MIEKKKSFAKGMGVKSTLVSGSKVYMTTFAEGSDARLEKIVEGDSIRSVNEGEAFSAEMADKNAGYKIGNAKFSHPKGYAVVAN
NPLYTGPVQQDMLGLKETLEKRYFGESADGNDNICIQVIHNILDIEKILAEYITNAAYAVNNISGLDKDIIGFGKFSTVYTYDE
FKDPEHHRAAFNNNDKLINAIKAQYDEFDNFLDNPRLGYFGQAFFSKEGRNYIINYGNECYDILALLSGLRHWVVHNNEEESRI
SRTWLYNLDKNLDNEYISTLNYLYDRITNELTNSFSKNSAANVNYIAETLGINPAEFAEQYFRFSIMKEQKNLGFNITKLREVM
LDRKDMSEIRKNHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDNIQSFLKVMPL
IGVNAKFVEEYAFFKDSAKIADELRLIKSFARMGEPIADARRAMYIDAIRILGTNLSYDELKALADTFSLDENGNKLKKGKHGM
RNFIINNVISNKRFHYLIRYGDPAHLHEIAKNEAVVKFVLGRIADIQKKQGQNGKNQIDRYYETCIGKDKGKSVSEKVDALTKI
ITGMNYDQFDKKRSVIEDTGRENAEREKFKKIISLYLTVIYHILKNIVNINARYVIGFHCVERDAQLYKEKGYDINLKKLEEKG
FSSVTKLCAGIDETAPDKRKDVEKEMAERAKESIDSLESANPKLYANYIKYSDEKKAEEFTRQINREKAKTALNAYLRNTKWNV
IIREDLLRIDNKTCTLFRNKAVHLEVARYVHAYINDIAEVNSYFQLYHYIMQRIIMNERYEKSSGKVSEYFDAVNDEKKYNDRL
LKLLCVPFGYCIPRFKNLSIEALFDRNEAAKFDKEKKKVSGNS (SEQ ID NO: 26)
>WP_009985792.1
[Ruminococcus flavefaciens FD-1]
MKKKMSLREKREAEKQAKKAAYSAASKNTDSKPAEKKAETPKPAEIISDNSRNKTAVKAAGLKSTIISGDKLYMTSFGKGNAAV
IEQKIDINDYSFSAMKDTPSLEVDKAESKEISFSSHHPFVKNDKLTTYNPLYGGKDNPEKPVGRDMLGLKDKLEERYFGCTFND
NLHIQIIYNILDIEKILAVHSANITTALDHMVDEDDEKYLNSDYIGYMNTINTYDVFMDPSKNSSLSPKDRKNIDNSRAKFEKL
LSTKRLGYFGFDYDANGKDKKKNEEIKKRLYHLTAFAGQLRQWSFHSAGNYPRTWLYKLDSLDKEYLDTLDHYFDKRFNDINDD
FVTKNATNLYILKEVFPEANFKDIADLYYDFIVIKSHKNMGFSIKKLREKMLECDGADRIKEQDMDSVRSKLYKLIDFCIFKYY
HEFPELSEKNVDILRAAVSDTKKDNLYSDEAARLWSIFKEKFLGFCDKIVVWVTGEHEKDITSVIDKDAYRNRSNVSYFSKLMY
AMCFFLDGKEINDLLTTLINKFDNIANQIKTAKELGINTAFVKNYDFFNHSEKYVDELNIVKNIARMKKPSSNAKKAMYHDALT
ILGIPEDMDEKALDEELDLILEKKTDPVTGKPLKGKNPLRNFIANNVIENSRFIYLIKFCNPENVRKIVNNTKVTEFVLKRIPD
AQIERYYKSCTDSEMNPPTEKKITELAGKLKDMNFGNFRNVRQSAKENMEKERFKAVIGLYLTVVYRVVKNLVDVNSRYIMAFH
SLERDSQLYNVSVDNDYLALTDTLVKEGDNSRSRYLAGNKRLRDCVKQDIDNAKKWFVSDKYNSITKYRNNVAHLTAVRNCAEF
IGDITKIDSYFALYHYLIQRQLAKGLDHERSGFDRNYPQYAPLFKWHTYVKDVVKALNAPFGYNIPRFKNLSIDALFDRNEIKK
NDGEKKSDD (SEQ ID NO: 27)
>CDC65743.1
[Ruminococcus sp. CAG:57]
MAKKNKMKPRELREAQKKARQLKAAEINNNAAPAIAAMPAAEVIAPVAEKKKSSVKAAGMKSILVSENKMYITSFGKGNSAVLE
YEVDNNDYNKTQLSSKDNSNIELGDVNEVNITFSSKHGFGSGVEINTSNPTHRSGESSPVRGDMLGLKSELEKRFFGKTFDDNI
HIQLIYNILDIEKILAVYVTNIVYALNNMLGIKDSESYDDFMGYLSARNTYEVFTHPDKSNLSDKVKGNIKKSLSKFNDLLKTK
RLGYFGLEEPKTKDTRASEAYKKRVYHMLAIVGQIRQCVFHDKSGAKRFDLYSFINNIDPEYRDTLDYLVEERLKSINKDFIEG
NKVNISLLIDMMKGYEADDIIRLYYDFIVLKSQKNLGFSIKKLREKMLEEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKDEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRNESSNLFLKKNKRLRKCVEVDINNADSSMTRKYRNCIAHLTVVRELKEYIG
DIRTVDSYFSIYHYVMQRCITKRGDDTKQEEKIKYEDDLLKNHGYTKDFVKALNSPFGYNIPRFKNLSIEQLFDRNEYLTEK
(SEQ ID NO: 28)
>DJXDO1000002_3
[Ruminococcus sp. UBA7013]
MKKQKSKKTVSKTSGLKEALSVQGTVIMTSFGKGNMANLSYKIPSSQKPQNLNSSAGLKNVEVSGKKIKFQGRHPKIATTDNPL
FKPQPGMDLLCLKDKLEMHYFGKTFDDNIHIQLIYQILDIEKILAVHVNNIVFTLDNVLHPQKEELTEDFIGAGGWRINLDYQT
LRGQTNKYDRFKNYIKRKELLYFGEAFYHENERRYEEDIFAILTLLSALRQFCFHSDLSSDESDHVNSFWLYQLEDQLSDEFKE
TLSILWEEVTERIDSEFLKTNTVNLHILCHVFPKESKETIVRAYYEFLIKKSFKNMGFSIKKLREIMLEQSDLKSFKEDKYNSV
RAKLYKLFDFIITYYYDHHAFEKEALVSSLRSSLTEENKEEIYIKTARTLASALGADFKKAAADVNAKNIRDYQKKANDYRISF
EDIKIGNTGIGYFSELIYMLTLLLDGKEINDLLTTLINKFDNIISFIDILKKLNLEFKFKPEYADFFNMTNCRYTLEELRVINS
IARMQKPSADARKIMYRDALRILGMDNRPDEEIDRELERTMPVGADGKFIKGKQGFRNFIASNVIESSRFHYLVRYNNPHKTRT
LVKNPNVVKFVLEGIPETQIKRYFDVCKGQEIPPTSDKSAQIDVLARIISSVDYKIFEDVPQSAKINKDDPSRNFSDALKKQRY
QAIVSLYLTVMYLITKNLVYVNSRYVIAFHCLERDAFLHGVTLPKMNKKIVYSQLTTHLLTDKNYTTYGHLKNQKGHRKWYVLV
KNNLQNSDITAVSSFRNIVAHISVVRNSNEYISGIGELHSYFELYHYLVQSMIAKNNWYDTSHQPKTAEYLNNLKKHHTYCKDF
VKAYCIPFGYVVPRYKNLTINELFDRNNPNPEPKEEV (SEQ ID NO: 29)
>SCH71549.1
[gut metagenome]
MAKKNKMKPRELREAQKKARQLKAAEINNNAAPAIAAMPAAEVIAPVAEKKKSSVKAAGMKSILVSENKMYITSFGKGNSAVLE
YEVDNNDYNKTQLSSKDNSNIELGDVNEVNITFSSKHGFGSGVEINTSNPTHRSGESSPVRGDMLGLKSELEKRFFGKTFDDNI
HIQLIYNILDIEKILAVYVTNIVYALNNMLGIKDSESYDDFMGYLSARNTYEVFTHPDKSNLSDKVKGNIKKSLSKFNDLLKTK
RLGYFGLEEPKTKDTRASEAYKKRVYHMLAIVGQIRQCVFHDKSGAKRFDLYSFINNIDPEYRDTLDYLVEERLKSINKDFIEG
NKVNISLLIDMMKGYEADDIIRLYYDFIVLKSQKNLGFSIKKLREKMLEEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRNESSNLFLKKNKRLRKCVEVDINNADSSMTRKYRNCIAHLTVVRELKEYIG
DIRTVDSYFSIYHYVMQRCITKRGDDTKQEEKIKYEDDLLKNHGYTKDFVKALNSPFGYNIPRFKNLSIEQLFDRNEYLTEK
(SEQ ID NO: 30)
>SCJ27598.1
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNQTQLSSKDNSNIQLGGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDNRVSQAYKKRVYHMLAIVGQIRQCVFHDKSGAKRFDLYSFINNIDPEYRDTLDYLVEERLKSINKDFIED
NKVNISLLIDMMKGYEADDIIRLYYDFIVLKSQKNLGFSIKKLREKMLDEYGFRFKDKQYDSVRSKMYKLMDFLLFCNYYRNDI
AAGESLVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISGILKLKEKGKGIHGLRNFITNNVIESSRFVYLIKYANAQKIREVAKNEKVVMFVLGGIPDTQIERYYKS
CVEFPDMNSSLGVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIC
TVDSYFSIYHYVMQRCITKRENDTKQEEKIKYEDDLLKNHGYTKDFVKALNSPFGYNIPRFKNLSIEQLFDRNEYLTEK (SEQ
ID NO: 31)
>NZ_ACOK01000100_5
[Ruminococcus flavefaciens FD-1]
MSAMTKGLRNCKGCVNMKKKMSLREKREAEKQAKKAAYSAASKNTDSKPAEKKAETPKPAEIISDNSRNKTAVKAAGLKSTIIS
GDKLYMTSFGKGNAAVIEQKIDINDYSFSAMKDTPSLEVDKAESKEISFSSHHPFVKNDKLTTYNPLYGGKDNPEKPVGRDMLG
LKDKLEERYFGCTFNDNLHIQIIYNILDIEKILAVHSANITTALDHMVDEDDEKYLNSDYIGYMNTINTYDVFMDPSKNSSLSP
KDRKNIDNSRAKFEKLLSTKRLGYFGFDYDANGKDKKKNEEIKKRLYHLTAFAGQLRQWSFHSAGNYPRTWLYKLDSLDKEYLD
TLDHYFDKRFNDINDDFVTKNATNLYILKEVFPEANFKDIADLYYDFIVIKSHKNMGFSIKKLREKMLECDGADRIKEQDMDSV
RSKLYKLIDFCIFKYYHEFPELSEKNVDILRAAVSDTKKDNLYSDEAARLWSIFKEKFLGFCDKIVVWVTGEHEKDITSVIDKD
AYRNRSNVSYFSKLMYAMCFFLDGKEINDLLTTLINKFDNIANQIKTAKELGINTAFVKNYDFFNHSEKYVDELNIVKNIARMK
KPSSNAKKAMYHDALTILGIPEDMDEKALDEELDLILEKKTDPVTGKPLKGKNPLRNFIANNVIENSRFIYLIKFCNPENVRKI
VNNTKVTEFVLKRIPDAQIERYYKSCTDSEMNPPTEKKITELAGKLKDMNFGNFRNVRQSAKENMEKERFKAVIGLYLTVVYRV
VKNLVDVNSRYIMAFHSLERDSQLYNVSVDNDYLALTDTLVKEGDNSRSRYLAGNKRLRDCVKQDIDNAKKWFVSDKYNSITKY
RNNVAHLTAVRNCAEFIGDITKIDSYFALYHYLIQRQLAKGLDHERSGFDRNYPQYAPLFKWHTYVKDVVKALNAPFGYNIPRF
KNLSIDALFDRNEIKKNDGEKKSDD (SEQ ID NO: 200)
>33000062261Ga0099364_10024192_5
[arthropoda-digestive system-cubitermes and nasutitermes termite gut]
MGVKSVLAHGKDEKGHIKLAITAFGKGNKAELAIQTDEKGSNLAKTYKERNITANKIVSEGIQTSGTIAGEGHATFLNNPAEHV
GTDYLKLKETLEMEFFGKSFPGDSVRIQIIHQILDIQKLLGIYITDIIYCINNLRDETHLDHESDIVGLSMSNTNVNLALNQMR
PYFGFFGEAFRPVGDDKVKEITLSDEVRKNIEKIIALEEQKRNPSTPRFKQENINLEIENAMGKFKSKDAFETAKKKYNRIVAD
ETNAKTLRILGAMRQITAHFKDQATLFMSDVELPKILKKEFSKADWQTVEDYYAKLVDRINEGFCKNAATNVHFLTELLPEESK
KQLTEDYFRFAILKEGKNLGVNMKRLREVMFALFVPELTAPETKKRYDSYRAKIYGLTDFLLFKHIHNTKQLEEWVAVLRETSN
EDAKENLYDEFARTAWNTVGDSAKQLIENMQSYFTKKEKEITKTAQPVLSTSSIAHTSKKITQFSSFAKLLAFLCNFWEGKEIN
ELLSAYIHKFENIQEFINLLEKLEGKKPQFTENYALFNEAAGQRAGEIAQNLRILASIGKMKPDLGDAKRQLYKAAIEMLGIDT
EEYISDEWLEPNMLLAQPPKEPKKDNEKYRKEPHKYSYEKDMETYRKKLREYEETWRSLIDYEYLMPETNPFRNFVAKQVIESR
RFMYLVRYTKPKTVRALMSNRAIVHYVLSRIADIQDHHMTESQIDRYYQNLPQYNEQQHKNVSLETKIDALADYLCKYTFEKNV
LKQKNGIVLNTKSATKNVEIEHLKALTGLYLTVAYIAVKNLVKANARYYIAFSIFERDYALFEKKLGKDTLEKYVKPFKYIDKG
EEKEGKNNFFALTEYLLDKDNSLRYQWNNDLSDEENKQALRKHLDKKEIRSQRHFSQYWLDIFARQIENAKKTSESGYLLTAAR
NCALHLNVLTALPEFVGEFRKTGDKMTSYFELYHFLLQKLMLAEAGLNLDEYRERIDTYQTACKDLINITYVSLGYNLPRYKNL
TCEPLFDEESATGKERQTRLDEKSKEKKQRKGGQK (SEQ ID NO: 201)
>CDZK01015063_14
[gut metagenome]
MAKKNKMKPRERREAQKKARQLKAAEINNNAVPAIAAMHAAEVIAPAAEKKKSSVKAAGMKSILVSENKMYITSFGKGNSAVLE
YEVDNNDYNQTQLSSKDNSNIELCGVTKVNITFSSKHGFESGVEINTSNPTHRSGESSPVRGDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIYPEYRDTLDYLVEERLKSINKDFIQGN
KVNISLLIDMMKGYEADDIIRLYYDFIVLKSQKNLGFSIKKLREKMLDEYGFRFKDKQYDSVRSKMYKLMDFLLFCNYYRNDVV
AGEALVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLEAKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDDRDESPNLFLKKNKRLRKCVEVDINNADSSMTRKYRNCIAHLTVVRELKKYIGD
IRTVDSYFSIYHYVMQRCITKREDDTKQEEKIKYEDDLLKNHGYTKDFVKALNSPFGYNIPRFKNLSIEQLFDRNEYLTEK
(SEQ ID NO: 202)
>CEAA01017658_2
[gut metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELCGVNEVNITFSSKHGFESGVEINTSNPTHRSGESSPVRWDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNVLLKTK
RLGYFGLEEPKTKDTRVSQAYKKRVYHMLAIVGQIRQCVFHDKSGAKRFDLYSFINNIDPEYRETLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEASKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDNGDESPNLFLKKNKRLRKCVEVDINNADSNMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 203)
>OCTW011587266_5
[gut metagenome]
MKQNDRENNNKIKKSAAKAVGVKSLARLSDGSTVVSSFGKGAAAELESLITGGEIRKLSDKAILEITDDTQNKNAYNVKSSRIP
NLTARTDKLSDKSGMDDLGFKRELELEVFGQCFDDSIHIQIAHAVFDIQKSLAAVIPNVLYTLNNLDRSYSTDNTSDKKDIIGN
TLNYQHSYESFNVEKRGEFTEYYNAAKDRFSYFPDILCVLEKVNGKDRYQPKSEKDAFNVLSSVNMLRNSLFHFAPKSNDGKAR
IAVFKNQFDSDFSHITSTVNKIYSAKIAGVNENFLNNEGNNLYIILKATNWDIKKIVPQLYRFSVLKSDKNMGFNMRKLREFAV
ESKNIDLSRLNDKFLTNNRKKLYKVIDFIIYYHLNKVLKDSFVDDFVAALRASQSEEEKEKLYAQYSERLFADEGLKSAIKKAV
DMISDTKSNIFKMKTPLDKALIENIKVNSDASDFCKLIYVFTRFLDGKEINILLNSLIKKFQDIHSFNTTVKKLSENNLIINAD
YVDDYSLFEQSGTVARELMLIKSISKMDFGLDNINLSFMYDDALRTLGVSDENLPEVKREYFGKTKNLSAYIRNNVLENRRFKY
VIKYIHPSDVQKIACNKAIAGFVLNRMPDTQIKRYYDSLINKGATDIQAQAKALLDCITGISFDAIKDDKHLHKSKEKSPQRSA
DRERKKAMLTLYYTIVYIFVKQMLHINSLYTIGFFYLERDQRFIYSRAKKENKNPSKNSYLNDFRSVTAYFIPSEIMKRIEKNE
NKGFLEDFEALWNSCGKTSRLRKEDVLLYARYISPDHALKNYKMILNSYRNKIAHINVIMSAGKYTGGIKRMDSYFSVFQHLVQ
CDILSNPNNKGKCFESESLKPLLLDMKFDGTDEKLYSKRLTRALNIPFGYNVPRYKNLTFEKIYLKSSINE (SEQ ID NO:
204)
>OCVV011003687_3
[gut metagenome]
MAKKSKGMKPKEKRELEKQKRIQKAVVKSADDTPVKAEATKAVSVNTDLSVENKHNKKSAAKALGLKSGLVIGDDLYLTSFGRG
NEAKLEKKISGETVENLGIGAFEVTERDESTLTLESGRIKDKTARPKDPRHITVDAQGKFKEDMLGIRSVLEEKIFVGKKFNDN
IHVQLAYNILDIEKIMAQYVSDIVYMLHNTDKTERNDNLMGYMSIQNPYSVFCNPNFSAAKTQRNVVRQKQELDNIIKSGRLGY
FGEAFMVYSGNSSKLRPEKEIYHIFALMASLRQSYFHGYVKDTDYQGPTWPYTLEDKLKDPSHEFRETLDKIFDEGFSKISKNF
GKQNNVNLQILEEMLGELYGSTDSKSLACDYYDFIQLKKHKYLGFSIKRLRETMLETTPAACYKAECYNSIRHKLYLLIDFLIY
DLYYNRKPARIEEIVDKLRESVNDEEKESIYSAETKYVYEALGKVLVRSLKKYLNGATIRDLKNRYDAKTANRIWDISEHSKSG
HVNCFCKLIYMMTLMLDGKEINDLLTTLVNKFDNIASFIDVMDELGLEHSFTDNYKMFADSKAICLDLQFINSFARMSKIDDEK
SKRQLFRDALVVLDIGDKNEDWIEKYLTSDIFKRDENGNKIDGEKRDFRNFIANNVIKSARFKYLVKYSSADGMIKLKKNEKLI
SFVLEQLPETQIDRYYESCGLDCAVADRKVRIEKLTGLIRDMRFDNFRGVNYSNDACKKDKQAKAKYQAIISLYLMVLYQIVKN
MIYVNSRYVIAFHCLERDLLFFNIELDNSYQYSNCNELTDMFIKDKYMKEGALGFNMKAGRYLTKNIGNCSNELRKIYRNQVDH
FAVVRKIGNYAADIKSIGSWFELYHYVMQRIVFDEYRFALNNTESNYKNSIIKHHTYCKDMVKALNTPFGYDLPRYQNLSIGDL
FDRNNYLNKTKESIETKSPIDNP (SEQ ID NO: 205)
>OCVV011003687_3
[gut metagenome]
MVQREGCVMAKKSKGMKPKEKRELEKQKRIQKAVVKSADDTPVKAEATKAVSVNTDLSVENKHNKKSAAKALGLKSGLVIGDDL
YLTSFGRGNEAKLEKKISGETVENLGIGAFEVTERDESTLTLESGRIKDKTARPKDPRHITVDAQGKFKEDMLGIRSVLEEKIF
VGKKFNDNIHVQLAYNILDIEKIMAQYVSDIVYMLHNTDKTERNDNLMGYMSIQNPYSVFCNPNFSAAKTQRNVVRQKQELDNI
IKSGRLGYFGEAFMVYSGNSSKLRPEKEIYHIFALMASLRQSYFHGYVKDTDYQGPTWPYTLEDKLKDPSHEFRETLDKIFDEG
FSKISKNFGKQNNVNLQILEEMLGELYGSTDSKSLACDYYDFIQLKKHKYLGFSIKRLRETMLETTPAACYKAECYNSIRHKLY
LLIDFLIYDLYYNRKPARIEEIVDKLRESVNDEEKESIYSAETKYVYEALGKVLVRSLKKYLNGATIRDLKNRYDAKTANRIWD
ISEHSKSGHVNCFCKLIYMMTLMLDGKEINDLLTTLVNKFDNIASFIDVMDELGLEHSFTDNYKMFADSKAICLDLQFINSFAR
MSKIDDEKSKRQLFRDALVVLDIGDKNEDWIEKYLTSDIFKRDENGNKIDGEKRDFRNFIANNVIKSARFKYLVKYSSADGMIK
LKKNEKLISFVLEQLPETQIDRYYESCGLDCAVADRKVRIEKLTGLIRDMRFDNFRGVNYSNDACKKDKQAKAKYQAIISLYLM
VLYQIVKNMIYVNSRYVIAFHCLERDLLFFNIELDNSYQYSNCNELTDMFIKDKYMKEGALGFNMKAGRYLTKNIGNCSNELRK
IYRNQVDHFAVVRKIGNYAADIKSIGSWFELYHYVMQRIVFDEYRFALNNTESNYKNSIIKHHTYCKDMVKALNTPFGYDLPRY
QNLSIGDLFDRNNYLNKTKESIETKSPIDNP (SEQ ID NO: 206)
>ODAI010069496_4
[gut metagenome]
MAKKIKPRDLRESKRQEKLAAYSVKANEKKTVHTTEEKPAAVLTVTASENKKNKKTSNKAAGLKSTLVYGNKLYITSFGKGNEA
IIEQKVDTSDYSFSDVRSDPSLKIKSADDVSISFSSERPFINKSLLTAVNPLHSGKDKPKRAAGQDMLGLKSELEKRYFGKTFD
DNIHIQLIHNILDIEKIFAVYSANIVAALDHMIDGDDKEYLENDFIGYMNTLNTYEVFMDPSKVFSDCDNRKKNIDKSREKFET
LIDSKRLRYFGFEYDPDGKNKNEEMKKRLYHLVAFAGQLRQWSFHSEGNFQLEWLYKLDDSRIAQEYRDTLDYFFDRRFDELNN
NFVEQNATNLFILKETFPGEDLKAVTDLYYDFIIVKSQKNIGFSIKKLREKMLGTEEAAPIKAHDMDSYRPKLYKLIDFCIFKH
YHEYTEISEKNVDTLRAAVSEEQKESFYADEAKRLWGIFDKQFLGFCKKINVWVNGSHEKEILGYIDKDAYRRKSDVSYFSKFL
YAMSFFLDGKEINDLLTTLINKFDNIASFISTAKELDAEIDRILEKKLDPVTGKPLKGKNSFRNFIANNVIENKRFIYVIKFCN
PKNVLKLVKNTKVTEFVLKRMPESQIDRYYSSCIDTEKNPSVDKKISDLAEMIKKIAFDDFRNVRQKTRTREESLEKERFKAVI
GLYLTVVYLLIKNLVNVNSRYVMAFHCLERDAKLYGINIGKNYIELTEDLCRENENSRSAYLARNKRLRDCVKQNIDNAKNMKS
KEKQRVFFKDYSTVVPFLEKVFYYGSFSSADFEEMDMMKKSKYSYYKRILEYAFGDLLFERKNISKTN (SEQ ID NO:
207)
>ODAI011611274_2
[gut metagenome]
MKKKISLKEQRNTKKAENKLKYQKAQAERAAAAQQTAAGAESEENPCFDVVKDTKRKALNPLHVEIEAPSAKKSSVKANGLKSL
LLTDGKTVMTSFGRGSEANVEKRFDETGTKTFDRDPELFSAKPLETGYRIQRFNASPKDAGLAYRPAGVRPDQIGAKAALEKRY
FGKETPGDNIHVQIAYQIQDIEKLLAVYISNIIYAVNNVTGVSAMKDSKGRPVDLLGDYGILGEEGLTKRLQRIPEQADEEAKA
LQAFLCSERLSYFGKEFCLVRNSPKQPDKEEKRQYKLMRVLCLLGELRQFLVHGKKKEKEFAWLYRLDRQLSQEYRKLLGEFYD
AQVDKVNKSFLTNSTVNLEVLFRALKTGTDPERKTVTQEYYQFTVRKEDGNLGFSLKTLREILLSAYKHEVRDKEYDSIRHKLY
QLFSFALYHYYKTGVGAERREAFVAKLRAVMTAEAKQRAYADEAAEIWNDEGSGIRAAFLEILEAVDFGSAVKGIKARSSVAGD
KRFAEWLEEVRIRPEGVSCFTKLMYLLTRFLDGKEINELLTGLINKLENIQSFLDVMQQEHAETGLSDAFSFFEYSGEIAAELR
MTRSFARMAAADPEAKRFMVVDGAKLLGFNPKDTESEDEGIIRAIYGDACAEYLQFSEEEKEAFYVQEGLYGKEREKFSPYAYF
HTDTSLRNFIAKNVVESARFRYVIRYVSPEIARKYARQEALVRFALHRVPLLQLRRYYQSCCGPKKDPDAAECVDFLAGVVNRV
DFANFTDVRTGDSSKSEQEKKQKYQAIVGLYLTVVYWIVKNLVNVNSRYVMAFHILERDTVLLEGKRLFVGGMKAEDPFLLTDG
YVSRQDAYVRKRIGENKRANRHGLNCVLENRNALGSDPASTDAAASLIWSYRNAAAHLTAVAAAQEYVSELREIHSYFEVYHYA
MQRYLKSGAEFAELVSKNGPASGKIAAWANAVDRCHSFCKDWLWLLNVPFAYNPARYKNLSIANLFDKNEAAPVTEDASEQKED
E (SEQ ID NO: 208)
>OATA01000148_47
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGFRFKDKQYDSVRSKMYKLMDFLLFCNYYRNDIA
AGESLVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLGVKRSELARMIKNISFDDFKNVKQQSKGRENVAKERAKAVIGLYLTVMYLLVKNLVNVNARYVIAIHCLERDFG
LYKEIIPELASKNLKNDYRILSQTLCELCDKSPNLFLKKNERLRKCVEVDINNADSSMTRKYRNCIAHLTVVRELKEYIGDICT
VDSYFSIYHYVMQRCITKRENDTKQEEKIKYEDDLLKNHGYTKDFVKALNSPFGYNIPRFKNLSIEQLFDRNEYLTEK (SEQ
ID NO: 209)
>OAVJ01001264_7
[human gut metagenome]
MAKKNKMKPRELREAQKKARQLKAAEINNNAAPAIAAMPAAEAEKKKSSVKAAGMKSILVSENKMYITSFGKGNSAVLEYEVDK
VDNNVYNQTQLSSKGSSNIKLCGVNEVNITFSSKHGFESGVEINTSNPTHRSGESSPVRGDMLGLKSELEKRFFGKTFDDNIHI
QLIYNILDIEKILAVYVTNIVYALNNMLGIKGSESYDDFMGYLSARNTYEVFTHPDKSSLSDDKKANVRKSLSKFNVLLKTKRL
GYFGLEEPKTKDTRVSQAYKKRVYHMLAIVGQIRQSVFHDKSSKLHEDLYSFIDIIDSEYRETLDYLVDERFDSINKGFIQGNK
VNISLLIDMMKDDYEADDIIRLYYDFIVLKSQKNLGFSIKKLREKMLDEYGFRFKDKQYDSVRSKMYKLMDFLLFCNYYRNDVI
AGEALVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAMLTMFRDALT
ILGIDDKITDDRISEILKLKEKGKGIHGLRNFITNNVIESSRFVYLIKYANAQKIREVAKNEKVVMFVLGGIPDTQIERYYKSC
VEVPDMNSSLEAKRSELARMIKSISFDDFKNVKQQAKGRENVAKEMAKAVIGLYLTVMYLLVKNLVNVNARYVIAIHCLERDFG
LYKEIISELASKNLKNDYRILSQTLCELCDNCDESPNLFLKKNKRLRKCVEVDINNADSSMTRKYRNCIAHLTVVRELKEYIGD
IRTVDSYFSIYHYVMQRCITKREDDKKQEEKIKYEDDLLKNHGYTKDFVKALNSPFGYNIPRFKNLSIEQLFDRNEYLTEK
(SEQ ID NO: 210)
>OBAE01000973_3
[human gut metagenome]
MAKKNKMKPRELREAQKKARQLKAVEINNNAVPEIAAMPAAEVIAPVAEKKKSSVKAAGMKSILVSENKMYITSFGKGNSAVLE
YEVDNNDYNKTQLSSKNSSNIELRGVNEVNITFSSKHGFESGVEINTSNPTHRSGESSPVRWDMLGLKSELEKRFFGKTFDDNI
HIQLIYNILDIEKILAVYVTNIVYALNNMLGIKGSESYDDFMGYLSARNTYEVFTHPDKSSLSDDKKANVRKSLSKFNVLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VIAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDKITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNIRFDDFKNVKQQAKGRENVAKERAKAVIGLYLTVMYLLVKNLVNVNARYVIAIHCLERD
FGLYKEIIPELASKNLKNDYRILSQTLCELCDDRDESPNLFLKKNKRLRKCVEVDINNADSSMTRKYRNCIAHLTVVRELKEYI
GDIYAVDSYFSIYHYVMQRCITKRENDTKQEEKIKYEDNLLKNHGYTKDFVKALNSPFGYNIPRFKNLSIEQLFDRNEYLTEK
(SEQ ID NO: 211)
>OBAR01000289_55
[human gut metagenome]
MAKKNKMKPRELREAQKKARQLKAAEINNNAAPAIAAMPAAEVIAPAAEKKKSSVKAAGMKSIFVSENKMYITSFGKGNSAVLE
YEVDNNDYNQTQLSSKGSSNIELHGVNEVNITFSSKHGFGSGVEINTSNPTHRSGESSPVRWDMLGLKSELEKRFFGKTFDDNI
HIQLIYNILDIEKILAVYVTNIVYALNNMLGVKGSESYDDFMGYLSARNTYEVFTHPDKSNLSDKAKGNIKKSFSTFNDLLKTK
RLGYFGLEEPKTKDTRVSQAYKKRVYHMLAIVGQIRQSVFHDKSSKLDEDLYSFIDIIDPEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAENEKVVMFVLGGIPDTQIERYYKS
CVEVPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDKKQEEKIKYEDDLLKNHGYTKDFVKALNSPFGYNIPRFKNLSIEQLFDRNEYLTEK
(SEQ ID NO: 212)
>OBCV01000332_2
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEGIYADEASKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPASSAKLTMFR
DALTILGIDDNITDDRISEILKLKEKGKGIHGLRNFITNNVIESSRFVYLIKYANAQKIRKVAKNEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNRRLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 213)
>OBDE01000870_1
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNQTQLSSKGSSNIELHGVNEVNITFSSKHGFESGVEINTSNPTHRSGESSPVRWDMLGLKSELEKRFFGKTFDDNI
HIQLIYNILDIEKILAVYVTNIVYALNNMLGVKGSESHDDFIGYLSTNNTYDVFIDPDNSSLSDDKKANVRKSLSKFNVLLKTK
RLGYFGLEEPKTKDTRVSQAYKKRVYHMLAIVGQIRQCVFHDKSGAKRFDLYSFINNIDPEYRETLDYLVDERFDSINKGFIEG
NKINISLLIDMMKGYEADDIIRLYYDFIVLKSQKNLGFSIKKLREKMLDEYGFRFKDKQYDPVRSKMYKLMDFLLFCNHYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLVNFVMIVMSRRICS (SEQ ID NO: 214)
>OBII01002626_5
[human gut metagenome]
MKSILVSKNKMYITSFGKGNSAVLEYEVDNNDYNKTQLSSKDNSNIELRGVTKVNITFSSKHGLESGVEINTSNPTHRSGESSP
VRWDMLGLKSELEKRFFGKTFDDNIHIQLIYNILDIEKILAVYVTNIVYALNNMLGVKGSESHDDFIGYLSTNNTYDVFIDPDN
SSLSDDKKANVRKSLSKFNALLKTKRLGYFGLEEPKTKDNRVSEAYKKRVYHMLAIVGQIRQCVFHDKSGAKRFDLYSFINNID
PEYRETLDYLVDERFDSINKDFIQGNKVNISLLIDMMKGYEADDIIRLYYDFIVLKSQKNLGFSIKKLREKMLDEYGFRFKDKQ
YDSVRSKMYKLMDFLLFCNYYRNDVVAGEALVRKLRFSMTDDEKEGIYADEAAKLWGKFRNDFENIADHMNGDVIKELGKADMD
FDEKILDSEKKNASDILYFSKMIYMLTYFLDGKEINDLLTTLISKFDNIKEFLKIMKSSAVDVECELTAGYKLFNDSQRITNEL
FIVKNIASMRKPAASAKLTMFRDALTILGIDDNITDDRISEILKLKEKGKGIHGLRNFITNNVIESSRFVYLIKYANAQKIREV
AKNEKVVMFVLGGIPDTQIERYYKSCVEFPDMNSSLEVKRSELARMIKNISFDDFKNVKQQAKGRENVAKERAKAVIGLYLTVM
YLLVKNLVNVNARYVIAIHCLERDFGLYKEIIPELASKNLKNDYRILSQTLCDDRDESPNLFLKKNKRLRKCVEVDINNADSSM
TRKYRNCIAHLTVVRELKEYIGDIRTVDTYFSIYHYVMQRCITKREDDTKQEEKIKYEDDLLKNHGYTKDFVKALNSPFGYNIP
RFKNLSIEQLFDRNEYLTEK (SEQ ID NO: 215)
>OBII01002626_3
[human gut metagenome]
MYITSFGKGNSAVLEYEVDNNDYNKTQLSSKDNSNIELRGVTKVNITFSSKHGLESGVEINTSNPTHRSGESSPVRWDMLGLKS
ELEKRFFGKTFDDNIHIQLIYNILDIEKILAVYVTNIVYALNNMLGVKGSESHDDFIGYLSTNNTYDVFIDPDNSSLSDDKKAN
VRKSLSKFNALLKTKRLGYFGLEEPKTKDNRVSEAYKKRVYHMLAIVGQIRQCVFHDKSGAKRFDLYSFINNIDPEYRETLDYL
VDERFDSINKDFIQGNKVNISLLIDMMKGYEADDIIRLYYDFIVLKSQKNLGFSIKKLREKMLDEYGFRFKDKQYDSVRSKMYK
LMDFLLFCNYYRNDVVAGEALVRKLRFSMTDDEKEGIYADEAAKLWGKFRNDFENIADHMNGDVIKELGKADMDFDEKILDSEK
KNASDILYFSKMIYMLTYFLDGKEINDLLTTLISKFDNIKEFLKIMKSSAVDVECELTAGYKLFNDSQRITNELFIVKNIASMR
KPAASAKLTMFRDALTILGIDDNITDDRISEILKLKEKGKGIHGLRNFITNNVIESSRFVYLIKYANAQKIREVAKNEKVVMFV
LGGIPDTQIERYYKSCVEFPDMNSSLEVKRSELARMIKNISFDDFKNVKQQAKGRENVAKERAKAVIGLYLTVMYLLVKNLVNV
NARYVIAIHCLERDFGLYKEIIPELASKNLKNDYRILSQTLCDDRDESPNLFLKKNKRLRKCVEVDINNADSSMTRKYRNCIAH
LTVVRELKEYIGDIRTVDTYFSIYHYVMQRCITKREDDTKQEEKIKYEDDLLKNHGYTKDFVKALNSPFGYNIPRFKNLSIEQL
FDRNEYLTEK (SEQ ID NO: 216)
>OBJF01000033_8
[human gut metagenome]
MAKKKRITAKERKQNHRELLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFGLNYRVPYSEYGGGKDSNGEPKNKFKWEKRDNFSKFYNESK
PHLGYYENIFYDHGEPISEEKFYNYLNILNFIRNNTFHYKDNDIELYSENYSEEYVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKILLEIVEALRLSKNDDEKENVYKKYAEKLFKADDVINPIKAISKLFVEKGNKLFKEKIIIKKEYIEDVSIDKNIYDFT
KVIFFMTCFLDGKEINDLLTNIISKLQIIEDHNNVIKFISHNKDAVYKDYSDKYAIFRNAGKIATELEAIKSIARMENKIENAS
QEPLLNDALLSLGVSDDTKVLENTYKKYFDSKEKTDKQSQKVSTFLMNNVINNNRFKYVIKYINPADINGLAKNRYLVKFVLSK
IPEEQIDSYYKLFSNEEEPGCEEKIKLLTKKISKLNFQTLFENNKIPNVEKEKKKAIITLYFTIVYILVKNLVNINGLYTLALY
FVERDGYFYKDICGKKDKKKSYNDVDYLLLPEIFSGSKYREETKNLKLPKEKDRDIMKKYLPNDKDREKYNKFFTAYRNNIVHL
NIIAKLSELTKNIDKDINSYFDIYHYCTQRVMFNYCKEKNDVVLAKMKDLAHIKSDCNEFSSKHTYPFSSAVLRFMNLPFAYNV
PRFKNLSYKKFFDKQWLKHYENLNDFIRILY (SEQ ID NO: 217)
>OBJF01000033_8
[human gut metagenome]
MAKKKRITAKERKQNHRELLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFGLNYRVPYSEYGGGKDSNGEPKNKFKWEKRDNFSKFYNESK
PHLGYYENIFYDHGEPISEEKFYNYLNILNFIRNNTFHYKDNDIELYSENYSEEYVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKILLEIVEALRLSKNDDEKENVYKKYAEKLFKADDVINPIKAISKLFVEKGNKLFKEKIIIKKEYIEDVSIDKNIYDFT
KVIFFMTCFLDGKEINDLLTNIISKLQIIEDHNNVIKFISHNKDAVYKDYSDKYAIFRNAGKIATELEAIKSIARMENKIENAS
QEPLLNDALLSLGVSDDTKVLENTYKKYFDSKEKTDKQSQKVSTFLMNNVINNNRFKYVIKYINPADINGLAKNRYLVKFVLSK
IPEEQIDSYYKLFSNEEEPGCEEKIKLLTKKISKLNFQTLFENNKIPNVEKEKKKAIITLYFTIVYILVKNLVNINGLYTLALY
FVERDGYFYKDICGKKDKKKSYNDVDYLLLPEIFSGSKYREETKNLKLPKEKDRDIMKKYLPNDKDREKYNKFFTAYRNNIVHL
NIIAKLSELTKNIDKDINSYFDIYHYCTQRVMFNYCKEKNDVVLAKMKDLAHIKSDCNEFSSKHTYPFSSAVLRFMNLPFAYNV
PRFKNLSYKKFFDKQ (SEQ ID NO: 218)
>OBKG01000025_26
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGDVDEVNITFSSKHGFGSGVEINTSNPTHRSGESSPVRWDMLGLKSELEKRFFGKTFDDNI
HIQLIYNILDIEKILAVYVTNIVYALNNMLGVKGSESHDDFIGYLSTNNTYDVFIDPDNSSLSDDKKANVRKSLSKFNVLLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDNRDESPNLFLKKNKRLRKCVEVDINNADSSMTRKYRNCIAHLTVVRELKEYIG
DIRTVDSYFSIYHYVMQRCITKREDDKKQEEKIKFEDDLLKNHGYTKDFVKALNSPFGYNIPRFKNLSIEQLFDRNEYLTEK
(SEQ ID NO: 219)
>OBKR01000858_3
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKYNSNIELGDVNEVNITFSSKHGFGSGMKINTSNPTHRSGESSPVRWDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNVLLKTK
RLGYFGLEEPKTKDNRVSEAYKKRVYHMLAIVGQIRQCVFHDKSGAKRFDLYSFINNIDPEYRDTLDYLVEERLKSINKDFIQG
NKVNISLLIDMMKGYEADDIIRLYYDFIVLKSQKNLGFSIKKLREKMLEEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 220)
>OBVH01003037_1
[human gut metagenome]
MAKKKRITAKERKQNHRELLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFGLNYRVPYSEYGGGKDSNGEPKNQSKWEKRDNFIKFYNESK
PHLGYYENIFYDHGEPISEEKFYNYLNILNFIRNNTFHYKDDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKLLLEIVETLRLSKNDDEKENVYKKYAEKLFKADDVINPIKAISKLFARKGNKLFKEKIIIKKEYIEDVSIDKNIYDFT
KVIFFMTCFLDGKEINDLLTNIISKLQVIEDHNNVIKFISNNKDAVYKDYSDKYAIFRNAGKIATELEAIKSIARMENKIENAP
QEPLLKDALLSLGVSDDTKVLENTYNKYFDSKEKTDKQSQKVSTFLMNNVINNNRFKYVIKYINPADINGLAKNRYLVKFVLSK
IPEEQIDSYYKLFSNEEEPGCEEKIKLLTKKISKLNFQTLFENNKIPNVEKEKKKAIITLYFTIVYILVKNLVNINGLYTLALY
FVERDGYFYKDICGKKDKKKSYNDVDYLLLPEIFSGSKYREETKNLKLPKEKDRDIMKKYLPNDKDREKYNKFFTAYRNNIVHL
NIIAKLSELTKNIDKDINSYFDIYHYCTQRVMFNYCKEKNDVVLAKMKDLAHIKSDCNEFSSKHTYPFSSAVLRFMNLPFAYNV
PRFKNLSYKKFFDKQWLKHYENLNDFIRILY (SEQ ID NO: 221)
>OBVH01003037_2
[human gut metagenome]
MAKKKRITAKERKQNHRELLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFGLNYRVPYSEYGGGKDSNGEPKNQSKWEKRDNFIKFYNESK
PHLGYYENIFYDHGEPISEEKFYNYLNILNFIRNNTFHYKDDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKLLLEIVETLRLSKNDDEKENVYKKYAEKLFKADDVINPIKAISKLFARKGNKLFKEKIIIKKEYIEDVSIDKNIYDFT
KVIFFMTCFLDGKEINDLLTNIISKLQVIEDHNNVIKFISNNKDAVYKDYSDKYAIFRNAGKIATELEAIKSIARMENKIENAP
QEPLLKDALLSLGVSDDTKVLENTYNKYFDSKEKTDKQSQKVSTFLMNNVINNNRFKYVIKYINPADINGLAKNRYLVKFVLSK
IPEEQIDSYYKLFSNEEEPGCEEKIKLLTKKISKLNFQTLFENNKIPNVEKEKKKAIITLYFTIVYILVKNLVNINGLYTLALY
FVERDGYFYKDICGKKDKKKSYNDVDYLLLPEIFSGSKYREETKNLKLPKEKDRDIMKKYLPNDKDREKYNKFFTAYRNNIVHL
NIIAKLSELTKNIDKDINSYFDIYHYCTQRVMFNYCKEKNDVVLAKMKDLAHIKSDCNEFSSKHTYPFSSAVLRFMNLPFAYNV
PRFKNLSYKKFFDKQ (SEQ ID NO: 222)
>OBVY01000267_8
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGDVNEVNITFSSKHGFGSGMKINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNIRFDDFKNVKQQAKGRENVAKERAKAVIGLYLTVMYLLVKNLVNVNARYVIAIHCLER
DFGLYKEIIPELASKNLKNDYRILSQTLCELCDNGDESPNLFLKKNKRLRKCVEVDINNADSNMTRKYRNCIAHLTVVRELKEY
IGDIRTVDSYFSIYHYVMQRCITKREDDTKQEEIIKYEDDLLKNHGYTKDFVKALNSPFGYNIPRFKNLSIEQLFDRNEYLTEK
(SEQ ID NO: 223)
>OBXZ01000094_20
[human gut metagenome]
MAKKKRITAKERKQNHRESLMKKADSNAEKEKAKKPVVENKPDTAISKDNIPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRVPYSEYGGGKDSNGEPKNKSKWEKRKNFIKFYNKSK
PHLGYYENIFYDHGEPISEEKFYNYLNILNFIRNNTFHYKNDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSIKHKLYKTYDFVITHY
LNSNDKLLLEIVEALRLSKNDDEKENVYKKYAEKLFKADDVINPIKAISKLFAEKGNKLFKEMVIIKKEYVEDISIDKNIYDFT
KVIFFMTCFLDGKEINDLLTNIISKLQVIEDHNNVIKFISNNKDAVYKDYSDKYAIFRNAGKIATELEAIKSIVRMENKIENAP
QEPLLKDALLSLGVSDDTKVLENTYKKYFDSKEKADKQSQKVSTFLMNNVINNNRFKYVIKYINPADINGLAKNRYLVKFVLSN
IPEEQIDSYYKLFSNEEEPSCEEKIKLLTKKISKLNFQTLFENNKIPNVEKERKKAIITLYFTIVYILVKNLVNINGLYTLALY
FVERDGYFYKDICGKKDKKKSYNGVDYLLLPEIFSGSKYREQTKNLKLPKEKDRDIMKKYLPNDKDREGYNKFFRAYRNNIVHL
NIIAKLSELTSNIDKDINSYFDIYHYCTQRVMFNYCKENNNIVLAKMKDLAHIKSDCDEFSSKHTYPFSSAVLRFMNLPFAYNV
PRFKNLSYKKFFDKQWLNH (SEQ ID NO: 224)
>OBXZ01000094_20
[human gut metagenome]
MAKKKRITAKERKQNHRESLMKKADSNAEKEKAKKPVVENKPDTAISKDNIPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRVPYSEYGGGKDSNGEPKNKSKWEKRKNFIKFYNKSK
PHLGYYENIFYDHGEPISEEKFYNYLNILNFIRNNTFHYKNDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSIKHKLYKTYDFVITHY
LNSNDKLLLEIVEALRLSKNDDEKENVYKKYAEKLFKADDVINPIKAISKLFAEKGNKLFKEMVIIKKEYVEDISIDKNIYDFT
KVIFFMTCFLDGKEINDLLTNIISKLQVIEDHNNVIKFISNNKDAVYKDYSDKYAIFRNAGKIATELEAIKSIVRMENKIENAP
QEPLLKDALLSLGVSDDTKVLENTYKKYFDSKEKADKQSQKVSTFLMNNVINNNRFKYVIKYINPADINGLAKNRYLVKFVLSN
IPEEQIDSYYKLFSNEEEPSCEEKIKLLTKKISKLNFQTLFENNKIPNVEKERKKAIITLYFTIVYILVKNLVNINGLYTLALY
FVERDGYFYKDICGKKDKKKSYNGVDYLLLPEIFSGSKYREQTKNLKLPKEKDRDIMKKYLPNDKDREGYNKFFRAYRNNIVHL
NIIAKLSELTSNIDKDINSYFDIYHYCTQRVMFNYCKENNNIVLAKMKDLAHIKSDCDEFSSKHTYPFSSAVLRFMNLPFAYNV
PRFKNLSYKKFFDKQ (SEQ ID NO: 225)
>OCHB01002119_1
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEGIYADEASKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPASSAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDTKQEEKIKYEDDLLKNHGYTKDFVKALNSPFGYNIPRFKNLSIEQLFDRNEYLTE
K (SEQ ID NO: 226)
>OCHC01000012_250
[human gut metagenome]
MAKKNKMKPRELREAQKKARQFKAAEINNNAVPAIAAMPAAEAAAPAAEKKKSSVKAAGMKSILVSENKMYITSFGKGNSAVLE
YEVDNNDYNKTQLSSEDSSNIELCGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLGVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKREDDIKQEEKIKYEDDLLKNHGYTKDFVKALNSPFGYNIPRFKNLSIEQLFDRNEYLTEK (SEQ
ID NO: 227)
>OCHN01000290_35
[human gut metagenome]
MLCLKPTLEKKFFGKEFNDNIHIQLIYNILDIEKILAVYSTNAIYALNNMSADENIENSDFFMKRTTDETFDDFEKKKESTNSR
EKADFDAFEKFIGNYRLAYFADAFYVDKNKSKSKPKDKAKGIQRGEKEIYSILALIAKLRHWCVHSEEGRAEFWLYKLDELKSD
FKNVLDVVYNRPVEKINNRFIENNKVNIQILGSVYKNTDIAELVRSYYEFLITKKYKNMGFSIKKLRESMLEGKGYADKEYDSV
RNKLYQMTDFILYTGYINEDSDRADDLVNTLRSSLKEDDKTTVYCKEADYLWKKYRESIREVAASLDVKNINELKNNAFTIPDN
ELRKCFISYADSVSEFTKLIYLLTRFLSGKEINDLVTTLINKFDNIRSFLEVMDELGLERTFTDEYSFFEGSTKYLAELVELNS
FVKSCSFDINAKRTMYRDALDILGIESGKTEEDIEKMIDNIVQFDANGKKLPNKNHGLRNFIASNVIDSNRFEYLVRYGNPKKI
RETAKCKPAVRFVLNEIPDAQIERYYKACYPDEKSLCFANMQRDKLAGVIANIKFDDFSDAGSYQKANATSTKITSEAEIKRKN
QAIIRLYLTVMYIMLKNLVNVNARYVIAFHCVERDTKLYAESGLEVGNIEKNKTNLTMAVMGVKLENGIIKTEFDKSLAENAAN
RYFRNARWYKLILDNLKKSERAVVNEFRNTVCHLNAIRNININIKEIKEVENYFALYHYLIQKHLEKRFADNGGSTGDFISKLE
EHKTYCKDFVKAYCTPFGYNLVRYKNLTIDGLFDKNYPGKDDSDKQK (SEQ ID NO: 228)
>OCPQ01000020_138
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVNEVNITFSSKHGFESGVEINTSNPTHRSGESSPVRWDMLGLKSELEKRFFGKTFDDNI
HIQLIYNILDIEKILAVYITNIVYALNNMLGVKGSESHDDFIGYLSTNNIYDVFIDPDNSSLSDDKKANVRKSLSKFNALLKTK
RLGYFGLEEPKTKDNRVSEAYKKRVYHMLAIVGQIRQSVFHDKSSKLHEDLYSFIDIIDSEYRETLDYLVDERFDSINKGFIQG
NKVNISLLIDMMKGYEADDIIRLYYDFIVIKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSPLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKREDDIKQEEKIKYEDDLLKNHGYTKDFVKALNSPFGYNIPRFKNLSIEQLFDRNEYLTEK (SEQ
ID NO: 229)
>OCPU01001206_17
[human gut metagenome]
MAKKNKMKPRELREAQKKARQFKAAEINNNAVPAIAAMPAAEAAAPAAEKKKSSVKAAGMKSILVSENKMYITSFGKGNSAVLE
YEVDNNDYNKTQLSSEDSSNIELCGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLGVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFCASALKSILIMQTAA (SEQ ID NO: 230)
>OFMU01000310_31
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNQTQLSSKGSSNIELHGVNEVNITFSSKHGFESGVEINTSNPTHRSGESSPVRWDMLGLKSELEKRFFGKTFDDNI
HIQLIYNILDIEKILAVYVTNIVYALNNMLGVKGSESHDDFIGYLSTNNTYDVFIDPDNSSLSDDKKANVRKSLSKFNVLLKTK
RLGYFGLEEPKTKDTRVSQAYKKRVYHMLAIVGQIRQCVFHDKSGAKRFDLYSFINNIDPEYRETLDYLVDERFDSINKGFIEG
NKINISLLIDMMKGYEADDIIRLYYDFIVLKSQKNLGFSIKKLREKMLDEYGFRFKDKQYDPVRSKMYKLMDFLLFCNHYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 231)
>OFMV01000268_25
[human gut metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELCGVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAIDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNIRFDDFKNVKQQAKGRENVAKERAKAVIGLYLTVMYLLVKNLVNVNARYVIAIHCLER
DFGLYKEIIPELASKNLKNDYRILSQTLCELCDNGDESPNLFLKKNKRLRKCVEVDINNADSSMTRKYRNCIAHLTVVRELKEY
IGDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 232)
>OGCM01002738_3
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDNNDYNQTQLSSDGSSNIELRGVNEVNITFSSKHGFESGVEINTSNPTHRSGESSPVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSNELDEYLYSFIDIIDSEYRDTLDYLVDERFDSINKGFVQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAENEKVVMFVLGGIPDTQIERYYKS
CVEFPDMNSPLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKREDDTKQEEKIKYEDDLLKNHGYTKDFVKALNSPFGYNIPRFKNLSIEQLFDRNEYLTEK (SEQ
ID NO: 233)
>OGCO01000353_15
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGVKGSESHDDFIGYLSTNNTYDVFIDPDNSSLSDDKKANVRKSLSKFNVLL
KTKRLGYFGLEEPKTKDNRVSEAYKKRVYHMLAIVGQIRQCVFHDKSGAKRFDLYSFINNIDPEYRDTLDYLVEERLKSINKDF
IQGNKVNISLLIDMMKGYEADDIIRLYYDFIVLKSQKNLGFSIKKLREKMLEEYGFRFKDKQYDSVRSKMYKLMDFLLFCNYYR
NDIAAGEALVRKLRFSMTDDEKEGLYADEAAKLWGKFRNDFENIADHMNGDVIKELGKADMDFDEKILDSEKKNASDLLYFSKM
IYMLTYFLDGKEINDLLTTLISKFDNIKEFLKIMKSSAVNVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIRKVAENEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNERLRKCVEVDINNADSSMTRKYRNCIAHLTVVRELKEYIG
DIRTVDSYFSIYHYVMQRCITKREDDKKQEEKIKFEDDLLKNHGYTKDFVKALNSPFGYNIPRFKNLSIEQLFDRNEYLTEK
(SEQ ID NO: 234)
>OGOK01000323_15
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGFRFKDKQYDSVRSKMYKLMDFLLFCNYYRNDIA
AGESLVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDKITDDRISEILKLKEKGKGIHGLRNFVTNNVIESSRFVYLIKYANAQKIREVAKNEKVVMFVLGGIPDTQIERYYKSC
VEFPDMNSSLEAKRSELARMIKNIRFDDFKNVKQQAKGRENVAKERAKAVIGLYLTVMYLLVKNLVNVNARYVIAIHCLERDFG
LYKEIIPELASKNLKNDYRILSQTLCELCDNPDESPNLFLKKNRRLRKCVEVDINNADSSMTRKYRNCIAHLTVVRELKEYIGD
ICTVDSYFSIYHYVMQRCITKRENDTKQEEKIKYEDDLLKNHGYTKDFVKALNSPFGYNIPRFKNLSIEQLFDRNEYLTEK
(SEQ ID NO: 235)
>OGOL01000786_27
[human gut metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSPVRGDMLGLKSELEKRFFGKTFDDNI
HIQLIYNILDIEKILAVYVTNIVYALNNMLGEDDDESHDDFMGYLSAKNTYDVFTDPDESDLSKNIKGNIKKSLSKFNDLLKTK
RLGYFGLEEPKTKDTNALEAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLEEYGFRFKDKQYDSVRSKMYKLMDFLLFCNYYRNDV
AAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIRKVAENEKVVMFVLGGIPDTQIERYYKS
CVEFPDMNSSLEAKYSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNRRLRKCVEVDINNADSSMTRKYRNCIAHLTVVRELKEYIG
DIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 236)
>OGOO01001137_18
[human gut metagenome]
MAKKNKMKPRELREAQKKARQLKAVEINNNAVPEIAAMPAAEVIAPVAEKKKSSVKAAGMKSILVSENKMYITSFGKGNSAVLE
YEVDNNDYNKTQLSSKNSSNIELRGVNEVNITFSSKHGFESGVEINTSNPTHRSGESSPVRWDMLGLKSELEKRFFGKTFDDNI
HIQLIYNILDIEKILAVYVTNIVYALNNMLGIKGSESYDDFMGYLSARNTYEVFTHPDKSSLSDDKKANVRKSLSKFNVLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VIAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDKITDDRISEILKLKEKGKGIHGLRNFITNNVIESSRFVYLIKYANAQKIREVAENEKVVMFVLGGIPDTQIERYYK
SCVEFPDMNSPLEAKRSELARMIKNIRFDDFKNVKQQAKGRENVAKERAKAVIGLYLTVMYLLVKNLVNVNARYVIAIHCLERD
FGLYKEIIPELASKNLKNDYRILSQTLCELCDKSPNLFLKKNERLRKCVEVDINNADSSMTRKYRNCIAHLTVVRELKEYIGDI
RTVDSYFSIYHYVMQRCITKREDDIKQEEKIKYEDDLLKNHGYTKDFVKALNSPFGYNIPRFKNLSIEQLFDRNEYLTEK
(SEQ ID NO: 237)
>OGOP01001824_10
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVDEVNITFSSKHGFESGVKINTSNPTHRSGESSSVRGDMLGLKSELEKRFFGKTFDDNI
HIQLIYNILDIEKILAVYVTNIVYALNNMLGLENESNNDFMGYLSAKNTYDVFTDPDESDLSKNIKGNIKKSLSKFNDLLKTKR
LGYFGLEEPKTKDKRVSEAYKKRVYHMLAIVGQIRQSVFHDKSNELDEYLYSFIDIIDSEYRETLDYLIDERFDSINKGFIQGN
KVNISLLIDMMKGYEADDIIRLYYDFIVLKSQKNLGFSIKKLREKMLDEYGFRFKDKQYDSVRSKMYKLMDFLLFCNYHRKDVV
AGEALVRKLRFSMTDEEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVNVECELTTGYKLFNDSQRITNELFIVKNIASMRKPAASAKLTMFRDALT
ILGIEDNITDDRISEILKLKEKGKGIHGLRNFITNNVIESSRFVYLIKYANAQKIREVAENEKVVMFVLGGIPDTQIERYYKSC
VEFPDMNSSMGAKRRELAKMIKSISFENFKDVKQQAKGRENVAKERAKAVIGLYLTVMYLLVKNLVNVNARYVIAIHCLERDFG
LYKEIIPELASKNLKNDYRILSQTLCELCDNGDESPNLFLKKNKRLRKCVEVDINNADSNMTRKYRNCIAHLTVVRELKEYIGD
IYAVDSYFSIYHYVMQRCITKRENDTEQAEKIKYEDDLFKNHGYTRDFVKALNSPFGYNIPRFKNLSIKQMFDRNEYLTEK
(SEQ ID NO: 238)
>OGPB01000314_7
[human gut metagenome]
MAKKNKMKPRELREAQKKARQFKAAEINNNAVPAIAAMPAAEAAAPAAEKKKSSVKAAGMKSILVSENKMYITSFGKGNSAVLE
YEVDNNDYNKTQLSSEDSSNIELCGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEVKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKKYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 239)
>OGPJ01000449_26
[human gut metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNQTQLSSDGSSNIELRGVNEVNITFSSKHGFESGVEINTSNPTHRSGESSPVRWDMLGLKSELEKRFFGKTFDDNI
HIQLIYNILDIEKILAVYVTNIVYALNNMLGEGDDESHDDFMGYLSAKNTYDVFTDPDESDLSKNIKGNIKKSLSKFNDLLKTK
RLGYFGLEEPKTKDTNALEAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAENEKVVMFVLGGIPDTQIERYYKS
CVEFPDMNSPLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKREDDIKQEEKIKYEDDLLKNHGYTKDFVKALNSPFGYNIPRFKNLSIEQLFDRNEYLTEK (SEQ
ID NO: 240)
>OGPS01000624_23
[human gut metagenome]
MGKKIHARDLREQRKTDRTEKFADQNKKREAERAVQKKDAAVSVKSVSSVSSKKDNVTKSMAKAAGVKSVFAVENTVYMTSFGR
GNDAVLEQKIVDTSHEQLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKARNVLREDKELYSVLTLIGKLRHWCVHSEEGRAEFWLYKLNELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKRLSKSNIEIQEDKLRKCFISYADSVSEFT
KLIYLLTRFLSGKEINDLVTTLINKFDNIRSFLEIMDELGLDRTFTAEYSFFEDSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPENTALCSANKKREKLADMIAEIEFENFSDAGNYQKANVTSKTHEAEIKRKNQSIIRLYLTVMYIMLKNLV
NVNARYVIAFHCVERDTKLYAESGLEVGNIEKNKTNLTMAVMGVKLENGIIKTEFDKSFAENAANRYLRNARWYKLILDNLKKS
ERAVVNEFRNTVCHLNAIRNININIDGIKEVENYFALYHYLIQKHLENRFADKKVERDTGDFISKLEEHKTYCKDFVKAYCTPF
GYNLVRYKNLTIDGLFDKNYPGKDDSDKQK (SEQ ID NO: 241)
>OGQH01000331_48
[human gut metagenome]
MAKKNKMKPRELREAQKKARQLKAAEINNNAAPAIAAMPAAEVIAPAAEKKKSSVKAAGMKSILVSKNKMYITSFGKGNSAVLE
YEVDNNDYNQTQLSSKNSSNIELHGVNEVNITFSSKHGFESGVEINTSNPTHRSGESSPVRGDMLGLKSELEKRFFGKTFDDNI
HIQLIYNILDIEKILAVYVTNIVYALNNMLGEGDDESHDDFMGYLSAKNTYDVFTDPDESDLSKNIKGNIKKSLSKFNDLLKTK
RLGYFGLEEPKTKDTNALEAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAENEKVVMFVLGGIPDTQIERYYKS
CVEFPDMNSPLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKREDDIKQEEKIKYEDDLLKNHGYTKDFVKALNSPFGYNIPRFKNLSIEQLFDRNEYLTEK (SEQ
ID NO: 242)
>OGQ001007270_2
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNKTQLSSKGSSNIELHGVNEVNITFSSKHGFESGVEINTSNPTHRSGESSPVRGDMLGLKSELEKRFFGKTFDDNI
HIQLIYNILDIEKILAVYVTNIVYALNNMLGEGDDESHDDFMGYLSTFNTYKVFTNPNGSTLSDDKKENIRKSLSKFNALLKTK
RLGYFGLEEPKTKDTRASEAYKKRVYHMLAIVGQIRQSVFHDKSNELDEYLYSFIDIIDSEYRETLDYLIDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDNRDESPNLFLKKNKRLRKCVEVDINNADSSMTRKYRNCIAHLTVVRELKEYIG
DIRTVDSYFSIYHYVMQRCITKREDDKKQEEKIKFEDDLLKNHGYTKDFVKALNSPFGYNIPRFKNLSIEQLFDRNEYLTEK
(SEQ ID NO: 243)
>OGQW01001429_6
[human gut metagenome]
MAKKNKMKPRELREAQKKARQLKAAEINNNAAPAIAAAPAAEKKKSSVKAAGMKSILVSENKMYITSFGKGNSAVLEYEVDNND
YNQTQLSSKGSSNIELHGVNEVNITFSSKHGFESGVEINTSNPTHRSGESSPVRWDMLGLKSELEKRFFGKTFDDNIHIQLIYN
ILDIEKILAVYVTNIVYALNNMLGVKGSESHDDFIGYLSTNNTYDVFIDPDNSSLSDDKKANVRKSLSKFNVLLKTKRLGYFGL
EEPKTKDTRVSQAYKKRVYHMLAIVGQIRQCVFHDKSGAKRFDLYSFINNIDPEYRETLDYLVDERFDSINKGFIEGNKINISL
LIDMMKGYEADDIIRLYYDFIVLKSQKNLGFSIKKLREKMLDEYGFRFKDKQYDPVRSKMYKLMDFLLFCNHYRNDVAAGEALV
RKLRFSMTDDEKEGIYADEAAKLWGKFRNDFENIADHMNGDVIKELGKADMDFDEKILDSEKKNASDLLYFSKMIYMLTYFLDG
KEINDLLTTLISKFDNIKEFLKIMKSSAVDVECELTAGYKLFNDSQRITNELFIVKNIASMRKPAASAKLTMFRDALTILGIDD
NITDDRISEILKLKEKGKGIHGLRNFITNNVIESSRFVYLIKYANAQKIREVAKNEKVVMFVLGGIPDTQIERYYKSCVEFPDM
NSSLEAKRSELARMIKNISFDDFKNVKQQAKGRENVAKERAKAVIGLYLTVMYLLVKNLVNVNARYVIAIHCLERDFGLYKEII
PELASKNLKNDYRILSQTLCELCDDRDESPNLFLKKNKRLRKCVEVDINNADSSMTRKYRNCIAHLTVVRELKEYIGDIRTVDS
YFSIYHYVMQRCITKRENDTKQEEKIKYEDDLLKNHGYTKDFVKALNSPFGYNIPRFKNLSIEQLFDRNEYLTEK (SEQ ID
NO: 244)
>OGRA01000610_24
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELRDVNEVNITFSSKHGFESGVEINTSNPTHRSGESSPVRWDMLGLKSELEKRFFGKTFD
DNIHIQLIYNILDIEKILAVYVTNIVYALNNMLGEGDDESHDDFMGYLSAKNTYDVFTDPDESDLSKNIKGNIKKSLSKFNDLL
KTKRLGYFGLEEPKTKDTNALEAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLEEYGYRFKDKQYDSVRSKMYKLMDFLLFCNYYR
NDVAAGEALVRKLRFSMTDDEKEGIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIRKVAKNEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNRRLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 245)
>OGRE01001635_6
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDKVDNNVYNQTQLSSKGSSNIKLCGVNEVNITFSSKHGFESGVEINTSNPTHRSGESSPVRGDMLGLKSELEKRFFGKTFD
DNIHIQLIYNILDIEKILAVYVTNIVYALNNMLGIKGSESYDDFMGYLSARNTYEVFTHPDKSSLSDDKKANVRKSLSKFNVLL
KTKRLGYFGLEEPKTKDTRVSQAYKKRVYHMLAIVGQIRQSVFHDKSSKLHEDLYSFIDIIDSEYRETLDYLVDERFDSINKGF
IQGNKVNISLLIDMMKDDYEADDIIRLYYDFIVLKSQKNLGFSIKKLREKMLDEYGFRFKDKQYDSVRSKMYKLMDFLLFCNYY
RNDVIAGEALVRKLRFSMTDDEKEGIYADEAAKLWGKFRNDFENIADHMNGDVIKELGKADMDFDEKILDSEKKNASDLLYFSK
MIYMLTYFLDGKEINDLLTTLISKFDNIKEFLKIMKSSAVDVECELTAGYKLFNDSQRITNELFIVKNIASMRKPAASAMLTMF
RDALTILGIDDKITDDRISEILKLKEKGKGIHGLRNFITNNVIESSRFVYLIKYANAQKIREVAKNEKVVMFVLGGIPDTQIER
YYKSCVEVPDMNSSLEAKRSELARMIKSISFDDFKNVKQQAKGRENVAKEMAKAVIGLYLTVMYLLVKNLVNVNARYVIAIHCL
ERDFGLYKEIISELASKNLKNDYRILSQTLCELCDNCDESPNLFLKKNKRLRKCVEVDINNADSSMTRKYRNCIAHLTVVRELK
EYIGDIRTVDSYFSIYHYVMQRCITKREDDKKQEEKIKYEDDLLKNHGYTKDFVKALNSPFGYNIPRFKNLSIEQLFDRNEYLT
EK (SEQ ID NO: 246)
>OGRF01000967_2
[human gut metagenome]
MAKKNKMKPRELREAQKKARQLKAVEINNNAVPEIAAMPAAEVIAPVAEKKKSSVKAAGMKSILVSENKMYITSFGKGNSAVLE
YEVDNNDYNKTQLSSKNSSNIELRGVNEVNITFSSKHGFESGVEINTSNPTHRSGESSPVRWDMLGLKSELEKRFFGKTFDDNI
HIQLIYNILDIEKILAVYVTNIVYALNNMLGIKGSESYDDFMGYLSARNTYEVFTHPDKSSLSDDKKANVRKSLSKFNVLLKTK
RLGYFGLEEPKTKDTRVSQAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAENEKVVMFVLGGIPDTQIERYYKS
CVEFPDMNSSLEAKRSELARMIKNISFDNFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELNKYIK
DIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 247)
>OGRN01001989_2
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDKVDDNDYNQTQLSSKDNSNIELGDVNEVNITFSSKHGFESGVEINTSNPTHRSGESSPVRGDMLGLKSELEKRFFGKTFD
DNIHIQLIYNILDIEKILAVYVTNIVYALNNMLGVKGSESHDDFIGYLSTNNIYDVFIDPDNSSLSDDKKANVRKSLSKFNALL
KTKRLGYFGLEEPKTKDNRVSQAYKKRVYHMLAIVGQIRQCVFHDKSGAKRFDLYSFINNIDPEYRDTLDYLVEERLKSINKDF
IEDNKVNISLLIDMMKDYEADDIIRLYYDFIVLKSQKNLGFSIKKLREKMLDEYGYRFKDKQYDSVRSKMYKLMDFLLFCNYYR
NDVVAGESLVRKLRFSMTDDEKEGIYADEASKLWGKFRNDFENIADHMNGDVIKELGKADMDFDEKILDSEKKNASDLLYFSKM
IYMLTYFLDGKEINDLLTTLISKFDNIKEFLKIMKSSAVNVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLKAKRSELARMIKNISFEDFKDVKQQAKGRENVAKERAKAVIGLYLTVMYLLVKNLVNVNARYVIAIHCLE
RDFGLYKEIIPELASKNLKNDYRILSQMLCELCDDRDKSPNLFLKKNKRLRKCVEVDINNADSSMTRKYRNCIAHLTVVRELKE
YIGDIYAVDSYFSIYHYVMQRCITKRENDTKQEEKIKYEDNLLKNHGYTKDFVKALNSPFGYNIPRFKNLSIEQLFDRNEYLTE
K (SEQ ID NO: 248)
>OGRQ01003333_5
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNQTQLSSKGSSNIELHGVNEVNITFSSKHGFESGVEINTSNPTHRSGESSPVRWDMLGLKSELEKRFFGKTFDDNI
HIQLIYNILDIEKILAVYVTNIVYALNNMLGIKKSESYDDFMGYLSARNTYEVFTHPDKSNLSDKVKGNIKKSLSKFNVLLKTK
RLGYFGLEEPKTKDTNALEAYKKRVYHMLAIVGQIRQSVFHDKSSKLHEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VAAGEALVRKLRFSMTDDEKEGIYADEAEKLWGKFRNDFENIADHMNGDVIKELGKADMDFDEKILDSEKKNASDLLYFSKMIY
MLTYFLDGKEINDLLTTLISKFDNIKEFLKIMKSSAVNVECELTAGYKLFNDSQRITNELFIVKNIASMRKPAASAKLTMFRDA
LTILGIDDKITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDDRDESPNLFLKKNKRLRKCVEVDINNADSSMTRKYRNCIAHLTVVRELKEYI
GDIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 249)
>OGRU01000829_2
[human gut metagenome]
MKPRELREAQKKARQLKAAEINNNAAPAIAAMPAAEVIAPAAEKKKSSVKAAGMKSILVSENKMYITSFGKGNSAVLEYEVDNN
DYNKTQLSSKDNSNIELGDVNEVNITFSSKHGFGSGMKINTSNPTHRSGESSPVRWDMLGLKSELEKRFFGKTFDDNIHIQLIY
NILDIEKILAVYVTNIVYALNNMLGEGDDSNYDFMGYLSTFNTYKVFTNPNGSTLSDDKKKNIRKSLRKFNDLLKTKRLGYFGL
EEPKTKDNRVSEAYKKRVYHMLAIVGQIRQCVFHDKSGAKRFDLYSFINNIYPEYRDTLDYLVDERFDSINKGFIQGNKVNISL
LIDMMKGYEPDDIIRLYYDFIVLKSQKNLGFSIKKLREKMLDEYGFRFKDKQYDSVRSKMYKLMDFLLFCNYYRNDIAAGESLV
RKLRFSMTDDEKEGIYADEAAKLWGKFRNDFENIADHMNGDVIKELGKADMDFDEKILDSEKKNASDLLYFSKMIYMLTYFLDG
KEINDLLTTLISKFDNIKEFLKIMKSSAVDVECELTVGYKLFNDSQRITNELFIVKNIASMRKPAASAKLTMFRDALTILGIDD
KITDDRISEILKLKEKGKGIHGLRNFITNNVIESSRFVYLIKYANAQKIRKVAENEKVVMFVLGGIPDAQIERYYKSCVEFPDM
NSSLEVKRSELARMIKNISFDDFKNVKQQAKGRENVAKERAKAVIGLYLTVMYLLVKNLVNVNARYVIAIHCLERDFGLYKEII
PELASKNLKNDYRILSQTLCELCDNGDESPNLFLKKNKRLRKCVEVDINNADSNMTRKYRNCIAHLTVVRELNKYIKDIRTVDS
YFSIYHYVMQRCITKREDDKKQEEKIKYEDDLLKNHGYTKDFVKALNSPFGYNIPRFKNLSIEQLFDRNEYLTEK (SEQ ID
NO: 250)
>OGSD01001176_18
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSESSSNIELCGVTKVNITFSSKHGFGSGVEINTSNPTHRSGESSPVRWDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDTRVSQAYKKRVYHMLAIVGQIRQCVFHDKSGAKRFDIYSFINNIDPEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFEYIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAENEKVVMFVLGGIPDTQIERYYKS
CVEVPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDKKQEEKIKYEDDLLKNHGYTKDFVKALNSPFGYNIPRFKNLSIEQLFDRNEYLTEK
(SEQ ID NO: 251)
>OGWY01002732_3
[human gut metagenome]
MGKKIHARDLREQRKTDRTEKFADQNKKREAQRAVQKKDAAVSVKSVSSVSSKKDNVTKSMAKAAGVKSVFAVGNTVYMTSFGR
GNDAVLEQKIVDTSHEPLNIDDPAYQLNVVTMNGYSVTGHRGETVSAVTDNPLRRFNGRKKDEPEQSVPTDMLCLKPTLEKKFF
GKEFDDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKARNVLREDKELYSVLTLIGKLRHWCVHSEEGRAEFWLYKLNELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYCESIREVAEALDGDNIKRLSKSNIEIRDNELRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIKSGKTEEDIEKMIDNILQIDANGDKKLEKNNGLRNFIASNVIDSNRFKYLVRYGNPKKIRETAKCKPAVRFVLNEI
PDAQIERYYEACCPKNTALCSANKRREKLADMIAEIKFENFSDAGNYQKANVTSKTHEAEIKRKNQAIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNININIDGIKEVENYFALYHYLIQKHLENRFADNGGSTGDYIGKLEEHKTYCKDFVKAYCTPFGY
NLVRYKNLTIDGLFDKNYPGKDDSDEQK (SEQ ID NO: 252)
>OGXI01000433_6
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVDEVNITFSSKHGFESGVKINTSNPTHRSGESSSVRGDMLGLKSELEKRFFGKTFDDNI
HIQLIYNILDIEKILAVYVTNIVYALNNMLGLENESNNDFMGYLSAKNTYDVFTDPDESDLSKNIKGNIKKSLSKFNDLLKTKR
LGYFGLEEPKTKDKRVSEAYKKRVYHMLAIVGQIRQSVFHDKSNELDEYLYSFIDIIDSEYRETLDYLIDERFDSINKGFIQGN
KVNISLLIDMMKDDYEADDIIRLYYDFIVLKSQKNLGFSIKKLREKMLDEYGFRFKDKQYDSVRSKMYKLMDFLLFCNYYRNDV
IAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAENEKVVMFVLGGIPDTQIERYYKS
CVEFPDMNSSLEVKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDNGDESPNLFLKKNRRLRKCVEVDINNADSSMTRKYRNCIAHLTVVRELKEYIG
DIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 253)
>OGXJ01002463_5
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDKVDNNNYNKTQLSSKDNSNIELGDVNEVNITFSSKRGNESGVEINTSNPTHRSGESSPVRWDMLGLKSELEKRFFGKTFD
DNIHIQLIYNILDIEKILAVYVTNIVYALNNMLGIKKSESYDDFMGYLSARNTYEVFTHPDKSNLSDKAKGNIKKSFSTFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYETDDIIRLYYDFIVLKSQKNLGFSIKKLREKMLDEYGFRFKDKQYDSVRSKMYKLMDFLLFCNYYR
NDVVAGEALVRKLRFSMTDDEKEGTYADEAEKLWGKFRNDFENIADHMNGDAIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNERLRKCVEVDIYNADSSMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 254)
>OGXL01002096_10
[human gut metagenome]
MAKKNKMKPRELREAQKKARQLKAAEINNNAVPAIAAMPAAEVIAPAAEKKKSSVKAAGMKSILVSENKMYITSFGKGNSAVLE
YEVDKVDDNDYNKTQLSSKDNSNIELGNVNEVNITFSSRRGFESGVEINTSNPTHRSGESSSVRGDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDKRVSEAYKKRVYHMLAIVGQIRQSVFHDKSNELDEYLYSFIDIIDSEYRDTLDYLVDERFDSINKGF
IQGNKVNISLLIDMMKDDYEADDIIRLYYDFIVLKSQKNLGFSIKKLREKMLEEYGFRFKDKQYDSVRSKMYKIMDFLLFCNYY
RNDVVAGEALVRKLRFSMTDDEKEGIYADEAAKLWGKFRNDFENIADHMNGDVIKELGKADMDFDEKILDSEKKNASDLLYFSK
MIYMLTYFLDGKEINDLLTTLISKFDNIKEFLKIMKSSAVDVECELTAGYKLFNDSQRITNELFIVKNIASMRKPAASAKLTMF
RDALTILGIDDKITDDRISEILKLKEKGKGIHGLRNFITNNVIESSRFVYLIKYANAQKIREVAKNEKVVMFVLGGIPDTQIER
YYKSCVEFPDMNSSLEVKRSELARMIKNISFDDFKNVKQQAKGRENVAKERAKAVIGLYLTVMYLLVKNLVNVNARYVIAIHCL
ERDFGLYKEIIPELASKNLKNDYRILSQTLCELCDKSPNLFLKKNERLRKCVEVDINNADSSMTRKYRNCIAHLTVVRELKEYI
GDIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 255)
>OGYD01000683_23
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVNEVNITFSSKHGFESGVEINTSNPTHRSGESSPVRWDMLGLKSELEKRFFGKTFDDNI
HIQLIYNILDIEKILAVYITNIVYALNNMLGVKGSESHDDFIGYLSTNNIYDVFIDPDNSSLSDDKKANVRKSLSKFNALLKTK
RLGYFGLEEPKTKDNRVSEAYKKRVYHMLAIVGQIRQSVFHDKSSKLHEDLYSFIDIIDSEYRETLDYLVDERFDSINKGFIQG
NKVNISLLIDMMKGYEADDIIRLYYDFIVIKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNKRLRKCVEVDINNADSSMTRKYRNCIAHLTVVRELKEYIGDIR
TVDSYFSIYHYVMQRCITKRENDTKQEDKIKYEDDLLKNHGYTKDFVKALNSPFGYNIPRFKNLSIEQLFDRNEYLTEK (SEQ
ID NO: 256)
>OGYL01002810_3
[human gut metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELCGVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASNRFILFSTM
IYMLTYFLDGKEINDLLTTLISKFDNIKEFLKIMKSSAIDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRILSQTLCELCDNGDESPNLFLKKNKRLRKCVEVDINNADSSMTRKYRNCIAHLTVVRELKE
YIGDIRTVDSYFSIYHYVMQRCITKREDDTKQEEKIKYEDDLLKNHGYTKDFVKALNSPFGYNIPRFKNLSIEQLFDRNEYLTE
K (SEQ ID NO: 257)
>OGYY01000371_37
[human gut metagenome]
MAKKNKMKPRELREAQKKARQLKAAEINNNATPTIAAMPAAEVIAPAAEKKKSSVKAAGMKSILVSKNKMYITSFGKGNSAVLE
YEVDNNDYNQTQLSSKNSSNIELRGVNEVNITFSSKHGFESGVEINTSNPTHRSGESSSVRGDMLGLKSELEKRFFGKTFDDNI
HIQLIYNILDIEKILAVYVTNIVYALNNMLGIKDSESYDDFIGYLSARNTYKVFTHPDKSNLSDKVKGNIKKSFSTFNDLLKTK
RLGYFGLEEPKTKDTRVSQAYKKRVYHMLAIVGQIRQSVFHDKSSKLDEDLYSFIDIIDPEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAAKLWGKFRNDFENIADHMNGEAIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNIGFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 258)
>OGZC01000639_10
[human gut metagenome]
MKKKNIRATREALKAQKIKKSQENEALKKQKLAEEAAQKRREELEKKNLAQWEETSAEGRRSRVKAVGVKSVFVVGDDLYLATF
GNGNETVLEKKITPDGKITTFPEEETFTAKLKFAQTEPTVATSIGISNGRIVLPEISVDNPLHTTMQKNTIKRSAGEDILQLKD
VLENRYFDRSFNDDLHIRLIYNILDIEKILAEYTTNAVFAIDNVSGCSDDFLSNFSTRNQWDEFQNPEQHREHFGNKDNVICSV
KKQQDLFFNFFKNNRIGYFGKAFFHAESERKIVKKTEKEVYHILTLIGSLRQWITHSTEGGISRLWLYQLEDALSREYQETMNN
CYNSTIYGLQKDFEKTNAPNLNFLAEILGKNASELAEPYFRFIITKEYKNLGFSIKTLREMLLDQPDLQEIRENHNVYDSIRSK
LYKMIDFVLVYAYSNERKSKADALASNLRSAITEDAKKRIYQNEADQLWTSYQELFKRIRGFKGAQVKEYSSKNMPIPIQKQIQ
NILKPAEQVTYFTKLMYLLTMFLDGKEINDLLTTLINKFDNISSLLKTMEQLELQTTFKEDYTFFQQSSRLCKEITQLKSFARM
GNPISNLKEVMMVDAIQILGTEKSEQELQSMACFFFRDKNGKKLNTGEHGMRNFIGNNVISNTRFQYLIRYGNPQKLHTLSQNE
TVVRFVLSRIAKNQRVQGMNGKNQIDRYYETCGGTNSWSVSEEEKINFLCKILTNMSYDQFQDVKQSGAEITAEEKRKKERYKA
IISLYLTVLYQLIKNLVNINARYIIAFHCLERDAILYSSKFNTSINLKKRYTALTEMILGYETDEKARRKDTRTVYEKAEAAKN
RHLKNVKWNCKTRENLENADKNAIVAFRNIVAHLWIIRDADRFITGMGAMKRYFDCYHYLLQRELGYILEKSNQGSEYTKKSLE
KVQQYHSYCKDFLHMLCLPFAYCIPRYKNLSIAELFDRHEPEAEPKEEASSVNNSQFITT (SEQ ID NO: 259)
>OHAI01000724_7
[human gut metagenome]
MAKKNKMKPRELREAQKKARQLKAAEINNNAVPAIAAMPAVEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSNELDEYLYSFIDIIDSEYRDTLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAENEKVVMFVLGGIPDTQIERYYKS
CVEVPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDNGDESPNLFLKKNKRLRKCVEVDINNADSSMTRKYRNCIAHLTVVRELKEYIG
DIRTVDSYFSIYHYVMQRCITKRENDTKQEEKIKYEDDLLKNHGYTRDFVKALNSPFGYNIPRFKNLSIKQLFDRNEYLTEK
(SEQ ID NO: 260)
>OHAJ01000052_20
[human gut metagenome]
MAKKKRITAKERKQNHRESLMKKADSNAEKEKAKKPVVENKPDTAISKDNTPKPNKEIKKSKAKLAGVKWVIKANDDVAYISSF
GKGNNSVLEKRIIGDVSSDVNKDSHMYVNPKYTKKNYEIKNGFSSGSSLTTHPNKPDKNSGMDALCLKIYFEKEIFKDKFNDNM
HIQTIYNIFDIEKTLAKHITNIIYAVNSLDRSYIQSGNDTIGFGLNYRIPYAKYGRGKDSNGKPNNSNLKKRESFIKFYNNAKD
RFGYFESVFYQNGKPISREKLYIYLNILNFVRNSTFHYNNTSTYLYRKEYKYTDKDNCSVKEFEFVSYLNEFVKNKFKNVNKNF
ISNEKNNLYIILNAYGEDIEDVEVVKKYSKELYKLSVLKTNKNLGVNVKKLRESAIEYGYCPLPYDKEKEVAKLSSIKHKLYKT
YDFVITHYLNSNDKLLLEIVEALRLSKNDDEKENVYKIYAEKIFKAEYVINPIKTISNLFAEKGDKLFNEKVSISEEYVEDIRI
DKNIHNFTKVIFFLTCFLDGKEINDLLTNIISKLQVIEDHNNVIKAIANNNDAVYKDYSDKYAVFKNSGKIATKLEAIKSIARM
ENKINKAFKEPLLKDAMLALGVSPNDLDEKYEKYFKTDVDADKDHQKVSTFLMNNVINNSRFKYVVKYINPADINRLAKNKHLV
KFVLDQIPHKQIDSYYNSVCTVEEPSYKGKIQLLTKKITGLNFYSLFENCKIPNVEKEKKKAVITLYFTIIYILVKNLVNINGL
YTLALYFVERDGFFYKKICEKKDKKKTNKDVDYLLLPEIFSGSKYREETKNLKLPKEKDREIMKKYLPNDEDRKEYNKFFKQYR
NNIVHLNIIANLSKLTSTIDKEINSYFEIFHYCAQRVMFDYCKNNNKVVLAKMKDLAHIKSDCDEFSSKYTYPYSSAVLRFMNL
PFAYNVPRFKNLSYQKFFDKQRLEALEKNLNI (SEQ ID NO: 261)
>OHAN01001071_11
[human gut metagenome]
MAKKNKMKPRELREAQKKARQLKAAEINNNAAPAIAAMPAAEAAAPAAEKKKSSVKAAGMKSILVSENKMYITSFGKGNSAVLE
YEVDKVDNNDYNQTQLSSKGSSNIELHGVNEVNITFSSKHGFESGVEINTSNPTHRSGESSPVRWDMLGLKSELEKRFFGKTFD
DNIHIQLIYNILDIEKILAVYVTNIVYALNNMLGIKDSESYDDFMGYLSAKNTYDVFTDPDESDLSKNIKGNIKKSFSTFNDLL
KTKRLGYFGLEEPKTKDTRVSEAYKKRVYHMLAIVGQIRQCVFHDKSGAKRFDLYSFINNIDPEYRDTLDYLVEERLKSINKDF
IEGNKVNISLLIDMMKGYEADDIIRLYYDFIVLKSQKNLGFSIKKLREKMLDEYGFRFKDKQYDSVRSKMYKLMDFLLFCSYYR
NDVVAGEALVRKLRFSMTDDEKEGIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRTLSQTLCELCDKSPNLFLKKNERLRKCVEVDINNADSIMTRKYRNCIAHLTVVRELKEYIG
DIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 262)
>OHAR01000226_9
[human gut metagenome]
MAKKNKMKPRELREAQKKARQLKVAEINNNAVPAIAAMPAAEAAAPAAEKKKSSVKAAGMKSILVSKNKMYITSFGKGNSAVLE
YEVDKVDNDNYNKTQLSSKDNSNIELGDVDEVNITFSSKHGFESGVEINTSNPTHRSGESSPVRWDMLGLKSELEKRFFGKTFD
DNIHIQLIYNILDIEKILAVYVTNIVYALNNMLGIKKSESYDDFMGYLSARNTYEVFTHPDKSNLSDKVKGNIKKSLSKFNVLL
KTKRLGYFGLEEPKTKDTNALEAYKKRVYHMLAIVGQIRQSVFHDKSSKLHEDLYSFIDIIDSEYRETLDYLVDERFDSINKGF
IQGNKVNISLLIDMMKDDYEADDIIRLYYDFIVLKSQKNLGFSIKKLREKMLDEYGFRFKDKQYDSVRSKMYKLMDFLLFCNYY
RNDVVAGEALVRKLRFSMTDDEKEGIYADEAEKLWGKFRNDFENIADHMNGDVIKELGKADMDFDEKILDSEKKNASDLLYFSK
MIYMLTYFLDGKEINDLLTTLISKFDNIKEFLKIMKSSAVNVECELTAGYKLFNDSQRITNELFIVKNIASMRKPAASAKLTMF
RDALTILGIDDKITDDRISEILKLKEKGKGIHGLRNFITNNVIESSRFVYLIKYANAQKIREVAKNEKVVMFVLGGIPDTQIER
YYKSCVEFPDMNSSLEAKRSELARMIKNISFDDFKNVKQQAKGRENVAKERAKAVIGLYLTVMYLLVKNLVNVNARYVIAIHCL
ERDFGLYKEIIPELASKNLKNDYRILSQTLCELCDDRDESPNLFLKKNRRLRKCVEVDINNADSSMTRKYRNCIAHLTVVRELK
EYIGDIRTVDSYFSIYHYVMQRCITKREDDTKQEDKIKYEDDLLKNHGYTKDFVKALNSPFGYNIPRFKNLSIEQLFDRNEYLT
EK (SEQ ID NO: 263)
>OHBL01000590_7
[human gut metagenome]
MAKKNKMKPRERREAQKKARQLKAAEINNNAVPAIAAMPAAEVIAPAAEKKKSSVKAAGMKSILVSENKMYITSFGKGNSAVLE
YEVDNNDYNKTQLSSKDNSNIELGDVNEVNITFSSRRGFESGVEINTSNPTHRSGESSSVRGDMLGLKSELEKRFFGKNFDDNI
HIQLIYNILDIEKILAVYVTNIVYALNNMLGVKGSESYDDFMGYLSAKNTYEVFTHPDKSNLSDKVKGNIKKSFSTFNDLLKTK
RLGYFGLEEPKTKDTRVSQAYKKRVYHMLAIVGQIRQSVFHDKSSKLHEDLYSFIDIIDSEYRETLDYLVEERLKSINKDFIEG
NKVNISLLIDMMKGYEADDIIRLYYDFIVLKSQKNLGFSIKKLREKMLDEYGFRFKDKQYDPVRSKMYKLMDFLLFCNYYRNDV
VTGEALVRKLRFSMTDDEKEGIYADEASKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKRENDTKQEEKIKYEDDLLKNHGYTKDFVKALNSPFGYNIPRFKNLSIEQLFDRNEYLTEK (SEQ
ID NO: 264)
>OHBP01000023_129
[human gut metagenome]
MAKKNKMKPRELREAQKKARQLKAAEINNNAAPAIAAMPAAEVIAPVAEKKKSSVKAAGMKSILVSENKMYITSFGKGNSAVLE
YEVDNNDYNKTQLSSKDNSNIELGDVNEVNITFSSKHGFGSGVEINTSNPTHRSGESSPVRGDMLGLKSELEKRFFGKTFDDNI
HIQLIYNILDIEKILAVYVTNIVYALNNMLGIKDSESYDDFMGYLSARNTYEVFTHPDKSNLSDKVKGNIKKSLSKFNDLLKTK
RLGYFGLEEPKTKDTRASEAYKKRVYHMLAIVGQIRQCVFHDKSGAKRFDLYSFINNIDPEYRDTLDYLVEERLKSINKDFIEG
NKVNISLLIDMMKGYEADDIIRLYYDFIVLKSQKNLGFSIKKLREKMLEEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 265)
>OHBQ01000429_2
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNVYNQTQLSSKGSSNIKLCGVNEVNITFSSKHGFESGVEINTSNPTHRSGESSPVRGDMLGLKSELEKRFFGKTFD
DNIHIQLIYNILDIEKILAVYVTNIVYALNNMLGVKDSESYDDFMGYLSARNTYEVFTHPDKSNLSDKVKGNIKKSLSKFNDLL
KTKRLGYFGLEEPKTKDTRVSQAYKKRVYHMLAIVGQIRQCVFHDLSEHSEYDLYSFIDNSKKVYRECRETLDYLVDERFDSIN
KGFIQGNKVNISLLIDMMKGYEADDIIRLYYDFIVLKSQKNLGFSIKKLREKMLDEYGYRFKDKQYDSVRSKMYKLMDFLLFCN
YYRNDVVAGEALVRKLRFSMTDDEKEGIYADEAEKLWGKFRNDFENIADHMNGDVIKELGKADMDFDEKIIDSEKKNASDLLYF
SKMIYMLTYFLDGKEINDLLTTLISKFDNIKEFLKIMKSSAVNVECELTAGYKLFNDSQRITNELFIVKNIASMRKPAASAKLT
MFRDALTILGIDDNITDDRISEILKLKEKGKGIHGLRNFITNNVIESSRFVYLIKYANAQKIREVAENEKVVMFVLGGIPDTQI
ERYYKSCVEFHDMNSSLEAKRSELARMIKNISFDDFKNVKQQAKGRENVAKERAKAVIGLYLTVMYLLVKNLVNVNARYVIAIH
CLERDFGLYKEIIPELASKNLKNDYRILSQTLCELCDDRDESPNLFLKKNKRLRKCVEVDINNADSSMTRKYRNCIAHLTVVRE
LKEYIGDIRTVDSYFSIYHYVMQRCITKREDDTKQEDKIKYEDDLLKNHGYTKDFVKALNSPFGYNIPRFKNLSIEQLFDRNEY
LTEK (SEQ ID NO: 266)
>OHBW01001448_1
[human gut metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNKTQLSSKDNSNIELGNVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNIRFDDFKNVKQQAKGRENVAKERAKAVIGLYLTVMYLLVKNLVNVNARYVIAIHCLER
DFGLYKEIIPELASKNLKNDYRILSQTLCELCDNGDESPNLFLKKNKRLRKCVEVDINNADSNMTRKYRNCIAHLTVVRELKEY
IGDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 267)
>OHCE01000125_17
[human gut metagenome]
MAKKNKMKPRERREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLEEYGYRFKDKQYDSVRSKMYKLMDFLLFCNYYRNDVV
AGEALVRKLRFSMTDDEKEGIYADEASKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPASSAKLTMFRDALT
ILGIDDNITDDRISEILKLKEKGKGIHGLRNFITNNVIESSRFVYLIKYANAQKIRKVAKNEKVVMFVLGGIPDTQIERYYKSC
VEFPDMNSSLEVKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDESPNLFLKKNERLRKCVEVDINNADSSMTRKYRNCIAHLTVVRELKEYIGDIRT
VDSYFSIYHYVMQRCITKREDDKKQEEKIKFEDDLLKNHGYTKDFVKALNSPFGYNIPRFKNLSIEQLFDRNEYLTEK (SEQ
ID NO: 268)
>OHCH01000211_3
[human gut metagenome]
MAKKNKMKPRELREAQKKARQFKAAEINNNAVPAIAAMPAAEAAAPAAEKKKSSVKAAGMKSILVSENKMYITSFGKGNSAVLE
YEVDNNDYNKTQLSSEDSSNIELCGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFVVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNERLRKCVEVDINNADSSMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 269)
>OHCP01000044_27
[human gut metagenome]
MAKKITAKQKREEKERLNKQKWAKNDSVIIVPETKEEIKTGEIQDNNRKRSRQKSQAKAMGLKAVLSFDNKIAIASFVSSKNAK
SSHIERITDKEGTTISVNSKMFESSVNKRDINIEKRITIEEPQQDGTIKKEEKGVKSTTCNPYFKVGGKDYIGIKEIAEEHFFG
RAFPNENLRVQIAYNIFDVQKILGTFVNNIIYSFYNLSRDEVQSDNDVIGMLYSISDYDRQKETETFLQAKSLLKQTEAYYAYF
DDVFKKNKKPDKNKEGDNSKQYQENLRHNFNILRVLSFLRQICMHAEVHVSDDEGCTRTQNYTDSLEALFNISKAFGKKMPELK
TLIDNIYSKGINAINDEFVKNGKNNLYILSKVYPNEKREVLLREYYNFVVCKEGSNIGISTRKLKETMIAQNMPSLKEENTYRN
KLYTVMNFILVRELKNCATIREQMIKELRANMDEEEGRDRIYSKYAKEIYLYVKDKLKLMLNVFKEEAEGIIIPGKEDPVKFSH
GKLDKKEIESFCLTTKNTEDITKVIYFLCKFLDGKEINELCCAMMNKLDGISDLIETAKQCGEDVEFVDQFKCLSKCATMSNQI
RIVKNISRMKKEMTIDNDTIFLDALELLGRKIEKYQKDKNGDYVKDEKGKKVYTKDYNNFQDMFFEGKNHRVRNFVSNNVIKSK
WFSYVVRYNKPAECQALMRNSKLVKFALDELPDSQIEKYYISVFGEKSSSSNEEMRRELLKKLCDFSVRGFLDEIVLLSEDEMK
QKDKFSEKEKKKSLIRLYLTIVYLITKSMVKINTRFSIACATYERDYILLCQSEKAERAWEKGATAFALTRKFLNHDKPTFEQY
YTREREISAMPQEKRKELRKENDQLLKKTHYSKHAYCYIVDNVNNLTGAVANDNGRGLPCLSEKNDNANLFLEMRNKIVHLNVV
HDMVKYINEIKNITSYYAFFCYVLQRMIIGNNSNEQNKFKAKYSKTLQEFGTYSKDLMWVLNLPFAYNLPRYKNLSNEQLFYDE
EERMEKIVGRKNDSR (SEQ ID NO: 270)
>OHCW01000317_3
[human gut metagenome]
MAKKNKMKPRELREAQKKARQLKAAEINNNAAPAIAAMPVAEAAAPAAEKKKSSVKAAGMKSILVSENKMYITSFGKGNSAVLE
YEVDNNDYNKTQLSSKDNSNIELGDVNEVNITFSSKHGFESGVEINTSNPTHRSGESSPVRGDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYYMLAIVGQIRQCVFHDLSEHSEYDLYSFIDNSKKVYRECRETLDYLVDERFDSINKGFI
QGNKVNISLLIDMMKGYEPDDIIRLYYDFIVLKSQKNLGFSIKKLREKMLDEYGFRFKDKQYDSVRSKMYKLMDFLLFCNYYRN
DVAAGEALVRKLRFSMTDDEKEGIYADEAAKLWGKFRNDFENIADHMNGDVIKELGKADMNFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNISFDDFKNVKQQAKGRENVAKERAKAVIGLYLTVMYLLVKNLVNVNARYVIAIHCLER
DFGLYKEIIPELASKNLKNDYRILSQTLCELCDKSPNLFLKKNERLRKCVEVDINNADSSMTRKYRNRIAHLTVVRELKEYIGD
IRTVDSYFSIYHYVMQRCITKREDDTKQGEKIKYEDDLLKNHGYTKDFVKALNSPFGYNIPRFKNLSIEQLFDRNEYLTEK
(SEQ ID NO: 271)
>OHDP01000241_4
[human gut metagenome]
MGKKIHARDLREQRKTDRTEKFADQNKKREAERAVPKKDAAVSVKSVSSVSSKKDNVTKSMAKAAGVKSVFAVENTVYMTSFGR
GNDAVLEQKIVDTSHEQLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKAKNVLREDKELYSVLTLIGKLRHWCVHSEDGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKRLSKSNIEIQEDKLRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPKNTALCSANKRREKLADMIAEIKFENFSDAGNYQKANVTSRTSEAEIKRKNQAIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNININIDGIKEVENYFALYHYLIQKHLENRFADKKVERDTGDFISKLEEHKTYCKDFVKAYCTPF
GYNLVRYKNLTIDGLFDKNFPGKDDSDEQK (SEQ ID NO: 272)
>OHDT01000502_2
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEWIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTASYKLFNDSQRITNELFIVKNIASMRKPAASAKLTMFR
DALTILGIDDKITDDRISEILKLKEKGKGIHGLRNFITNNVIESSRFVYLIKYANAQKIREVAENEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDKKQEEKIKYEDDLLKNHGYTKDFVKALNSPFGYNIPRFKNLSIEQLFDRNEYLTE
K (SEQ ID NO: 273)
>OHFA01000290_5
[human gut metagenome]
MGKKIHARDLREQRKTDRTVKFADQNKKREAERAVQKKDAAVSVKSVPSVSSKKDNVTKSMAKAAGVKSVFAVGNTVYMTSFGR
GNDAVLEQKIVDTSHEPLNIDDPAYQLNVVTMNGYSVIGHRGETVSAVTDNPLRRFNGGKKDEPEQSVPTDMLCLKPTLEKKFF
GKEFDDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKAKNVLREDKELYSVLTLIGKLRHWCVHSEEGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDVDNIKNLSGSNIEIRDNELRKCFISYADSVSEFT
KLIYLLTRFLSGKEINDLVTTLINKFDNIRSFLEIMDELGLDRTFTAEYSFFEDSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPENTALCSANKKREKLADMIAEIEFENFSDAGNYQKANVTSKTHEAEIKRKNQSIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNININIKEIKEVENYFALYHYLIQKHLENRFADKKVERDTGDFISKLEEHKTYCKDFVKAYCTPF
GYNLVRYKNLTIDGLFDKNYPGKDDSDEQK (SEQ ID NO: 274)
>OHGX01000264_3
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSSVRGDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDKRVSEAYKKRVYHMLAIVGQIRQSVFHDKSNELDEYLYSFIDIIDSEYRDTLDYLVDERFDSINKGF
VQGNKVNISLLIDMMKGYEADDIIRLYYDFIVLKSQKNLGFSIKKLREKMLDEYGFRFKDKQYDSVRSKMYKLMDFLLFCNYYR
NDIAAGEALVRKLRFSMTDDEKEGLYADEAAKLWGKFRNDFENIADHMNGDVIKELGKADMDFDEKILDSEKKNASDLLYFSKM
IYMLTYFLDGKEINDLLTTLISKFDNIKEFLKIMKSSAVNVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLEVKRSELARMIKNIRFDDFKNVKQQAKGRENVAKERAKAVIGLYLTVMYLLVKNLVNVNARYVIAIHCLE
RDFGLYKEIIPELASKNLKNDYRILSQTLCELCDNRDESPNLFLKKNKRLRKCVEVDINNADSSMTRKYRNCIAHLTVVRELKE
YIGDIRTVDSYFSIYHYVMQRCITKREDDTKQEDKIKYEDDLLKNHGYTKDFVKALNSPFGYNIPRFKNLSIEQLFDRNEYLTE
K (SEQ ID NO: 275)
>OHIB01002708_3
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEWIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTASYKLFNDSQRITNELFIVKNIASMRKPAASAKLTMFR
DALTILGIDDKITDDRISEILKLKEKGKGIHGLRNFITNNVIESSRFVYLIKYANAQKIREVAENEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVISIMQTAA (SEQ ID NO: 276)
>OHJK01001285_9
[human gut metagenome]
MGKKIHARDLREQRKNDRTAKFAVQNKKCEAQRAVQKKDAAVSAKSVSSVSSKKDNATKSMAKAAGVKSVFAVGNTVYMTSFGR
GNDAVLEQKIVDTSHEPLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAVYALNNTIADENDENWDLFANFSTDNTYYELRNAAAYKESADDESTDDEKRREA
EKKKREAKKAEKILADYEKFRKNNRLAYFADAFYVDKNKSKSKSKDKAEGIQRGKKEIYSILALIAKLRHWCVHSEDGRAEFWL
YKLDELKDDFKNVLDVVYNRPVEEINNRFIENNKVNIQILDSVYENTDIAELTRSYYEFLITKKYKNMGFSIKKLRESMLEGKG
YADKEYDSVRNKLYQMTDFILYTGYINEDSDRADDLVNTLRSSLKEDDKTTVYCKEADYLWKKYRESIREVADALDGDNIKKLS
KSNIEIQEDKLRKCFISYADSVSEFTKLIYLLTRFLSGKEINDLVTTLINKFDNIRSFLEIMDELGLDRTFTAEYSFFEGSTKY
LAELVELNSFVKSCSFDMSAKRTMYRDALDILGIESDKTEEDIEKMIDNILQVDANGKKLPNKNHGLRNFIASNVIDSNRFEYL
VRYGNPKKIRETAKCEPAVRFVLNEIPDAQIERYYKAYYPDEKSLCLANMQRDKLADMIAEIKFENFSDAGSYQEANATSTRIT
SEAEIKRKNQAIIRLYLTVMYIMLKNLVNVNARYVIAFHCLERDAKLYSESVLKVGNTNEESRLQTGNTNEEKNKVKLTNLTNL
TMAVMGVKLENGTIKTEFDKSLAENAANRYLRNARWYKLILDNLKKSERAVVTEFRNTVCHLNAIRNININIKEVKEVENYFAL
YHYLIQKHLEKRFADKKVERDTGDFISKLEEHKTYCKDFVKAYCTPFGYNLVRYKNLTIDGLFDKNYPGKDDSDEQK (SEQ
ID NO: 277)
>OHJS01001864_3
[human gut metagenome]
MAKKNKMKPRERREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVDEVNITFSSKHGFESGVKINTSNPTHRSGESSSVRGDMLGLKSELEKRFFGKTFDDNI
HIQLIYNILDIEKILAVYVTNIVYALNNMLGLENESNNDFMGYLSAKNTYDVFTDPDESDLSKNIKGNIKKSLSKFNDLLKTKR
LGYFGLEEPKTKDKRVSEAYKKRVYHMLAIVGQIRQSVFHDKSNELDEYLYSFIDIIDSEYRETLDYLIDERFDSINKGFIQGN
KVNISLLIDMMKGYEADDIIRLYYDFIVLKSQKNLGFSIKKLREKMLEEYGYRFKDKQYDSVRSKMYKLMDFLLFCNYYRNDVV
AGEALVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVNVECELTAGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLEVKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDNRDESPNLFLKKNKRLRKCVEVDINNADSSMTRKYRNCIAHLTVVRELKEYIGD
IRTVDSYFSIYHYVMQRCITKREDDKKQEEKIKFEDDLLKNHGYTKDFVKALNSPFGYNIPRFKNLSIEQLFDRNEYLTEK
(SEQ ID NO: 278)
>OHJT01001977_4
[human gut metagenome]
MGKKIHARDLRERRKTDRTEKFADQNKKREAERAVQKKDAAVSVKSVSSVSSKKDNVTKSMAKAAGVKSVFAVGNTVYMTSFGR
GNDAVLEQKIVDTSHEPLNIDDPAYQLNVVTMNGYSVTGHRGETVSAVTDNPLRRFNGGKKDEPEQSVPTDMLCLKPTLEKKFF
GKEFDDNIHIQLIYNILDIEKILAVYSTNAIYALNNMSADENIESSDFFMKRTTDETFDDFEKKKESTNSREKADFDAFEKFIG
NYRLAYFADAFYVNKKNPKGKARNVLREDKELYSVLTLIGKLRHWCVHSEEGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKRLSKSNIEIQEDKLRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPKNTALCSANKRREKLADMIAEIEFENFSDAGNYQKANVTSRTSEAEIKRKNQAIIRLYLTVMYIMLKNLV
NVNARYVIAFHCVERDTKLYAESGLEVGNIEKNKTNLTMAVMGVKIENGIIKTEFDKSLAENAANRYLRNARWYKLILDNLKKS
ERAVVNEFRNTVCHLNAIRNININIDGIKEVENYFALYHYLIQKHLENRFADKKVERDTGDFISKLEEHKTYCKDFVKAYCTPF
GYNLVRYKNLTIDGLFDKNYPGKDDSDKQK (SEQ ID NO: 279)
>OHMF01000395_24
[human gut metagenome]
MAKKNKMKPRELREAQKKARQLKAVEINNNAVPEIAAMPAAEVIAPVAEKKKSSVKAAGMKSILVSENKMYITSFGKGNSAVLE
YEVDNNDYNKTQLSSKNSSNIELRGVNEVNITFSSKHGFESGVEINTSNPTHRSGESSPVRWDMLGLKSELEKRFFGKTFDDNI
HIQLIYNILDIEKILAVYVTNIVYALNNMLGIKGSESYDDFMGYLSARNTYEVFTHPDKSSLSDDKKANVRKSLSKFNVLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VIAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDNITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDNGDESPNLFLKKNKRLRKCVEVDINNADSSMTRKYRNCIAHLTVVRELKEYI
GDIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 280)
>OHUY01000263_2
[human gut metagenome]
MAKKNKMKPRELREAQKKARQLKAAEINNNAAPAIAAMPAAEVIAPAAEKKKSSVKAAGMKSILVSKNKMYITSFGKGNSAVLE
YEVDNNNYNKTQLSSKDNSNIELGDVDEVNITFSSKHGFGSGVEINTSNPTHRSGESSPVRWDMLGLKSELEKRFFGKTFDDNI
HIQLIYNILDIEKILAVYVTNIVYALNNMLGIKDSESYDDFMGYLSARNTYEVFTHPDKSNLSDKVKGNIKKSFSTFNDLLKTK
RLGYFGLEEPKTKDTRVSQAYKKRVYHMLAIVGQIRQSVFHDKSSKLDEDLYSFIDIIDSEYRETLDYLVDERFDSINKGFIQG
NKVNISLLIDMMKGFEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSQMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKRENDTKQEDKIKYEDDLLKNHGYTKDFVKALNSPFGYNIPRFKNLSIEQLFDRNEYLTEK (SEQ
ID NO: 281)
>OIBN01003740_1
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNQTQLSSKDNSNIQLGGVNEVNITFSSKHGFESGVEINTSNPTHRSGESSPVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDNRVSQAYKKRVYHMLAIVGQIRQCVFHDKSGAKRFDLYSFINNIDPEYRDTLDYLVEERLKSINKDFIED
NKVNISLLIDMMKGYEADDIIRLYYDFIVLKSQKNLGFSIKKLRDKMLDEYGFRFKDKQYDSVRSKMYKLMDFLLFCNYYRNDI
AAGESLVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISGILKLKEKGKGIHGLRNFITNNVIESSRFVYLIKYANAQKIREVAKNEKVVMFVLGGIPDTQIERYYKS
CVEFPDMNSSLGVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIC
TVDSYFSIYHYVMQRCITKRENDTKQEEKIKYEDDLLKNHGYTKDFVKALNSPFGYNIPRFKNLSIEQLFDRNEYLTEK (SEQ
ID NO: 282)
>OIEE01000042_11
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGDVNEVNITFSSKHGFGSGMKINTSNPTHRSGESSPVRWDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNVLLKTK
RLGYFGLEEPKTKDNRVSEAYKKRVYHMLAIVGQIRQCVFHDKSGAKRFDLYSFINNIDPEYRDTLDYLVEERLKSINKDFIQG
NKVNISLLIDMMKGYEADDIIRLYYDFIVLKSQKNLGFSIKKLREKMLEEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 283)
>OIEL01000292_3
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFGSGVEINTSNPTHRSGESSPVRWDMLGLKSELEKRFFGKTFD
DNIHIQLIYNILDIEKILAVYVTNIVYALNNMLGEGDDESHDDFMGYLSAQNTYYIFTHPDKSNLSDKVKGNIKKSLSKFNDLL
KTKRLGYFGLEEPKTKDKRVSEAYKKRVYHMLAIVGQIRQSVFHDKSNELDEYLYSFIDIIDSEYRDTLDYLVDERFDSINKGF
VQGNKVNISLLIDMMKGYEADDIIRLYYDFIVLKSQKNLGFSIKKLREKMLDEYGFRFKDKQYDSVRSKMYKLMDFLLFCNYYR
NDVVAGEVLVRKLRFSMTDDEKEWIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAENEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDKKQEEKIKYEDDLLKNHGYTKDFVKALNSPFGYNIPRFKNLSIEQLFDRNEYLTE
K (SEQ ID NO: 284)
>OIEN01002196_3
[human gut metagenome]
MERQKRKMKSKSKMAGVKSVFVIGDELLMTSFGDGDDAVLEKDIDENGVVNDCRNPAAYDAVYGTDSIRVKKTNNNIRAKVNNP
LAKSNIRSEESALFRTRVNEYKREQKDKYETLFFGKTFDDNIHIQLISKILDIEKTFSVVIGNIVYAINNLSLEQSIDRPIDIF
GDKNTQGISLREDNDYLKTMLPRCEYLFHNILNSDSDNNSKMNYNKVNKGKEEKDNRNNENIEKLKKALEVIKIIRVDSFHGVD
GIKGDQKFPRSKYNLAVNYNEEIQKTISEPFNRKVEEVQQDFYRNSCVNIDFLKEIMYGSNYTDRGSDSLECSYFNFAILKQNK
NMGFSITSIRECLLDLYELNFESMQNLRPRANSFCDFLIYDYYCKNESERANLVDCLRSAASEEEKKNIYFQTAERVKEKFRNA
FNRISRFDASYIKNSREKNLSGGSSLPKYSFIEGFTKRSKKINDNDEKNADLFCNMLYYLAQFLDGKEINIFLTSIHNIFQNID
SFLKVMKEKGMECKFQKDFKMFSHAGHVAKKIEIVISLAKMKKTLDFYNAQALKDAVTILGVSKKHQYLDMNSYLDFYMFDNRS
GATGKNAGKDHNLRNFLVSNVIRSRKFNYLSRYSNLAEVKKLAQNPSLVQFVLSRIEPSLICRYYESSQGISSEGITIDEQIKK
LTGIIVDMNIDSFENINNGEIGMRYSKATPQSIERRNQMRVCVGLYLNVLYQIEKNLMNVNARYVLAFAFAERDALMLNFTLEE
CKKNKKRSSGGFSFIEMTQFFIDKKLFKVATEAIKKNVLKYNGNPESLNHIPGEYICKNMEGYHENTVRNFRNMVAHLTAVARV
PLYISEVTQIDSYYALYHYCMQMNILQGIEQSGKILDNIKLKNALENARVHRTYSKDAVKYLCLPFAYNISRYKALTIKDLFDW
TEYSCKKDE (SEQ ID NO: 285)
>OIXA01002812_3
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNQTQLSSKDNSNIQLGGVNEVNITFSSKHGFESGVEINTSNPTHRSGESSPVRWDMLGLKSELEKRFFGKTFDDNI
HIQLIYNILDIEKILAVYVTNIVYALNNMLGIKDSESYDDFMGYLSARNTYEVFTHPDKSNLSDKVKGNINKVKCNIKKSFSTF
NDLLKTKRLGYFGLEEPKTKDTRVSQAYKKRVYHMLAIVGQIRQCVFHDKSSKLDEDLYSFIDIIDSEYRETLDYLVDERFDSI
NKGFIQGNKVNISLLIDMMKGYEADDIIRLYYDFIVLKSQKNLGFSIKKLREKMLDEYGFRFKDKQYDSVRSKMYKLMDFLLFC
NYYRNDVVAGEALVRKLRFSMTDDEKEGIYADEAAKLWGKFRNDFENIADHMNGDVIKELGKADMDFDEKILDSEKKNASDLLY
FSKMIYMLTYFLDGKEINDLLTTLISKFDNIKEFLKIMKSSAVDVECELTAGYKLFNDSQRITNELFIVKNIASMRKPAASAKL
TMFRDALTILGIDDNITDDRISEILKLKEKGKGIHGLRNFITNNVIESSRFVYLIKYANAQKIREVAKNEKVVMFVLGGIPDTQ
IERYYKSCVEFPDMNSSLEAKRSELARMIKNISFDDFKNVKQQAKGRENVAKERAKAVIGLYLTVMYLLVKNLVNVNARYVIAI
HCLERDFGLYKEIIPELASKNLKNDYRILSQTLCELCDDRDESPNLFLKKNKRLRKCVEVDINNADSSMTRKYRNCIAHLTVVR
ELKEYIGDIRTVDSYFSIYHYVMQRCITKREDDKKQEEKIKYEDDLLKNHGYTKDFVKALNSPFGYNIPRFKNLSIEQLFDRNE
YLTEK (SEQ ID NO: 286)
>OIXU01000818_5
[human gut metagenome]
MAKKKRITAKERKQNHRESLMKKADSNAEKEKAKKPVVENKPDTAISKDNTPKPNKEIKKSKAKLAGVKWVIKANNDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRIPYLKYGGGKDSKGNPKNKSKWKKRENFINFYNEAK
PHLGYYENIFYDHGEPISEEKFYNYLNILNFIRNNTFHYKDNDIELYSENYSEEYVFINCLNEFVKNKFKNVNKNFISNEKNNL
YIILNAYGEDTENVEVVKKYSKELYKLSVLKTNKNLGVNVKKLRESAIENGYCPLPYDKENDVAKLSSVKHKLYKTYDFVITHY
LNSNDKPLLEIVEALRLSKNDDEKEIVYKKYAEKLFKADDVINPIKAISKLFVEKGNKLFREKVRINKEYIEDVSIDKNIYDFT
KVIFFMTCFLDGKEINDLLTNIISKLQIIEDHNNVIKFIAENDDAVYKDYSDKYAIFRNAGKIATELEAIKSIARMENKIENAP
NEPLLKDALLSLGVSDDTKVLENTYKKYFDSKEKTDKQSQKVSTFLMNNVINNNRFKYVIKYINPADINGLAKNRYLVKFVLSK
IPEEQIDSYYKLFSNEEEPSCEEKIKLLTKKISKLNFQTLFENYKIPNVEKEKKKAIITLYFTIVYILVKNLVNINGLYTLALY
FVERDGFFYKDICEKKDKKQLYKDDDYLLLPEIFSGSKYREETKNLKLPKEKDRDIMKKYLPNDEDRKEYNKFFKQYRNNIVHL
NIIAKLSELTKNIDKDINSYFDIYHYCTQRVMFDYCKKNNNVVLAKMKDLAHIKSDCDEFSSKHTYPYSSAVLRFMNLPFAYNV
PRFKNLSYKKFFDKQWLNHYENLNDFIRILY (SEQ ID NO: 287)
>OIXU01000818_6
[human gut metagenome]
MAKKKRITAKERKQNHRESLMKKADSNAEKEKAKKPVVENKPDTAISKDNTPKPNKEIKKSKAKLAGVKWVIKANNDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRIPYLKYGGGKDSKGNPKNKSKWKKRENFINFYNEAK
PHLGYYENIFYDHGEPISEEKFYNYLNILNFIRNNTFHYKDNDIELYSENYSEEYVFINCLNEFVKNKFKNVNKNFISNEKNNL
YIILNAYGEDTENVEVVKKYSKELYKLSVLKTNKNLGVNVKKLRESAIENGYCPLPYDKENDVAKLSSVKHKLYKTYDFVITHY
LNSNDKPLLEIVEALRLSKNDDEKEIVYKKYAEKLFKADDVINPIKAISKLFVEKGNKLFREKVRINKEYIEDVSIDKNIYDFT
KVIFFMTCFLDGKEINDLLTNIISKLQIIEDHNNVIKFIAENDDAVYKDYSDKYAIFRNAGKIATELEAIKSIARMENKIENAP
NEPLLKDALLSLGVSDDTKVLENTYKKYFDSKEKTDKQSQKVSTFLMNNVINNNRFKYVIKYINPADINGLAKNRYLVKFVLSK
IPEEQIDSYYKLFSNEEEPSCEEKIKLLTKKISKLNFQTLFENYKIPNVEKEKKKAIITLYFTIVYILVKNLVNINGLYTLALY
FVERDGFFYKDICEKKDKKQLYKDDDYLLLPEIFSGSKYREETKNLKLPKEKDRDIMKKYLPNDEDRKEYNKFFKQYRNNIVHL
NIIAKLSELTKNIDKDINSYFDIYHYCTQRVMFDYCKKNNNVVLAKMKDLAHIKSDCDEFSSKHTYPYSSAVLRFMNLPFAYNV
PRFKNLSYKKFFDKQ (SEQ ID NO: 288)
>OIYU01000175_4
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELCDVGKVNITFSSRRGFESGVEINTSNPTHRSGESSSVRGDMLGLKSELEKRFFGKNFD
DNIHIQLIYNILDIEKILAVYVTNIVYALNNMLGEGDESNYDFMGYLSTFNTYKVFTNPNGSTLSDDKKENIRKSLSKFNALLK
TKRLGYFGLEEPKTKDTRASEAYKKRVYHMLAIVGQIRQCVFHDKSGAKRFDLYSFINNIDPEYRETLDYLVDERFDSINKGFI
QGNKVNISLLIDMMKGYEADDIIRLYYDFIVLKSQKNLGFSIKKLREKMLEEYGYRFKDKQYDSVRSKMYKLMDFLLFCNYYRN
DVVAGEALVRKLRFSMTDDEKEGIYADEASKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPASSAKLTMFRD
ALTILGIDDNITDDRISEILKLKEKGKGIHGLRNFITNNVIESSRFVYLIKYANAQKIRKVAKNEKVVMFVLGGIPDTQIERYY
KSCVEFPDMNSSLEVKRSELARMIKNISFDDFKNVKQQAKGRENVAKERAKAVIGLYLTVMYLLVKNLVNVNARYVIAIHCLER
DFGLYKEIIPELASKNLKNDYRILSQTLCELCDESPNLFLKKNERLRKCVEVDINNADSSMTRKYRNCIAHLTVVRELKEYIGD
IRTVDSYFSIYHYVMQRCITKREDDKKQEEKIKFEDDLLKNHGYTKDFVKALNSPFGYNIPRFKNLSIEQLFDRNEYLTEK
(SEQ ID NO: 289)
>OIZA01000315_9
[human gut metagenome]
MAKKKRMTAKERKQNHRESLMKKADSNAEKEKAKKPVVENKPDTAISKDNKPKPNKEIKKSKAKLAGVKWIIKANDDVTYISSF
GKGNNSVLEKRIIGDVSGDVNKDSHMYVNPKYTKKNYEIKNGFSSGSSLVTYPNKPDKNSGMDALCLKPYFEIDFFGHIFTDNM
HIQAIYNIFDIEKILAKHITNIIYTVNSFDRNYNQSGNDTIGFDLNYRVPYLEYGGGKDSNGKPNKISAWKKRENFINFYNEAK
PHLGYYENIFYDHGEPISEEKFYNYLNILNFIRNNTFHYKDNDIELYSENYSEEYVFINCLNEFVKNKFKNVNKNFISNEKNNL
YIILKAYGEDTENVEVVKKYSKELYKLSVLKTNKNLGVNVKKLRESAIEYGYCPLPYDKEKEVAKLSSVKHKLYKTYDFVITHY
LNSNDKLLLEIVETLRLSKNDDEKENVYKKYAEKLFKADDVINPIKAISKLFAEKGNKLFKEKIIIKKEYIEDVSIDKNIYDFT
KVIFFMTCFLDGKEINDLLTNIISKLQVIEDHNNVIKFIFHNKDAVYKDYSDKYAIFRNAGKIATELEAIKSIARMENKIENAP
KEPLLKDALLALGVSSNDFDEKYEKYFKTDVDADKDHQKVSTFLMNNVINNSRFKYVVKYINPADINGLAKNRYLVKFVLSKIP
EEQIDSYYKLFSNEEEPSCEEKIKLLTKKISKLNFQTLFENNKIPNVEKERKKAIITLYFTIVYILVKNLVNINGLYTLALYFV
ERDGFFYKQICEKKLIETLKKKDKKQLYNDDDYLLLPEIFSGSKYREETKNLKLPKEKDRDIMKKYLPNDKDREEYNKFFKQYR
NNIVHLKIIAKLSELTKNIDKDINSYFDIYHYCTQRVMFDYCKKNNNVVLAKMKDLAHIKSDCDEFSSKHTYPYSSAVLRFMNL
PFAYNVPRFKNLSYKKFFDKQ (SEQ ID NO: 290)
>OIZI01000180_12
[human gut metagenome]
MAKKKRMTAKERKQNHRDSLMKKADSNAEKEKAKKPVVENKPDTAISKDNKPKPNKEIKKSKAKLAGVKWIIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSLDRSYNQSGNDTIGFDLNYCIPYSEYGGGKDSNGKPNKISAWKKRENFIKFYNEAK
PHLGYYENIFYDHGEPISEEKFYNYLNILNFIRNNTFHYKDNDIELYSENYSEEYVFINCLNEFVKNKFKNVNKNFISNEKNNL
YIILNAYGEDTENVEVVKKYSKELYKLSVLKTNKNLGVNVKKLRESAIENGYCPLPYDKENDVAKLSSVKHKLYKTYDFVITHY
LNSNDKPLLEIVEALRLSKNDDEKEIVYKKYAEKLFKADDVINPIKAISKLFVEKGNKLFREKVRINKEYIEDVSIDKNIYDFT
KVIFFMTCFLDGKEINDLLTNIISKLQIIEDHNNVIKFIAENDDAVYKDYSDKYAIFRNAGKIATELEAIKSIARMENKIENAP
NEPLLKDALLSLGVSDDTKVLENTYKKYFDSKEKADKQSQKVSTFLMNNVINNNRFKYVIKYINPADINGLAKNRYLVKFVLSK
IPEEQIDSYYKLFSNEEEHSCEEKIKLLTKKISKLNFQTLFENNKIPNVEKERKKAIITLYFTIVYILVKNLVNINGLYTLALY
FVERDGFFYKQICEKKLIETLKKKDKKQLYKDDDYLLLPEIFSGSKYREETKNLKLPKEKDRDIMKKYLPNDKDREDYNDFFTA
YRNNIVHLNIIAKLSKLTKNIDKDINSYFDIYHYCTQRVMFDYCKKNNNVVLAKMKDLAHIKSDCDEFSSKHTYPYSSAVLRFM
NLPFAYNVPRFKNLSYKKFFDKQWLNHYENLNDFIRILY (SEQ ID NO: 291)
>OIZI01000180_12
[human gut metagenome]
MAKKKRMTAKERKQNHRDSLMKKADSNAEKEKAKKPVVENKPDTAISKDNKPKPNKEIKKSKAKLAGVKWIIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSLDRSYNQSGNDTIGFDLNYCIPYSEYGGGKDSNGKPNKISAWKKRENFIKFYNEAK
PHLGYYENIFYDHGEPISEEKFYNYLNILNFIRNNTFHYKDNDIELYSENYSEEYVFINCLNEFVKNKFKNVNKNFISNEKNNL
YIILNAYGEDTENVEVVKKYSKELYKLSVLKTNKNLGVNVKKLRESAIENGYCPLPYDKENDVAKLSSVKHKLYKTYDFVITHY
LNSNDKPLLEIVEALRLSKNDDEKEIVYKKYAEKLFKADDVINPIKAISKLFVEKGNKLFREKVRINKEYIEDVSIDKNIYDFT
KVIFFMTCFLDGKEINDLLTNIISKLQIIEDHNNVIKFIAENDDAVYKDYSDKYAIFRNAGKIATELEAIKSIARMENKIENAP
NEPLLKDALLSLGVSDDTKVLENTYKKYFDSKEKADKQSQKVSTFLMNNVINNNRFKYVIKYINPADINGLAKNRYLVKFVLSK
IPEEQIDSYYKLFSNEEEHSCEEKIKLLTKKISKLNFQTLFENNKIPNVEKERKKAIITLYFTIVYILVKNLVNINGLYTLALY
FVERDGFFYKQICEKKLIETLKKKDKKQLYKDDDYLLLPEIFSGSKYREETKNLKLPKEKDRDIMKKYLPNDKDREDYNDFFTA
YRNNIVHLNIIAKLSKLTKNIDKDINSYFDIYHYCTQRVMFDYCKKNNNVVLAKMKDLAHIKSDCDEFSSKHTYPYSSAVLRFM
NLPFAYNVPRFKNLSYKKFFDKQ (SEQ ID NO: 292)
>OIZU01000200_48
[human gut metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSFSTFNDLL
KTKRLGYFGLEEPKTKDTRVSEAYKKRVYHMLAIVGQIRQCVFHDLSEHSEYDLYSFIDNSKKVYRECRETLNYLVDERFDSIN
KGFIQGNKVNISLLIDMMKDDYEADDIIHLYYDFIVLKSQKNLGFSIKKLREKMLDEYGFRFKDKQYDSVRSKMYKLMDFLLFC
NYYRNDVVAGEALVRKLRFSMTDDEKEGIYADEAEKLWGKFRNDFENIADHMNGDVIKELGKADMDFDEKILDSEKKNASDLLY
FSKMIYMLTYFLDGKEINDLLTTLISKFDNIKEFLKIMKSSAVDVECELTAGYKLFNDSQRITNELFIVKNIASMRKPAASAKL
TMFRDALTILGIDDKITDDRISEILKLKEKGKGIHGLRNFITNNVIESSRFVYLIKYANAQKIREVAKNEKVVMFVLGGIPDTQ
IERYYKSCVEFPDMNSSLKVKRSELARMIKNIRFDDFKNVKQQAKGRENVAKERAKAVIGLYLTVMYLLVKNLVNVNARYVIAI
HCLERDFGLYKEIIPELASKNLKNDYRILSQTLCELCDDRDESPNLFLKKNKRLRKCVEVDINNADSSMTRKYRNCIAHLTVVR
ELKEYIGDIRTVDSYFSIYHYVMQRCITKREDDKKQEEKIKYEDDLLKNHGYTKDFVKALNSPFGYNIPRFKNLSIEQLFDRNE
YLTEK (SEQ ID NO: 293)
>OIZW01000344_20
[human gut metagenome]
MAKKNKMKPRELREAQKKARQLKAAEINNNAAPAIAAMPAAEVIAPAAEKKKSSVKAAGMKSILVSKNKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNALLKTK
RLGYFGLEEPKTKDNRVSEAYKKRVYHMLAIVGQIRQCVFHDKSGAKRFDLYSFINNIDPEYRDTLDYLVEERLKSINKDFIED
NKVNISLLIDMMKGYEADDIIRLYYDFIVLKSQKNLGFSIKKLREKMLDEYGFRFKDKQYDSVRSKMYKLMDFLLFCNYYRNDV
VADEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEVSDMNSSLEAKRSELARMIKNIRFDDFKNVKQQANGRENVAKERAKAVIGLYLTVMYLLVKNLVNVNARYVIAIHCLERDF
GLYKEIIPELASKNLKNDYRILSQTLCELCDNGDESPNLFLKKNKRLRKCVEVDINNADSSMIRKYRNCIAHLTVVRELNKYIN
DIYVVNSYFSICHYVMQRCITKRENDTKQEDKIKYEDDLLKNHGYTKDFVKALNSPFGYNIPRFKNLSIKQLFDRNEYLTEK
(SEQ ID NO: 294)
>OIZX01000427_25
[human gut metagenome]
MAKKKKTARQLREEMQQQRKQAIQKQQEQRQEKAAAARETAAPEQPAAAPVPKRQRKSLAKAAGLKSNFILDPQRRTTVMTAFG
QGSTAILEKQIVDRAISDLQPVQQFQVEPASAAKYRLKNSRVRFPNVTADDPLYRRKDGGFVPGMDALRRKNVLEQRFFGKSFA
DNIHIQMIYSILDIHKILAAASGHIVHLLNIVNGSKDRDFIGMLAAHVLYNELNEEAKRSIADFCKSPRLIYYSAAFYETLDNG
KSERRSNEDIFNILALMTCLRNFSSHHSIAIKVKDYSAAGLYNLRRLGPDMKKMLDTFYTEAFIQLNQSFQDHNTTNLTCLFDI
LNISDSARQKQLAEEFYRYVVFKEQKNLGFSVRKLREEMLLLPDAAVIADKRYDTCRSKLYNLMDFLILRVYRTGRADRCDKLP
EALRAALTDEEKAVVYHKEALSLWNEMRTLILDGLLPQMTPENLSRLSGQKRKGELSLDDAMLKECLYEPGPVPEDAAPEEANA
EYFCRMIYLATLFMDGKEINTLLTTLISKFENIAAFLQTMEQLNIEAELGPEYAMFTRSRAVAEQLRVINSFALMKKPQVNAKQ
QLYRAAVTLLGTEDPDGVTDEMLCIDPVTGKMLPPNQRHHGDTGLRNFIANNVVESRRFQYLIRYSDPAQLHQLASNKKLVRFV
LSSIPDTQINRYYETCGQTRLAGRAAKVEFLTDMIAAIRFDQFRDVNQKERGANTQKERYKAMLGLYQTVLYLAVKNLVNINAR
YVMAFHCVERDMFLYDGELTDPKGESVSAFLAVNGKKGVQPQYLLLTQLFIRRDYLKRSACEQIQHNMENISDRLLREYRNAVA
HLNVIAHLADYSADMREITSYYGLYHYLMQRHLFKRHAWQIRQPERPTEEEQKLIEQEQKQLAWEKALFDKTLQYHSYNKDLVK
ALNAPFGYNLARYKNLSIEPLFSKEAAPAAEIKATHA (SEQ ID NO: 295)
>OIZX01000427_26
[human gut metagenome]
MLLSEELYKWGKAGSTMAKKKKTARQLREEMQQQRKQAIQKQQEQRQEKAAAARETAAPEQPAAAPVPKRQRKSLAKAAGLKSN
FILDPQRRTTVMTAFGQGSTAILEKQIVDRAISDLQPVQQFQVEPASAAKYRLKNSRVRFPNVTADDPLYRRKDGGFVPGMDAL
RRKNVLEQRFFGKSFADNIHIQMIYSILDIHKILAAASGHIVHLLNIVNGSKDRDFIGMLAAHVLYNELNEEAKRSIADFCKSP
RLIYYSAAFYETLDNGKSERRSNEDIFNILALMTCLRNFSSHHSIAIKVKDYSAAGLYNLRRLGPDMKKMLDTFYTEAFIQLNQ
SFQDHNTTNLTCLFDILNISDSARQKQLAEEFYRYVVFKEQKNLGFSVRKLREEMLLLPDAAVIADKRYDTCRSKLYNLMDFLI
LRVYRTGRADRCDKLPEALRAALTDEEKAVVYHKEALSLWNEMRTLILDGLLPQMTPENLSRLSGQKRKGELSLDDAMLKECLY
EPGPVPEDAAPEEANAEYFCRMIYLATLFMDGKEINTLLTTLISKFENIAAFLQTMEQLNIEAELGPEYAMFTRSRAVAEQLRV
INSFALMKKPQVNAKQQLYRAAVTLLGTEDPDGVTDEMLCIDPVTGKMLPPNQRHHGDTGLRNFIANNVVESRRFQYLIRYSDP
AQLHQLASNKKLVRFVLSSIPDTQINRYYETCGQTRLAGRAAKVEFLTDMIAAIRFDQFRDVNQKERGANTQKERYKAMLGLYQ
TVLYLAVKNLVNINARYVMAFHCVERDMFLYDGELTDPKGESVSAFLAVNGKKGVQPQYLLLTQLFIRRDYLKRSACEQIQHNM
ENISDRLLREYRNAVAHLNVIAHLADYSADMREITSYYGLYHYLMQRHLFKRHAWQIRQPERPTEEEQKLIEQEQKQLAWEKAL
FDKTLQYHSYNKDLVKALNAPFGYNLARYKNLSIEPLFSKEAAPAAEIKATHA (SEQ ID NO: 296)
>OJMJ01002228_5
[human gut metagenome]
MAKKNKMKPRELREAQKKARQLKAAEINNNAVPAIAAMPAAQVIAPAAEKKKSSVKAAGMKSILVSKNKMYITSFGKGNSAVLE
YEVDNNDYNKTQLSSKDNSNIELRGVTKVNITFSSKHGLESGVEINTSNPTHRSGESSPVRWDMLGLKSELEKRFFGKTFDDNI
HIQLIYNILDIEKILAVYVTNIVYALNNMLGVKGSESHDDFIGYLSTNNTYDVFIDPDNSSLSDDKKANVRKSLSKFNALLKTK
RLGYFGLEEPKTKDNRVSEAYKKRVYHMLAIVGQIRQCVFHDKSGAKRFDLYSFINNIDPEYRETLDYLVDERFDSINKDFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDILYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCDDRDESPNLFLKKNKRLRKCVEVDINNADSSMTRKYRNCIAHLTVVRELKEYIGDIR
TVDTYFSIYHYVMQRCITKREDDTKQEEKIKYEDDLLKNHGYTKDFVKALNSPFGYNIPRFKNLSIEQLFDRNEYLTEK (SEQ
ID NO: 297)
>OJMM01002900_7
[human gut metagenome]
MMGKHLNAKQRKLEKKLKNQQKDMMYTKSTDAVSVPTLKAAPTKAEMSQDTAEASTLITPGTLKTKAKAMGLKSTLVFDDKIVV
TSFLNSKTEENEKCAHIEKIADCNGQTIVERPRMFNTSINAKKVDLSKDNDETNYPNPAFEDCGRDYINVKSALEKRVFGKTYN
KDNLHVQIAYNIFDIKKIIGAYINNIIYIFYNLGREEYDAKKDIIGTQDSAYISKILNNTSAYFTYFDGVFKQITDRDSNKDRE
IKNSYNALVLKVLYYLRQFCMHGNTYTKRNEESFLSDTALYNAKEFFAKADPQINELIDAVYADGIKTINSDFMAHAKNNMYII
CEVYKNEAEDSLMKEYYDFVVRKEGNNLGFNTRQLREILIDKYVGNLRGKKYNTFRNKLYTVLGFILVKEIKRNPKIQDSFIAK
LRANQNGDEGKLNIYNEFAPKIWSVVSSKLNSAITCFDEESLSKFKGYKDIDESLISRYGITVANTDTLVKILYFLCKFLDGKE
INELCCAMINKFDNINDLIKTAAQCGEDIEFVKEYKLFINSNDLSDQIRIVKSISKMKPELSKIGEALILDAIDILGYKINKYK
YDAAGNRLVDSNNKPVYSEEYCAFKKDFFETCELDEFGRVKYNKKGKPVINHRRRNFIINNVLSSKWFFYVAKYNRPSECQKFM
KSKKLIALVLKDVPETQIARYYQSVTGGRTQANSEAMRMTLIKLLHEFSIKNVLSDVGTMTASENKRQIENSRKERMKAIVKLY
LTVVYLIAKSLVKVNTRFSIAFSAYERDVSLLADENELIALANNEDDKWKKGNYVFALTKHFWDNDEPYFDKYNNALQQIRSIV
DPNERRLAYRANDKVVKHTHFNLHSYKYVKHNYEEISKASKIITAYRNNVQHLNVMNSITKYLGDISEVTSYYSLYCYTLQRLL
LDDNNNDKFASIKGNLRKFGIYNKDFMWLLNIPFAYNLPRYKNLSNEEIFYDELQK (SEQ ID NO: 298)
>OJMM01002900_7
[human gut metagenome]
MGKHLNAKQRKLEKKLKNQQKDMMYTKSTDAVSVPTLKAAPTKAEMSQDTAEASTLITPGTLKTKAKAMGLKSTLVFDDKIVVT
SFLNSKTEENEKCAHIEKIADCNGQTIVERPRMFNTSINAKKVDLSKDNDETNYPNPAFEDCGRDYINVKSALEKRVFGKTYNK
DNLHVQIAYNIFDIKKIIGAYINNIIYIFYNLGREEYDAKKDIIGTQDSAYISKILNNTSAYFTYFDGVFKQITDRDSNKDREI
KNSYNALVLKVLYYLRQFCMHGNTYTKRNEESFLSDTALYNAKEFFAKADPQINELIDAVYADGIKTINSDFMAHAKNNMYIIC
EVYKNEAEDSLMKEYYDFVVRKEGNNLGFNTRQLREILIDKYVGNLRGKKYNTFRNKLYTVLGFILVKEIKRNPKIQDSFIAKL
RANQNGDEGKLNIYNEFAPKIWSVVSSKLNSAITCFDEESLSKFKGYKDIDESLISRYGITVANTDTLVKILYFLCKFLDGKEI
NELCCAMINKFDNINDLIKTAAQCGEDIEFVKEYKLFINSNDLSDQIRIVKSISKMKPELSKIGEALILDAIDILGYKINKYKY
DAAGNRLVDSNNKPVYSEEYCAFKKDFFETCELDEFGRVKYNKKGKPVINHRRRNFIINNVLSSKWFFYVAKYNRPSECQKFMK
SKKLIALVLKDVPETQIARYYQSVTGGRTQANSEAMRMTLIKLLHEFSIKNVLSDVGTMTASENKRQIENSRKERMKAIVKLYL
TVVYLIAKSLVKVNTRFSIAFSAYERDVSLLADENELIALANNEDDKWKKGNYVFALTKHFWDNDEPYFDKYNNALQQIRSIVD
PNERRLAYRANDKVVKHTHFNLHSYKYVKHNYEEISKASKIITAYRNNVQHLNVMNSITKYLGDISEVTSYYSLYCYTLQRLLL
DDNNNDKFASIKGNLRKFGIYNKDFMWLLNIPFAYNLPRYKNLSNEEIFYDELQK (SEQ ID NO: 299)
>OJMN01000417_22
[human gut metagenome]
MAKKNKMKPRELREAQKKARQLKVAEINNNAAPAIAAMPAVEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGIKGSESYDDFMGYLSARNTYEVFTNPNGSTLSDDKKENIRKSLSKFNALLKTK
RLGYFGLEEPKTKDTRVSQAYKKRVYHMLAIVGQIRQSVFHDKSSKLDEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VVAGEALVRKLRFSMTDDEKEGTYADEAEKLWGKFRNDFENIADHMNGDAIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDNITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDKSPNLFLKKNERLRKCVEVDINNADSSMTRKYRNCIAHLTVVRELKEYIGDI
RTVDSYFSIYHYVMQRCITKRENDTKQEDKIKYEDDLLKNHGYTKDFVKALNSPFGYNIPRFKNLSIEQLFDRNEYLTEK
(SEQ ID NO: 300)
>OJNR01001167_9
[human gut metagenome]
MGKKIHARDLREQRKTDRTVKFADQNKKREAQRAVQKKDAAVSVKSVSSVSSKKDNATKSMAKAAGVKSVFAVENTVYMTSFGR
GNDAVLEQKIVDTSHEQLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKARNVLREDKELYSVLTLIGKLRHWCVHSEEGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDVDNIKNLSGSNIEIRDNELRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIESDKTEEDIEKMIDNILQIDANGDKKLKKNNGLRNFIASNVIDSNRFKYLVRYGNPKKIRETAKCEPAVRFVLNEI
PDAQIERYYEACCPKNTALCSANKRREKLADMIAEIEFENFSDAGNYQKANVTSRTSEAEIKRKNQAIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNININIDGIKEVENYFALYHYLIQKHLENRFADKKVERDTGDFISKLEEHKTYCKDFVKAYCTPF
GYNLVRYKNLTIDGLFDKNSPGKDDSDEQK (SEQ ID NO: 301)
>OJPG01000139_73
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELGDVNEVNITFSSKRGNESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGFRFKDKQYDSVRSKMYKLMDFLLFCNYYRNDIA
AGESLVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLGVKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDKSPNLFLKKNERLRKCVEVDINNADSSMTRKYRNCIAHLTVVRELKEYIGDIRT
VDSYFSIYHYVMQRCITKREDDIKQEEKIKYEDDLLKNHGYTKDFVKALNSPFGYNIPRFKNLSIEQLFDRNEYLTEK (SEQ
ID NO: 302)
>OJPX01000614_4
[human gut metagenome]
MAKKNKMKPRELREAQKKARQLKVAEINNNAAPAIAAMPAVEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGIKGSESYDDFMGYLSARNTYEVFTNPNGSTLSDDKKENIRKSLSKFNALLKTK
RLGYFGLEEPKTKDTRVSQAYKKRVYHMLAIVGQIRQSVFHDKSSKLDEDLYSFIDIIDSEYRETLDYLVDERFDSINKGFVQG
NKVNISLLIDMMKDDYEADDIIRLYYDFIVLKSQKNLGFSIKKLREKMLDEYGFRFKDKQYDSVRSKMYKLMDFLLFCNYYRND
VVAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDNITDDRISEILKLKEKGKGIHGLRNFITNNVIESSRFVYLIKYANAQKIREVAKNEKVVMFVLGGIPDTQIERYYK
SCVEFPDMNSSLEVKRSELARMIKNICFDDFKNVKQQAKGRENVAKERAKAVIGLYLTVMYLLVKNLVNVNARYVIAIHCLERD
FGLYKEIVSELASKNLKNDYRILSQTLCELCDKSPNLFLKKNERLRKCVEVDINNADSSMTRKYRNCIAHLTVVRELKEYIGDI
RAVDSYFSIYHYVMQRCITKRGNDTKQEDKIKYEDDLLKNHGYTKDFVKALNSPFGYNIPRFKNLSIEQLFDRNEYLTEK
(SEQ ID NO: 303)
>OKRZ01002949_5
[human gut metagenome]
MAKKNKMKPRELREAQKKARQLKAAEINNNAAPAIAAMPAAEVIAPAAEKKKSSVKAAGMKSIFVSENKMYITSFGKGNSAVLE
YEVDKVDNNVYNQTQLSSEDSSNIELCGVTKVNITFSSKHGLESGVEISTSNPTHRSGESSPVRWDMLGLKSELEKRFFGKTFD
DNIHIQFIYNILDIEKILAVYVTNSVYALNNMLGVKGSESHDDFIGYLSTNNIYDVFIDPDNSSLSDDKKANVRKSLSKFNALL
KTKRLGYFGLEEPKTKDTRVSEAYKKRVYHMLAIVGQIRQCVFHDKSSKLDEDLYSFIDIIDPEYRETLDYLVDERFDSINKGF
IQGNKVNISLLIDMMKGYEADDIIRLYYDFIVLKSQKNLGFSIKKLREKMLDEYGFRFKDKQYDPVRSKMYKLMDFLLFCNYYR
NDVVAGEALVRKLRFSMTDDEKEGIYADEAEKLWGKFRNDFENIADHMNGDVIKELGKADMDFDEKILDSEKKNASDLLYFSKM
IYMLTYFLDGKEINDLLTTLISKFDNIKEFLKIMKSSAVNVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIRKVAENEKVVMFVLGGIPDTQIERY
YKSCVEFPDMNSSLEAKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNERLRKCVEVDINNADSSMTRKYRNCIAHLTVVRELKEYIG
DIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 304)
>OKSB01002689_10
[human gut metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDTRVSQAYKKRVYHMLAIVGQIRQCVFHDKSGAKRFDIYSFINNIDPEYRETLDYLVDERFDSINKGFIQG
NKVNISLLIDMMKDYEADDIIRLYYDFIVLKSQKNLGFSIKKLREKMLDEYGFRFKDKQYDSVRSKMYKLMDFLLFCNYYRNDV
VAGETLVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTEGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKREDDTKQEEKIKYEDVLLKNHGYTKDFVKALNSPFGYNIPRFKNLSIEQLFDRNEYLTEK
(SEQ ID NO: 305)
>OKSC01004083_2
[human gut metagenome]
MEREVKKPPKKSLAKAAGLKSTFVISPQEKELAMTAFGRGNDALLQKRIVDGVVRDVAGEKQQFQVQRQDESRFRLQNSRLADR
TVTADDPLHRAETPRRQPLGAGMDQLRRKAILEQKYFGRTFDDNIHIQLIYNILDIHKMLAVPANHIVHTLNLLGGYGETDFVG
MLPAGLPYDKLRVVKKKNGDTVDIKADIAAYAKRPQLAYLGAAFYDVTPGKSKRDAARGRVKREQDVYTILSLMSLLRQFCAHD
SVRIWGQNTPAALYHLQALPQDMKDLLDDGWRRALGGVNDHFLDTNKVNLLTLFEYYGAETKQARVALTQDFYRFVVLKEQKNM
GFSLRRLREELLKLPDAAYLTGQEYDSVRQKLYMLLDFLLCRLYAQERTGRCEELVSALRCALSDEEKDAVYQAEAAALWQALG
DTLRRELLPLLKGKKLQDKDKKKLDELGLSRDVLDGVLFRPAQQGNRANADYFCRLMHLSTWFMDGKEINTLLTTLISKLENID
SLRNVLESMGLACSFVPAYAMFDHSRYIAGQLRVVNNIARMRKPAITAKREMYRAAVVLLGVDSPEAAAAITDDLLQIDPETGK
VRPRGDSARDTGLRNFIANNVVESRRFTYLLRYMTPEQARVLAQNEKLIAFVLSTVPDTQLERYCRTCGREDITGRPAQIRYLT
AQIMGVRYESFTDVEQRGRGDNPKKERYKALIDLYLTVLYLAVKNMVNCNARYVIAFYCRDRDTALYQKEVCWYDLEEDKKSGK
QRQVEDYTALTRYFVSQGYLNRHACGYLRSNMNGISNSLLTAYRNAVDHLNVIPPLGSLCRDIGRVDSYFALYHYAVQRYLNGR
YYRKTPREQELFAAMAQHRTWCSDLVKALNTPFGYNLARYKNLSIDGLFDREGDHVVREDGEKPAE (SEQ ID NO: 306)
>OKSD01002505_11
[human gut metagenome]
MAKKNKMKPRELREAQKKARQLKAAEINNNAAPAIAAMPAEEVIAPAAEKKKSSVKAAGMKSILVSKNKMYITSFGKGNSAVLE
YEVDKVDNDNYNKTQLSSEDSSNIELCGVTKVNITFSSKHGLESGVEISTSNPTHRSGESSPVRWDMLGLKSELEKRFFGKTFD
DNIHIQFIYNILDIEKILAVYVTNIVYALNNMLGVKGSESYDDFMGYLSARNTYEVFTHPDKSNLSDKVKGNIKKSFSTFNDLL
KTKRLGYFGLEEPKTKDNRVSEAYKKRVYHMLAIVGQIRQSVFHDKSSKLHEDLYSFIDIIDPEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEGIYADEAAKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNERLRKCVEVDINNADSSMTRKYRNCIAHLTVVRELKEYIG
DIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 307)
>OLGN01000304_32
[human gut metagenome]
MAKKNKMKPRELREAQKKARQLKAAEINNNAVPAIAAMPAAQVIAPAAEKKKSSVKAAGMKSILVSENKMYITSFGKGNSAVLE
YEVDNNDYNKTQLSSKDSSNIELRGVNEVNITFSSKHGFGSGVEINTSNPTHRSGESSPVRWDMLGLKSELEKRFFGKTFDDNI
HIQLIYNILDIEKILAVYVTNIVYALNNMLGIKKSESHDDFMGYLSAKNTYDVFTNPNGSTLSDDKKKNIRKSLRKFNDLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKDDYEADDIIRLYYDFIVLKSQKNLGFSIKKLREKMLDEYGFRFKDKQYDSVRSKMYKLMDFLLFCNYYRND
VIAGEALVRKLRFSMTDDEKEGIYADEAAKLWGKFRNDFENIADHMNGDVIKELGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDNITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDKSPNLFLKKNRRLRKCVEVDINNADSSMTRKYRNCIAHLTVVRELKEYIGDI
RTVDSYFSIYHYVMQRCITKRENDTKQEEKIKYEDDLLKNHGYTKDFVKALNSPFGYNIPRFKNLSIEQLFDRNEYLTEK
(SEQ ID NO: 308)
>OLHE01000257_41
[human gut metagenome]
MAKKNKMKPRELREAQKKARQLKVAEINNNAAPAIAAMPAAEAAAPAVEKKKSSVKAAGMKSILVSENKMYITSFGKGNSAVLE
YEVDNNDYNKTQLSSKDNSNIELGDVDEVNITFSSKHGFGSGVEINTSNPTHRSGESSPVRWDMLGLKSELEKRFFGKTFDDNI
HIQLIYNILDIEKILAVYVTNIVYALNNMLGIKGSESYDDFMGYLSAKNTYDVFTDPDESDLSKNIKGNIKKSLSKFNDLLKTK
RLGYFGLEEPKTKDKRVSEAYKKRVYHMLAIVGQIRQSVFHDKSSKLDEDLYSFIDIIDSEYRETLDYLVNERFDSINKGFIQG
NKVNISLLIDMMKGDYEADDIIRLYYDFIVLKSQKNLGFSIKKLREKMLDEYGFRFKDKQYDSVRSKMYKLMDFLLFCNYYRND
VVAGEALVRKLRFSMTDDEKEGIYADEAAKLWGKFRNDFENIADHMNSDVIKQLGKADMDFDEKILDSEKKNASDLLYFSKMIY
MLTYFLDGKEINDLLTTLISKFDNIKEFLKIMKSSAVDVECELTAGYKLFNDSQRITNELFIVKNIASMRKPAASAKLTMFRDA
LTILGIDDKITDDRISEILKLKEKGKGIHGLRNFITNNVIESSRFVYLIKYANAQKIREVAKNEKVVMFVLGGIPDTQIERYYK
SCVEFPDMNSSLEAKRSELARMIKNISFDDFKNVKQQAKGRENVAKERAKAVIGLYLTVMYLLVKNLVNVNARYVIAIHCLERD
FGLYKEIIPELASKNLKNDYRILSQTLCELCDNCDESPNLFLKKNKRLRKCVEVDINNADSSMTRKYRNCIAHLTVVRELKEYI
GDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 309)
>PPYE01106492_34
[human gut metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELGDVDEVNITFSSKHGFGSGVEINTSNPTHRSGESSPVRWDMLGLKSELEKRFFGKTFDDNI
HIQLIYNILDIEKILAVYVTNIVYALNNMLGEGDDESHDDFMGYLSTFNTYKVFTNPNGSTLSDDKKENIRKSLSKFNALLKTK
RLGYFGLEEPKTKDTRASEAYKKRVYHMLAIVGQIRQCVFHDKSGAKRFDLYSFINNIDPEYRDTLDYLVDERFDSINKGFIQG
NKVNISLLIDMMKGYEADDIIRLYYDFIVLKSQKNLGFSIKKLREKMLEEYGYRFKDKQYDSVRSKMYKLMDFLLFCNYYRNDV
VAGESLVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFVVKNIASMRKPAVSAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNERLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 310)
>PPYE01385196_3
[human gut metagenome]
MAKKNKMKPRELREAQKKARQLKVAEINNNAAPAIAAMPAAEVIAPAAEKKKSSVKAAGMKSILVSENKMYITSFGKGNSAVLE
YEVDKVDNNNYNKTQLSSKNSSNIELCGVTKVNITFSSKHGFGSGVKINTSNPTHRSGESSPVRWDMLGLKSELEKRFFGKTFN
DNIHIQLIYNILDIEKILAVYVTNIVYALNNMLGIKDSESYDDFMGYLSARNTYKVFTRPDKSNLSDKAKGNIKKSFSTFNDLL
KTKRLGYFGLEEPKTKDTRVSQAYKKRVYHMLAIVGQIRQSVFHDKSSKLDEDLYSFIDIIDPEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVAAGEALVRKLRFSMTDDEKEGIYADEAEKLWVKFRNDFENIADHMNGDVIKELGKADMDFDEKILDSEKKNASDLLYLSKM
IYMLTYFLDGKEINDILTTLISKFDNIKEFLKIMKSSAVNVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKRENDTKQEDKIKYEDDLLKNHGYTKDFVKALNSPFGYNFPRFKNLSIEQLFDRNEYLTE
K (SEQ ID NO: 311)
>PPYE01512733_3
[human gut metagenome]
MAKKNKMKPRELREAQKKARQLKAAEINNNAAPAIAAMSAAEVIAPAAEKKKSSVKAAGMKSILVSENKMYITSFGKGNSAVLE
YEVDNNDYNKTQLSSKDNSNIELGDVNEVNITFSSKHGFGSGVEINTSNPTHRSGESSPVRWDMLGLKSELEKRFFGKTFDDNI
HIQLIYNILDIEKILAVYVTNIVYALNNMLGVKGSESYDDFMGYLSAKNTYEVFTHPDKSNLSDKVKGNIKKSFSTFNDLLKTK
RLGYFGLEEPKTKDTRVSQAYKKRVYHMLAIVGQIRQSVFHDKSSKLHEDLYSFIDIIDSEYRETLDYLVEERLKSINKDFIEG
NKVNISLLIDMMKGYEADDIIRLYYDFIVLKSQKNLGFSIKKLREKMLDEYGFRFKDKQYDSVRSKMYKLMDFLLFCNYYRNDV
VAGETLVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNRRLRKCVEVDINNADSSMTRKYRNCIAHLTVVRELKEYIGDIR
TVDSYFSIYHYVMQRCITKREDDTKQEEKIKYEDDLLKNHGYTKDFVKALNSPFGYNIPRFKNLSIEQLFDRNEYLTEK (SEQ
ID NO: 312)
>PPYF01670242_39
[human gut metagenome]
MAKKNKMKPRELREAQKKARQLKAAEINNNAVPAIAAMPAAEAAAPAAEKKKSSVKAAGMKSILVSENKMYITSFGKGNSAVLE
YEVDNNDYNKTQLSSKDNSNIELCDVNEVNITFSSKHGFESGVEINTSNPTHRSGESSPVRWDMLGLKSELEKRFFGKTFDDNI
HIQLIYNILDIEKILAVYITNIVYALNNMLGVKGSESHDDFIGYLSTNNIYDVFIDPDNSSLSDDKKANVRKSLSKFNALLKTK
RLGYFGLEEPKTKDNRVSEAYKKRVYHMLAIVGQIRQSVFHDKSSKLHEDLYSFIDIIDSEYRETLDYLVDERFDSINKGFIQG
NKVNISLLIDMMKGYEADDIIRLYYDFIVIKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNKRLRKCVEVDINNADSSMTRKYRNCIAHLTVVRELKEYIGDIR
TVDSYFSIYHYVMQRCITKRENDTKQEEKIKYEDDLLKNHGYTKDFVKALNSPFGYNIPRFKNLSIEQLFDRNEYLTEK (SEQ
ID NO: 313)
>ODFW01000112_43
[human metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELCGVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNIRFDDFKNVKQQAKGRENVAKERAKAVIGLYLTVMYLLVKNLVNVNARYVIAIHCLER
DFGLYKEIIPELASKNLKNDYRILSQTLCELCDNGDESPNLFLKKNKRLRKCVEVDINNADSNMTRKYRNCIAHLTVVRELKEY
IGDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 314)
>ODGN01000188_50
[human metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNQTQLSSDGSSNIELRGVNEVNITFSSKHGFESGVEINTSNPTHRSGESSPVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSNELDEYLYSFIDIIDSEYRDTLDYLVDERFDSINKGFVQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNRRLRKCVEVDINNADSSMTRKYRNCIAHLTVVRELKEYIGDIR
TVDSYFSIYHYVMQRCITKRENDTKQEEKIKYEDDLLKNHGYTKDFVKALNSPFGYNIPRFKNLSIEQLFDRNEYLTEK (SEQ
ID NO: 315)
>ODHH01000275_14
[human metagenome]
MAKKNKMKPRELREAQKKARQLKAAEINNNAAPAIAAMSAAEVIAPAAEKKKSSVKAAGMKSILVSENKMYITSFGKGNSAVLE
YEVDNNDYNKTQLSSKDNSNIELGDVNEVNITFSSKHGFGSGVEINTSNPTHRSGESSPVRWDMLGLKSELEKRFFGKTFDDNI
HIQLIYNILDIEKILAVYVTNIVYALNNMLGVKGSESYDDFMGYLSAKNTYEVFTHPDKSNLSDKVKGNIKKSFSTFNDLLKTK
RLGYFGLEEPKTKDTRVSQAYKKRVYHMLAIVGQIRQSVFHDKSSKLHEDLYSFIDIIDSEYRETLDYLVEERLKSINKDFIEG
NKVNISLLIDMMKGYEADDIIRLYYDFIVLKSQKNLGFSIKKLREKMLDEYGFRFKDKQYDPVRSKMYKLMDFLLFCNYYRNDV
VTGEALVRKLRFSMTDDEKEGIYADEASKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYMILSQTLCELCDKSPNLFLKKNERLRKCVEVDINNADSSMTRKYRNCIAHLTVVRELKEYIGDIR
TVDSYFSIYHYVMQRCITKREDDTKQEEKIKYEDDLLKNHGYTKDFVKALNSSFGYNIPRFKNLSIEQLFDRNEYLTEK (SEQ
ID NO: 316)
>ODHP01001712_3
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLEEYGYRFKDKQYDSVRSKMYKLMDFLLFCNYYRNDVV
AGEALVRKLRFSMTDDEKEGIYADEASKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPASSAKLTMFRDALT
ILGIDDNITDDRISEILKLKEKGKGIHGLRNFITNNVIESSRFVYLIKYANAQKIRKVAKNEKVVMFVLGGIPDTQIERYYKSC
VEFPDMNSSLEVKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDKSPNLFLKKNRRLRKCVEVDINNADSSMTRKYRNCIAHLTVVRELKEYIGDIRT
VDSYFSIYHYVMQRCITKREDDTKQEEKIKYEDDLLKNHGYTKDFVKALNSPFGYNIPRFKNLSIEQLFDRNEYLTEK (SEQ
ID NO: 317)
>ODHV01000466_16
[human metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSLSKFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEGIYADEASKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKCSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDKKQEEKIKYEDDLLKNHGYTKDFVKALNSPFGYNIPRFKNLSIEQLFDRNEYLTE
K (SEQ ID NO: 318)
>ODJZ01000182_13
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELCDVGKVNITFSSRRGFESGVEINTSNPTHRSGESSSVRGDMLGLKSELEKRFFGKNFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGYRFKDKQYDSVRSKMYKLMDFLLFCNYYRNDVV
AGEALVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDNITDDRISEILKLKEKGKGIHGLRNFITNNVIESSRFVYLIKYANAQKIREVAKNEKVVMFVLGGIPDTQIERYYKSC
VEFPDMNSSLEAKRSELARMIKNISFDDFKNVKQQAKGRENVAKERAKAVIGLYLTVMYLLVKNLVNVNARYVIAIHCLERDFG
LYKEIIPELASKNLKNDYRILSQTLCELCDDRDESPNLFLKKNKRLRKCVEVDINNADSSMTRKYRNCIAHLTVVRELKEYIGD
IRTVDSYFSIYHYVMQRCITKREDDKKQEEKIKFEDDLLKNHGYTKDFVKALNSPFGYNIPRFKNLSIEQLFDRNEYLTEK
(SEQ ID NO: 319)
>ODLN01002572_7
[human metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNKTQLSSKDNSNIELGNVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDKITDDRISEILKLKEKGKGIHGLRNFITNNVIESSRFVYLIKYANAQKIREVAKNEKVVMFVLGGIPDTQIERYY
KSCVEFPDMNSSLEAKRSELARMIKNISFDDFKNVKQQAKGRENVAKERAKAVIGLYLTVMYLLVKNLVNVNARYVIAIHCLER
DFGLYKEIIPELASKNLKNDYRILSQTLCELCDDRDESPNLFLKKNRRLRKCVEVDINNADSSMTRKYRNCIAHLTVVRELKEY
IGDIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 320)
>ODQJ01000729_25
[human metagenome]
MAKKNKMKPRELREAQKKARQLKAAEINNNAVPAIAAMPAAEVIAPAAEKKKSSVKAAGMKSILVSENKMYITSFGKGNSAVLE
YEVDNNDYNKTQLSSKGSSNIELHGVNEINITFSSKHGFESGVEINTSNPTHRSGESSPVRWDMLGLKSELEKRFFGKTFDDNI
HIQLIYNILDIEKILAVYVTNIVYALNNMLGIKDSESYDDFMGYLSAKNTYEVFTHPDKSNLSDKVKGNIKKSFSTFNDLLKTK
RLGYFGLEEPKTKDTRVSQAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLGVKRSELARMIKNISFDDFKNVKQQSKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIC
TVDSYFSIYHYVMQRCITKRENDTKQEEKIKYEDDLLKNHGYTKDFVKALNSPFGYNIPRFKNLSIEQLFDRNEYLTEK (SEQ
ID NO: 321)
>ODUN01000242_23
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGDVDEVNITFSSKHGFGSGVEINTSNPTHRSGESSPVRWDMLGLKSELEKRFFGKTFDDNI
HIQFIYNILDIEKILAVYVTNIVYALNNMLGIKDSESYDDFIGYLSARNTYKVFTHPDKSNLSDKAKGNIKKSFSTFNDLLKTK
RLGYFGLEEPKTKDTRVLEAYKKRVYYMLAIVGQIRQCVFHDLSEHSEYDLYSFIDNSKKVYRECRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVVAGEALVRKLRFSMTDDEKEGIYADEAAKLWVKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAENEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDKSPNLFLKKNERLRKCVEVDINNADSSMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 322)
>ODVQ01003982_3
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDSSNIELRGVNEVNITFSSKHGFGSGVEINTSNPTHRSGESSPVRWDMLGLKSELEKRFFGKTFDDNI
HIQLIYNILDIEKILAVYVTNIVYALNNMLGIKKSESHDDFMGYLSAKNTYDVFTNPNGSTLSDDKKKNIRKSLRKFNDLLKTK
RLGYFGLEEPKTKDTRVSQAYKKRVYHMLAIVGQIRQSVFHDKSSKLHEDLYSFIDIIDSEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
AAGEALVRKLRFSMTDDEKEGIYADEAAKLWGKFRNDFENIADHMNGDVIKELGKADMDFDEKIIDSEKKNASDLLYFSKMIYM
LTYFLDGKEINDLLTTLISKFDNIKEFLKIMKSSAVDVECELTAGYKLFNDSQRITNELFIVKNIASMRKPASSAKLTMFRDAL
TILGIDDNITDDRISEILKLKEKGKGIHGLRNFITNNVIESSRFVYLIKYANAQKIRKVAKNEKVVMFVLGGIPDTQIERYYKS
CVEFPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDESPNLFLKKNERLRKCVEVDINNADSSMTRKYRNCIAHLTVVRELKEYIGDIR
TVDSYFSIYHYVMQRCITKREDDKKQEEKIKFEDDLLKNHGYTKDFVKALNSPFGYNIPRFKNLSIEQLFDRNEYLTEK (SEQ
ID NO: 323)
>ODVR01002077_3
[human metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNKTQLSSKDSSNIELHGVNEVNITFSSKHGFESGVEINTSNPTHRSGESSPVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSSKLDEDLYSFIDIIDSEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEVPDMNSSLEVKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDKSSNLFLKKNKRLRKCVEVDINNADSRMTRKYRNCIAHLTVVRELKEYIG
DIRTVDSYFSIYHYVMQRCITKREDDTKREEKIKYEDDLLKNHGYTKDFVKALNSPFGYNIPRFKNLSIEQLFDRNEYLTEK
(SEQ ID NO: 324)
>ODXC01000747_3
[human metagenome]
MAKKNKMKPRELREAQKKARQLKAAEINNNAAPAIAAMPAAEAAAPAAEKKKSSVKAAGMKSILVSENKMYITSFGKGNSAVLE
YEVDNNDYNKTQLSSKGSSNIELHGVNEVNITFSSKHGFESGVEINTSNPTHRSGESSPVRGDMLGLKSELEKRFFGKTFDDNI
HIQLIYNILDIEKILAVYVTNIVYALNNMLGEGDDESHDDFMGYLSTFNTYKVFTNPNGSTLSDDKKENIRKSLSKFNALLKTK
RLGYFGLEEPKTKDTRASEAYKKRVYHMLAIVGQIRQSVFHDKSNELDEYLYSFIDIIDSEYRETLDYLIDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGESLVRKLRFSMTDDEKEGIYADEAEKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFVVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNERLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 325)
>ODXO01005124_2
[human metagenome]
MAKKNKMKPRELREAQKKARQLKAVEINNNAVPEIAAMPAAEVIAPVAEKKKSSVKAAGMKSILVSENKMYITSFGKGNSAVLE
YEVDNNDYNKTQLSSKDNSNIELCDVDEVNITFSSKHGFESGVKINTSNPTHRSGESSSVRGDMLGLKSELEKRFFGKTFDDNI
HIQLIYNILDIEKILAVYVTNIVYALNNMLGVKDSESYDDFMGYLSAKNTYDVFTDPDESDLSKNIKGNIKKSLSKFNALLKTK
RLGYFGLEEPKTKDTRASEAYKKRVYHMLAIVGQIRQCVFHDKSGAKRFDLYSFINNIDPEYRETLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDI
AAGESLVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLKVKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNRRLRKCVEVDINNADSSMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 326)
>ODYC01000377_16
[human metagenome]
MAKKNKMKPRELREAQKKARQFKAAEINNNAAPAIAAMPAAEVIAPVAEKKKSSVKAAGMKSILVSENKMYITSFGKGNSAVLE
YEVDNNDYNKTQLSSKDNSNIELCGVNEVNITFSSKHGFESGVEINTSNPTHRSGESSPVRWDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRVLEAYKKRVYHMLAIVGQIRQCVFHDLSEHSEYDLYSFIDNSKKVYRECRETLDYLVDERFDSINKGFI
QGNKVNISLLIDMMKGYEADDIIRLYYDFIVLKSQKNLGFSIKKLREKILDEYGFRFKDKQYDSVRSKMYKLMDFLLFCNYYRN
DIAAGESLVRKLRFSMTDDEKEGIYADEAAKLWGKFRNDFENIADHMNGDVIKELGKADMDFDEKILDSEKKNASDLLYFSKMI
YMLTYFLDGKEINDLLTTLISKFDNIKEFLKIMKSSAVDVECELTAGYKLFNDSQRITNELFIVKNIASMRKPAASAKLTMFRD
ALTILGIDDNITDDRISEILKLKEKGKGIHGLRNFITNNVIESSRFVYLIKYANAQKIREVAKNEKVVMFVLGGIPDTQIERYY
KSCVEFPDMNSSLEVKRSELARMIKNIRFDDFKNVKQQAKGRENVAKERAKAVIGLYLTVMYLLVKNLVNVNARYVIAIHCLER
DFGLYKEIIPELASKNLKNDYRILSQTLCELCDNRDESPNLFLKKNKRLRKCVEVDINNADSSMTRKYRNCIAHLTVVRELKEY
IGDIRTVDSYFSIYHYVMQRCITKREDDTKQEDKIKYEDDLLKNHGYTKDFVKALNSPFGYNIPRFKNLSIEQLFDRNEYLTEK
(SEQ ID NO: 327)
>OEJW01000623_11
[human metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNQTQLSSDGSSNIELRGVNEVNITFSSKHGFESGVEINTSNPTHRSGESSPVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSNELDEYLYSFIDIIDSEYRDTLDYLVDERFDSINKGFVQG
NKVNISLLIDMMKGYEVDDIIRLYYDFIVLKSQKNLGFSIKKLREKMLDEYGFRFKDKQYDSVRSKMYKLMDFLLFCNYYRNDV
VAGESLVRKLRFSMTDDEKEGIYADEAAKLWGKFRNDFENIADHMNGDVIKELGKADMGFDEKILDSEKKNASDLLYFSKMIYM
LTYFLDGKEINDLLTTLISKFDNIKEFLKIMKSSAVNVECELTAGYKLFNDSQRITNELFIVKNIASMRKPAASAKLTMFRDAL
TILGIDDKITDDRISEILKLKEKGKGIHGLRNFITNNVIESSRFVYLIKYANAQKIREVAKNEKVVMFVLGGIPDTQIERYYKS
CVEFPDMNSSLKAKRSELARMIKNISFEDFKDVKQQAKGRENVAKERAKAVIGLYLTVMYLLVKNLVNVNARYVIAIHCLERDF
GLYKEIIPELASKNLKNDYRILSQMLCELCDDRDKSPNLFLKKNKRLRKCVEVDINNADSSMTRKYRNCIAHLTVVRELKEYIG
DIYAVDSYFSIYHYVMQRCITKRENDTKQEEKIKYEDNLLKNHGYTKDFVKALNSPFGYNIPRFKNLSIEQLFDRNEYLTEK
(SEQ ID NO: 328)
>33000193761Ga0187899_10021543_4
[mammals-digestive system-feces]
MNKIHKKQGKTTAKSLGLKSVLKIENDLVVTTFGKKDNPMVVEQSINKASGEKELYVDEDQVKFDSSLIKEKNILSLDSIQHSN
HQIIVNIDQKDASEIGMDYLRLKPELEKEFFGKTFYDNVHIQIAYNLLDLKKIIGLHIGNAIQALENLGRDGSDLVGICDATKP
LNYLDDVKQKADIGFMNRLKPYFMYFDGVLKLDNSKNKNGELNQLDIENWDVIRILSLIRQGCAHAGAYSSLLYTAQNNKVYAD
LINKALSIFSDDLDKFNKSFLKQSKMNLFILFDLYNCRFDRSLQEKIIKEYYRYVLYKDNKNLGFSLKNVRNLIIEGKYDEQER
SGKLQTIRSKLNTLLDFYLYGYYQKNPTFVENIVAKLRESKNDEDKEKVYEEEYHRLLSENNYLVDKKCSDIVYRINEAVKNRK
IFVNANINAVVEKVSCSCFPSLIYVLCKFLDGKEVNELTTAIINKLENIASLINALVTLKSYGGFSEQYKIFDYPNINGLIDDF
RMVKNLTSTKRKLKKASGGEDRIGRQLYADAINIFKEDSFVSANDEKGTGLDQYVNKFFSKDDLGARKVRNLLLNNIIKNRRFV
YLIKYIDPKDCYKLVHNEKIVRFALGQYDESQMPLNQLQKYYDAVIENREGFRKCNDRKKIIDTLVSEINRVSIDGILDIGNRL
VNRGNNDYINHQKQIISLYLTIAYLIVKGVVHTNSLYFIAWHAYERDNNFKFGNDGKDYLALTKEYLTNKKKRVKQLLDHNIEE
ANNSLDSKYFSAYRNKVVHLNFCNIFVNYLDGIGDIHSYYDIYQYVIQKWSIAERSKDFIDPQYLTKLSNDLKQYRTYQRNFLK
IINLPFAYNLARYKNLTIGDLFNDKYPLPKETVKEFYNEE (SEQ ID NO: 329)
>OGCZ01001955_1
[metagenome]
MAKKNKMKPRELREAQKKARQLKAAEINNNAAPAIAAMPAAQVIAPAAEKKKSSVKAAGMKSILVSENKMYITSFGKGNSAVLE
YEVDKVDNNNYNKTQLSSKDNSNIELGDVNEVNITFSSKHGFESGVEINTSNPTHRSGESSPVRWDMLGLKSELEKRFFGKTFD
DNIHIQLIYNILDIEKILAVYVTNIVYALNNMLGIKGSESYDDFMGYLSARNTYEVFTHPDKSNLSDKVKGNIKKSFSTFNDLL
KTKRLGYFGLEEPKTKDTRVSEAYKKRVYHMLAIVGQIRQCVFHDLSEHSEYDLYSFIDNSKKVYRECRETLNYLVDERFDSIN
KGFIQGNKVNISLLIDMMKDDYEADDIIHLYYDFIVLKSQKNLGFSIKKLREKMLDEYGFRFKDKQYDSVRSKMYKLMDFLLFC
NYYRNDVVAGEALVRKLRFSMTDDEKEGIYADEAEKLWGKFRNDFENIADHMNGDVIKELGKADMDFDEKILDSEKKNASDLLY
FSKMIYMLTYFLDGKEINDLLTTLISKFDNIKEFLKIMKSSAVDVECELTAGYRLFNDSQRITNELFIVKNIASMRKPAASAKL
TMFRDALTILGIDDKITDDRISEILKLKEKGKGIHGLRNFITNNVIESSRFVYLIKYANAQKIREVAKNEKVVMFVLGGIPDTQ
IERYYKSCVEFPDMNSSLEAKRSELARMIKNISFDDFKNVKQQAKGRENVAKERAKAVIGLYLTVMYLLVKNLVNVNARYVIAI
HCLERDFGLYKEIIPELASKNLKNDYRILSQTLCELCDESPNLFLKKNERLRKCVEVDINNADSSMTRKYRNCIAHLTVVRELK
EYIGDIRTVDSYFSIYHYVMQRCITKREDDKKQEEKIKFEDDLLKNHGYTKDFVKALNSPFGYNIPRFKNLSIEQLFDRNEYLT
EK (SEQ ID NO: 330)
>OGDY01002059_17
[metagenome]
MGKKIHARDLREQRKTDRTEKFADQNKKREAERAVQKKDAAVSVKSVSSVSSKKDNVTKSMAKAAGVKSVFAVGNTVYMTSFGR
GNDAVLEQKIVDTSHEQLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKAKNVLREDKELYSVLTLIGKLRHWCVHSEEGRAEFWLYKLNELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKRLSKSNIEIQEDKLRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPENTALCSANKKREKLADMIAEIEFENFSDAGNYQKANVTSKTHEAEIKRKNQSIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNINIKEIKEVENYFALYHYLIQKHLENRFADKKVERDTGDFISKLEEHKTYCKDFVKAYCTPFGY
NLVRYKNLTIDGLFDKNYPGKDDSDKQK (SEQ ID NO: 331)
>OGEU01000713_24
[metagenome]
MAKKNKMKPRELREAQKKARQLKAAEINNNAVPAIAAMPAAEVIAPAAEKKKSSVKAAGMKSILVSENKMYITSFGKGNSAVLE
YEVDKVDDNDYNKTQLSSKDNSNIELGNVNEVNITFSSRRGFESGVEINTSNPTHRSGESSSVRGDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDKRVSEAYKKRVYHMLAIVGQIRQSVFHDKSNELDEYLYSFIDIIDSEYRDTLDYLVDERFDSINKGF
IQGNKVNISLLIDMMKDDYEADDIIRLYYDFIVLKSQKNLGFSIKKLREKMLEEYGFRFKDKQYDSVRSKMYKIMDFLLFCNYY
RNDVVAGEALVRKLRFSMTDDEKEGIYADEAAKLWGKFRNDFENIADHMNGDVIKELGKADMDFDEKILDSEKKNASDLLYFSK
MIYMLTYFLDGKEINDLLTTLISKFDNIKEFLKIMKSSAVDVECELTAGYKLFNDSQRITNELFIVKNIASMRKPAASAKLTMF
RDALAILGIDDKITDDRISEILKLKEKGKGIHGLRNFITNNVIESSRFVYLIKYANAQKIREVAKNEKVVMFVLGGIPDTQIER
YYKSCVEVPDMNSSLEAKRSELARMIKNIRFDDFKNVKQQAKGRENVAKERAKAVIGLYLTVMYLLVKNLVNVNARYVIAIHCL
ERDFGLYKEIIPELASKNLKNDYRILSQTLCELCDKSPNLFLKKNERLRKCVEVDINNADSSMTRKYRNCIAHLTVVRELKEYI
GDIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 332)
>OGFM01002125_3
[metagenome]
MAKKNKMKPRELREAQKKARQLKAAEINNNAAPAIAAMPAAEAAAPAAEKKKSSVKAAGMKSILVSENKMYITSFGKGNSAVLE
YEVDKVDNNDYNQTQLSSKGSSNIELHGVNEVNITFSSKHGFESGVEINTSNPTHRSGESSPVRWDMLGLKSELEKRFFGKTFD
DNIHIQLIYNILDIEKILAVYVTNIVYALNNMLGIKDSESYDDFMGYLSAKNTYDVFTDPDESDLSKNIKGNIKKSFSTFNDLL
KTKRLGYFGLEEPKTKDTRVSEAYKKRVYHMLAIVGQIRQCVFHDLSEHSEYDLYSFIDNSKKVYRECRETLNYLVDERFDSIN
KGFIQGNKVNISLLIDMMKGYEADDIIRLYYDFIVLKSQKNLGFSIKKLREKMLDEYGFRFKDKQYDSVRSKMYKLMDFLLFCN
YYRNDVVAGEALVRKLRFSMTDDEKEGIYADEAAKLWGKFRNDFENIADHMNGDVIKELGKADMDFDEKILDSEKKNASDLLYF
SKMIYMLTYFLDGKEINDLLTTLISKFDNIKEFLKIMKSSAVDVECELTAGYKLFNDSQRITNELFVVKNIASMRKPAASAKLT
MFRDALTILGIDDKITDDRISEILKLKEKGKGIHGLRNFITNNVIESSRFVYLIKYANAQKIREVAKNEKVVMFVLGGIPDTQI
ERYYKSCVEFPDMNSSLEAKRSELARMIKNISFDDFKNVKQQAKGRENVAKERAKAVIGLYLTVMYLLVKNLVNVNARYVIAIH
CLERDFGLYKEIIPELASKNLKNDYRILSQTLCELCDDRDESPNLFLKKNERLRKCVEVDINNADSSMTRKYRNCIAHLTVVRE
LKEYIGDIRTVDSYFSIYHYVMQRCITKREDDTKQEEKIKYEDDLLKNHGYTKDFVKALNSPFGYNIPRFKNLSIEQLFDRNEY
LTEK (SEQ ID NO: 333)
>OGHWO1002048_1
[metagenome]
MAKKNKMKPRELREAQKKARQLKAAEINNNAVPAIAAMPAAEVIAPAAEKKKSSVKAAGMKSILVSENKMYITSFGKGNSAVLE
YEVDKVDNNNYNKTQLSSESSSNIELCGVTKVNITFSSKHGFGSGVEINTSNPTHRSGESSPVRWDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDTRVSQAYKKRVYHMLAIVGQIRQCVFHDKSGAKRFDLYSFINNIDPEYRDTLDYLVDERFDSINKGF
IQGNKVNISLLIDMMKGYEVDDIIRLYYDFIVLKSQKNLGFSIKKLREKMLDEYGFRFKDKQYDSVRSKMYKLMDFLLFCNYYR
NDVAAGEALVRKLRFSMTDDEKEGIYADEAEKLWGKFRNDFENIADHMNGDAIKELGKADMDFDEKILDSEKKNASDLLYFSKM
IYMLTYFLDGKEINDLLTTLISKFDNIKEFLKIMKSYAVDVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRILSQKLCELCDKSPNLFLKKNERLRKCVEVDINNADSIMTRKYRNCIAHLTVVRELKEYIG
DIRTVDSYFSIYHYVMQRCITKRENDTKQEDKIKYEDDLLKNHGYTKDFVKALNSPFGYNIPRFKNLSIEQLFDRNEYLTEK
(SEQ ID NO: 334)
>OGIE01002059_21
[metagenome]
MAKKNKMKPRELREAQKKARQLKAAEINNNAAPAIAAMPAAEVIAPAAEKKKSSVKAAGMKSILVSENKMYITSFGKGNSAVLE
YEVDKVDNNNYNKTQLSSKDNSNIELGDVNEVNITFSSKRGNESGVEINTSNPTHRSGESSPVRWDMLGLKSELEKRFFGKTFD
DNIHIQLIYNILDIEKILAVYVTNIVYALNNMLGIKKSESYDDFMGYLSARNTYEVFTHPDKSNLSDKAKGNIKKSFSTFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYETDDIIRLYYDFIVLKSQKNLGFSIKKLREKMLDEYGFRFKDKQYDSVRSKMYKLMDFLLFCNYYR
NDVVAGEALVRKLRFSMTDDEKEGTYADEAEKLWGKFRNDFDNIAGHMNGDAIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRTLSQTLCGLCDKSPNLFLKKNKRLRKCVEVDINNADSIMTRKYRNCIAHLTVVRELKEYIG
DIRTVDSYFSIYHYVMQRCITKREDDTKQEEKIKYEDDLLKNHGYTKDFVKALNSPFGYNIPRFKNLSIEQLFDRNEYLTEK
(SEQ ID NO: 335)
>OGJI01000038_151
[metagenome]
MAKKNKMKPRELREAQKKARQLKAAEINNNAAPAIAAMPAAEVIAPAAEKKKSSVKAAGMKSILVSENKMYITSFGKGNSAVLE
YEVDKVDNDDYNKTQLSSKGSSNIELHGVNEVNITFSSKHGFESGVEISTSNPTHRSGESSPVRWDMLGLKSELEKRFFGKTFD
DNIHIQFIYNILDIEKILAVYVTNIVYALNNMLGVKDSESYDDFMGYLSARNTYKVFTHPDKSNLSDKVKGNIKKSFSTFNDLL
KTKRLGYFGLEEPKTKDTRVSQAYKKRVYHMLAIVGQIRQCVFHDKSSKLHEDLYSFINNIDPEYRDTLDYLVEERLKSINKDF
IEGNKVNISLLIDMMKDDYEADDIIRLYYDFIVLKSQKNLGFSIKKLREKMLDEYGFRFKDKQYDSVRSKMYKLMDFLLFCNYY
RNDVVAGEALVRKLRFSMTDDEKEGIYADEAAKLWGKFRNDFENIADHMNGDVIKELGKADMDFDEKILDSEKKNASDLLYFSK
MIYMLTYFLDGKEINDLLTTLISKFDNIKEFLKIMKSSAVDVECKLTAGYKLFNDSQRITNELFIVKNIASMRKPAASAKLTMF
RDALTILGIDDKITDDRISEILKLKEKGKGIHGLRNFITNNVIESSRFVYLIKYANAQKIREVAKNEKVVMFVLGGIPDTQIER
YYKSCVEFPDMNSSLEAKRSELARMIKNISFDDFKNVKQQAKGRENVAKERAKAVIGLYLTVMYLLVKNLVNVNARYVIAIHCL
ERDFGLYKEIIPELASKNLKNDYRILSQTLCELCDDRDESPNLFLKKNKRLRKCVEVDINNADSSMTRKYRNCIAHLTVVRELK
EYIGDIRTVDSYFSIYHYVMQRCITKRENDTKQEDKIKYEDDLLKNHGYTKDFVKALNSPFGYNIPRFKNLSIEQLFDRNEYLT
EK (SEQ ID NO: 336)
>OGJK01007642_2
[metagenome]
MAKKNKMKPRELREAQKKARQLKAAEINNNAAPAIAAMPAAEVIAPAAEKKKSSVKAAGMKSILVSENKMYITSFGKGNSAVLE
YEVDKVDNNNYNKTQLSSKDNSNIELGDVNEVNITFSSKRGNESGVEINTSNPTHRSGESSPVRWDMLGLKSELEKRFFGKTFD
DNIHIQLIYNILDIEKILAVYVTNIVYALNNMLGIKKSESYDDFMGYLSARNTYEVFTHPDKSNLSDKAKGNIKKSFSTFNDLL
KTKRLGYFGLEEPKTKDTRVSQAYKKRVYHMLAIVGQIRQSVFHDKSSKLDEDLYSFIDIIDSEYRETLDYLVDERFDSINKGF
IQGNKVNISLLIDMMKGYEADDIIRLYYDFIVLKSQKNLGFSIKKLREKMLEEYGYRFKDKQYDSVRSKMYKLMDFLLFCNYYR
NDVIAGEALVRKLRFSMTDDEKEGIYADEAAKLWGKFRNDFENIADHMNGDAIKELGKADMDFDEKILDSEKKYASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEFPDMNSSLEAKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDERDKSPNLFLKKNERLRKCVEVDINNADSIMTRKYRNCIAHLTVVRELKE
YIGDIRTVDSYFSIYHYVMQRCITKREDDTKQEEKIKYEDDLLKNHGYTKDFVKALNSPFGYNIPRFKNLSIEQLFDRNEYLTE
K (SEQ ID NO: 337)
>OGJY01000516_18
[metagenome]
MAKKNKMKPRELREAQKKARQLKAAEINNNAAPAIAAMPAAEVIAPVAGKKKSSVKAAGMKSILVSENKMYITSFGKGNSAVLE
YEVDKVDNNDYNQTQLSSKGSSNIELCGVNEVNITFSSKHGFESGVEINTSNPTHRSGESSPVRWDMLGLKSELEKRFFGKTFD
DNIHIQLIYNILDIEKILAVYVTNIVYALNNMLGIKKSESYDDFMGYLSARNTYEVFTHPDKSNLSDKAKGNIKKSFSTFNDLL
KTKRLGYFGLEEPKTKDTRVSQAYKKRVYHMLAIVGQIRQSVFHDKSSKLDEDLYSFIDIIDSEYRETLDYLVEERLKSINKDF
IEGNKVNISLLIDMMKGFEADDIIRLYYDFIVLKSQKNLGFSIKKLREKMLEEYGYRFKDKQYDSVCSKMYKLMDFLLFCNYYR
NDVVAGEALVRKLRFSMTDDEKEGIYADEAAKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDKITDDRISEILKLKEKGKGIHGLRNFITNNVIESSRFVYLIKYANAQKIREVAKNEKVVMFVLGGIPDTQIERY
YKSCVEFPDMNSSLEVKRSELARMIKNISFDDFKNVKQQAKGRENVAKERAKAVIGLYLTVMYLLVKNLVNVNARYVIAIHCLE
RDFGLYKEIIPELASKNLKNDYRILSQTLCELCDDRDKSSNLFLKKNKRLRKCVEVDINNADSSMTRKYRNCIAHLTVVRELKE
YIGDIRTVDSYFSIYHYVMQRCITKREDDTKQEDKIKYEDNLLKNHGYTKDFVKALNSPFGYNIPRFKNLSIEQLFDRNEYLTE
K (SEQ ID NO: 338)
>OGKA01000617_2
[metagenome]
MAKKNKMKPRELREAQKKARQLKAAEINNNAVPAIAAMPAVEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGVKGSESYDDFMGYLSAQNTYYIFTHPDKSNLSDKVKGNIKKSLSKFNDLLKTK
RLGYFGLEEPKTKDKRVSEAYKKRVYHMLAIVGQIRQSVFHDKSNELDEYLYSFIDIIDSEYRDTLDYLVDERFDSINKGFIQG
NKVNISLLIDMMKGYEADDIIRLYYDFIVLKSQKNLGFSIKKLREKMLDEYGFRFKDKQYDSVRSKMYKLMDFLLFCNYYRNDV
VAGEALVRKLRFSMTDDEKEGIYADEAAKLWGKFKNDFENIADHMNGDVIKEFGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNIRFDDFKNVKQQAKGRENVAKERAKAVIGLYLTVMYLLVKNLVNVNARYVIAIHCLERDF
GLYKEIIPELASKNLKNDYRILSQTLCELCDKSPNLFLKKNERLRKCVEVDINNADSSMTRKYRNCIAHLTVVRELKEYIGDIR
TVDSYFSIYHYVMQRCITKRENDTKQEDKIKYEDDLLKNHGYTKDFVKALNSPFGYNIPRFKNLSIEQLFDRNEYLTEK (SEQ
ID NO: 339)
>OGKG01002483_14
[metagenome]
MGKKIHARDLREQRKTDRTEKFADQNKKREAERAVQKKDAAVSVKSVSSVSSKKDNVTKSMAKAAGVKSVFAVGNTVYMTSFGR
GNDAVLEQKIVDTSHEPLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKARNVLREDKELYSVLTLIGKLRHWCVHSEEGRAEFWLYKLDELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDVDNIKNLSGSNIEIRDNELRKCFISYADSVSEFT
KLIYLLTRFLSGKEINDLVTTLINKFDNIRSFLEILDELGLDRTFTAEYSFFEGSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPKNTALCSANKRREKLADMIAEIKFENFSDAGNYQKANVTSRTSEAEIKRKNQAIIRLYLTVMYIMLKNLV
NVNARYVIAFHCVERDTKLYAESGLEVGNIEKNKTNLTMAVIGVKLENGIIKTEFDKSLAENAANRYLRNARWYKLILDNLKKS
ERAVVNEFRNTVCHLNAIRNININIKEIKEVENYFALYHYLIQKHLENRFADKKVERDTGDFISKLEEHKTYCKDFVKAYCTPF
GYNLVRYKNLTIDGLFDKNYPGKDDSDKQK (SEQ ID NO: 340)
>OGKW01000585_4
[metagenome]
MAKKNKMKPRELREAQKKARQLKAAEINNNATPTIAAMPAAEVIAPAAEKKKSSVKAAGMKSILVSKNKMYITSFGKGNSAVLE
YEVDNNDYNQTQLSSKNSSNIELRGVNEVNITFSSKHGFESGVEINTSNPTHRSGESSSVRGDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNALLKTKR
LGYFGLEEPKTKDTRASEAYKKRVYHMLAIVGQIRQCVFHDKSGAKRFDLYSFINNIDPEYRETLDYLVDERFDSINKGFIQGN
KVNISLLIDMMKGYEADDIIRLYYDFIVLKSQKNLGFSIKKLREKMLDEYGFRFKDKQYDSVRSKMYKIMDFLLFCNYYRNDVV
AGEALVRKLRFSMTDDEKEGIYADEAAKLWGKFRNDFENIADHMNGDVIKELGKADMDFDEKILDSEKKNASDLLYFSKMIYML
TYFLDGKEINDLLTTLISKFDNIKEFLKIMKSSAVNVECELTVGYKLFNDSQRITNELFIVKNIASMRKPAASAKLTMFRDALT
ILGIDDKITDDRISEILKLKEKGKGIHGLRNFITNNVIESSRFVYLIKYANAQKIREVAKNEKVVMFVLGGIPDTQIERYYKSC
VEFPDMNSSLEAKRSELARMIKNISFDDFKNVKQQAKGRENVAKERAKAVIGLYLTVMYLLVKNLVNVNARYVIAIHCLERDFG
LYKEIIPELVSKNLKNDYRILSQTLCELCDKSPNLFLKKNERLRKCVEVDINNADSVMTRKYRNCIAHLTVVRELKEYIGDIRT
VDSYFSIYHYVMQRCITKREDDTKQEEKIKYEDDLLKNHGYTKDFVKALNSPFGYNIPRFKNLSIEQLFDRNEYLTEK (SEQ
ID NO: 341)
>OGLJ01000192_54
[metagenome]
MAKKNKMKPRELREAQKKARQLKAAEINNNAAPAIAAMPAVEVIAPAAEKKKSSVKAAGMKSILVSENKMYITSFGKGNSAVLE
YEVDKMDNNNYNKTQLSSESSSNIKLCGVTKVNITFSSKHGFESGVEINTSNPTHRSGESSPVRWDMLGLKSELEKRFFGKTFD
DNIHIQLIYNILDIEKILAVYVTNIVYALNNMLGVKGSESHDDFIGYLSTNNTYDVFIDPDNSSLSDDKKANVRKSLSKFNALL
KTKRLGYFGLEEPKTKDNRVSEAYKKRVYHMLAIVGQIRQCVFHDKSGAKRFDLYSFINNIYPEYRETLDYLVDERFDSINKGF
IQGNKVNISLLIDMMKGYEADDIIRLYYDFIVLKSQKNLGFSIKKLREKMLDEYGFRFKDKQYDSVRSKMYKLMDFLLFCNYYR
NDVAAGEALVRKLRFSMTDDEKEGIYAGEAAKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTAGYKLFNDSQRITNELFIVKNIASMRKPAASAKLTMFR
DALTILGIDDNITDDRISEILKLKEKGKGIHGLRNFITNNVIESSRFVYLIKYANAQKIREVAKNEKVVMFVLGGIPDTQIERY
YKSCVEVPDMNSSLEAKRSELARMIKNIRFDDFKNVKQQAKGRENVAKERAKAVIGLYLTVMYLLVKNLVNVNARYVIAIHCLE
RDFGLYKEIIPELASKNLKNDYRILSQTLCELCDDRDKSPNLFLKKNKRLRKCVEVDINNADSSMTRKYRNCIAHLTVVRELKE
YIGDIRTVDSYFSIYHYVMQRCITKRENDTKQEDKIKYEDDLLKNHGYTKDFVKALNSPFGYNIPRFKNLSIEQLFDRNEYLTE
K (SEQ ID NO: 342)
>OGLM01001314_21
[metagenome]
MGKKIHARDLREQRKTDRTEKFADQNKKREAERAVQKKDAAVSVKSVSSVSSKKDNVTKSMAKAAGVKSVFAVGNTVYMTSFGR
GNDAVLEQKIVDTSHEPLNIDDPAYQLNVVTMNGYSVTGHRGETVSAVTDNPLRRFNGGKKDEPEQSVPTDMLCLKPTLEKKFF
GKEFNDNIHIQLIYNILDIEKILAVYSTNAIYALNNMSADENIENSDFFMKRTTDETFDDFEKKKESTNSREKADFDAFEKFIG
NYRLAYFADAFYVNKKNPKGKAKNVLREDKELYSVLTLIGKLRHWCVHSEEGRAEFWLYKLNELKDDFKNVLDVVYNRPVEEIN
NRFIENNKVNIQILGSVYKNTDIAELVRSYYEFLITKKYKNMGFSIKKLRESMLEGKGYADKEYDSVRNKLYQMTDFILYTGYI
NEDSDRADDLVNTLRSSLKEDDKTTVYCKEADYLWKKYRESIREVADALDGDNIKRLSKSNIEIQEDKLRKCFISYADSVSEFT
KLIYLLTRFLSGKEINDLVTTLINKFDNIRSFLEIMDELGLDRTFTAEYSFFEGSTKYLAELVELNSFVKSCSFDINAKRTMYR
DALDILGIKSGKTEEDIEKMIDNILQIDANGDKKLKKNNGLRNFIASNVIDSNRFKYLVRYGNPKKIRETAKCKPAVRFVLNEI
PDAQIERYYEACCPKNTALCSANKRREKLADMIAEIKFENFSDAGNYQKANVTSKTHEAEIKRKNQAIIRLYLTVMYIMLKNLV
NVNARYVIAFHCVERDTKLYAESGLEVGNIEKNKTNLTMAVMGVKLENGIIKTEFDKSLAENAANRYLRNARWYKLILDNLKKS
ERAVVNEFRNTVCHLNAIRNININIDGIKEVENYFALYHYLIQKHLENRFADKKVERDTGDFISKLEEHKTYCKDFVKAYCTPF
GYNLVRYKNLTIDGLFDKNYPGKDDSDKQK (SEQ ID NO: 343)
>OGMO01000062_69
[metagenome]
MAKKNKMKPRELREAQKKARQLKAAEINNNAAPAIAAMPAAEVIAPAAEKKKSSVKAAGMKSILVSKNKMYITSFGKGNSAVLE
YEVDKVDNDNYNKTQLSSEDSSNIELCGVTKVNITFSSKHGLESGVEINTSNPTHRSGESSPVRWDMLGLKSELEKRFFGKTFD
DNIHIQLIYNILDIEKILAVYVTNIVYALNNMLGVKGSESYDDFMGYLSAQNTYYIFTHPDKSNLSDKVKGNIKKSLSKFNDLL
KTKRLGYFGLEEPKTKDTRVSQAYKKRVYHMLAIVGQIRQSVFHDKSSKLDEDLYSFINNIDPEYRETLDYLVDERFDSINKGF
IQGNKVNISLLIDMMKGYEADDIIRLYYDFIVLKSQKNLGFSIKKLREKMLDEYGYRFKDKQYDSVRSKMYKLMDFLLFCNYYR
NDVVAGEALVRKLRFSMTDDEKEGIYADEAAKLWGKFRNDFENIADHMNGDVIKELGKADMDFDEKILDSEKKNASDLLYFSKM
IYMLTYFLDGKEINDLLTTLISKFDNIKEFLKIMKSSAVDVECELTVGYKLFNDSQRITNELFIVKNIASMRKPAASAKLTMLR
DALTILGIDDKITDDRISEILKLKEKGKGIHGLRNFITNNVIESSRFVYLIKYANAQKIREVAENEKVVMFVLGGIPDTQIERY
YKSCVEFPDMNSSLEAKRSELARMIKNIRFDDFKNVKQQANGRENVAKERAKAVIGLYLTVMYLLVKNLVNVNARYVIAIHCLE
RDFGLYKEIIPELASKNLKNDYRILSQTLCELCDDRDESPNLFLKKNKRLRKCVEVDINNADSSMTRKYRNCIAHLTVVRELKE
YIGDIRTVDSYFSIYHYVMQRCITKREDDTKQEEKIKYEDDLLKNHGYTKDFVKALNSPFGYNIPRFKNLSIEQLFDRNEYLTE
K (SEQ ID NO: 344)
>OGMP01001167_15
[metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGNVNEVNITFSSRRGFESGVEINTSNPTHRSGESSSVRGDMLGLKSELEKRFFGKTFDDNI
HIQLIYNILDIEKILAVYVTNIVYALNNMLGEGDESNYDFMGYLSTFNTYKVFTNPNGSTLSDDKKENIRKSLSKFNVLLKTKR
LGYFGLEEPKTKDNRVSEAYKKRVYHMLAIVGQIRQCVFHDKSGAKRFDLYSFINNIDPEYRDTLDYLVEERLKSINKDFIQGN
KVNISLLIDMMKGYEADDIIRLYYDFIVLKSQKNLGFSIKKLREKMLEEYGFRFKDKQYDSVRSKMYKLMDFLLFCNYYRNDIA
AGEALVRKLRFSMTDDEKEGIYAGEAAKLWGKFRNDFENIADHMNGDVIKELGKADMDFDEKILDSEKKNASDLLYFSKMIYML
TYFLDGKEINDLLTTLISKFDNIKEFLKIMKSSAVDVECELTAGYKLFNDSQRITNELFIVKNIASMRKPAASAKLTMFRDALT
ILGIDDNITDDRISEILKLKEKGKGIHGLRNFITNNVIESSRFVYLIKYANAQKIREVAKNEKVVMFVLGGIPDTQIERYYKSC
VEVPDMNSSLEAKRSELARMIKNIRFDDFKNVKQQAKGRENVAKERAKAVIGLYLTVMYLLVKNLVNVNARYVIAIHCLERDFG
LYKEIIPELASKNLKNDYRILSQTLCELCDDRDKSPNLFLKKNKRLRKCVEVDINNADSNMTRKYRNCIAHLTVVRELKEYIGD
IRTVDSYFSIYHYVMQRRITKRKDDTKQEEKIKYEDDLLKNHGYTKDFVKALNSPFGYNIPRFKNLSIEQLFDRNEYLTEK
(SEQ ID NO: 345)
>OGUJO1000114_43
[metagenome]
MAKKKRITAKERKQNHRESLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRVPYSEYGGGKDSNGEPKNQSKWKKRKNFIKFYNKSK
PHLGYYENIFYDHGEPISEEKFYNYLNILNFIRNNTFHYKDDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKILLEIVEVLRLSKNDDEKENVYKKYAEKLFKADDVINPIKAISKLFAEKGNKLFKEKIIIKKEYIEDVSIDKNIYDFT
KVIFFMTCFLDGKEINDLLTNIISKLQVIEDHNNVIKFISHNKDAVYKDYSDKYAIFRNAGKIATELEAIKSIARMENKIENAP
QEPLLNDALLALGVSKTDLENTYNKYFDSKEKTDKQSQKVSTFLMNNVINNNRFKYVIKYINPADINGLAKNRYLVKFVLSKIP
EEQIDSYYKLFSNEEEPSCEEKIKLLTKKISKLNFQTLFENNKIPNVEKERKKAIITLYFTIVYILVKNLVNINGLYTLALYFV
ERDRYFYKKICGKALRRKVGDKYDYLLLPEIFSGSKYREETKNLKLPKEKDRDIMKKYLPNDKDREGYNDFFTAYRNNIVHLNI
IAKLSELTKNIDKDINSYFDIYHYCTQRVMFDYCKMNNNVVLAKMKDLAHIKSDCDEFSSKHTYPFSSAVLRFMNLPFAYNVPR
FKNLSYKKFFDKQWLNH (SEQ ID NO: 346)
>OGUJO1000114_45
[metagenome]
MAKKKRITAKERKQNHRESLMKKADSNAEKEKAKKPVVENKPDTAISKDNTPKPNKEIKKSKAKLAGVKWVIKANDDVAYISSF
GKGNNSVLEKRIMGDVSSNVNKDSHMYVNPKYTKKNYEIKNGFSSGSSLVTYPNKPDKNSGMDALCLKPYFEKDFFGHIFTDNM
HIQAIYNIFDIEKILAKHITNIIYTVNSFDRNYNQSGNDTIGFDINYRVPYSEYGGGKDSNGEPKNQSKWKKRKNFIKFYNKSK
PHLGYYENIFYDHGEPISEEKFYNYLNILNFIRNNTFHYKDDDIELYSENYSEEFVFINCLNKFVKNKFKNVNKNFISNEKNNL
YIILNAYGKDTENVEVVKKYSKELYKLSVLKTNKNLGVNVKKLRESAIEYGYCPLPYDKEKEVAKLSSVKHKLYKTYDFVITHY
LNSNDKILLEIVEVLRLSKNDDEKENVYKKYAEKLFKADDVINPIKAISKLFAEKGNKLFKEKIIIKKEYIEDVSIDKNIYDFT
KVIFFMTCFLDGKEINDLLTNIISKLQVIEDHNNVIKFISHNKDAVYKDYSDKYAIFRNAGKIATELEAIKSIARMENKIENAP
QEPLLNDALLALGVSKTDLENTYNKYFDSKEKTDKQSQKVSTFLMNNVINNNRFKYVIKYINPADINGLAKNRYLVKFVLSKIP
EEQIDSYYKLFSNEEEPSCEEKIKLLTKKISKLNFQTLFENNKIPNVEKERKKAIITLYFTIVYILVKNLVNINGLYTLALYFV
ERDRYFYKKICGKALRRKVGDKYDYLLLPEIFSGSKYREETKNLKLPKEKDRDIMKKYLPNDKDREGYNDFFTAYRNNIVHLNI
IAKLSELTKNIDKDINSYFDIYHYCTQRVMFDYCKMNNNVVLAKMKDLAHIKSDCDEFSSKHTYPFSSAVLRFMNLPFAYNVPR
FKNLSYKKFFDKQ (SEQ ID NO: 347)
>OJKY01000879_3
[metagenome]
MAKKITAKQKREEKERLNKQKWAKNDSVIIVPETKEEIKTGEIQDNNRKRSRQKSQAKAMGLKAVLSFDNKIAIASFVSSKNAK
SSHIERITDKEGTTISVNSKMFESSVNKRDINIEKRITIEEPQQDGTIKKEEKGVKSTTCNPYFKVGGKDYIGIKEIAEEHFFG
RAFPNENLRVQIAYNIFDVQKILGTFVNNIIYSFYNLSRDEVQSDNDVIGMLYSISDYDRQKETETFLQAKSLLKQTEAYYAYF
DDVFKKNKKPDKNKEGDNSKQYQENLRHNFNILRVLSFLRQICMHAEVHVSDDEGCARTQNYTDSLEALFNISKAFGKKMPELK
TLIDNIYSKGINAINDEFVKNGKNNLYILSKVYPNEKREVLLREYYNFVVCKEGSNIGISTRKLKETMIAQNMPSLKEENTYRN
KLYTVMNFILVRELKNCATIREQMIKELRANMDEEEGRDRIYSKYAKEIYLYVKDKLKLMLNVFKEEAEGIIIPGKEDPVKFSH
GKLDKKEIESFCLTTKNTEDITKVIYFLCKFLDGKEINELCCAMMNKLDGISDLIETAKQCGEDVEFVDQFKCLSKCATMSNQI
RIVKNISRMKKEMTIDNDTIFLDALELLGRKIEKYQKDKNGDYVKDEKGKKVYTKDYNNFQDMFFEGKNHRVRNFVSNNVIKSK
WFSYVVRYNKPAECQALMRNSKLVKFALDELPDSQIEKYYISVFGEKSSSSNEEMRRELLKKLCDFSVRGFLDEIVLLSEDEMK
QKDKFSEKEKKKSLIRLYLTIVYLITKSMVKINTRFSIACATYERDYILLCQSEKAERAWEKGATAFALTRKFLNHDKPTFEQY
YTREREISAMPQEKRKELRKENDQLLKKTHYSKHAYCYIVDNVNNLTGAVANDNGRGLPCLSEKNDNANLFVEMRNKIVHLNVV
HDMVKYINEIKNITSYYAFFCYVLQRMIIGNNSNEQNKFKAKYSKTLQEFGTYSKDLMWVLNLPFAYNLPRYKNLSNEQLFYDE
EERMEKIVGRKNDSR (SEQ ID NO: 348)
>OLJF01000187_58
[metagenome]
MAKKNKMKPRELREAQKKARQLKAAEINNNAAPAIAAMPAAEVIAPAAEKKKSSVKAAGMKSILVSENKMYITSFGKGNSAVLE
YEVDNNDYNKTQLSSKDNSNIELGDVNEVNITFSSKHGFGSGMKINTSNPTHRSGESSPVRWDMLGLKSELEKRFFGKTFDDNI
HIQLIYNILDIEKILAVYVTNIVYALNNMLGVKGSESHDDFIGYLSTNNIYDVFIDPDNSSLSDDKKANVRKSLSKFNVLLKTK
RLGYFGLEEPKTKDNRVSEAYKKRVYHMLAIVGQIRQCVFHDKSGAKRFDLYSFINNIDPEYRDTLDYLVEERLKSINKDFIQG
NKVNISLLIDMMKGYEADDIIRLYYDFIVLKSQKNLGFSIKKLREKMLEEYGFRFKDKQYDSVRSKMYKLMDFLLFCNYYRNDV
VAGEALVRKLRFSMTDDEKEGIYADEAAKLWGKFRNDFENIADHMNGDVIKELGKADMDFDEKILDSEKKNASDLLYFSKMIYM
LTYFLDGKEINDLLTTLISKFDNIKEFLKIMKSSAVDVECELTAGYKLFNDSQRITNELFIVKNIASMRKPAASAKLTMFRDAL
TILGIDDNITDDRISEILKLKEKGKGIHGLRNFITNNVIESSRFVYLIKYANAQKIREVAKNEKVVMFVLGGIPDTQIERYYKS
CVEFPDMNSSLEAKRSELARMIKNISFDDFKNVKQQAKGRENVAKERAKAVIGLYLTVMYLLVKNLVNVNARYVIAIHCLERDF
GLYKEIIPELASKNLKNDYRILSQTLCELCDDRDESPNLFLKKNKRLRKCVEVDINNADSSMTRKYRNCIAHLTVVRELKEYIG
DIRTVDSYFSIYHYVMQRCITKRENDTKQEEKIKYEDDLLKNHGYTKDFVKALNSPFGYNIPRFKNLSIEQLFDRNEYLTEK
(SEQ ID NO: 349)
>OMWO01000091_3
[uncultured Clostridiales bacterium]
MAKKKRMSAKERKQQQINLRIKKATEDSTKKVNTTVAVNNKPISKEIKKSKAKLAGVKWVIKANDDVAYISSFGKGNNSVLEKR
IIGDVSSDVNKDSHMYVNPKYTKKNYEIKNGFSSGSSLTTHPNKPDKNSGMDALCLKTYFEKEIFKDKFNDNMHIQAIYNIFDI
EKTLAKHITNIIYAVNSLDRSYIQSGNDTIGFGLNFNIPYAEYGGGKDSNGKPENKSAWEKRESFIKFYNNAKDRFGYFESVFY
QNGKQISEEKFYIYLNILNFVRNSTFHYNNTSSHLYKERYCKINPKNNLKTDFEFVSYLNEFVKNKFKNVNKNFISNEKNNLYI
ILNAYGEDIEDVEVVKKYSKELYKLSVLKTNKNLGVNVKKLRESAIEYGYCPLPYDKEKEVAKLSSIKHKLYKTYDFVITHYLN
SNDKLLLEIVEALRLSKNDDKKENVYKIYAEKIFKAEYVINPIKTISNLFAEKGDKLFNEKVSISEEYVEDIRIDKNIHNFTKV
IFFLTCFLDGKEINDLLTNIISKLQVIEDHNNVIKAIANNNDAVYKDYSDKYAVFKNSGKIATELEAIKSIARMENKINKAFKE
PLLKDAMLALGVSPNDLDEKYEKYFKTDVDADKDHQKVSTFLMNNVINNSRFKYVVKYINPADINRLAKNKHLVKFVLDQIPHK
QIDSYYNSVSTVEEPSYKGKIQLLTKKITGLNFYSLFENCKIPNVEKEKKKAVITLYFTIIYILVKNLVNINGLYTLALYFVER
DGFFYKKICEKKDKKKTNKDVDYLLLPEIFSGSKYREETKNLKLPKEKDREIMKKYLPNDEDRKEYNKFFKQYRNNIVHLNIIA
NLSKLTSTIDKEINSYFEIFHYCAQRVMFDYCKNNNKVVL (SEQ ID NO: 350)

TABLE 3
Representative Type VI-D Direct Repeat Nucleotide Sequences

| Cas13d Effector Protein Accession | |
|---|---|
| Number | Direct Repeat Nucleotide Sequence |
| WP_005358205.1 (SEQ ID NO: 1) | GAACTACACCCGTGCAAAAATGCAGGGGTCTAAAAC (SEQ ID NO: 32) |
| WP_005358205.1 (SEQ ID NO: 1) | GAATTACACCCGTGCAAAAATGCAGGGGTCTAAAAC (SEQ ID NO: 33) |
| WP_005358205.1 (SEQ ID NO: 1) | GAACTACACCCGTGCAAAATTGCAGGGGTCTAAAAC (SEQ ID NO: 34) |
| LARF01000048_8 (SEQ ID NO: 2) | CTACTACACTGGTGCGAATTTGCACTAGTCTAAAAC (SEQ ID NO: 62) |
| LARF01000048_8 (SEQ ID NO: 2) | CTACTACACTGGTGCAAATTTGCACTAGTCTAAAAC (SEQ ID NO: 72) |
| 33000102661Ga0129314_1001134_19 | GAACTACACCCGTGCAAAAATGCAGGGGTCTAAAAC (SEQ ID NO: 43) |
| (SEQ ID NO: 3) | |
| 33000062261Ga0099364_10024192_5 | GTGCAGTAGCCTTACAGATTCGTAGGGTTCTGAGAC (SEQ ID NO: 37) |
| (SEQ ID NO: 4) | |
| NZ_NFLV01000009_111 (SEQ ID NO: 5) | GAACTACACCCTGGCTGAAAGTCAGGGTCTAAAAC (SEQ ID NO: 53) |
| NFIR01000008_78 (SEQ ID NO: 6) | GAACTACACTCTGGCTGAAAGTCAGGGTCTAAAAC (SEQ ID NO: 52) |
| NFIR01000008_78 (SEQ ID NO: 6) | GAACTACACTCTGGCTGAAAGTCAGGGTCTA (SEQ ID NO: 351) |
| CDYU01023067_140 (SEQ ID NO: 7) | CAGCACTACACCCCCCTGAAACAGGAGGGGTCTAAAAC (SEQ ID NO: 56) |
| CDY501033339_14 (SEQ ID NO: 7) | TAGCACTACACCCCCCTGAAACATGAGGGGTCTAAAAC (SEQ ID NO: |
| | 359) |
| CDYU01023067_140 (SEQ ID NO: 7) | TAGCACTACACCCCCCTGAAACATGAGGGGTCTAAAAC (SEQ ID NO: |
| | 360) |
| CDYU01004315_2 (SEQ ID NO: 8) | CTACTACACTGGTGCAAATTTGCACTAGTCTAAAAC (SEQ ID NO: 54) |
| CDYU01004315_2 (SEQ ID NO: 8) | CTACTACACTAGTGCGAATTTGCACTAGTCTAAAAC (SEQ ID NO: 55) |
| CDYU01004315_2 (SEQ ID NO: 8) | CTACTACACTGGTGCGAATTTGCACTAGTCTAAAAC (SEQ ID NO: 62) |
| OLFT01003273_1 (SEQ ID NO: 8) | CTACTACACTGGTGCGAATTTGCACTAGTCTAAAAC (SEQ ID NO: 62) |
| CDZE01002059_22 (SEQ ID NO: 9) | CTACTACACTGGTGCGAATTTGCACTAGTCTAAAAC (SEQ ID NO: 62) |
| CDYX01024884_4 (SEQ ID NO: 9) | CTACTACACTGGTGCGAATTTGCACTAGTCTAAAACC (SEQ ID NO: 361) |
| CDTW01032418_55 (SEQ ID NO: 10) | CAGCACTACACCCCCCTGAAACATGAGGGGTCTAAAAC (SEQ ID NO: |
| | 358) |
| CDZD01043528_308 (SEQ ID NO: 10) | CAGCACTACACCCCCCTGAAACATGAGGGGTCTAAAAC (SEQ ID NO: |
| | 362) |
| CDZF01024873_75 (SEQ ID NO: 10) | CAGCACTACACCCCCCTGAAACATGAGGGGTCTAAAAC (SEQ ID NO: |
| | 363) |
| CDZF01043927_109 (SEQ ID NO: 10) | CAGCACTACACCCCCCTGAAACATGAGGGGTCTAAAAC (SEQ ID NO: |
| | 364) |
| CDZT01047721_3 (SEQ ID NO: 11) | CTACTATACTGGTGCGAATTTGCACTAGTCTAAAATG (SEQ ID NO: 368) |
| CDZU01022944_3 (SEQ ID NO: 11) | CTACTATACTGGTGCGAATTTGCACTAGTCTAAAATG (SEQ ID NO: 369) |
| CDZVO1031905_3 (SEQ ID NO: 11) | CTACTATACTGGTGCGAATTTGCACTAGTCTAAAATG (SEQ ID NO: 370) |
| OGPA01000243_2 (SEQ ID NO: 11) | CTACTATACTGGTGCGAATTTGCACTAGTCTAAAATG (SEQ ID NO: 410) |
| 33000072961Ga0104830_100502_31 | CTACTACACTGGTGCGAATTTGCACTAGTCTAAAAC (SEQ ID NO: 38) |
| (SEQ ID NO: 12) | |
| 33000072961Ga0104830_100502_31 | CTACTACACTAGTGCGAATTTGCACTAGTCTAAAAC (SEQ ID NO: 74) |
| (SEQ ID NO: 12) | |
| ODXP01000624_4 (SEQ ID NO: 12) | CTACTACACTAGTGCGAATTTGCACTAGTCTAAAACTA (SEQ ID NO: |
| | 547) |
| 33000072991Ga0104319_1000623_29 | CTACTACACTGGTGCGAATTTGCACTAGTCTAAAAC (SEQ ID NO: 62) |
| (SEQ ID NO: 13) | |
| ODKA01005851_3 (SEQ ID NO: 13) | CTACTACACTGGTGCGAATTTGCACTAGTCTAAAAC (SEQ ID NO: 62) |
| OGPQ01001037_3 (SEQ ID NO: 14) | CTACTACACTGGTGCAAATTTGCACTA (SEQ ID NO: 414) |
| 33000084961Ga0115078_100057_51 | CTACTACACTGGTGCAAATTTGCACTA (SEQ ID NO: 557) |
| (SEQ ID NO: 14) | |
| CDZKO1015063_14 (SEQ ID NO: 15) | TACTGGTGCGAATTTGCACTAA (SEQ ID NO: 365) |
| 33000015981EMG_10000232_1 | GGACAATAACCTGCGAATTTTGGCAGGTTCTATGAC (SEQ ID NO: 36) |
| (SEQ ID NO: 16) | |
| 33000015981EMG_10003641_1 | GAACTACACCCCTGCAGAAATGCTGGGGTCTGAAAC (SEQ ID NO: 35) |
| (SEQ ID NO: 17) | |
| 33000184941Ga0187911_10005861_19 | GAACTACAGCCCTGTGAAATAACGGGGTTCTAAAAC (SEQ ID NO: 46) |
| (SEQ ID NO: 18) | |
| 33000184941Ga0187911_10005861_19 | GAACTACAGCCCTGTGAAATAACAGGGTTCTAAAAC (SEQ ID NO: 47) |
| (SEQ ID NO: 18) | |
| 33000184941Ga0187911_10005861_19 | CATGTAAACCCCTAACAAATGGTAGGGGTTTGAAAC (SEQ ID NO: 562) |
| (SEQ ID NO: 18) | |
| 33000184951Ga0187908_10006038_18 | CATGTAAACCCCTAACAAATGGTAGGGGTTTGAAAC (SEQ ID NO: 565) |
| (SEQ ID NO: 18) | |
| 33000184751Ga0187907_10006632_17 | CATGTAAACCCCTAACAAATGATAGGGGGTTGAAAC (SEQ ID NO: 44) |
| (SEQ ID NO: 19) | |
| 33000184941Ga0187911_10005861_18 | GAACTACAGCCCTGTGAAATAACGGGGTTCTAAAAC (SEQ ID NO: 46) |
| (SEQ ID NO: 19) | |
| 33000184941Ga0187911_10005861_18 | GAACTACAGCCCTGTGAAATAACAGGGTTCTAAAAC (SEQ ID NO: 47) |
| (SEQ ID NO: 19) | |
| 33000184751Ga0187907_10006632_17 | CATGTAAACCCCTAACAAATGGTAGGGGTTTGAAAC (SEQ ID NO: 558) |
| (SEQ ID NO: 19) | |
| 33000184751Ga0187907_10006632_17 | CATGTAAACCCCTAACAAATGGTAGGGGTTTGAAAC (SEQ ID NO: 559) |
| (SEQ ID NO: 19) | |
| 33000184931Ga0187909_10005433_18 | CATGTAAACCCCTAACAAATGGTAGGGGTTTGAAAC (SEQ ID NO: 560) |
| (SEQ ID NO: 19) | |
| 33000184931Ga0187909_10005433_18 | CATGTAAACCCCTAACAAATGGTAGGGGTTTGAAAC (SEQ ID NO: 561) |
| (SEQ ID NO: 19) | |
| 33000184941Ga0187911_10005861_18 | CATGTAAACCCCTAACAAATGGTAGGGGTTTGAAAC (SEQ ID NO: 563) |
| (SEQ ID NO: 19) | |
| 33000184951Ga0187908_10006038_19 | CATGTAAACCCCTAACAAATGGTAGGGGTTTGAAAC (SEQ ID NO: 566) |
| (SEQ ID NO: 19) | |
| 33000188781Ga0187910_10006931_17 | CATGTAAACCCCTAACAAATGGTAGGGGTTTGAAAC (SEQ ID NO: 567) |
| (SEQ ID NO: 19) | |
| 33000188781Ga0187910_10006931_17 | CATGTAAACCCCTAACAAATGGTAGGGGTTTGAAAC (SEQ ID NO: 568) |
| (SEQ ID NO: 19) | |
| 33000184941Ga0187911_10069260_3 | GAACTACAGCCCTGTGAAATAACAGGG (SEQ ID NO: 564) |
| (SEQ ID NO: 20) | |
| 33000184931Ga0187909_10030832_9 | CTACTACTACCCTGTTATTTGACAGGGTTCAAAAAC (SEQ ID NO: 45) |
| (SEQ ID NO: 21) | |
| 33000184941Ga0187911_10019634_9 | CTACTACTACCCTGTTATTTGACAGGGTTCAAAAAC (SEQ ID NO: 45) |
| (SEQ ID NO: 21) | |
| 33000188781Ga0187910_10040531_1 | GTTTCTGAACCCTGCCATTTGGCAGGGTAGTAGTTG (SEQ ID NO: 569) |
| (SEQ ID NO: 21) | |
| 33000184931Ga 0187909_10024847_5 | GAACGACGTCACTACACACCGAGAGGTGTCTAAAAC (SEQ ID NO: 48) |
| (SEQ ID NO: 22) | |
| 33000184941Ga0187911_10037073_4 | GAACGACGTCACTACACACCGAGAGGTGTCTAAAAC (SEQ ID NO: 48) |
| (SEQ ID NO: 22) | |
| 33000184951Ga0187908_10013323_2 | GAACGACGTCACTACACACCGAGAGGTGTCTAAAAC (SEQ ID NO: 48) |
| (SEQ ID NO: 22) | |
| 33000188781Ga0187910_10015336_15 | GAACGACGTCACTACACACCGAGAGGTGTCTAAAAC (SEQ ID NO: 48) |
| (SEQ ID NO: 22) | |
| 33000188781Ga0187910_10015336_15 | CAACTACTACCCTGCCAAATGGCAGGGTTCAGAAAC (SEQ ID NO: 49) |
| (SEQ ID NO: 22) | |
| WP_074833651.1 (SEQ ID NO: 23) | CCCTTTGTACTATACCTGTTTTACACAGGTCTAAAAC (SEQ ID NO: 60) |
| WP_074833651.1 (SEQ ID NO: 23) | GTACTATACCTGTTTTACACAGGATAATAACCAAAAT (SEQ ID NO: 61) |
| WP_074833651.1 (SEQ ID NO: 23) | CTACTATACTAGTGTGATTTTACACTAGTCTAAAAC (SEQ ID NO: 352) |
| WP_041337480.1 (SEQ ID NO: 24) | CTACTACACTAGTGCGAATTTGCACTAGTCTAAAAC (SEQ ID NO: 63) |
| WP_041337480.1 (SEQ ID NO: 24) | TCTCTTGGCGGAAAGAAAACAGAAAGACGAAGAACAGGACAAATGGCTATC |
| | (SEQ ID NO: 353) |
| DBYI01000091_43 (SEQ ID NO: 25) | GAACTATACCCCTACCAAATGGTCGGGGTCTGAAAC (SEQ ID NO: 64) |
| WP_075424065.1 (SEQ ID NO: 26) | CAAGTAAACCCCTACCAACTGGTCGGGGTTTGAAAC (SEQ ID NO: 65) |
| WP_075424065.1 (SEQ ID NO: 26) | CAAGTAAACCCTTACCAACTGGTCGGGGTTTGAAAC (SEQ ID NO: 66) |
| WP_009985792.1 (SEQ ID NO: 27) | GAACTATAGTAGTGTAAATTTGCACTACTATAAAAC (SEQ ID NO: 67) |
| WP_009985792.1 (SEQ ID NO: 27) | GAACTATAGTAGTGTGAATTTACACTACTCTAAAAC (SEQ ID NO: 354) |
| CDC65743.1 (SEQ ID NO: 28) | CTACTACACTAGTGCGAATTTGCACTAGTCTAAAAC (SEQ ID NO: 68) |
| CDC65743.1 (SEQ ID NO: 28) | CTACTACACTAGTGCGAATTTGCGCTAGTCTAAAAC (SEQ ID NO: 69) |
| CDC65743.1 (SEQ ID NO: 28) | CTACTACACTAGTGCGAATTTGCACTAGTCTAAAAC (SEQ ID NO: 70) |
| CDC65743.1 (SEQ ID NO: 28) | CTACTACACTGGTGCAAATTTGCACTAGTCTAAAAC (SEQ ID NO: 71) |
| CDC65743.1 (SEQ ID NO: 28) | GTGCGAATTTGCGCTAGTCTAAAAC (SEQ ID NO: 356) |
| DJXD01000002_3 (SEQ ID NO: 29) | CAACTACAACCCCGTAAAAATACGGGGTTCTGAAAC (SEQ ID NO: 73) |
| DJXD01000002_3 (SEQ ID NO: 29) | CAACTACAACCCCGTAAAAATACGGGGTTCTGAAACC (SEQ ID NO: 357) |
| SCH71549.1 (SEQ ID NO: 30) | CTACTACACTAGTGCGAATTTGCACTAGTCTAAAAT (SEQ ID NO: 57) |
| SCH71549.1 (SEQ ID NO: 30) | CTACTACACTAGTGCGAATTTGCGCTAGTCTAAAAC (SEQ ID NO: 58) |
| SCH71549.1 (SEQ ID NO: 30) | CTACTACACTAGTGCGAATTTGCGCTAGTCTAAAAC (SEQ ID NO: 69) |
| SCH71549.1 (SEQ ID NO: 30) | GTGCGAATTTGCGCTAGTCTAAAA (SEQ ID NO: 367) |
| SCH71549.1 (SEQ ID NO: 30) | GTGCGAATTTGCGCTAGTCTAAAAC (SEQ ID NO: 409) |
| SCH71549.1 (SEQ ID NO: 30) | GTGCGAATTTGCGCTAGTCTAAAAC (SEQ ID NO: 415) |
| SCH71549.1 (SEQ ID NO: 30) | GTGCGAATTTGCGCTAGTCTAAAAC (SEQ ID NO: 488) |
| SCH71549.1 (SEQ ID NO: 30) | GTGCGAATTTGCGCTAGTCTAAAAC (SEQ ID NO: 514) |
| SCH71549.1 (SEQ ID NO: 30) | GTGCGAATTTGCGCTAGTCTAAAAC (SEQ ID NO: 526) |
| SCJ27598.1 (SEQ ID NO: 31) | CTACTACACTGGTGCAAATTAGCACTAGTCTAAAAC (SEQ ID NO: 76) |
| SCJ27598.1 (SEQ ID NO: 31) | CTACTACACTGGTGCAAATTAGCACTAGTCTAAAAC (SEQ ID NO: 77) |
| SCJ27598.1 (SEQ ID NO: 31) | CTACTACACTGGTGTGAATTTGCAC (SEQ ID NO: 487) |
| NZ_ACOK01000100_5 (SEQ ID NO: 200) | GAACTATAGTAGTGTAAATTTGCACTACTATAAAAC (SEQ ID NO: 67) |
| NZ_ACOK01000100_5 (SEQ ID NO: 200) | GAACTATAGTAGTGTGAATTTACACTACTCTAAAAC (SEQ ID NO: 355) |
| 33000062261Ga0099364_10024192_5 | GTGCAGTAGCCTTACAGATTCGTAGGGTTCTGAGAC (SEQ ID NO: 37) |
| (SEQ ID NO: 201) | |
| 33000073611Ga0104787_100954_14 | CTACTACACAGGTGCAATTTTGCACTAGTCTAAAAC (SEQ ID NO: 40) |
| (SEQ ID NO: 202) | |
| 33000073611Ga0104787_100954_14 | CTACTACACTGGTGCGAATTTGCACTAGTCTAAAAC (SEQ ID NO: 41) |
| (SEQ ID NO: 202) | |
| CDZKO1015063_14 (SEQ ID NO: 202) | TACTGGTGCGAATTTGCACTAA (SEQ ID NO: 366) |
| OIZB01000622_13 (SEQ ID NO: 202) | TACTGGTGCGAATTTGCACTAA (SEQ ID NO: 498) |
| OIZB01000622_13 (SEQ ID NO: 202) | TACTGGTGCGAATTTGCACTAA (SEQ ID NO: 499) |
| ODHZ01001211_7 (SEQ ID NO: 202) | TACTGGTGCGAATTTGCACTAA (SEQ ID NO: 537) |
| 33000073611Ga0104787_100954_14 | TACTGGTGCGAATTTGCACTAA (SEQ ID NO: 554) |
| (SEQ ID NO: 202) | |
| 33000073611Ga0104787_100954_14 | TACTGGTGCGAATTTGCACTAA (SEQ ID NO: 555) |
| (SEQ ID NO: 202) | |
| 33000082721Ga0111092_1001379_1 | TACTGGTGCGAATTTGCACTAA (SEQ ID NO: 556) |
| (SEQ ID NO: 202) | |
| CEAA01017658_2 (SEQ ID NO: 203) | CTACTACACTGGTGCGAATTTGCACTAGTCTAAAAC (SEQ ID NO: 62) |
| OCHE01000387_10 (SEQ ID NO: 203) | GGAGGTGATAAAAATGGGAAA (SEQ ID NO: 392) |
| OCTW011587266_5 (SEQ ID NO: 204) | CTTATACAACACCCATTTTCACAGTGGGT (SEQ ID NO: 371) |
| OCVV011003687_3 (SEQ ID NO: 205) | GTTTGAGAGTAGTGTAATTTTATAGGGTAGTAAAAC (SEQ ID NO: 372) |
| OCVV011003687_3 (SEQ ID NO: 206) | GTTTGAGAGTAGTGTAATTTTATAGGGTAGTAAAAC (SEQ ID NO: 373) |
| ODA1010069496_4 (SEQ ID NO: 207) | GAACTATAGTAGTGTTTTTTTACACT (SEQ ID NO: 374) |
| ODA1011611274_2 (SEQ ID NO: 208) | GTACTACACCCCTGCAGTTTTGCAGGGGTCTGAAAC (SEQ ID NO: 375) |
| OATA01000148_47 (SEQ ID NO: 209) | CTACTATACTGGTGCGATTTTGCACTAGTCTAAAAC (SEQ ID NO: 376) |
| OBAI01000753_39 (SEQ ID NO: 209) | CTACTATACTGGTGCGATTTTGCACTAGTCTAAAAC (SEQ ID NO: 379) |
| OBAQ01000162_41 (SEQ ID NO: 209) | CTACTATACTGGTGCGATTTTGCACTAGTCTAAAAC (SEQ ID NO: 380) |
| OCHUO1001749_1 (SEQ ID NO: 209) | CTACTATACTGGTGCGATTTTGCACTAGTCTAAAAC (SEQ ID NO: 393) |
| OCPV01000148_47 (SEQ ID NO: 209) | CTACTATACTGGTGCGATTTTGCACTAGTCTAAAAC (SEQ ID NO: 396) |
| OFMN01000509_2 (SEQ ID NO: 209) | CTACTATACTGGTGCGATTTTGCACTAGTCTAAAAC (SEQ ID NO: 397) |
| OFRY01000077_43 (SEQ ID NO: 209) | CTACTATACTGGTGCGATTTTGCACTAGTCTAAAAC (SEQ ID NO: 400) |
| OGRH01000378_2 (SEQ ID NO: 209) | CTACTATACTGGTGCGATTTTGCACTAGTCTAAAAC (SEQ ID NO: 427) |
| OGUL01000592_19 (SEQ ID NO: 209) | CTACTATACTGGTGCGATTTTGCACTAGTCTAAAAC (SEQ ID NO: 432) |
| OIGD01000177_59 (SEQ ID NO: 209) | CTACTATACTGGTGCGATTTTGCACTAGTCTAAAAC (SEQ ID NO: 492) |
| OIXV01006344_7 (SEQ ID NO: 209) | CTACTATACTGGTGCGATTTTGCACTAGTCTAAAAC (SEQ ID NO: 495) |
| PPYF01129432_15 (SEQ ID NO: 209) | CTACTATACTGGTGCGATTTTGCACTAGTCTAAAAC (SEQ ID NO: 531) |
| OAVJ01001264_7 (SEQ ID NO: 210) | CTACTACACTGGTGCAAATTTGCACTA (SEQ ID NO: 377) |
| OBAE01000973_3 (SEQ ID NO: 211) | GTGCGAATTTGCACTAGTCTAAAAC (SEQ ID NO: 378) |
| OBAR01000289_55 (SEQ ID NO: 212) | CTACTACACTAGTGCGAATTTGCACTAGTCTAAAAC (SEQ ID NO: 74) |
| OBA501000138_55 (SEQ ID NO: 212) | CTACTACACTAGTGCGAATTTGCACTAGTCTAAAAC (SEQ ID NO: 74) |
| OCHD01001741_1 (SEQ ID NO: 212) | CTACTACACTAGTGCGAATTTGCACTAGTCTAAAAC (SEQ ID NO: 74) |
| OCHK01000325_37 (SEQ ID NO: 212) | CTACTACACTAGTGCGAATTTGCACTAGTCTAAAAC (SEQ ID NO: 74) |
| OCH501000450_6 (SEQ ID NO: 212) | CTACTACACTAGTGCGAATTTGCACTAGTCTAAAAC (SEQ ID NO: 74) |
| OCQA01000142_55 (SEQ ID NO: 212) | CTACTACACTAGTGCGAATTTGCACTAGTCTAAAAC (SEQ ID NO: 74) |
| OBCV01000332_2 (SEQ ID NO: 213) | CTACTACACTGGTGCAAATTTGCACTAGTCTAAAAC (SEQ ID NO: 75) |
| OBDE01000870_1 (SEQ ID NO: 214) | CTACTACACTGGTGCGAATTTGCACTAG (SEQ ID NO: 381) |
| OBII01002626_5 (SEQ ID NO: 215) | CTACTACACTAGTGCGAATTTGCACTAGTCTAAAAC (SEQ ID NO: 74) |
| OBII01002626_3 (SEQ ID NO: 216) | CTACTACACTAGTGCGAATTTGCACTAGTCTAAAAC (SEQ ID NO: 74) |
| OBJF01000033_8 (SEQ ID NO: 217) | GATTGAAAGGATTGTAAATTTGCAAGGTCTTAAAAC (SEQ ID NO: 382) |
| OBJF01000033_8 (SEQ ID NO: 218) | GATTGAAAGGATTGTAAATTTGCAAGGTCTTAAAAC (SEQ ID NO: 383) |
| OJMK01000275_31 (SEQ ID NO: 218) | GATTGAAAGGATTGTAAATTTGCAAGGTCTTAAAAC (SEQ ID NO: 508) |
| OBKG01000025_26 (SEQ ID NO: 219) | CTACTACACTGGTGCGAATTTGCACTAGTCTAAAAC (SEQ ID NO: 62) |
| OBKR01000858_3 (SEQ ID NO: 220) | CTACTACACTGGTGCGATTTTGCACTAGTCTAAAACT (SEQ ID NO: 384) |
| OJM101000733_4 (SEQ ID NO: 220) | CTACTACACTGGTGCGATTTTGCACTAGTCTAAAACT (SEQ ID NO: 507) |
| OBVH01003037_1 (SEQ ID NO: 221) | GATTGAAAGGATTGTAAATTTACAAGGTCTTAAAAC (SEQ ID NO: 385) |
| OBVH01003037_2 (SEQ ID NO: 222) | GATTGAAAGGATTGTAAATTTACAAGGTCTTAAAAC (SEQ ID NO: 386) |
| OBVY01000267_8 (SEQ ID NO: 223) | CTACTACACTGGTGCGATTTTGCACTAGTCTAAAACT (SEQ ID NO: 387) |
| OG0001002653_3 (SEQ ID NO: 223) | CTACTACACTGGTGCGATTTTGCACTAGTCTAAAACT (SEQ ID NO: 403) |
| OBXZ01000094_20 (SEQ ID NO: 224) | GATTGAATGGATTGTAAATTT (SEQ ID NO: 388) |
| OBXZ01000094_20 (SEQ ID NO: 225) | GATTGAATGGATTGTAAATTT (SEQ ID NO: 389) |
| OCHB01002119_1 (SEQ ID NO: 226) | ACTGGTGCAAATTTGCACTAGTCTAAAAC (SEQ ID NO: 390) |
| OCHC01000012_250 (SEQ ID NO: 227) | TCTCTTGGCGGAAAGAAAACAGAAAGACGAAGAACAGGACAAATGGCTATC |
| | (SEQ ID NO: 391) |
| OCP501000464_4 (SEQ ID NO: 227) | GCTACTACACTGGTGCGAATTTGCACTAGTCTAAAAC (SEQ ID NO: 394) |
| OCHN01000290_35 (SEQ ID NO: 228) | GAACTACACCCGTGCAAAATTGCAGGGGTCTAAAAC (SEQ ID NO: 34) |
| OGP501000672_3 (SEQ ID NO: 229) | CTACTACACTAGTGCAAATTTGCACTAGTCTAAAAC (SEQ ID NO: 39) |
| OCPQ01000020_138 (SEQ ID NO: 229) | CTACTACACTGGTGCGAATTTGCACTAGTCTAAAAC (SEQ ID NO: 62) |
| OCPU01001206_17 (SEQ ID NO: 230) | GCTACTACACTGGTGCGAATTTGCACTAGTCTAAAAC (SEQ ID NO: 395) |
| OEHT01000244_15 (SEQ ID NO: 231) | CTACTACACTGGTGCGAATTTGCACTAGTCTAAAAC (SEQ ID NO: 62) |
| OGPU01000173_30 (SEQ ID NO: 231) | CTACTACACTGGTGCGAATTTGCACTAGTCTAAAAC (SEQ ID NO: 62) |
| OHHR01000227_3 (SEQ ID NO: 231) | CTACTACACTGGTGCGAATTTGCACTAGTCTAAAAC (SEQ ID NO: 62) |
| OJOL01000697_12 (SEQ ID NO: 231) | CTACTACACTGGTGCGAATTTGCACTAGTCTAAAAC (SEQ ID NO: 62) |
| OFMU01000310_31 (SEQ ID NO: 231) | CTACTACACTGGTGCGAATTTGCACTAG (SEQ ID NO: 398) |
| OGOI01001249_5 (SEQ ID NO: 231) | CTACTACACTGGTGCGAATTTGCACTAG (SEQ ID NO: 404) |
| OGQV01000794_21 (SEQ ID NO: 231) | CTACTACACTGGTGCGAATTTGCACTAGTCTAAAACT (SEQ ID NO: 419) |
| OGQZ01000194_33 (SEQ ID NO: 231) | CTACTACACTGGTGCGAATTTGCACTAGTCTAAAACT (SEQ ID NO: 422) |
| OHPC01000165_40 (SEQ ID NO: 231) | CTACTACACTGGTGCGAATTTGCACTAG (SEQ ID NO: 473) |
| OHUN01000170_40 (SEQ ID NO: 231) | CTACTACACTGGTGCGAATTTGCACTAG (SEQ ID NO: 486) |
| OJNT01000812_6 (SEQ ID NO: 231) | CTACTACACTGGTGCGAATTTGCACTAG (SEQ ID NO: 512) |
| OJOF01000269_30 (SEQ ID NO: 231) | CTACTACACTGGTGCGAATTTGCACTAG (SEQ ID NO: 513) |
| OKSV01000264_32 (SEQ ID NO: 231) | CTACTACACTGGTGCGAATTTGCACTAG (SEQ ID NO: 520) |
| OKVF01000105_32 (SEQ ID NO: 231) | CTACTACACTGGTGCGAATTTGCACTAG (SEQ ID NO: 525) |
| OEBA01002798_7 (SEQ ID NO: 231) | CTACTACACTGGTGCGAATTTGCACTAG (SEQ ID NO: 550) |
| OFMV01000268_25 (SEQ ID NO: 232) | GGAGGTGATAAAAATGGGAAA (SEQ ID NO: 399) |
| OGQU01002289_9 (SEQ ID NO: 232) | GGAGGTGATAAAAATGGGAAA (SEQ ID NO: 418) |
| OLGH01000826_1 (SEQ ID NO: 232) | GGAGGTGATAAAAATGGGAAA (SEQ ID NO: 527) |
| ODVS01001471_9 (SEQ ID NO: 232) | GGAGGTGATAAAAATGGGAAA (SEQ ID NO: 543) |
| OGCM01002738_3 (SEQ ID NO: 233) | CTACTATACTGGTGCGAATTTGCACTAGTCTAAAATG (SEQ ID NO: 401) |
| OGC001000353_15 (SEQ ID NO: 234) | ACTGGTGCAAATTTGCACTAGTCTAAAAC (SEQ ID NO: 402) |
| OGOK01000323_15 (SEQ ID NO: 235) | CTACTATACTGGTGCGATTTTGCACTAGTCTAAAAC (SEQ ID NO: 405) |
| OGOL01000786_27 (SEQ ID NO: 236) | GTGCGAATTTGCACTAGTCTAAAAC (SEQ ID NO: 406) |
| OGOO01001137_18 (SEQ ID NO: 237) | GAATTTGCACTAGTCTAAAAC (SEQ ID NO: 407) |
| OGOP01001824_10 (SEQ ID NO: 238) | GGAGGTGATAAAAATGGGAAAGACGATCCTTACGGCTATC (SEQ ID NO: |
| | 408) |
| OGRT01000617_3 (SEQ ID NO: 238) | GGAGGTGATAAAAATGGGAAAGACGATCCTTACGGCTATC (SEQ ID NO: |
| | 430) |
| OGPB01000314_7 (SEQ ID NO: 239) | CTACACTAGTGCGAATTTGCACTAGTCTAAAAC (SEQ ID NO: 411) |
| OGPJ01000449_26 (SEQ ID NO: 240) | CTGGTGCGAATTTGCACTAGTCTAAAAC (SEQ ID NO: 412) |
| OGPK01001709_2 (SEQ ID NO: 240) | CTGGTGCGAATTTGCACTAGTCTAAAAC (SEQ ID NO: 413) |
| OGP501000624_23 (SEQ ID NO: 241) | GAACTACACCCGTGCAAAATTGCAGGGGTCTAAAAC (SEQ ID NO: 34) |
| OGQH01000331_48 (SEQ ID NO: 242) | CCTACTACACTGGTGCGAATTTGCACTA (SEQ ID NO: 416) |
| OGQX01000605_8 (SEQ ID NO: 242) | TCTCTTGGCGGAAAGAAAACAGAAAGACGAAGAACAGGACAAATGGCTATC |
| | (SEQ ID NO: 421) |
| OGRG01000028_3 (SEQ ID NO: 242) | GCTACTACACTGGTGCGAATTTGCACTAGTCTAAAAC (SEQ ID NO: 426) |
| ODEE01001565_1 (SEQ ID NO: 242) | TCTCTTGGCGGAAAGAAAACAGAAAGACGAAGAACAGGACAAATGGCTATC |
| | (SEQ ID NO: 532) |
| ODIH01000145_73 (SEQ ID NO: 242) | GCTGAAAGAAAACAGAAAGACGAGGAGCAGGACAAATGGCTTTC (SEQ ID |
| | NO: 538) |
| OGQ001007270_2 (SEQ ID NO: 243) | CTACTACACTGGTGCGAATTTGCACTA (SEQ ID NO: 417) |
| OEFH01000394_40 (SEQ ID NO: 243) | CTACTACACTGGTGCGAATTTGCACTA (SEQ ID NO: 552) |
| OGQW01001429_6 (SEQ ID NO: 244) | CTACTACACTGGTGCGAATTTGCACTAG (SEQ ID NO: 420) |
| OGRA01000610_24 (SEQ ID NO: 245) | ACTGGTGCGATTTTGCACTAGTCTAAAAC (SEQ ID NO: 423) |
| OGRE01001635_6 (SEQ ID NO: 246) | GCTGAAAGAAAACAGAAAGACGAGGAGCAGGACAAATGGCTTTC (SEQID |
| | NO: 424) |
| OGRF01000967_2 (SEQ ID NO: 247) | GATTTTGCACTAGTCTAAAAC (SEQ ID NO: 425) |
| OGRN01001989_2 (SEQ ID NO: 248) | CTACTATACTGGTGCGATTTTGCACTAGTCTAAAAC (SEQ ID NO: 428) |
| OGRQ01003333_5 (SEQ ID NO: 249) | CTACTATACTGGTGCGATTTTGCACTAGTCTAAAAC (SEQ ID NO: 429) |
| OGRU01000829_2 (SEQ ID NO: 250) | CTACTACACTGGTGCGATTTTGCACTAGTCTAAAACT (SEQ ID NO: 431) |
| OGSD01001176_18 (SEQ ID NO: 251) | CTACTATACTGGTGCGAATTTGCACTAGTCTAAAAC (SEQ ID NO: 42) |
| OGWY01002732_3 (SEQ ID NO: 252) | GAACTACACCCGTGCAAAATTGCAGGGGTCTAAAAC (SEQ ID NO: 34) |
| OGXI01000433_6 (SEQ ID NO: 253) | GGAGGTGATAAAAATGGGAAA (SEQ ID NO: 433) |
| OGYU01002161_4 (SEQ ID NO: 253) | GGAGGTGATAAAAATGGGAAA (SEQ ID NO: 436) |
| OGG501001705_3 (SEQ ID NO: 253) | GGAGGTGATAAAAATGGGAAA (SEQ ID NO: 574) |
| OGXJ01002463_5 (SEQ ID NO: 254) | CTACTACACTGGTGCGAATTTG (SEQ ID NO: 434) |
| OGXL01002096_10 (SEQ ID NO: 255) | CTACTATACTGGTGCGAATTTGCACTAGTCTAAAAC (SEQ ID NO: 42) |
| OGYD01000683_23 (SEQ ID NO: 256) | GGAGGTGATAAAAATGGGAAA (SEQ ID NO: 435) |
| OGYL01002810_3 (SEQ ID NO: 257) | CTACTACACTGGTGCGAATTTGCACTAGTCTAAAAC (SEQ ID NO: 62) |
| OGYY01000371_37 (SEQ ID NO: 258) | TTTGCACTAGTCTAAAAC (SEQ ID NO: 437) |
| OHBM01000552_13 (SEQ ID NO: 258) | TTTTGCACTAGTCTAAAACTT (SEQ ID NO: 443) |
| OGGV01005531_2 (SEQ ID NO: 258) | TTTTGCACTAGTCTAAAACTT (SEQ ID NO: 575) |
| OGZC01000639_10 (SEQ ID NO: 259) | GTTTTAGTATCCACGATAAACGTGGATTGTAGT (SEQ ID NO: 438) |
| OHAI01000724_7 (SEQ ID NO: 260) | CTACTACACTGGTGCGAATTTGCACTAGTCTAAAAC (SEQ ID NO: 62) |
| OHAJ01000052_20 (SEQ ID NO: 261) | GATTGAAAGCTATGCGAATTTGCACAGTCTTAAAAC (SEQ ID NO: 439) |
| OGDS01000069_10 (SEQ ID NO: 261) | GATTGAAAGCTATGCGAATTTGCACAGTCTTAAAAC (SEQ ID NO: 572) |
| OHAN01001071_11 (SEQ ID NO: 262) | CTACTACACTAGTGCAAATTTGCGCTAGTCTAAAACT (SEQ ID NO: 440) |
| OHAR01000226_9 (SEQ ID NO: 263) | CTACTACACTAGTGCGAATTTGCACTA (SEQ ID NO: 441) |
| OHGN01001355_3 (SEQ ID NO: 263) | CTACTACACTAGTGCGAATTTGCACTA (SEQ ID NO: 454) |
| OHHD01000480_3 (SEQ ID NO: 263) | CTACTACACTAGTGCGAATTTGCACTA (SEQ ID NO: 456) |
| OHKC01000402_5 (SEQ ID NO: 263) | CTACTACACTAGTGCGAATTTGCACTA (SEQ ID NO: 460) |
| OHBL01000590_7 (SEQ ID NO: 264) | CTACTACACTGGTGCGATTTTGCACTAGTCTAAAA (SEQ ID NO: 442) |
| OHL001000586_3 (SEQ ID NO: 264) | CTACTACACTGGTGCGATTTTGCACTAGTCTAAAAC (SEQ ID NO: 463) |
| OHSZ01000559_4 (SEQ ID NO: 264) | CTACTACACTGGTGCGATTTTGCACTAGTCTAAAAC (SEQ ID NO: 482) |
| OHBP01000023_129 (SEQ ID NO: 265) | CTACTACACTAGTGCGAATTTGCACTAGTCTAAAAC (SEQ ID NO: 74) |
| OHDS01000019_133 (SEQ ID NO: 265) | CTACTACACTAGTGCGAATTTGCACTAGTCTAAAAC (SEQ ID NO: 74) |
| OHMH01000024_3 (SEQ ID NO: 265) | CTACTACACTAGTGCGAATTTGCACTAGTCTAAAAC (SEQ ID NO: 74) |
| OHBQ01000429_2 (SEQ ID NO: 266) | CTACTACACTGGTGCGAATTTGCACTAGTCTAAAAC (SEQ ID NO: 62) |
| OHEL01001488_6 (SEQ ID NO: 266) | CTACTACACTGGTGCGAATTTGCACTAGTCTAAAAC (SEQ ID NO: 62) |
| OHKH01000861_3 (SEQ ID NO: 266) | CTACTACACTGGTGCGAATTTGCACTAGTCTAAAAC (SEQ ID NO: 62) |
| OHBW01001448_1 (SEQ ID NO: 267) | ACTGGTGCGATTTTGCACTAGTCTAAAAC (SEQ ID NO: 444) |
| OHEG01001211_2 (SEQ ID NO: 267) | CTACTATACTGGTGCGATTTTGCACTAGTCTAAAAC (SEQ ID NO: 451) |
| OHSG01000119_6 (SEQ ID NO: 267) | CTACTATACTGGTGCGATTTTGCACTA (SEQ ID NO: 479) |
| OHSQ01001407_1 (SEQ ID NO: 267) | ACTGGTGCGATTTTGCACTAGTCTAAAAC (SEQ ID NO: 481) |
| OHJG01000198_33 (SEQ ID NO: 268) | CTACTATACTGGTGCGAATTTGCACTAGTCTAAAAT (SEQ ID NO: 59) |
| OHCE01000125_17 (SEQ ID NO: 268) | GCTGAAAGAAAACAGAAAGACGAGGAGCAGGACAAATGGCTTTC(SEQID |
| | NO: 445) |
| OHJJ01000127_35 (SEQ ID NO: 268) | GCTGAAAGAAAACAGAAAGACGAGGAGCAGGACAAATGGCTTTC(SEQID |
| | NO: 458) |
| OHRD01000126_17 (SEQ ID NO: 268) | TCTCTTGGCGGAAAGAAAACAGAAAGACGAAGAACAGGACAAATGGCTATC |
| | (SEQ ID NO: 477) |
| OHCH01000211_3 (SEQ ID NO: 269) | CTACTACACTAGTGCGAATTTGCACTAGTCTAAAAC (SEQ ID NO: 74) |
| OHPE01000834_1 (SEQ ID NO: 269) | CTACTACACTAGTGCGAATTTGCACTAGTCTAAAAC (SEQ ID NO: 74) |
| OHFX01001477_3 (SEQ ID NO: 269) | CTACACTGGTGCGAGTTTGCACTAGTCTAAAAC (SEQ ID NO: 453) |
| OHIJ01000315_7 (SEQ ID NO: 269) | CTACACTAGTGCGAATTTGCACTAGTCTAAAAC (SEQ ID NO: 457) |
| OHMQ01000465_4 (SEQ ID NO: 269) | CTACACTAGTGCGAATTTGCACTAGTCTAAAAC (SEQ ID NO: 467) |
| OHMW01000451_18 (SEQ ID NO: 269) | CTACACTGGTGCGAGTTTGCACTAGTCTAAAAC (SEQ ID NO: 468) |
| OHNF01001864_4 (SEQ ID NO: 269) | CTACACTGGTGCGAGTTTGCACTAGTCTAAAAC (SEQ ID NO: 469) |
| OHQE01002584_3 (SEQ ID NO: 269) | CTACACTGGTGCGAGTTTGCACTAGTCTAAAAC (SEQ ID NO: 476) |
| OKSK01000361_17 (SEQ ID NO: 269) | CTACACTGGTGCGAGTTTGCACTAGTCTAAAAC (SEQ ID NO: 519) |
| OKTU01000352_17 (SEQ ID NO: 269) | CTACACTGGTGCGAGTTTGCACTAGTCTAAAAC (SEQ ID NO: 523) |
| OHCP01000044_27 (SEQ ID NO: 270) | GTACTAAAGCCCGCTAGTATAGACGGGTTCTAAGAC (SEQ ID NO: 446) |
| OHSM01000196_10 (SEQ ID NO: 270) | GTACTAAAGCCCGCTAGTATAGACGGGTTCTAAGAC (SEQ ID NO: 480) |
| OKTR01000164_10 (SEQ ID NO: 270) | GTACTAAAGCCCGCTAGTATAGACGGGTTCTAAGAC (SEQ ID NO: 522) |
| OHCW01000317_3 (SEQ ID NO: 271) | GGTGCGATTTTGCACTAGTCTAAAAC (SEQ ID NO: 447) |
| OHDC01002972_3 (SEQ ID NO: 271) | GGTGCGATTTTGCACTAGTCTAAAAC (SEQ ID NO: 448) |
| OHKW01000215_41 (SEQ ID NO: 271) | GGAGGTGATAAAAATGGGAAA (SEQ ID NO: 461) |
| OHPP01000240_36 (SEQ ID NO: 271) | GGAGGTGATAAAAATGGGAAA (SEQ ID NO: 475) |
| OHRM01001189_3 (SEQ ID NO: 271) | GGTGCGATTTTGCACTAGTCTAAAAC (SEQ ID NO: 478) |
| OHTG01000221_40 (SEQ ID NO: 271) | GGAGGTGATAAAAATGGGAAA (SEQ ID NO: 483) |
| OHTH01000201_42 (SEQ ID NO: 271) | GGAGGTGATAAAAATGGGAAA (SEQ ID NO: 484) |
| OKTJ01001834_4 (SEQ ID NO: 271) | GGTGCGATTTTGCACTAGTCTAAAAC (SEQ ID NO: 521) |
| ODFV01004017_1 (SEQ ID NO: 271) | GGTGCGATTTTGCACTAGTCTAAAAC (SEQ ID NO: 533) |
| OHDP01000241_4 (SEQ ID NO: 272) | TGAACTACACCCGTGCAAAATTGCAGGGGTCTAAAAC (SEQ ID NO: 449) |
| OHFV01000201_5 (SEQ ID NO: 272) | TGAACTACACCCGTGCAAAATTGCAGGGGTCTAAAAC (SEQ ID NO: 452) |
| OHLY01001101_3 (SEQ ID NO: 272) | TGAACTACACCCGTGCAAAATTGCAGGGGTCTAAAAC (SEQ ID NO: 464) |
| OHPD01001131_4 (SEQ ID NO: 272) | TGAACTACACCCGTGCAAAATTGCAGGGGTCTAAAAC (SEQ ID NO: 474) |
| OHDT01000502_2 (SEQ ID NO: 273) | GCTGAAAGAAAACAGAAAGACGAGGAGCAGGACAAATGGCTTTC (SEQ ID |
| | NO: 450) |
| OHFA01000290_5 (SEQ ID NO: 274) | GAACTACACCCGTGCAAAATTGCAGGGGTCTAAAAC (SEQ ID NO: 34) |
| OHJZ01000157_5 (SEQ ID NO: 274) | GAACTACACCCGTGCAAAATTGCAGGGGTCTAAAAC (SEQ ID NO: 34) |
| OHST01000977_4 (SEQ ID NO: 274) | GAACTACACCCGTGCAAAATTGCAGGGGTCTAAAAC (SEQ ID NO: 34) |
| OKSP01001453_2 (SEQ ID NO: 274) | GAACTACACCCGTGCAAAATTGCAGGGGTCTAAAAC (SEQ ID NO: 34) |
| OHGX01000264_3 (SEQ ID NO: 275) | CTACTATACTGGTGCGAATTTGCACTAGTCTAAAATG (SEQ ID NO: 455) |
| OHME01000303_3 (SEQ ID NO: 275) | CTACTATACTGGTGCGAATTTGCACTAGTCTAAAATG (SEQ ID NO: 465) |
| OHNP01000278_34 (SEQ ID NO: 275) | CTACTATACTGGTGCGAATTTGCACTAGTCTAAAATG (SEQ ID NO: 470) |
| OH0101000307_2 (SEQ ID NO: 275) | CTACTATACTGGTGCGAATTTGCACTAGTCTAAAATG (SEQ ID NO: 471) |
| OHIB01002708_3 (SEQ ID NO: 276) | CTACTACACTGGTGCGAATTTGCACTAGTCTAAAAC (SEQ ID NO: 62) |
| OHJK01001285_9 (SEQ ID NO: 277) | GAACTACACCCGTGCAAAATTGCAGGGGTCTAAAAC (SEQ ID NO: 34) |
| OHS101000544_10 (SEQ ID NO: 277) | GAACTACACCCGTGCAAAATTGCAGGGGTCTAAAAC (SEQ ID NO: 34) |
| OKSN01001169_3 (SEQ ID NO: 277) | GAACTACACCCGTGCAAAATTGCAGGGGTCTAAAAC (SEQ ID NO: 34) |
| OHJ501001864_3 (SEQ ID NO: 278) | CTACTACACTGGTGCGATTTTGCACTAGTCTAAAAC (SEQ ID NO: 459) |
| OHLH01003112_3 (SEQ ID NO: 278) | CTACTACACTGGTGCGATTTTGCACTAGTCTAAAA (SEQ ID NO: 462) |
| OHJT01001977_4 (SEQ ID NO: 279) | GAACTACACCCGTGCAAAATTGCAGGGGTCTAAAAC (SEQ ID NO: 34) |
| OHPW01002065_2 (SEQ ID NO: 279) | GAACTACACCCGTGCAAAATTGCAGGGGTCTAAAAC (SEQ ID NO: 34) |
| OHMF01000395_24 (SEQ ID NO: 280) | CTACTACACTGGTGCGAATTTGCACTAGTCTAAAACT (SEQ ID NO: 466) |
| OHOK01001322_2 (SEQ ID NO: 280) | GTGCGAATTTGCACTAGTCTAAAAC (SEQ ID NO: 472) |
| OHUA01000395_26 (SEQ ID NO: 280) | GTGCGAATTTGCACTAGTCTAAAAC (SEQ ID NO: 485) |
| OHUY01000263_2 (SEQ ID NO: 281) | CTACTACACTAGTGCGAATTTGCACTAGTCTAAAAC (SEQ ID NO: 74) |
| OHVU01001109_1 (SEQ ID NO: 281) | CTACTACACTAGTGCGAATTTGCACTAGTCTAAAAC (SEQ ID NO: 74) |
| OHXZ01000057_25 (SEQ ID NO: 281) | CTACTACACTAGTGCGAATTTGCACTAGTCTAAAAC (SEQ ID NO: 74) |
| OHYU01000376_4 (SEQ ID NO: 281) | CTACTACACTAGTGCGAATTTGCACTAGTCTAAAAC (SEQ ID NO: 74) |
| OICI01000194_18 (SEQ ID NO: 281) | CTACTACACTAGTGCGAATTTGCACTAGTCTAAAAC (SEQ ID NO: 74) |
| OIDC01000397_3 (SEQ ID NO: 281) | CTACTACACTAGTGCGAATTTGCACTAGTCTAAAAC (SEQ ID NO: 74) |
| OIDU01000174_25 (SEQ ID NO: 281) | CTACTACACTAGTGCGAATTTGCACTAGTCTAAAAC (SEQ ID NO: 74) |
| OKUL01000400_17 (SEQ ID NO: 281) | CTACTACACTAGTGCGAATTTGCACTAGTCTAAAAC (SEQ ID NO: 74) |
| OKUR01000327_17 (SEQ ID NO: 281) | CTACTACACTAGTGCGAATTTGCACTAGTCTAAAAC (SEQ ID NO: 74) |
| OKVB01000375_17 (SEQ ID NO: 281) | CTACTACACTAGTGCGAATTTGCACTAGTCTAAAAC (SEQ ID NO: 74) |
| OKVC01000355_17 (SEQ ID NO: 281) | GGAGGTGATAAAAATGGGAAA (SEQ ID NO: 524) |
| OIBN01003740_1 (SEQ ID NO: 282) | CTACTACACTGGTGCAAATTAGCACTAGTCTAAAAC (SEQ ID NO: 77) |
| OIEE01000042_11 (SEQ ID NO: 283) | CTACTACACTGGTGCGATTTTGCACTAGTCTAAAACT (SEQ ID NO: 489) |
| OIEL01000292_3 (SEQ ID NO: 284) | CTACTATACTGGTGCGAATTTGCACTAGTCTAAAATG (SEQ ID NO: 490) |
| OJMG01000332_24 (SEQ ID NO: 284) | GCTGAAAGAAAACAGAAAGACGAGGAGCAGGACAAATGGCTTTC(SEQID |
| | NO: 506) |
| OIEN01002196_3 (SEQ ID NO: 285) | GCCCCTTGACCTTACGAAATGGTAAGGTTCCAAAAC (SEQ ID NO: 491) |
| OIXA01002812_3 (SEQ ID NO: 286) | CTACTACACTGGTGCGAATTTGCACTAGTCTAAAAC (SEQ ID NO: 62) |
| 01XU01000818_5 (SEQ ID NO: 287) | GATTGAAAGGATTGTAAATTT (SEQ ID NO: 493) |
| 01XU01000818_6 (SEQ ID NO: 288) | GATTGAAAGGATTGTAAATTT (SEQ ID NO: 494) |
| OIYU01000175_4 (SEQ ID NO: 289) | CTACTATACTGGTGCGAATTTGCACTAGTCTAAAATG (SEQ ID NO: 496) |
| OIZA01000315_9 (SEQ ID NO: 290) | GATTGAAAGGTTTGTAAATTTACAAGGTCTTAAAAC (SEQ ID NO: 497) |
| OIZ101000180_12 (SEQ ID NO: 291) | GATTGAAAGGATTGTAAATTTACAAGGTCTTAAAACA (SEQ ID NO: 500) |
| OIZ101000180_12 (SEQ ID NO: 292) | GATTGAAAGGATTGTAAATTTACAAGGTCTTAAAACA (SEQ ID NO: 501) |
| OIZU01000200_48 (SEQ ID NO: 293) | GAAAGAAAACAAAAAGACGAGAACAGGACAAATGGCTTTCTGAGCAGGCT |
| | (SEQ ID NO: 502) |
| OIZW01000344_20 (SEQ ID NO: 294) | GCTACTATACTGGTGCGAATTTGCACTAGTCTAAAAC (SEQ ID NO: 503) |
| OIZX01000427_25 (SEQ ID NO: 295) | ACTATAGCCCTGCCGGAAA (SEQ ID NO: 504) |
| OIZX01000427_26 (SEQ ID NO: 296) | ACTATAGCCCTGCCGGAAA (SEQ ID NO: 505) |
| OJMJ01002228_5 (SEQ ID NO: 297) | CTACTACACTAGTGCGAATTTGCACTAGTCTAAAAC (SEQ ID NO: 74) |
| OJMM01002900_7 (SEQ ID NO: 298) | GTACAATAGCCCTCTCGTAGTTGAGGGCTCTGAGAC (SEQ ID NO: 509) |
| OJMM01002900_7 (SEQ ID NO: 299) | GTACAATAGCCCTCTCGTAGTTGAGGGCTCTGAGAC (SEQ ID NO: 510) |
| OJMN01000417_22 (SEQ ID NO: 300) | CTACTATACTGGTGCGAATTTGCACTAGTCTAAAAC (SEQ ID NO: 42) |
| OJNI01000536_4 (SEQ ID NO: 300) | CTACTATACTGGTGCGAATTTGCACTAGTCTAAAAC (SEQ ID NO: 42) |
| OJNR01001167_9 (SEQ ID NO: 301) | GAACTACACCCGTGCAAAATTGCAGGGGTCTAAAAC (SEQ ID NO: 34) |
| OJOP01001093_3 (SEQ ID NO: 301) | GAACTACACCCGTGCAAAATTGCAGGGGTCTAAAAC (SEQ ID NO: 34) |
| OJNS01001527_9 (SEQ ID NO: 301) | GAACTACACCCGTGCAAAATTGCAGG (SEQ ID NO: 511) |
| OJPG01000139_73 (SEQ ID NO: 302) | CTACTACACTGGTGCGAATTTGCACTAGTCTAAAAC (SEQ ID NO: 62) |
| OJPS01000131_3 (SEQ ID NO: 302) | CTACTACACTGGTGCGAATTTGCACTAGTCTAAAAC (SEQ ID NO: 62) |
| OJQH01000635_3 (SEQ ID NO: 302) | CTACTACACTGGTGCGAATTTGCACTAGTCTAAAAC (SEQ ID NO: 62) |
| OJRP01000045_31 (SEQ ID NO: 302) | CTACTACACTGGTGCGAATTTGCACTAGTCTAAAAC (SEQ ID NO: 62) |
| OJPX01000614_4 (SEQ ID NO: 303) | GTGCGATTTTGCACTAGTCTAAAAC (SEQ ID NO: 515) |
| OJRG01001951_4 (SEQ ID NO: 303) | GTGCGATTTTGCACTAGTCTAAAAC (SEQ ID NO: 516) |
| OGNV01000836_4 (SEQ ID NO: 304) | CTACTACACTGGTGCGAATTTGCACTAGTCTAAAAC (SEQ ID NO: 62) |
| OKRZ01002949_5 (SEQ ID NO: 304) | CTACTACACTGGTGCGAATTTGCACTAGTCTAAAAC (SEQ ID NO: 62) |
| OKSB01002689_10 (SEQ ID NO: 305) | CTACTACACTAGTGCGAATTTGCACTAGTCTAAAAC (SEQ ID NO: 74) |
| OKSC01004083_2 (SEQ ID NO: 306) | GCACTACACCCCCCTGAAACATGAG (SEQ ID NO: 517) |
| OKSD01002505_11 (SEQ ID NO: 307) | CTACTACACTAGTGCGAATTTGCACTA (SEQ ID NO: 518) |
| OLGN01000304_32 (SEQ ID NO: 308) | GAAAGAAAACAAAAAGACGAGAACAGGACAAATGGCTTTCTGAGCAGGCT |
| | (SEQ ID NO: 528) |
| OLHE01000257_41 (SEQ ID NO: 309) | CTACTACACTGGTGCGAATTTGCACTAGTCTAAAAC (SEQ ID NO: 62) |
| PPYE01106492_34 (SEQ ID NO: 310) | GACGGGAGGTGATGAAAATG (SEQ ID NO: 529) |
| PPYE01385196_3 (SEQ ID NO: 311) | CTACTACACTGGTGCGAATTTGCACTAGTCTAAAAC (SEQ ID NO: 62) |
| PPYE01512733_3 (SEQ ID NO: 312) | CTACTATACTGGTGCGAATTTGCACTAGTCTAAAATG (SEQ ID NO: 530) |
| PPYF01670242_39 (SEQ ID NO: 313) | CTACTACACTGGTGCGAATTTGCACTAGTCTAAAAC (SEQ ID NO: 62) |
| ODFW01000112_43 (SEQ ID NO: 314) | GGAGGTGATAAAAATGGGAAA (SEQ ID NO: 534) |
| ODTU01003882_3 (SEQ ID NO: 314) | GGAGGTGATAAAAATGGGAAA (SEQ ID NO: 541) |
| ODGN01000188_50 (SEQ ID NO: 315) | CTACTACACTGGTGCGAATTTGCACTAGTCTAAAAC (SEQ ID NO: 62) |
| ODHH01000275_14 (SEQ ID NO: 316) | GCTGAAAGAAAACAGAAAGACGAGGAGCAGGACAAATGGCTTTC (SEQ ID |
| | NO: 535) |
| ODYJ01000298_33 (SEQ ID NO: 316) | GCTGAAAGAAAACAGAAAGACGAGGAGCAGGACAAATGGCTTTC (SEQ ID |
| | NO: 549) |
| ODHPO1001712_3 (SEQ ID NO: 317) | CTACTACACTGGTGCGAATTTGCACTAGTCTAAAAC (SEQ ID NO: 62) |
| ODHV01000466_16 (SEQ ID NO: 318) | CTAGTGCAAATTTGCACTAGTCTAAAACG (SEQ ID NO: 536) |
| ODXE01000717_15 (SEQ ID NO: 318) | CTAGTGCAAATTTGCACTAGTCTAAAACG (SEQ ID NO: 545) |
| ODJZ01000182_13 (SEQ ID NO: 319) | CTACTACACTGGTGCGAATTTGCACTA (SEQ ID NO: 539) |
| ODLN01002572_7 (SEQ ID NO: 320) | CTACTACACTGGTGCGAATTTGCACTAGTCTAAAAC (SEQ ID NO: 62) |
| ODQJ01000729_25 (SEQ ID NO: 321) | CTACTATACTGGTGCGATTTTGCACTAGTCTAAAAC (SEQ ID NO: 540) |
| ODUN01000242_23 (SEQ ID NO: 322) | CTACTATACTGGTGCGAATTTGCACTAGTCTAAAAC (SEQ ID NO: 42) |
| ODWX01000843_3 (SEQ ID NO: 322) | CTACTATACTGGTGCGAATTTGCACTAGTCTAAAAC (SEQ ID NO: 42) |
| ODVQ01003982_3 (SEQ ID NO: 323) | CCTACTACACTAGTGCGAATTTGCACTAGTCTAAAACT (SEQ ID NO: |
| | 542) |
| ODVR01002077_3 (SEQ ID NO: 324) | CTACTACACTGGTGCGAATTTGCACTAGTCTAAAAC (SEQ ID NO: 62) |
| ODXC01000747_3 (SEQ ID NO: 325) | CTACTACACTGGTGCGAATTTGCACTA (SEQ ID NO: 544) |
| OEEK01000163_43 (SEQ ID NO: 325) | TCTCTTGGCGGAAAGAAAACAGAAAGACGAAGAACAGGACAAATGGCTATC |
| | (SEQ ID NO: 551) |
| ODXO01005124_2 (SEQ ID NO: 326) | GTGCGAATTTGCACTAGTCTAAAAC (SEQ ID NO: 546) |
| OEFW01000634_7 (SEQ ID NO: 326) | GTGCGAATTTGCACTAGTCTAAAAC (SEQ ID NO: 553) |
| ODYC01000377_16 (SEQ ID NO: 327) | GGAGGTGATAAAAATGGGAAA (SEQ ID NO: 548) |
| OEJW01000623_11 (SEQ ID NO: 328) | CTACTACACTGGTGCGAATTTGCACTAGTCTAAAAC (SEQ ID NO: 62) |
| 33000193761Ga0187899_10021543_4 | TGAACGATAGCCTGCTGAAATATGCAGGTTCTAAGAC (SEQ ID NO: 570) |
| (SEQ ID NO: 329) | |
| OGCZ01001955_1 (SEQ ID NO: 330) | CTACTATACTGGTGCGAATTTGCACTAGTCTAAAATG (SEQ ID NO: 571) |
| OGDY01002059_17 (SEQ ID NO: 331) | GAACTACACCCGTGCAAAAATGCAGGGGTCTAAAAC (SEQ ID NO: 43) |
| OGEU01000713_24 (SEQ ID NO: 332) | CTACTACACTGGTGCGAATTTGCACTAGTCTAAAAC (SEQ ID NO: 62) |
| OGFM01002125_3 (SEQ ID NO: 333) | GACAGGAGGTGATAAAAATG (SEQ ID NO: 573) |
| OGHW01002048_1 (SEQ ID NO: 334) | CTACTACACTGGTGCAAATTTGCACTAGTCTAAAAC (SEQ ID NO: 75) |
| OGIE01002059_21 (SEQ ID NO: 335) | CTACTACACTAGTGCGAATTTGCACTAGTCTAAAACC (SEQ ID NO: 576) |
| OGII01000819_21 (SEQ ID NO: 335) | CTACTACACTAGTGCGAATTTGCACTAGTCTAAAACC (SEQ ID NO: 577) |
| OGJI01000038_151 (SEQ ID NO: 336) | CTACTACACTGGTGCGAATTTGCACTAGTCTAAAACTCA (SEQ ID NO: |
| | 578) |
| OGKE01000029_151 (SEQ ID NO: 336) | CTACTACACTGGTGCGAATTTGCACTAGTCTAAAACTCA (SEQ ID NO: |
| | 581) |
| OGKG01000020_152 (SEQ ID NO: 336) | CTACTACACTGGTGCGAATTTGCACTAGTCTAAAACTCA (SEQ ID NO: |
| | 582) |
| OGJK01007642_2 (SEQ ID NO: 337) | GTGCAAATTTGCACTAGTCTAAAAC (SEQ ID NO: 579) |
| OGJY01000516_18 (SEQ ID NO: 338) | CTACTACACTGGTGCGAATTTGCACTAGTCTAAAAC (SEQ ID NO: 62) |
| OGKA01000617_2 (SEQ ID NO: 339) | CTACTACACTGGTGCGAATTTGCACTAG (SEQ ID NO: 580) |
| OGKG01002483_14 (SEQ ID NO: 340) | GAACTACACCCGTGCAAAATTGCAGGGGTCTAAAAC (SEQ ID NO: 34) |
| OGKW01000585_4 (SEQ ID NO: 341) | ACTGGTGCGAATTTGCACTGGTCTAAAAC (SEQ ID NO: 583) |
| OGLJ01000192_54 (SEQ ID NO: 342) | CTACTACACTGGTGCAAATTTGCACTAGTCTAAAAC (SEQ ID NO: 75) |
| OGLM01001314_21 (SEQ ID NO: 343) | TGAACTACACCCGTGCAAAATTGCAGGGGTCTAAAAC (SEQ ID NO: 584) |
| OGMO01000062_69 (SEQ ID NO: 344) | CTACTACACTGGTGCAAATTTGCACTAGTCTAAAAC (SEQ ID NO: 75) |
| OGMP01001167_15 (SEQ ID NO: 345) | CTACTACACTAGTGCGAATTTGCACTAGTCTAAAAC (SEQ ID NO: 74) |
| OGUJ01000114_43 (SEQ ID NO: 346) | GATTGAAAGGATTGTAAATTTACAAGGTCTTAAAAC (SEQ ID NO: 585) |
| OGUJ01000114_45 (SEQ ID NO: 347) | GATTGAAAGGATTGTAAATTTACAAGGTCTTAAAAC (SEQ ID NO: 586) |
| OJKY01000879_3 (SEQ ID NO: 348) | GTACTAAAGCCCGCTAGTATAGACGGGTTCTAAGAC (SEQ ID NO: 587) |
| OLJF01000187_58 (SEQ ID NO: 349) | CTACTACACTGGTGCGATTTTGCACTAGTCTAAAACT (SEQ ID NO: 588) |
| OMWO01000091_3 (SEQ ID NO: 350) | GATTGAAAGCTATGCGAATTTGCACAGTCTTAAAAC (SEQ ID NO: 589) |

TABLE 4
Amino Acid Sequences of Cas13d Accessory Proteins WYLI

>SCH71532.1
[Ruminococcus sp. CAG:57]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNTVNEFISPPIQVKYYFNKFGKDGVILSPSDSFEEMRTLYVEGAEAYNREVEM (SEQ ID NO: 78)
>SCJ27525.1
[human gut metagenome]
MLILPSTFLPKRDKNVPYIAEVQSIPLSPSAYSVIIKDKSIFETSLSPNGSVSMSSFLTSIFDSAYIASLKYKSEKYNGIPLLN
AFVKWQIEEINDGLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 79)
>WP_041337479.1
[Ruminococcus bicirculans]
MSMTPSTFLPKREDGVPYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 80)
>LARF01000048_7
[Ruminococcus sp. N15.MGS-57]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 81)
>CDYU01004315_3
[gut metagenome]
MSMTPSTFLPKRDKNATYIAEVQSIPLSPSAYSVIIKDKSIFETSLSPNGSVSMSSFLTSIFDSAYIASLKYKSEKYNGIPLLN
AFVKWQIEEIDDGLDDKSKEIIKSYLISKLSAKYEKTKTENAVRVRLSICRDLYDTLSSDDLYYENKVYSSTLRRFLKAVYEDY
ALLSDCERERLIFADNIIKINEIIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKP
NTVNEFISPPIQVKYYFNKFGKDGVILSPSDSFEEMRTLYVEGAEAYNREVEM (SEQ ID NO: 82)
>CDYX01024884_5
[gut metagenome]
MFIPPSTFLPKREGGVPYIAEVQSIPLSPSAYSVIIKDKSIFETSLSPNGSVSMSSFLTSIFDSAYIASLKYKTDDNYKYIGIP
LLNAFIKWQIEEIDDGLDDKSKEIIKSYLISKFSAKYEKTKTENAVRVRLSICRDLYDTLSSDDLYYENKVYSSTLRRFLKAVY
EDYALLSDCERERLIFADNIIKINEVIKQNGNRYYSFIYAYSNMYSREKRRIRLIPYRIISDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 83)
>OGPQ01001037_4
[human gut metagenome]
MSMTPSTFLPKRDTNIPYIAEVQSIPLSPSAYAVIVKDKSIFETSLFPNGGSVSMSSFLTRIFDSAYIASLKYKSEEYNGIPLL
NAFVQWQIEEIDDSLDDKSKEIIKSYLISKLSAKYEKTKTENAVRVRLSICRDLYDTLSSDDLYYENKVYSSTLRRFLKAVYED
YALLSDCERERLIFADNIIKINEVIKQNGSRYYSFIYAYSNMYSREKRRIRLIPYRIVSDEYKMYNYLVCLSDEKSAGKEFKAD
SYRISRLSGLSIAEKLSQKEYSSVTEYERLKEGHVKSVKHLLSDPRFGSDESDISKVYLTEKGVEMFGKILYQRPILKGNEKPK
PNAVNEFISPPIQVKYYFNKFGKDGVILSPSDSFEEMRTLYVEGAEAYNREVEM (SEQ ID NO: 84)
>ODVQ01003982_4
[human metagenome]
MLIPPSTFLPKREGGVPYIAEVQSIPLSPSAYSVIIKDKSIFETSLFPNGSVSMSSFLTSIFDSAYIASLKYKSDDNYKYIGIP
LLNAFVKWQIEEIDDGLDDKSKEIIKSYLISKLSAKYKKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 85)
>33000072991Ga0104319_1000623_28
[human-digestive system-homo sapiens]
MLIPPSTFLPKRKDGVPYIAEVQSIPLSPSAYAVIVKDKSIFETSLSPNSSVSMSSFLTRIFDSAYRASLKYKSEEYNGIPLLN
AFVQWQIEEIDGSLDDKSKEIIRSYLISKLSAKYKKTKTENAVRVRLSICRDLYDTLSRVDLCYENKVYGSTLRRFLKAVYEDY
ALLSDCERERLIFADNIIKINEVIKQNSNRYDNFIYAYSSMYSREKCRIRLIPYRIVSDEYKMYNYLVCLSDEKSVGKEFKADS
YRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 86)
>CDY501033339_20
[gut metagenome]
MGTENSSNEYQEARQHLSLSDAAWAVLQDDRQDFGGGRSWAGILNYVFAEYRDKADASISVAVERRRAQYEEKLVGVAAPAVRK
AVLEALLADYTEELIKKAAQNGATPPDKESFKFRLDRDNYAFREQWLDSPDAQYYGGRFSRYLRAVLEEYAAKTVYQREAIYFD
PQMRLIQASAANGELLRIRLKKGSEFEVRPYGVLGDRQETYHYLVGLSRPDGTREPEKASSFRLSNIVKLEVSFRRSGRLTEKE
RTDIESSIRGKGVQFLVQQRETIRIRLTEDGRQNYGRQLHLRPAARERAEVDDGLYRWEYTFYCTEFQAKAYFLKFCGDAKVVE
PQSLRETFAQEYRSGLRACGEEP (SEQ ID NO: 87)
>CDTW01032418_59
[gut metagenome]
MGTENSSNEYQEARQHLSLSDAAWAVLQDDRRDFGGGRSWAGILNYVFTMYRDKADASVSVAVSRRREQLEEQLGGVVSPAARD
AVLDRLMEVYAGELAEKAMSDGAVAQQKEVFKFRLDRDNYAFREQWLDSPDAARYYGNRFSRYLRAVLEEYAAKTVYQREAIYF
DPQMRLIRAAAANGELLRIRMKTGSSFEVRPYGVLGDRQETYHYLVGLSRPDGTRGPEKEFNFRLSKIIKLDVSFRRSGRLTEK
ERTDIESSIRGKGVQFLAQQRETIRIRLTEEGRRDYGSQMHLRPPAQTRTAVDDGAYRWEYTFFCTEFQARAYFLKFCGEAKVV
EPQSLRDTLAQEYRSGLRACGEEP (SEQ ID NO: 88)
>OATA01000148_62
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIKEINDGLDDKSKEIIKSYLISKLSAKYEKTKTENAVRVRLSICRDLYDTLSSDDLYYENKVYSSTLRRFLKAVY
EVYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 590)
>OAVJ01001264_6
[human gut metagenome]
MSMTPSTFLPKREGGVPYIAEVQSIPLSPSAYSVIIKDKSIFETSLSPNGSVSMSSFLTSIFDSAYIASLKNKSDDNYKYNGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 591)
>OBAE01000973_4
[human gut metagenome]
MSMTPSTFLPKRDKNVPYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 592)
>OBAQ01000162_28
[human gut metagenome]
MSMTPSTFLPKREDGVPYIAEVQSIPLSPSAYSVIIRDKSIFETSLSPNGSVSMSSFLTSIFDSAYIASLKYKSDDNYKYIGIP
LLNAFVEWQIKEINDGLDDKSKEIIKSYLISKLSAKYEKTKTENAVRVRLSICRDLYDTLSSDDLYYENKVYSSTLRRFLKAVY
EVYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 593)
>OBA501000138_57
[human gut metagenome]
MSMTPSTFLPKREGSVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMHSREKRRIRLIPYRIVSDEYKMYNYLVCLSDEKGAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 594)
>OBCV01000332_3
[human gut metagenome]
MSMTPSTFLPKREDGVPYIAEVQSIPLSPTAYSVIVRDKSIFETSLSPNGGSVSMSSFLTRIFDSAYRASLKYKSEEYNGIPLL
NAFVQWQIEEIDGSLDDKSKEIIKSYLISKLSAKYKKTKTENAVRVRLSICRDLYDTLSRDDLCYENKVYGSTLRRFLKAVYED
YALLSDCERERLIFADNIIKINEVIKQNSNRYDNFIYAYSNMYSREKRRIRLIPYRIVSDEYKMYNYLICLSDEKSADKEFKAD
SYRISRLSGLSIAEKLSQKEYSSVTEYERLKEDHVKSVKHLLNDPRFGSDESDISKVYLTEKGVEMFGKILYQRPILKGNEKPK
PNAVNEFISPPIQVKYYFNKFGKDGVILSPSDSFEEMRTLYVEGAEAYNRVVEM (SEQ ID NO: 595)
>OBKG01000025_25
[human gut metagenome]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 596)
>OBKR01000858_4
[human gut metagenome]
MSMTPSTFLPKREGGVPYIAEVQSIPLSPSAYSVIIKDKSIFETSLSPNGSVSMSSFLTSIFDSSYIASLKYKSDDNYKYIGIP
LLNAFVKWQIEEIEDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYFSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 597)
>OBVY01000267_8
[human gut metagenome]
MSMTPSTFLPKREGGVPYIAEVQSIPLSPSAYSVIIKDKSIFETSLSPNGSVSMSSFLTSIFDSAYIASLKYKSGDNYKYIGIP
LLNAFVKWQIEEIDDGLDDKSKKIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 598)
>OCHC01000012_251
[human gut metagenome]
MSMTPSTFLPKREDGVPYIAEVQSIPLSPSAYSVIIKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 599)
>OCHE01000387_8
[human gut metagenome]
MLIPPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 600)
>OCPQ01000020_137
[human gut metagenome]
MSMTPSTFLPKRDTNIPYIAEVQSIPLSPSAYAVIVKDKSIFETSLFPNGGSVSMSSFLTRIFDSAYIASLKYKSEEYNGIPLL
NAFVQWQIEEIDDSLDDKSKEIIKSYLISKLSAKYEKTKTENAVRVRLSICRDLYDTLSSDDLYYENKVYSSTLRRFLKAVYED
YALLSDCERERLIFADNIIKINEVIKQNGSRYYSFIYAYSNMYSREKRRIRLIPYRIVSDEYKMYNYLVCLSDEKSAGKEFKAD
SYRISRLSGLSIAEKLSQKEYSSVTEYERLKEVHVKSVKHLLSDPRFGSDESDISKVYLTEKGVEMFGKILYQRPILKGNEKPK
PNTVNEFISPPIQVKYYFNKFGKDGVILSPSDSFEEMRTLYVEGAEAYNREVEK (SEQ ID NO: 601)
>OFMU01000310_30
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIKEINDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 602)
>OFMV01000268_23
[human gut metagenome]
MLIPPSTFLPKREDGVPYIAEVQSIPLSPSAYSVIIKDKSIFETSLSPNGSVSMSSFLTSIFDSAYIASLKYKSDDNYKYNGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 603)
>OGC001000353_16
[human gut metagenome]
MSMTPSTFLPKREDGVPYIAEVQSIPLSPTAYSVIVRDKSIFETSLSPNGGSVSMSSFLTRIFDSAYRASLKYKSEEYNGIPLL
NAFVQWQIEEIDGSLDDKSKEIIKSYLISKLSAKYKKTKTENAVRVRLSICRDLYDTLSRDDLCYENKVYGSTLRRFLKAVYED
YALLSDCERERLIFADNIIKINEVIKQNSNRYDNFIYAYSNMYSREKRRIRLIPYRIVSDEYKMYNYLICLSDEKSADKEFKAD
SYRISRLSGLSIAEKLSQKEYSSVTEYERLKEDHVKSVKHLLNDPRFGSDESDISKVYLTEKGVEMFGKILYQRPILKGNEKPK
PNAVNEFISPPIQVKYYFNKFGKDGVILSPSDSFEEMRTLYVEGAEAYNRVVEI (SEQ ID NO: 604)
>OGOP01001824_8
[human gut metagenome]
MSMTPSTFLPKREDGVPYIAEVQSIPLSPSAYSVIIRDKSIFETSLSPNGSVSMSSFLTSIFDSAYIASLKYKSDDNYKYIGIP
LLNAFVEWQIKEINDGLDDKSKEIIKSYLISKLSAKYEKTKTENAVRVRLSICRDLYDTLSSDDLYYENKVYSSTLRRFLKAVY
EVYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 605)
>OGPB01000314_5
[human gut metagenome]
MSMTPSTFLPKRDKNATYIAEVQSIPLSPSTYSVIIKDKSIFETSLSPNGSVSMSSFLTSIFDSAYIASLKYKSDDNYKYIGIP
LLNAFVEWQIEEIDDGLDDKSKEIIKSYLISKLSAKYKKTKTENAVRVRLSICRGLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFKEMRTLYVEGAEAYNREVEM (SEQ ID NO: 606)
>OGPJ01000449_25
[human gut metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRKFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKDGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 607)
>OGPU01000173_31
[human gut metagenome]
MSMTPSTFLPKREGGVPYIAEVQSIPLSPSAYSVIIKDKSIFETSLSPNGSVSMSSFLTSIFDSAYIASLKYKSGDNYKYIGIP
LLNAFVKWQIEEIDDGLDDKSKKIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 608)
>OGPY01000296_5
[human gut metagenome]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNTVNEFISPPIQVKYYFNKFGKDGVILSPSDSFEEMRTLYVEGAEAYNREVEI (SEQ ID NO: 609)
>OGQH01000331_47
[human gut metagenome]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSASKEFK
ADSCRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 610)
>OGQ001007270_1
[human gut metagenome]
MSMTPSTFLPKREGGVPYIAEVQSIPLSPSAYSVIIKDKSIFETSLSPNGSVSMSSFLTRIFDSAYIASLKYKSEEYNGIPLLN
AFVQWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEVHVKSVKHLLSDPRFGSDESDISKVYLTEKGVEMFGKILYQRPILKGNEKPKP
NTVNEFISPPIQVKYYFNKFGKDGVILSPSDSFEEMRTLYVEGAEAYNREVEK (SEQ ID NO: 611)
>OGRA01000610_25
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNRVVEM (SEQ ID NO: 612)
>OG5D01001176_17
[human gut metagenome]
MSMTPSTFLPKREDGVPYIAEVQSIPLSPSAYSVIIKDKSIFETSLSPNGSVSMSSFLTSIFDSAYIASLKYKSNDNYKYIGIP
LLNAFVQWQIEEIDDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKESHVKSVKHLLSDPRFGSDESDISKVYLTEKGVEMFGKILYQRPILKGNEK
PKPNAVNEFISPPIQVKYYFNKFGKDGVIISPSDSFEEMRTLYVEGAEAYNREVEM (SEQ ID NO: 613)
>OGXI01000433_8
[human gut metagenome]
MLIPPSTFLPKREDGVPYIAEVQSIPLSPSAYSVIIRDKSIFETSLSPNGSVSMSSFLTSIFDSAYIASLKYKSDDNYKYIGIP
LLNAFVEWQIKEINDGLDDKSKEIIKSYLISKLSAKYEKTKTENAVRVRLSICRDLYDTLSSDDLYYENKVYSSTLRRFLKAVY
EVYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 614)
>OGXJ01002463_4
[human gut metagenome]
MSMTPSTFLPKREKNATYIAEVQSIPLSPAAYSVIIKDKSIFETSLSPNGSVSMSSFLTSIFDSAYIASLKYKTDDNYKYIGIP
LLNAFIKWQIEEIDDGLDDKSKEIIKSYLISKFSAKYEKTKTENAVRVRLSICRDLYDTLSSDDLYYENKVYSSTLRRFLKAVY
EDYALLSDCERERLIFADNIIKINEVIKQNGNRYYSFIYAYSNMYSREKRRIRLIPYRIISDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 615)
>OGXL01002096_9
[human gut metagenome]
MSMTPSTFLPKRDTNIPYIAEVQSIPLSPSAYAVIVKDKSIFETSLFPNGGSVSMSSFLTRIFDSAYIASLKYKSEEYNGIPLL
NAFVQWQIEEIDDSLDDKSKEIIKSYLISKLSAKYEKTKTENAVRVRLSICRDLYDTLSSDDLYYENKVYSSTLRRFLKAVYED
YALLSDCERERLIFADNIIKINEVIKQNGSRYYSFIYAYSNMYSREKRRIRLIPYRIVSDEYKMYNYLVCLSDEKSAGKEFKAD
SCRISRLSGLSIAEKLSQKEYSCVTEYERLKEVHVKSVKHLLSDPRFGSDESDISKVYLTEKGVEMFGKILYQRPILKGNEKPK
PNAVNEFISPPIQVKYYFNKFGKDGVIISPSDSFEEMRTLYVEGAEAYNREVEM (SEQ ID NO: 616)
>OGYY01000371_36
[human gut metagenome]
MLIPPSTFLPKREGGVPYIAEVQSIPLSPSAYSVIIKDKSIFETSLSPNGSVSMSSFLTSIFDSAYIASLKYKSEKYNGIPLLN
AFVKWQIEEIDDGLDDKSKEIIKSYLISKLSAKYKKTKTENAVRVRLSISRDLYDTLSSDDLYYENKVYSSTLRRFLKAVYEDY
ALLSDCERERLIFADNIIKINEVIKQNGSRYYSFIYAYSNMYSREKHRIRLIPYRIVSDEYKMYNYLVCLSDEKSAGKEFKADS
CRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 617)
>OHAI01000724_6
[human gut metagenome]
MLIPTSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKNKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRLIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 618)
>OHAN01001071_10
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSLTLRRFLKAVY
EDYALLSDCERERLIFADNIIKINEIIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVKM (SEQ ID NO: 619)
>OHAR01000226_10
[human gut metagenome]
MLIPPSTFLPKRDKNATYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 620)
>OHBL01000590_6
[human gut metagenome]
MSMTPSTFLPKREDGVPYIAEVQSIPLSPSAYSVIIKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKS
NAVNEFISPPIQVKYYFNKFGKDGVIISPSDSFEEMRTLYVEGAEAYNRVVEM (SEQ ID NO: 621)
>OHBW01001448_2
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKHEKTKTENAVRVRLSICRDLYDTLSSDDLYYENKVYSSTLRRFLKAVY
EDYALLSDCERERLLFADNIIKINEVIKQNGSRYYSFIYAYSNMYSREKRLIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 622)
>OHCE01000125_19
[human gut metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYKKTKTENAVRVRLSICRGLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLNDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 623)
>OHCW01000317_6
[human gut metagenome]
MSMTPSTFLPKREDGVPYIAEVQSIPLSPSAYSVIIKDKSIFETSLSPNGSVSMSSFLTSIFDSAYIASLKYKSNDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 624)
>OHEL01001488_5
[human gut metagenome]
MLIPPSTFLPKREGGVPYIAEVQSIPLSPSAYSVIIKDKSIFETSLFPNGSVSMSSFLTSIFDSAYIASLKYKSDDNYKYIGIP
LLNAFVEWQIEEIDDGLDDKSKEIIKSYLISKLSAKYKKTKTENAVRVRLSICRDLYDTLSSDDLYYENKVYSSTLRRFLKAVY
EDYTLLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYSREVEM (SEQ ID NO: 625)
>OHFX01001477_2
[human gut metagenome]
MSMTPSTFLPKREDGVPYIAEVQSIPLSPSAYSVIIKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEVHVKSVKHLLSDPRFGSDESDISKVYLTEKGVEMFGKILYQRPILKGNEKPKP
NTVNEFISPPIQVKYYFNKFGKDGVILSPSDSFEEMRTLYVEGAEAYNREVEK (SEQ ID NO: 626)
>OHGX01000264_3
[human gut metagenome]
MSMTPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKHEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 627)
>OHJS01001864_5
[human gut metagenome]
MSMTPSTFLPKREGGVPYIAEVQSIPLSPSAYSVIIKDKSIFETSLSPNSSVSMSSFLTSIFDSAYIASLKYKSDDNYKYIGIP
LLNAFVKWQIEEIDDDIDDKSKEIIKSYLISKLSAKYKKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 628)
>OHKC01000402_6
[human gut metagenome]
MLIPPSTFLPKRDKNATYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGKAQ
AQRCQRVHLPAYPSQILFQ (SEQ ID NO: 629)
>OHMF01000395_25
[human gut metagenome]
MSMTPSTFLPKREDGVPYIAEVQSIPLSPSAYSVIIRDKSIFETSLSPNGNVSMSSFLTSIFDSAYIASLKYKSDDNYKYIGIP
LLNAFVKWQIEEIDDDLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNRVVEM (SEQ ID NO: 630)
>OHUY01000263_5
[human gut metagenome]
MLMTPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKKIIKSYLISKLSAKYKKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSEGKEFK
ADSYRISRLSGLSISEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 631)
>OIXA01002812_2
[human gut metagenome]
MLIPPSTFLPKREGGVPYIAEVQSIPLSPSAYSVIIKDKSIFETSLSPNGSVSMSSFLTSIFDSTYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 632)
>OIYU01000175_5
[human gut metagenome]
MSMTPSTFLPKREDGVPYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PNAVNEFISPPIQVKYYFNKFGKDGVILSPSDSFEEMRTLYVEGAEAYNREVEM (SEQ ID NO: 633)
>OIZW01000344_21
[human gut metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVQWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRLIRLIPYRIVSDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGIILSPSDSFEEMRTLYVEGAEAYNREVEM (SEQ ID NO: 634)
>OJMJ01002228_2
[human gut metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCKRERLLFAENIIKINEVIKQNGSRYYSFIYAYSNMYSREKRRISLIPYRIVSDEYKMYNYLVCLSDEKSAGKEFK
ADSYRISRLSGLSIAEKLSQKEYSSVTEYERLKEGRVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 635)
>OJMN01000417_21
[human gut metagenome]
MSMTPSTFLPKRDKNATYIAEVQSIPLSPSAYSVIIKDKSIFETSLSPNGSVSMSSFLTSIFDSAYIASLKYKSDDNYKYIGIP
LLNAFVKWQIEEINDSLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVIISPSDSFEEMRTLYVEGAEAYNREVEM (SEQ ID NO: 636)
>OJOH01001697_5
[human gut metagenome]
MLIPPSTFLPKRDKNVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNTVNEFISPPIQVKYYFNRFGKDGVILSPSDSFEEMRTLYVEGAEAYNREVEI (SEQ ID NO: 637)
>OJPG01000139_77
[human gut metagenome]
MSMTPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 638)
>OJPX01000614_6
[human gut metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSRLSIAEKLSQKEYSSVTEYERLKEGHVKSVKHLLSDPRFGSDESDISKVYLTEKGVEMFGKILYQRPILKDNEK
PKPNAVNEFISPPIQVKYYFNKFGKDGVILSPSDSFEEMRTLYVEGAEAYNREVEM (SEQ ID NO: 639)
>OKRZ01002949_4
[human gut metagenome]
MFIPPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKGYLISKLSAKYEKTKTENAVRVRLSICRDLYDTLSSDDLYYENKVYSSTLRRFLKAVY
EDYALLSDCERERLIFADNIIKINEVIKQNDSRYYSFIYAYSNMYSREKHRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 640)
>OKSD01002505_10
[human gut metagenome]
MLIPPSTFLPKRDKNATYIAEVQSIPLSPSAYSVIIKDKSIFETSLSPNGSVSMSSFLTSIFDSAYIASLKYKSEKYNDIPLLN
AFVKWQIEEIDDGLDDKSKEIIKSYLISKLSAKYEKTKTENAVRVRLSICRDLYDTLSSDDLYYENKVYSSTLRRFLKAVYEDY
ALLSDCERERLLFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEVHVKSVKHLLSDPRFGSDESDISKVYLTEKGVEMFGKILYQRPILKGNEKPKP
NAVNEFISQPIQVKYYFNKFGKDGVILSPSDSFEEMRTLYVEGAEAYNREVEM (SEQ ID NO: 641)
>OLGN01000304_31
[human gut metagenome]
MSMTPSTFLPKRDTNIPYIAEVQSIPLSPSAYAVIVKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 642)
>OLHE01000257_40
[human gut metagenome]
MSMTPSTFLPKREGGVPYIAEVQSIPLSPSAYSVIIKDKSIFETSLSPNGSVSMSSFLTSIFDSAYIASLKYKTDDNYKYIGIP
LLNAFVKWQIEEIGDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNRVVEM (SEQ ID NO: 643)
>PPYE01106492_32
[human gut metagenome]
MSMTPSTFLPKRDTNVPYIAEVQSIPLSPSAYSVIIKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 644)
>PPYE01385196_4
[human gut metagenome]
MLIPPSTFLPKRDKNATYIAEVQSIPLSPSAYSVIIKDKSIFETSLSTNGSVSMSSFLTSIFDSAYIASLKYKSDDNYKYIGIP
LLNAFVKWQIEEIDDDLDDKSKEIIKSYLISKLSAKYEKTKTENAVRVRLSISRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 645)
>PPYE01512733_2
[human gut metagenome]
MSMTPSTFLPKREDGVPYIAEVQSIPLSPSAYSVIIKDKSIFETSLSPNGSVSMSSFLTSIFDSAYIASLKYKSEKYNGIPLLN
AFVKWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKHRIRLIPYRIVSDEYKMYNYLVCLSDEKSAGKEFKADS
YRISRLSGLSIAEKLSQKEYSSVTEYERLKEVHVKSVKHLLSDPRFGSDESDISKVYLTEKGVEMFGKILYQRPILKGNEKPKP
NTVNEFISPPIQVKYYFNKFGKDGVILSPSDSFEEMRTLYVEGAEAYNREVEM (SEQ ID NO: 646)
>ODFW01000112_41
[human metagenome]
MLIPPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKHEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 647)
>ODGN01000188_49
[human metagenome]
MSMTPSTFLPKRDKNATYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 648)
>ODHH01000275_15
[human metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDSLDDKSKEIIKSYLISKLSAKYEKTKTENAVRVRLSICRDLYDTLSSDDLYYENKVYSLTLRRFLKAVY
EDYALLSDCERERLIFADNIIKINEIIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 649)
>ODHP01001712_4
[human metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRGLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 650)
>ODHV01000466_16
[human metagenome]
MLIPPSTFLPKREDGVPYIAEVQSIPLSPSAYSVIIKDKSIFETSLSTNGSVSMSSFLTSIFDSAYIASLKYKSDDNYKYIGLP
LLNAFVKWQIEEIDDGLDDKSKEIIKSYLISKLSAKYKKTKTENAVRVRLSICRDLYDTLSSDDLYYENKVYSSTLRRFLKAVY
EDYALLSDCERERLIFADNIIKINEVIKQNGSRYYSFIYAYSNMYSREKHRIRLIPYRIVSDEYKMYNYLVCLSDEKSAGKEFK
ADSYRISRLSGLSIAEKLSQKEYSSVTEYERLKEGHVKSLKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 651)
>ODHZ01001211_6
[human metagenome]
MSMTPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSTGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPNDSFEEMRTLYVEGAEAYNREVEM (SEQ ID NO: 652)
>ODJZ01000182_15
[human metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 653)
>ODVR01002077_4
[human metagenome]
MLIPPSTFLPKRDKNATYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEK (SEQ ID NO: 654)
>ODXC01000747_4
[human metagenome]
MSMTPSTFLPKREGGVPYIAEVQSIPLSPSAYSVIIKDKSIFETSLSPNGSVSMSSFLTRIFDSAYIASLKYKSEEYNGIPLLN
AFVQWQIEEIDDSLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
YRISRLSGLSIAEKLSQKEYSSVTEYERLKEVHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 655)
>ODXO01005124_1
[human metagenome]
MSMTPSTFLPKREDGVPYIAEVQSIPLSPSAYSVIIRDKSIFETSLSPNGNVSMSSFLTSIFDSAYIASLKYKSDDNYKYIGIP
LLNAFVKWQIEEIDDDLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 656)
>ODYC01000377_17
[human metagenome]
MLIPPSTFLPKREDGVPYIAEVQSIPLSPSAYSVIIKDKSIFETSLSPNGSVSMSSFLTSIFDSAYIASLKYKSDDNYKYIGIP
LLNAFVEWQIEEIDDSLDDKSKEIIKSYLISKLSAKHEKTKTENAVRVRLSICRDLYDTLSSDDLYYENKVYSSTLRRFLKAVY
EDYALLSDCERERLIFADNIIKINEIIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEM (SEQ ID NO: 657)
>OEJW01000623_13
[human metagenome]
MLIPPSTFLPKREGGVPYIAEVQSIPLSPSAYSVIIKDKSIFETSLSPNGSVSMSSFLTSIFDSAYIASLKYKSDDNYKYNGIP
LLNAFVEWQIEEIDDGLDDKSKEIIKSYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 658)
>OGHW01002048_2
[metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKDIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVIISPSDSFEEMRTLYVKGAEAYNREVEM (SEQ ID NO: 659)
>OGIE01002059_22
[metagenome]
MSMTPSTFLPKREKNATYIAEVQSIPLSPAAYSVIIKDKSIFETSLSPNGSVSMSSFLTSIFDSAYIASLKYKSDDNYKYNGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKHRIRLIPYRIVSDEYKMYNYLVCLSDEKSAGKEFK
ADSYRISRLSRLSIAEKLSQKEYSSVTEYERLKEGHVKSVKHLLSDPRFGSDESDISKVYLTEKGVEMFGKILYQRPILKGNER
PKHNAVNEFISPPIQVKYYFNKFGKDGVILSPSDSFEEMRTLYVEGAEAYNRVVEK (SEQ ID NO: 660)
>OGJI01000038_150
[metagenome]
MSMIPSTFLPKREGGVPYIAEVQSIPLSPSAYSVIIKDKSIFETSLSPYGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRRIRLIPYRIVSDEYKMYNYLVCLSDEKSAGKEFK
ADSCRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFDEMRTLYVEGAEAYNREVEM (SEQ ID NO: 661)
>OGJY01000516_19
[metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVEWQIEEIDDGLDDKSKEIIKGYLISKLSAKYEKTKTENAVRVRLSICRDLYDTLSSDDLYYENKVYSSTLRRFLKAVY
EDYALLSDCERERLIFADNIIKINEVIKQNGSRYYSFIYAYSNMYSREKRRIRLIPYRIVSDEYKMYNYLVCLSDEKSAGKEFK
ADSYRISRLSGLSIAEKLSQKEYSSVTKYERLKEVHVKSVKHLLSDPRFGSDESDISKVYLTEKGVEMFGKILYQRPILKGNEK
PKPNAVNEFISPPIQVKYYFNKFGKDGVIISPSDSFEEMRTLYVEGAEAYNREVEM (SEQ ID NO: 662)
>OGKA01000617_3
[metagenome]
MSMTPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCERERLLFADNIIKINEVIKQNGSRYYSFIYAYSNMYSREKRLIRLIPYRIVSDEYKMYNYLVCLSDEKSAGKEFK
ADSYRISRLSGLSIAEKLSQKEYFSVTEYERLKEGHVKSVKHLLSDPRFGSDESDISKVYLTEKGVEMFGKILYQRPILKGNEK
PKPNAVNEFISPPIQVKYYFNKFGKDGVILSPSDSFEEMRTLYVEGAEAYNREVEI (SEQ ID NO: 663)
>OGKE01000029_150
[metagenome]
MIPSTFLPKREGGVPYIAEVQSIPLSPSAYSVIIKDKSIFETSLSPYGSVSMSSFLTSIFDSAYIASLKYKSDDNYKYIGIPLL
NAFVKWQIEEIDDGLDDKSKEIIKSYLISKLSAKYEKTKTENAVRVRLSICRDLYDTLSSDDLYYENKVYSSTLRRFLKAVYED
YALLSDCERERLLFADNIIKINEVIKQNGSRYYSFIYAYSNMYSREKRRIRLIPYRIVSDEYKMYNYLVCLSDEKSAGKEFKAD
SCRISRLSGLSIAEKLSQKEYSSVTEYERLKEGHVKSVKHLLSDPRFGSDESDISKVYLTEKGVEMFGKILYQRPILKGNEKPK
PNAVNEFISPPIQVKYYFNKFGKDGVILSPSDSFDEMRTLYVEGAEAYNREVEM (SEQ ID NO: 664)
>OGLJ01000192_55
[metagenome]
MLIPPSTFLPKREGGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCKRERLLFADNIIKINEVIKQNGSRYYSFIYAYSNMYSREKRRIRLIPYRIVSDEYKMYNYLVCLSDEKSSGKEFK
ADSYRISRLSGLSIAEKLSQKEYSSVTEYERLKEVHVKSVKHLLSDPRFGSDESDISKVYLTEKGVEMFGKILYQRPILKGNEK
PKPNAVNEFISPPIQVKYYFNKFGKDGVIISPSDSFEEMRTLYVEGAEAYNREVEM (SEQ ID NO: 665)
>OGMO01000062_68
[metagenome]
MSMTPSTFLPKREGGVPYIAEVQSIPLSPSAYSVIIKDKSIFETSLSPNGSVSMSSFLTSIFDSAYIASLKYKSEKYNGIPLLN
AFVKWQIEEIDDGLDDKSKEIIKSYLISKLSAKYEKTKTENAVRVRLSICRDLYDTLSSDDLYYENKVYSSTLRRFLKAVYEDY
ALLSDCERERLIFADNIIKINEVIKQNGSRYYSFIYAYSNMYSREKRRIRLIPYRIVSDEYKMYNYLVCLSDEKSAGKEFKADS
CRISRLSGLSIAEKLSQKEYSSVTEYERLKEGHVKSVKHLLSDPRFGSDESDISKVYLTEKGVEMFGKILYQRPILKGNEKPKP
NAVNEFISPPIQVKYYFNKFGKDGVILSPSDSFEEMRTLYVEGAEAYNREVEM (SEQ ID NO: 666)
>OGMP01001167_14
[metagenome]
MSMAPSTFLPKREDGVPYIAEVQSIPLSPSAYSVIIKDKSIFETSLSPNGSVSMSSFLTSIFDSAYIASLKYKSDDNYKYIGIP
LLNAFVKWQIEEIDDGLDDKSKEIIKSYLISKLSAKYEKTKTENAVRVRLSICRDLYDTLSSDDLYYENKVYSSTLRRFLKAVY
EDYALLSDCKRERLLFADNIIKINEVIKQNGSRYYSFIYAYSNMYSREKRRIRLIPYRIVSDEYKMYNYLVCLSDEKSSGKEFK
ADSYRISRLSGLSIAEKLSQKEYSSVTEYERLKEGHVKSVKHLLSDPRFGSDESDISKVYLTEKGVEMFGKILYQRPILKGNEK
PKPNAVNEFISPPIQVKYYFNKFGKDGVIISPSDSFEEMRTLYVEGAEAYNREVEM (SEQ ID NO: 667)

TABLE 5
Amino Acid Sequences of Cas13d Accessory Proteins WYL-bl

>DBYI01000091_50
[Ruminococcus flavefaciens]
MENKGKQREFIKDYNKIVPFLEKVFYYGTFSSEDYEKMDMMKKSKYSDYKRILEFAFRDVLYEKKNINGKKALGLRIDHFYDPH
RAFLRFFTLKSFVSIERLFLTCYILKRISKKGKCTINDICIGLDEVSVDDEVKDRKSTISRIIKNMVDYGFLIKKGSAYSINTG
AKTLNNVALLNLIDICTNAYPISICGSCIQNKIDQNYQSPFLIKHLHLGQIFNDELIWKLLIYANEKKQLCIELKKGIKLRELL
PYRIITNRETGRQYLFAIYVGTNNFDEYLMLRLDKISDIKIEASECEIPDDTVLKEKYDTAFRYSFNGTTFLKRDQQPESGILV
YDKSFEWNIKKHFPYSDAVSVDEKHNKVSIKVNTLTELKPWLRRNYDKVSLVESSDDTVDKMCDELKKWRKMYGII (SEQ ID
NO: 89)
>SFX39521.1
[Ruminococcus flavefaciens]
MANEEKNRSFFKITTYENFRRFLKTNFYYCSLSQGQQGMFIKSIGTTKYNEYKNIIELIAGGKIEFPKINKRLAFRYNISQLES
DYNELANSFQLRTLTSLDACLTLYILLFLSDKEMGSSDIYNRIGDIDFDIDEKTIRGKLKNMCEYGMISYKNKKYSLNECSLYS
VDTSIMLSLLNMADFMKNLVYPEVLGYDLFAALKKIYEERTGNEYISPFQFKYSHLANILDDNVLWTLIEAIDNRQHVAFEYGG
KIKERLIPVKIFTENEYNRCYLFAVKRFRNKLKFFVFRLSKIYNLKITNSDEDITEADFKEYSELYDSEKKCSFFGKIDSSAQN
DTVELKYKRGIRSQLERDFSCIEFRKNYTAIVTVKSKKMMIPYLRANMGLIRTTDDELSGILNEDIEEMKKNYGII (SEQ ID
NO: 90)
>3300018494|Ga0187911_10005861_21
[mammals-digestive system-feces]
MNVIIKQGDIFMGNEERNRSFFKEDTYETFRKFLKTNFYYCTLSQKQQSEYVKYIGTTQYNHYRGIIERISEGKISFKKYNKKK
AFKYDVSQFASDYNVLANSFQLKTITASQTCLTIYILCVLAKSSLTRKGIVAAIADGIDEKTIVSRIKSMKEAGLISYDGEKYF
IEESIFYSMDESLLLRLLNMVDFMKNLVYPEALGYNLFDIIKKIYDDRLCVDYYSPFQLKYSHLANILDDNVLWSLIEAIEERQ
YISFIYKNEKKERIIPVKLFTENEYARRYLFAVKKFGNNYKKFIFRLSEIYNIKVMEKEVSVSKEEFGKLLEMYETESGYSFSG
KIAPSSKTVSIKLRYKGRLKNQIERDFSNVKFEKGNTAEILIKNKKMIIPYLRSNMQLIQSTDEELSQKINSEIMEMKKLYGII
(SEQ ID NO: 91)
>DBYI01000091_49
[Ruminococcus flavefaciens]
MQSAWGILSLYGRYGIIIVIRGCDMENKGKQREFIKDYNKIVPFLEKVFYYGTFSSEDYEKMDMMKKSKYSDYKRILEFAFRDV
LYEKKNINGKKALGLRIDHFYDPHRAFLRFFTLKSFVSIERLFLTCYILKRISKKGKCTINDICIGLDEVSVDDEVKDRKSTIS
RIIKNMVDYGFLIKKGSAYSINTGAKTLNNVALLNLIDICTNAYPISICGSCIQNKIDQNYQSPFLIKHLHLGQIFNDELIWKL
LIYANEKKQLCIELKKGIKLRELLPYRIITNRETGRQYLFAIYVGTNNFDEYLMLRLDKISDIKIEASECEIPDDTVLKEKYDT
AFRYSFNGTTFLKRDQQPESGILVYDKSFEWNIKKHFPYSDAVSVDEKHNKVSIKVNTLTELKPWLRRNYDKVSLVESSDDTVD
KMCDELKKWRKMYGII (SEQ ID NO: 668)
>3300018494|Ga0187911_10019634_8
[mammals-digestive system-feces]
MSADLGRNKLLLNENTLKIAKGAFYYGCFTVKHFEEQGISKSTYNRCKDFLLHVFQDRIEEINVPHSRTRMLRLKNDQFEDACN
LLLDLFTYQPASSIEIVTFLSVLRVFTVAAPETSYTFENINKPISHICEDRRTFKKKLHTLVDRGYLLCERRDKRSFQYRLAPV
IFDRLDEFALYRLNALVDLCKCIYHPATCGRYLLDTLAFFNQQKSVNDETIFFCKHMHMGQVFDDAVLWKLMTAIYEKKIISFT
VNGKSYRFQQPCRIIINESDGRRYLYSIGLNTYTKNGKMHRIDQISGIKEEKHTDEISVFSSEEADRRYHNSTQGSFNGISMPR
KKRETAVLVYKKESYPEIQRHFPDAVPEVYDDDHDQVQITVNSLKDIKPWLRLHLGEIRLQSTSNDVKDEFEKEMAEWRAMYGI
V (SEQ ID NO: 669)

TABLE 6
Amino Acid Sequences of Cas13d Accessory Proteins WYL-b2

>SFX39545.1
[Ruminococcus flavefaciens]
MELFNEYRNKSLRAFLKLAERISYGEELSIDEFEAEYYRLSGDNKKITSVFYKNTLYNDKLPIFDTREGKVRLFGEPDKCSNKH
ISDTLLKSEITWLHNALNDKLSKLFLSDEERISIDAKLSDYTEYYKNIDDMWRSNEDISEEVEKNFKIILKAINEKQALSYTFK
NKNCEGFPVRIEYDERTCRIYMIIYDGNRFVKSDISKLSDIYITENSIDTIPEIKDDMLNKKAYLPVVFTVTDDKNRKAIDRAL
LAFSVYDHVVEPIDEKTARFTIQYYTMDLDLLIKDILAFGSDIKVESPRYVVKRITDILRKV (SEQ ID NO: 92)
>3300018494|Ga0187911_10005861_20
[mammals-digestive system-feces]
MELFNEFRNKSFNAFITLAERIANDNAVFSKTEFETEYYRLSGDENRITSIFYNNVINNEKYQIFTIPKDSKDKVQLSIEFDNK
DDINIANIPITSEKKWLHSALHDKLSKLFLSDEEISYIDETISEFPLYYEHIDDSWRKGENISEESVINFRIILQAINEKKSLS
YKYNGKDSEGSPVKIEYDERTCKIYMILYNGSRFIKSDISGLSDICIKEQLYEKIPDIKEGMLEKKARHPIVFTVTDNKNRKSI
ERALLAFSVYEHYVEPIDKNTAKFTIHYYTMDLDILIKDILAFGADIKVEAPQFVVKKIINILENV (SEQ ID NO: 93)
>DBYI01000091_51
[Ruminococcus flavefaciens]
MELSKLELINVYNNCYFISLVNVLNSLTDGEKLDKYKLNNRIANVVNDSQGYFSGKIADEVFDKCSLLFDITPDKTFISRNKVP
IPTCFTVIERIYIKSLINSKYGKLFLSPKEAEEIISCLGDVPDVPINDYLISLPSRTYDYSDKYINNVRFLLMAIKENKEIIYS
NKTKEIVHKNKHGYPIRIEYSALYDLFQLSLWSSEGNRPVKINLHSIYGINLTGNVWGEKKSPIEMMETKRCQEPIVIEISNDN
NTLERANILFSMYNTETEKLKNGTYRKKLYYYYFDENEIVNSIFSFGPYVKVISPTVIVDKIKEKIISLSSISNIL (SEQ ID
NO: 670)
>ODAI011611274_5
[gut metagenome]
MKLFHKYYSRKLLFAIEVLDALQGAKEQTLNWGELTRLSNRLGMTADLRAEVLNVLTEESRIVRVEDTSNYRLDTSWTTTTPKL
PTSKIEEDYLQMILRLPQAEQFLSRELRDRLTDPQASILNTDAIQTIEPNGEQTQLKLSQPEFRMILDAIEMGCAIRYRYISEQ
GKAAMEKHAVPWRLQYSAFDNRWWIILYTLKDHRCVKIALGSISDVQLEKHIQVKEADILKAREKDLAAEPAILQVKNTKNALE
RCFFVMDRQQFEDSELLEDGSAKLTYRYYHFETSDLLRRLLYLGPAVALIGPPKLRKALLEHVERALNHFRAEA (SEQ ID
NO: 671)

TABLE 7
Amino acid Sequences of Motifs in
Type VI-D CRISPR-Cas Effector Proteins

>MOTIF_1
RXXXXH (SEQ ID NO: 94)
>MOTIF_2
DXXXXQXXXXJLDXXK (SEQ ID NO: 95)
>MOTIF_3
FXXXXXXXXXGXXXXXJR (SEQ ID NO: 96)
>MOTIF_4
KEXNXXXXXXXXXXXNI (SEQ ID NO: 97)
>MOTIF_5
YXXXRXKBLXXXXLF (SEQ ID NO: 98)
>MOTIF_6
DXXXXQXXXXXXDIXK (SEQ ID NO: 672)
>MOTIF_7
KXXKNXGXXXXXLRE (SEQ ID NO: 673)

### REFERENCES

Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
Bateman, A., Martin, M. J., O'Donovan, C., Magrane, M., Alpi, E., Antunes, R., Bely, B., Bingley, M., Bonilla, C., Britto, R., et al. (2017). UniProt: the universal protein knowledgebase. Nucleic Acids Res. 45, D158-D169.
Benson, D. A., Cavanaugh, M., Clark, K., Karsch-Mizrachi, I., Lipman, D. J., Ostell, J., and Sayers, E. W. (2013). GenBank. Nucleic Acids Res. 41, D36-42.
Eddy, S. R. (2011). Accelerated Profile HMM Searches. PLoS Comput. Biol. 7, e1002195.
Edgar, R. C. (2004). MUSCLE: multiple sequence alignment with high accuracy and high throughput. Nucleic Acids Res. 32, 1792-1797.
Edgar, R. C. (2010). Search and clustering orders of magnitude faster than BLAST. Bioinformatics 26, 2460-2461.
Finn, R. D., Bateman, A., Clements, J., Coggill, P., Eberhardt, R. Y., Eddy, S. R., Heger, A., Hetherington, K., Holm, L., Mistry, J., et al. (2014). Pfam: the protein families database. Nucleic Acids Res. 42, D222-D230.
Hein, S., Scholz, I., VoB, B., and Hess, W. R. (2013). Adaptation and modification of three CRISPR loci in two closely related cyanobacteria. RNA Biol. 10, 852-864.
Hyatt, D., Chen, G.-L., LoCascio, P. F., Land, M. L., Larimer, F. W., and Hauser, L. J. (2010). Prodigal: prokaryotic gene recognition and translation initiation site identification. BMC Bioinformatics 11, 119.
Makarova, K. S., Anantharaman, V., Grishin, N. V., Koonin, E. V., and Aravind, L. (2014). CARF and WYL domains: ligand-binding regulators of prokaryotic defense systems. Front. Genet. 5.
Peters, J. E., Makarova, K. S., Shmakov, S., and Koonin, E. V. (2017). Recruitment of CRISPR-Cas systems by Tn7-like transposons. Proc. Natl. Acad. Sci. U.S.A 114, E7358-E7366.
Pruitt, K. D., Tatusova, T., Brown, G. R., and Maglott, D. R. (2012). NCBI Reference Sequences (RefSeq): current status, new features and genome annotation policy. Nucleic Acids Res. 40, D130-135.
Shmakov, S., Abudayyeh, O. O., Makarova, K. S., Wolf, Y. I., Gootenberg, J. S., Semenova, E., Minakhin, L., Joung, J., Konermann, S., Severinov, K., et al. (2015). Discovery and Functional Characterization of Diverse Class 2 CRISPR-Cas Systems. Mol. Cell 60, 385-397.
Shmakov, S., Smargon, A., Scott, D., Cox, D., Pyzocha, N., Yan, W., Abudayyeh, O. O., Gootenberg, J. S., Makarova, K. S., Wolf, Y. I., et al. (2017). Diversity and evolution of class 2 CRISPR-Cas systems. Nat. Rev. Microbiol. 15, 169-182.
Smargon, A. A., Cox, D. B. T., Pyzocha, N. K., Zheng, K., Slaymaker, I. M., Gootenberg, J. S., Abudayyeh, O. A., Essletzbichler, P., Shmakov, S., Makarova, K. S., et al. (2017). Cas13b Is a Type VI-B CRISPR-Associated RNA-Guided RNase Differentially Regulated by Accessory Proteins Csx27 and Csx28. Mol. Cell 65, 618-630.e7.
Steinegger, M., and Söding, J. (2017). MMseqs2 enables sensitive protein sequence searching for the analysis of massive data sets.
Yu, J., Picord, G., Tuffery, P., and Guerois, R. (2015). HHalign-Kbest: exploring sub-optimal alignments for remote homology comparative modeling. Bioinforma. Oxf. Engl. 31, 3850-3852.
Zhu, W., Lomsadze, A., and Borodovsky, M. (2010). Ab initio gene identification in metagenomic sequences. Nucleic Acids Res. 38, e132-e132.

### Example 2. Accelerated In Vivo Functional Screening of Type VI-D CRISPR-Cas Systems

Having identified the minimal suite of Type VI-D CRISPR-Cas system components, we selected two loci for functional validation, those from Eubacterium siraeum DSM 15702 (EsCas13d) and Ruminococcus sp. N15. MGS-57 (RspCas13d). RspCas13d is a member of the largest subgroup of Cas13d proteins which contains 13 of the 31 unique members of the family and shows co-conservation with a putative WYL1 accessory protein (FIGS. 1, 6, 7). In contrast, there are no WYL-domain proteins (or other putative accessory proteins) encoded within 3 kb of the EsCas13d effector.

### DNA Synthesis and Effector Library Cloning

To test the activity of Type VI-D CRISPR-Cas, we designed and synthesized minimal systems containing RspCas13d or EsCas13d into the pET28a(+) vector. The synthesized Ruminococcus sp. RspCas13d system included RspCas13d and RspWYL1, codon optimized for E. coli expression under the control of a lac promoter and separated by an E. coli ribosome binding sequence (FIG. 8). Following the open reading frames for RspCas13d and RspWYL1, we included an acceptor site for a CRISPR array library driven by a J23119 promoter. The Eubacterium siraeum system was prepared similarly but included no gene for a WYL-domain containing protein.

The E. coli codon-optimized genes representing the minimal CRISPR effectors and accessory proteins were synthesized (Genscript) into a custom expression system derived from the pET-28a(+) (EMD-Millipore). Briefly, the Ruminococcus sp. synthesis product included Cas13d and WYL1 codon optimized for E. coli expression under the control of a Lac promoter and separated by an E. coli ribosome binding sequence. Following the open reading frames for Cas13d and WYL1, we included an acceptor site for a CRISPR array library driven by a J23119 promoter (Registry of Standard Biological Parts: parts.igem.org/Part:BBa_J23119). Our Eubacterium siraeum system was similarly constructed, but with only the effector protein.

In tandem with the effector gene synthesis, we first computationally designed an oligonucleotide library synthesis (OLS) pool containing “repeat-spacer-repeat” sequences, where “repeat” represents the consensus direct repeat sequence found in the CRISPR array associated with the effector, and “spacer” represents sequences tiling the pACYC184 plasmid. The spacer length was determined by the mode of the spacer lengths found in the endogenous CRISPR array. The repeat-spacer-repeat sequence was appended with restriction sites enabling the bi-directional cloning of the fragment into the aforementioned CRISPR array library acceptor site, as well as unique PCR priming sites to enable specific amplification of a specific repeat-spacer-repeat library from a larger pool. The library synthesis was performed by Agilent Genomics.

We next cloned the repeat-spacer-repeat library into the plasmid containing the minimal engineered locus using the Golden Gate assembly method. In brief, we first amplified each repeat-spacer-repeat from the OLS pool (Agilent Genomics) using unique PCR primers, and pre-linearized the plasmid backbone using BsaI to reduce potential background. Both DNA fragments were purified with Ampure XP (Beckman Coulter) prior to addition to Golden Gate Assembly Master Mix (New England Biolabs) and incubated as per manufacturer's instructions. We further purified and concentrated the Golden Gate reaction to enable maximum transformation efficiency in the subsequent steps of the bacterial screen.

### Accelerated Functional Screening for Cas13d

To accelerate functional screening of Type VI-D systems, we developed a strategy to derive the following functional information in a single screen: 1) crRNA expression direction and processing, 2) nucleic acid substrate type, and 3) targeting requirements such as protospacer adjacent motif (PAM), protospacer flanking sequence (PFS), or target secondary structure. We designed minimal CRISPR array libraries consisting of two consensus direct repeats, each flanking a unique natural-length spacer sequence targeting either the pACYC184 vector or an absent GFP sequence as a negative control. The CRISPR array libraries for EsCas13d and RspCas13d systems consisted of 4549 and 3972 pACYC184-targeting spacers respectively, in addition to 452 and 450 spacers targeting the GFP negative control sequence, respectively. We also designed a bidirectional array library cloning strategy to test both possible CRISPR array expression directions in parallel.

The CRISPR array libraries for RspCas13d and EsCas13d were cloned into acceptor sites on respective Type VI-D expression plasmids such that each plasmid contained a single library element and orientation (FIG. 8). The resulting plasmid libraries were transformed with pACYC184 into Stbl3 E. coli using electroporation, yielding a maximum of one plasmid library element per cell. Transformed E. coli cells were plated on bioassay plates containing Kanamycin (selecting for the library plasmid), Chloramphenicol (CAM; selecting for intact pACYC184 CAM expression), and Tetracycline (TET; selecting for intact pACYC184 TET expression), such that interruption of pACYC184 plasmid DNA or antibiotic resistance gene expression by the CRISPR-Cas system results in bacterial cell death. Screens were harvested 12 h after plating, and plasmid DNA was extracted (FIG. 9). We PCR amplified the CRISPR array region of the input plasmid library prior to transformation and the output plasmid library after bacterial selection on antibiotic plates.

The plasmid library containing the distinct repeat-spacer-repeat elements and Cas proteins was electroporated into Endura electrocompetent E. coli (Lucigen) using a Gene Pulser Xcell® (Bio-rad) following the protocol recommended by Lucigen. The library was either co-transformed with purified pACYC184 plasmid, or directly transformed into pACYC184-containing Endura electrocompetent E. coli (Lucigen), plated onto agar containing Chloramphenicol® (Fisher), Tetracycline (Alfa Aesar), and Kanamycin (Alfa Aesar) in BioAssay® dishes (Thermo Fisher), and incubated for 10-12 h. After estimation of approximate colony count to ensure sufficient library representation on the bacterial plate, the bacteria were harvested and DNA plasmid extracted using a QIAprep Spin Miniprep® Kit (Qiagen) to create the “output library.” By performing a PCR using custom primers containing barcodes and sites compatible with Illumina sequencing chemistry, we generated a barcoded next generation sequencing library from both the pre-transformation “input library” and the post-harvest “output library,” which were then pooled and loaded onto a Nextseq 550 (Illumina) to evaluate the effectors. At least two independent biological replicates were performed for each screen to ensure consistency.

### Bacterial Screen Sequencing Analysis

Next generation sequencing data for screen input and output libraries were demultiplexed using Illumina bcl2fastq. Reads in resulting fastq files for each sample contained the CRISPR array elements for the screening plasmid library. The direct repeat sequence of the CRISPR array was used to determine the array orientation, and the spacer sequence was mapped to the source plasmid pACYC184 or negative control sequence (GFP) to determine the corresponding target. For each sample, the total number of reads for each unique array element (rₐ) in a given plasmid library was counted and normalized as follows: (rₐ+1)/total reads for all library array elements. The depletion score was calculated by dividing normalized output reads for a given array element by normalized input reads.

To identify specific parameters resulting in enzymatic activity and bacterial cell death, we used next generation sequencing (NGS) to quantify and compare the representation of individual CRISPR arrays (i.e., repeat-spacer-repeat) in the PCR of the input and output plasmid libraries. We defined the array depletion ratio as the normalized output read count divided by the normalized input read count. An array was considered to be strongly depleted if the depletion ratio was less than 0.1 (more than 10-fold depletion). When calculating the array depletion ratio across biological replicates, we took the maximum depletion ratio value for a given CRISPR array across all experiments (i.e. a strongly depleted array must be strongly depleted in all biological replicates). We generated a matrix including array depletion ratios and the following features for each spacer target: target strand, transcript targeting, ORI targeting, target sequence motifs, flanking sequence motifs, and target secondary structure. We investigated the degree to which different features in this matrix explained target depletion for RspCas13d and EsCas13d systems, thereby yielding abroad survey of functional parameters within a single screen.

Distribution of Bacterial Screening Targets Indicates that Cas13d Targets ssRNA Transcripts

To identify the targeted substrate for Cas13d, we first identified a set of minimal CRISPR arrays that were strongly depleted in 2 screen biological replicates. For both RspCas13d and EsCas13d systems, these strongly depleted arrays primarily targeted pACYC184, with minimal depletion of the negative control (FIGS. 10 and 11). We observed 1119 and 806 strongly depleted arrays for the RspCas13d and EsCas13d systems, respectively (FIGS. 12A-B). The spatial distribution and strand preference of the strongly depleted target sites along pACYC184 (FIGS. 13A-B) indicate a preference for transcript targeting, suggesting that Cas13d targets single-stranded RNA transcripts. Additionally, the presence of strongly depleted targets within the non-coding region of pACYC184 between the Tet and CAM ORFs corresponds to the extension of RNA transcripts coding for these genes beyond the end of the open reading frame.

These results indicate that targeting of non-essential regions of transcripts might trigger additional catalytic activities of Cas13d enzymes resulting in toxicity and cell death

### Lack of PFS for Cas13d and a New Model for Analysis of Sequence Constraints

Previous RNA targeting CRISPR-Cas systems from subtypes VI-A-C have shown varying dependence on a protospacer flanking sequence (PFS) for efficient RNA targeting (Abudayyeh et al., 2016, 2017; Cox et al., 2017; East-Seletsky et al., 2016, 2017; Gootenberg et al., 2017; Smargon et al., 2017). Here we present evidence that RspCas13d and EsCas13d have no such flanking sequence requirements. For each enzyme, WebLogos® (Crooks et al., 2004) show that at each of 30 positions before and after the target sequences for strongly depleted arrays the nucleotide frequencies do not appreciably differ from a uniform distribution (FIGS. 14A-B).

To investigate possible flanking sequence requirements further, we developed a combinatorial model to search for up to 3 nucleotide locations distributed across the target or flanking sequences that might explain the observed strongly depleted arrays. We calculated a bit score to measure the degree to which the selected locations correspond to strongly biased outcomes (e.g. all hits or all non-hits). More specifically, we defined a targeting requirement to comprise a set of locations relative to a target sequence and the corresponding nucleotide sequences at those locations. For a given targeting requirement, we define the hit ratio (hr) as the ratio of the number of strongly depleted CRISPR arrays to the total number of library targets satisfying the requirement. When searching for a PAM or PFS of length k, we consider

$$( \begin{matrix}n \\ k\end{matrix} )$$

potential targeting requirement locations, where n=spacer length+2·flank length. The bit score for a potential targeting requirement is calculated as bitscore=Σ−hr log(hr) over all nucleotide sequences at the specified targeting requirement locations. For CRISPR-Cas systems with known PAM or PFS requirements, such as BzCas13b, high bit scores for targeting requirements of length 2 or 3 within 15 nt flanks of the target were obtained, and accurately recapitulate the location of the known PFS (FIG. 14C). Conversely, for RspCas13d and EsCas13d, our analysis shows no evidence of flanking or spacer sequences contributing to the targeting efficiency of strongly depleted arrays (FIG. 14C).

### Explaining Strongly Depleted Arrays for RspCas13d and EsCas13d

Cumulatively, transcript targeting explained 86% and 66% of the strongly depleted arrays for RspCas13d and EsCas13d, respectively (FIG. 15). Accordingly, little if any targeting was observed for the ORF template strand. Non-coding and origin of replication (ORI) targeting correspond to actively transcribed regions of the ORI and the extension of coding transcripts into the intergenic region, as corroborated by RNA sequencing of Stbl3 E. coli containing pACYC184 (FIGS. 14A-B). Secondary structure analysis of the transcripts further enhanced the explanation of targeting for Cas13d. We predicted RNA secondary structure (Lorenz et al., 2011) for all sub-sequences within 30 nt of transcript target sites, and found that sequences with no predicted stable secondary structure corresponded to a higher percentage of strongly depleted targets (FIGS. 16A-B). Accordingly, we selected several sub-sequence ranges around the target site (FIGS. 16A-B), and defined a minimal secondary structure targeting requirement to be satisfied if the target site exhibited no predicted stable secondary structure for any of the selected sequence ranges. Among the transcript target sites that satisfy the minimal secondary structure requirement, we can explain 93% and 84% of all strongly depleted arrays for RspCas13d and EsCas13d, respectively (FIG. 16C). Together, our results indicate that RspCas13d and EsCas13d are RNA-targeting effectors with no flanking sequence requirements and a preference for minimal secondary structure for RNA targeting in E. coli.

RNA-Sequencing Mature crRNA from In Vivo Bacterial Screen

Sequencing the small RNA from the in vivo bacterial screen began by extracting total RNA from harvested screen bacteria using the Direct-zol RNA MiniPrep® Plus w/TRI Reagent (Zymo Research). Ribosomal RNA was removed using a Ribo-Zero® rRNA Removal Kit for Bacteria, followed by cleanup using a RNA Clean and Concentrator-5 kit. The resultant ribosomal RNA depleted total RNA was treated with T4 PNK, RNA 5′ polyphosphatase, prepared for sequencing using the NEBNext® Small RNA Library Prep Set, and analyzed as described above.

We analyzed the pre-crRNA processing in the screen output samples for the direct repeat orientation that demonstrated successful targeting of pACYC184 and identified a mature 53 nt crRNA consisting of a 5′ direct repeat truncated by 6 nt (FIG. 17). The most common spacer length observed for EsCas13d was 23 nt, with length variation between 20 nt and 30 nt (length of the native spacer for EsCas13d).

### REFERENCES

Abudayyeh, O. O., Gootenberg, J. S., Konermann, S., Joung, J., Slaymaker, I. M., Cox, D. B. T., Shmakov, S., Makarova, K. S., Semenova, E., Minakhin, L., et al. (2016). C2c2 is a single-component programmable RNA-guided RNA-targeting CRISPR effector. Science 353, aaf5573.
Abudayyeh, O. O., Gootenberg, J. S., Essletzbichler, P., Han, S., Joung, J., Belanto, J. J., Verdine, V., Cox, D. B. T., Kellner, M. J., Regev, A., et al. (2017). RNA targeting with CRISPR-Cas13. Nature 550, 280-284.
Cox, D. B. T., Gootenberg, J. S., Abudayyeh, O. O., Franklin, B., Kellner, M. J., Joung, J., and Zhang, F. (2017). RNA editing with CRISPR-Cas13. Science 358, 1019-1027.
Crooks, G. E., Hon, G., Chandonia, J.-M., and Brenner, S. E. (2004). WebLogo: a sequence logo generator. Genome Res. 14, 1188-1190.
East-Seletsky, A., O'Connell, M. R., Knight, S. C., Burstein, D., Cate, J. H. D., Tjian, R., and Doudna, J. A. (2016). Two distinct RNase activities of CRISPR-C2c2 enable guide-RNA processing and RNA detection. Nature 538, 270-273.
East-Seletsky, A., O'Connell, M. R., Burstein, D., Knott, G. J., and Doudna, J. A. (2017). RNA Targeting by Functionally Orthogonal Type VI-A CRISPR-Cas Enzymes. Mol. Cell 66, 373-383.e3.
Gootenberg, J. S., Abudayyeh, O. O., Lee, J. W., Essletzbichler, P., Dy, A. J., Joung, J., Verdine, V., Donghia, N., Daringer, N. M., Freije, C. A., et al. (2017). Nucleic acid detection with CRISPR-Cas13a/C2c2. Science 356, 438-442.
Lorenz, R., Bernhart, S. H., Höner zu Siederdissen, C., Tafer, H., Flamm, C., Stadler, P. F., and Hofacker, I. L. (2011). ViennaRNA Package 2.0. Algorithms Mol. Biol. 6, 26.
Smargon, A. A., Cox, D. B. T., Pyzocha, N. K., Zheng, K., Slaymaker, I. M., Gootenberg, J. S., Abudayyeh, O. A., Essletzbichler, P., Shmakov, S., Makarova, K. S., et al. (2017). Cas13b Is a Type VI-B CRISPR-Associated RNA-Guided RNase Differentially Regulated by Accessory Proteins Csx27 and Csx28. Mol. Cell 65, 618-630.e7.

### Example 3. Validation of Type VI-D Effector Activity In Vitro (Biochemically)

### Effector and Accessory Protein Purification

The effector or accessory protein expression construct was transformed into an E. coli T7 expression strain, NiCo21(DE3)® (New England Biolabs). 1 mL of overnight culture was inoculated into 1 liter of Luria-Bertani broth growth media (10 g/L tryptone, 5 g/L yeast extract, 5 g/L NaCl, Sigma) supplemented with 50 μg/mL Kanamycin. Cells were grown at 37° C. to a cell density of 0.5-0.8 OD₆₀₀. Protein expression was then induced by supplementing with IPTG to a final concentration of 0.2 mM and the culture continued to grow for 14-18 hours at 20° C. The cells were harvested by centrifugation and cell paste was resuspended in 80 ml of freshly prepared Lysis Buffer (50 mM Hepes pH 7.6, 0.5M NaCl, 10 mM imidazole, 14 mM 2-mercaptoethanol and 5% glycerol) supplemented with protease inhibitors (cOmplete, EDTA-free, Roche Diagnostics Corporation). The resuspended cells were broken by passing through a cell disruptor (Constant System Limited). Lysate was cleared by centrifugation twice at 28,000 g for 30 min each. The clarified lysate was applied to a 5 ml HisTrap FF chromatography column (GE Life Sciences).

Protein purification was performed via FPLC (AKTA Pure, GE Healthcare Life Sciences). After washing with Lysis Buffer, protein was eluted with a gradient of 10 mM to 250 mM of imidazole. Fractions containing protein of the expected size were pooled, concentrated in Vivaspin 20 ultrafiltration unit (Sartorius) and either used directly for biochemical assays or frozen at −80° C. for storage. Protein purity was determined by SDS-PAGE analysis and protein concentration was determined by Qubit® protein assay kit (Thermo Fisher). FIG. 17 shows a Coomassie blue stained polyacrylamide gel of the purified recombinant proteins EsCas13d, RspCas13d, and RspWYL1 respectively.

crRNA and Substrate RNA Preparation

DNA oligo templates for crRNA and substrate RNA in vitro transcription were ordered from IDT (TABLES 8 and 9). Templates for crRNAs were annealed to a short T7 primer (final concentrations 4 M) and incubated with T7 RNA polymerase overnight at 37° C. using the HiScribe® T7 Quick High Yield RNA Synthesis kit (New England Biolabs). Annealing was performed by incubating T7 primer with templates for 2 minutes at 95° C. followed by a −5° C./s ramp down to 23° C. Templates for substrate RNA were PCR amplified to yield dsDNA and then incubated with T7 RNA polymerase at 37° C. overnight using the same T7 Quick High Yield RNA Synthesis kit. After in vitro transcription, samples were treated with DNase I (Zymo Research) and then purified using RNA Clean & Concentrator kit (Zymo Research).

5′ end labeling was accomplished using the 5′ end labeling kit (VectorLabs) and with a IR800® dye-maleimide probe (LI-COR Biosciences). Body labeling of RNA was performed during in vitro transcription using the HiScribe® T7 Quick High Yield RNA Synthesis kit (New England Biolabs). The in vitro transcription reactions contained 2.5 mM Fluorescein-12-UTP (Sigma Aldrich). Labeled RNA was purified to remove excess dyes using RNA Clean & Concentrator kit (Zymo Research). The RNA concentration was measured on Nanodrop® 2000 (Thermo Fisher).

The effectors were then incubated with their respective in vitro transcribed pre-crRNAs consisting of a minimal CRISPR array with the repeat-spacer-repeat construction used in the bacterial screening library, but with a single spacer instead of a library. Pre-crRNA cleavage assays were performed at 37° C. in processing buffer (20 mM Tris pH8.0, 50 mM KCl, 1 mM EDTA, 10 mM MgCl2, and 100 ug/ml BSA) unless otherwise indicated, with a final reaction concentration of 200 nM of pre-crRNA and varying enzyme concentrations and EDTA as indicated. Reactions were incubated for 30 minutes, and quenched with the addition of 1 ug/uL of proteinase K (Ambion) incubated for 10 minutes at 37° C. Afterwards, 50 mM of EDTA was added to the reaction, which was then mixed with equal parts 2×TBE-Urea Sample Buffer (Invitrogen) prior to denaturing at 65 C for 3 minutes. Samples were analyzed by denaturing gel electrophoresis on 15% TBE-Urea gels (Invitrogen) and stained using SYBR Gold nucleic acid stain (Invitrogen) for 10-20 minutes prior to imaging on a Gel Doc EZ (Biorad). We found that EsCas13d and RspCas13d effectors process pre-crRNAs to form mature crRNAs in the absence of any accessory proteins (FIGS. 20A-D).

### RNA-Sequencing of In Vitro Cleaved Pre-crRNA

Sequencing of in vitro cleaved pre-crRNA began with performing and quenching the cleavage assays as described above. The reactions were then column purified using a RNA Clean and Concentrator-5 kit (Zymo Research). The RNA samples were then PNK treated for 3 hours without ATP to enrich for 3′-P ends, after which ATP was added and the reaction incubated for another hour to enrich for 5′-OH ends. The samples were then column purified, incubated with RNA 5′ polyphosphatase (Lucigen) and column purified again prior to preparation for next-generation sequencing using the NEBNext® Multiplex Small RNA Library Prep Set for Illumina (New England Biolabs). The library was paired-end sequenced on a Nextseq 550® (Illumina), and the resulting paired end alignments were analyzed using Geneious 11.0.2 (Biomatters).

Performing next-generation sequencing of the in vitro cleaved RNA fragments enabled the exact identification of the processing intermediates and mature crRNA (FIG. 19) visualized by denaturing gel. For both EsCas13d and RspCas13d, sequencing the mature crRNA corroborated the 6 nt truncation from the 5′ end of the first direct repeat found in the in vivo small RNA sequencing. For the 3′ end, 6 nt of the second direct repeat remained attached to the 3′ end of the spacer, yielding a total product of 66 nt consistent with the mature crRNA visualized by denaturing gel. The difference between the well-defined 3′ end of the mature crRNA forms observed in vitro versus the various lengths identified in vivo may be the result of further truncation in vivo by endogenous RNases following the initial pre-crRNA cleavage. The effector's ability to cleave pre-crRNA at the same location relative to the predicted stem loop structure of either direct repeat (FIG. 19 intermediates 1 and 2) indicates that the Type VI-D CRISPR-Cas effectors are able to process pre-crRNAs containing multiple DRs and spacers.

Effect of EDTA on crRNA Processing

We next examined the dependence of pre-crRNA cleavage on divalent metal ions. We observed that the generation of mature crRNA for both EsCas13d and RspCas13d is substantially inhibited by the addition of EDTA (FIGS. 20A-D), while Cas13a from Leptotrichia wadei (LwaCas13a) is still able to generate mature crRNAs in the presence of EDTA (FIG. 21). This dependence of Cas13d on divalent cations to generate mature crRNA is a notable functional distinction from Cas13a crRNA processing (East-Seletsky et al., 2016; Knott et al., 2017).

Validation of ssRNA Cleavage Activities

We next sought to biochemically validate the RNA-guided ssRNA cleavage activities of the Cas13d enzymes observed in our bacterial screens. Target cleavage assays were performed at 37° C. in cleavage buffer (20 mM HEPES pH 7.1, 50 mM KCl, 5 mM MgCl2 and 5% glycerol). Cas13-crRNA complex formation was performed in cleavage buffer by incubating a 2:1 molar ratio of protein to crRNA at 37° C. for 5 minutes, and RspWYL1 was added to the Cas13-crRNA pre-incubation according to the experimental conditions. For the cleavage reactions at different Cas13 concentrations, the pre-formed Cas13-crRNA complexes were diluted on ice, keeping the Cas13-crRNA ratio constant at 2:1. The 5′ IR800 labeled target ssRNA and/or additional unlabeled and fluorescent body-labeled ssRNAs were then added to the pre-formed complex and incubated at 37° C. for 30 minutes. The final concentration of short substrate RNAs was 100 nM and the fluorescent body-labeled ssRNA for collateral effect visualization was 50 nM, unless otherwise indicated. Reactions were quenched by adding 1 ug/uL of proteinase K (Ambion) and incubating for 10 minutes at 37° C.

Afterwards, 50 mM of EDTA was added to the reaction, which was then mixed with equal parts 2×TBE-Urea Sample Buffer (Invitrogen) prior to denaturing at 65° C. for 3 minutes. Samples were analyzed by denaturing gel electrophoresis on 6% or 15% TBE-Urea gels (Invitrogen). Fluorescence images were obtained using a Gel Doc EZ® (Biorad), and near-infrared images were obtained using an Odyssey® CLx scanner (LI-COR Biosciences). Afterwards, the gels were stained for 10-20 minutes using SYBR Gold nucleic acid stain (Invitrogen) and imaged on the Gel Doc EZ® to verify the results from the fluorescence and IR images.

We titrated Apo EsCas13d and RspCas13d (100-0.4 nM) over a non-targeted ssDNA substrate (100 nM), with the denaturing gel (FIGS. 22A-B) showing minimal cleavage products. We then titrated EsCas13d and RspCas13d in complex with crRNA (100-0.4 nM) over non-targeted ssDNA substrates (100 nM), with the resulting denaturing gel (FIGS. 23A-B) showing minimal cleavage products.

We identified spacer sequences for several strongly depleted arrays from bacterial screens for each CRISPR-Cas system and generated pre-crRNAs with the repeat-spacer-repeat arrangement for each effector. We then titrated EsCas13d and RspCas13d in complex with crRNA (100-0.4 nM) over targeted ssDNA substrates (100 nM), with the resulting denaturing gel (FIGS. 24A-B) showing saturation of target cleavage activity at approx. 50 nM RspCas13d-crRNA complex and 100 nM EsCas13d-crRNA complex. In an additional experiment, we targeted EsCas13d and RspCas13d enzyme-crRNA complexes to 130 nt ssRNA substrates containing target sequences complementary to the crRNA spacer and demonstrated targeted RNA cleavage activity for both enzymes (FIGS. 25A-B).

To evaluate the collateral RNA cleavage activity, identical reactions were prepared and supplemented with 800 nt fluorescent body-labeled ssRNA fragments that did not contain the target sequence. Both EsCas13d and RspCas13d showed substantial collateral activity that occurs with the target cleavage (FIGS. 26A-B). We further demonstrated that both EsCas13d and RspCas13d show robust sequence-specific targeted and collateral RNA cleavage activities across multiple crRNAs with and without complementary substrates (FIGS. 26C-D).

TABLE 8
ssRNA Oligos Used in This Study

| ID | Type | Source | Description | Sequence | FIGS. |
|---|---|---|---|---|---|
| cr_F1 | ssRNA | IDT IVT | EsCas13d pre-crRNA #1 | GAACUACACCCGUGCAAAAUUGCAGGGGUCUAAAACUCAUCCGCUUA | 20A-B, 23A, |
| | | | | UUAUCACUUAUUCAGGCGUGAACUACACCCGUGCAAAAUUGCAGGGG | 24A, 25A, |
| | | | | UCUAAAAC (SEQ ID NO: 99) | 26A, 26C, |
| | | | | | 30A-B |
| cr_F4 | ssRNA | IDT IVT | EsCas13d pre-crRNA #2 | GAACUACACCCGUGCAAAAUUGCAGGGGUCUAAAACAUAGGUACAUU | 20B, 26C |
| | | | | GAGCAACUGACUGAAAUGCGAACUACACCCGUGCAAAAUUGCAGGGG | |
| | | | | UCUAAAAC (SEQ ID NO: 100) | |
| cr_F7 | ssRNA | IDT IVT | RspCas13d pre-crRNA #1 | CUACUACACUGGUGCAAAUUUGCACUAGUCUAAAACCAAGGGUGAAC | 20C-D, 23B, |
| | | | | ACUAUCCCAUAUCACCAGCUCUACUACACUGGUGCGAAUUUGCACUA | 24B, 25B, |
| | | | | GUCUAAAAC (SEQ ID NO: 101) | 26B, 26D, |
| | | | | | 29A-C |
| cr_F10 | ssRNA | IDT IVT | RspCas13d pre-crRNA #2 | CUACUACACUGGUGCAAAUUUGCACUAGUCUAAAACCCUGUGGAACA | 20D, 26D |
| | | | | CCUACAUCUGUAUUAACGAACUACUACACUGGUGCGAAUUUGCACUA | |
| | | | | GUCUAAAAC (SEQ ID NO: 102) | |
| cr_3 | ssRNA | IDT IVT | LwaCas13a pre-crRNA #1 | GAUUUAGACUACCCCAAAAACGAAGGGGACUAAAACAUUUUUUUCUC | 21 |
| | | | | CAUUUUAGCUUCCUUAGGAUUUAGACUACCCCAAAAACGAAGGGGAC | |
| | | | | UAAAAC (SEQ ID NO: 103) | |
| cr_4 | ssRNA | IDT IVT | LwaCas13a pre-crRNA #2 | GAUUUAGACUACCCCAAAAACGAAGGGGACuAAAACAGAAUCAUAAU | 21 |
| | | | | GGGGAAGGCCAUCCAGCGAUUUAGACUACCCCAAAAACGAAGGGGAC | |
| | | | | UAAAAC (SEQ ID NO: 104) | |
| sub_F1 | ssRNA | PCR IVT | EsCas13d substrate #1; | AUACGCUGUGGUUCGCCAAGUCCCAAUGGCAUCGUAAAGAACAUUUU | 24A, 25A, |
| | | | “target ssRNA” in FIGs. | GAGGCAUUUCAGUCAGUUGCUCAAUGUACCUAUAACCAGACCGUUCA | 26A, 26C, |
| | | | 24, 25, 26, 30; “A” in | GCUGGAUAUUACGGCCAAGAGAGCACGAAAGUGUUG (SEQ ID | 30A-B |
| | | | FIG. 26C | NO: 105) | |
| sub_F4 | ssRNA | PCR IVT | EsCasnd substrate #2; | AUACGCUGUGGUUCGCCAAGAGUUAUUGGUGCCCUUAAACGCCUGGU | 22A, 23A, |
| | | | “non target ssRNA” in | GCUACGCCUGAAUAAGUGAUAAUAAGCGGAUGAAUGGCAGAAAUUCG | 25A, 26A, |
| | | | FIGs. 22, 23, 25, 26, | AAAGCAAAUUCGACCCAAGAGAGCACGAAAGUGUUG (SEQ ID | 26C, 30A-B |
| | | | 30; “B” in FIG. 26C | NO: 106) | |
| sub_F7 | ssRNA | PCR IVT | RspCas13d substrate #1; | AUACGCUGUGGUUCGCCAAGCGGAAUUCCGUAUGGCAAUGAAAGACG | 24B, 25B, |
| | | | “target ssRNA” in FIGs. | GUGAGCUGGUGAUAUGGGAUAGUGUUCACCCUUGUUACACCGUUUUC | 26B, 26B, |
| | | | 24, 25, 26, 29; “A” in | CAUGAGCAAACUGAAACAAGAGAGCACGAAAGUGUUG (SEQ ID | 26D, 29A-C |
| | | | FIG. 26D | NO: 107) | |
| sub_F10 | ssRNA | PCR IVT | RspCas13d substrate #2; | AUACGCUGUGGUUCGCCAAGCUCCCAGAGCCUGAUAAAAACGGUUAG | 22g, 23g, |
| | | | “non target ssRNA” in | CGCUUCGUUAAUACAGAUGUAGGUGUUCCACAGGGUAGCCAGCAGCA | 25B, 26B, |
| | | | FIGs. 22, 23, 25, 26, | UCCUGCGAUGCAGAUCCAAGAGAGCACGAAAGUGUUG (SEQ ID | 26D, 29A-C |
| | | | 29; “B” in FIG. 26D | NO: 108) | |
| GFP | ssRNA | PCR IVT | Collateral ssRNA; when | GGGAAUUGUGAGCGGAUAACAAUUCCCCUCUAGAAAUAAUUUUGUUU | 26A-D, |
| | | | IVT completed with | AACUUUAAGAAGGAGAUUUAAAUAUGAAAAUCGAAGAAGGUAAAGGU | 29B-C, 30B |
| | | | Fluorescein-12-UTP, | CACCAUCACCAUCACCACGGAUCCAUGACGGCAUUGACGGAAGGUGC | |
| | | | produces body labeled | AAAACUGUUUGAGAAAGAGAUCCCGUAUAUCACCGAACUGGAAGGCG | |
| | | | ssRNA | ACGUCGAAGGUAUGAAAUUUAUCAUUAAAGGCGAGGGUACCGGUGAC | |
| | | | | GCGACCACGGGUACCAUUAAAGCGAAAUACAUCUGCACUACGGGCGA | |
| | | | | CCUGCCGGUCCCGUGGGCAACCCUGGUGAGCACCCUGAGCUACGGUG | |
| | | | | UUCAGUGUUUCGCCAAGUACCCGAGCCACAUCAAGGAUUUCUUUAAG | |
| | | | | AGCGCCAUGCCGGAAGGUUAUACCCAAGAGCGUACCAUCAGCUUCGA | |
| | | | | AGGCGACGGCGUGUACAAGACGCGUGCUAUGGUUACCUACGAACGCG | |
| | | | | GUUCUAUCUACAAUCGUGUCACGCUGACUGGUGAGAACUUUAAGAAA | |
| | | | | GACGGUCACAUUCUGCGUAAGAACGUUGCAUUCCAAUGCCCGCCAAG | |
| | | | | CAUUCUGUAUAUUCUGCCUGACACCGUUAACAAUGGCAUCCGCGUUG | |
| | | | | AGUUCAACCAGGCGUACGAUAUUGAAGGUGUGACCGAAAAACUGGUU | |
| | | | | ACCAAAUGCAGCCAAAUGAAUCGUCCGUUGGCGGGCUCCGCGGCAGU | |
| | | | | GCAUAUCCCGCGUUAUCAUCACAUUACCUACCACACCAAACUGAGCA | |
| | | | | AAGACCGCGACGAGCGCCGUGAUCACAUGUGUCUGGUAGAGGUCGUG | |
| | | | | AAAGCGGUUGAUCUGGACACGUAUCAGUAAUAAAAAGCCCGAAAGGA | |
| | | | | AGCUGAGUUGGCUGCUGCCACCGCUGAGCAAUAA (SEQ ID NO: | |
| | | | | 109) | |

TABLE 9
ssDNA Primers Used to Generate the ssRNA Targets Using in Vitro Transcription

| ID | Type | Source | Description | Sequence |
|---|---|---|---|---|
| T7_primer | ssDNA | IDT | annealing to | CCTCGAGTAATACGACTCACTATAGGG (SEQ ID NO: 110) |
| | | | different IVT rev | |
| | | | primers to create | |
| | | | double-stranded T7 | |
| | | | promoter region | |
| | | | for IVT | |
| cr_F1_IVT_rev | ssDNA | IDT | For IVT of cr_F1 | GTTTTAGACCCCTGCAATTTTGCACGGGTGTAGTTCGCATTTCA |
| | | | | GTCAGTTGCTCAATGTACCTATGTTTTAGACCCCTGCAATTTTG |
| | | | | CACGGGTGTAGTTCCCCTATAGTGAGTCGTATTACTCGAGGAAT |
| | | | | TCTTATTATTTCT (SEQ ID NO: 111) |
| cr_F4_IVT_rev | ssDNA | IDT | For IVT of cr_F4 | GTTTTAGACCCCTGCAATTTTGCACGGGTGTAGTTCACGCCTGA |
| | | | | ATAAGTGATAATAAGCGGATGAGTTTTAGACCCCTGCAATTTTG |
| | | | | CACGGGTGTAGTTCCCCTATAGTGAGTCGTATTACTCGAGGAAT |
| | | | | TCTTATTATTTCT (SEQ ID NO: 112) |
| cr_F7_IVT_rev | ssDNA | IDT | For IVT of cr_F7 | GTTTTAGACTAGTGCAAATTCGCACCAGTGTAGTAGAGCTGGTG |
| | | | | ATATGGGATAGTGTTCACCCTTGGTTTTAGACTAGTGCAAATTT |
| | | | | GCACCAGTGTAGTAGCCCTATAGTGAGTCGTATTACTCGAGGGA |
| | | | | TCCTTATTACATTT (SEQ ID NO: 113) |
| cr_F1O_IVT_rev | ssDNA | IDT | For IVT of cr_F10 | GTTTTAGACTAGTGCAAATTCGCACCAGTGTAGTAGTTCGTTAA |
| | | | | TACAGATGTAGGTGTTCCACAGGGTTTTAGACTAGTGCAAATTT |
| | | | | GCACCAGTGTAGTAGCCCTATAGTGAGTCGTATTACTCGAGGGA |
| | | | | TCCTTATTACATTT (SEQ ID NO: 114) |
| cr_3_IVT_rev | ssDNA | IDT | For IVT of cr_3 | GTTTTAGTCCCCTTCGTTTTTGGGGTAGTCTAAATCCTAAGGAA |
| | | | | GCTAAAATGGAGAAAAAAATGTTTTAGTCCCCTTCGTTTTTGGG |
| | | | | GTAGTCTAAATCCCCTATAGTGAGTCGTATTACTCGAGGGATCC |
| | | | | TTATTACATTT (SEQ ID NO: 115) |
| cr_4_IVT_rev | ssDNA | IDT | For IVT of cr_4 | GTTTTAGTCCCCTTCGTTTTTGGGGTAGTCTAAATCGCTGGATG |
| | | | | GCCTICCCCATTATGATTCTGITTTAGTCCCCTTCGTTTTTGGG |
| | | | | GTAGTCTAAATCCCCTATAGTGAGTCGTATTACTCGAGGGATCC |
| | | | | TTATTACATTT (SEQ ID NO: 116) |
| sub_F1_rev | ssDNA | IDT | For IVT of sub_F1 | ATACGCTGTGGTTCGCCAAGTCCCAATGGCATCGTAAAGAACAT |
| | | | | TTTGAGGCATTTCAGTCAGTTGCTCAATGTACCTATAACCAGAC |
| | | | | CGTTCAGCTGGATATTACGGCCAAGAGAGCACGAAAGTGTTG |
| | | | | (SEQ ID NO: 117) |
| sub_F4_rev | ssDNA | IDT | For IVT of sub_F4 | ATACGCTGTGGTTCGCCAAGAGTTATTGGTGCCCTTAAACGCCT |
| | | | | GGTGCTACGCCTGAATAAGTGATAATAAGCGGATGAATGGCAGA |
| | | | | AATTCGAAAGCAAATTCGACCCAAGAGAGCACGAAAGTGTTG |
| | | | | (SEQ ID NO: 118) |
| sub_F7_rev | ssDNA | IDT | For IVT of sub_F7 | ATACGCTGTGGTTCGCCAAGCGGAATTCCGTATGGCAATGAAAG |
| | | | | ACGGTGAGCTGGTGATATGGGATAGTGTTCACCCTTGTTACACC |
| | | | | GTTTTCCATGAGCAAACTGAAACAAGAGAGCACGAAAGTGTTG |
| | | | | (SEQ ID NO: 119) |
| sub_F10_rev | ssDNA | IDT | For IVT of sub_F10 | ATACGCTGTGGTTCGCCAAGCTCCCAGAGCCTGATAAAAACGGT |
| | | | | TAGCGCTTCGTTAATACAGATGTAGGTGTTCCACAGGGTAGCCA |
| | | | | GCAGCATCCTGCGATGCAGATCCAAGAGAGCACGAAAGTGTTG |
| | | | | (SEQ ID NO: 120) |
| PT7_Sub_fw | ssDNA | IDT | For PCR all target | CGAAATTAATACGACTCACTATAGGGATACGCTGTGGTTCGCCA |
| | | | substrates for IVT | AG (SEQ ID NO: 121) |
| Sub_ry | ssDNA | IDT | For PCR all target | CGAAATTATTTCGACTGAGATTATTCCCCAACACTTTCGTGCTC |
| | | | substrates for IVT | TCTT (SEQ ID NO: 122) |
| GFP_PCR_fwd | ssDNA | IDT | For PCR GFP gene | GATGCGTCCGGCGTAGAGGATCGAGATCTC (SEQ ID NO: |
| | | | for IVT | 123) |

Notes:
IDT IVT: ssDNA primers from IDT were directly annealed with the T7_primer and transcribed
PCR IVT: a PCR using the IDT oligo or GFP as a template was used first to create the dsDNA with the T7 promoter sequence, on which IVT was then performed
IDT: primers ordered from Integrated DNA Technologies

### REFERENCES

East-Seletsky, A., O'Connell, M. R., Knight, S. C., Burstein, D., Cate, J. H. D., Tjian, R., and Doudna, J. A. (2016). Two distinct RNase activities of CRISPR-C2c2 enable guide-RNA processing and RNA detection. Nature 538, 270-273.
Knott, G. J., East-Seletsky, A., Cofsky, J. C., Holton, J. M., Charles, E., O'Connell, M. R., and Doudna, J. A. (2017). Guide-bound structures of an RNA-targeting A-cleaving CRISPR-Cas13aenzyme. Nat. Struct. Mol. Biol. 24, 825-833.

### Example 4. Validation of Type VI-D CRISPR-Cas Systems Comprising Cas13d and WYL1 Activity In Vitro (Biochemically)

Putative accessory proteins containing WYL domains and additional predicted DNA-binding domains are present in the great majority of the Type VI-D loci (FIG. 1). We initially synthesized and screened the predicted minimal CRISPR-Cas system for RspCas13d including both the RspCas13d effector and RspWYL1 accessory protein. To investigate the modulation of Cas13d by WYL1, we screened both the RspCas13d effector and RspWYL1 accessory protein separately. Comparison of screening results for RspCas13d effector alone versus the RspCas13d system, including RspWYL1, shows that RspCas13d targeted RNA cleavage is increased in the presence of RspWYL1 (FIGS. 27A-B). Bacterial screening with RspWYL1 alone yielded a minimal number of hits, indicating that RspWYL1 has no individual activity (FIG. 28). Cumulatively, these results suggest that RspCas13d enzymatic activity is modulated either directly or indirectly by WYL1.

We further investigated whether WYL1 could modulate RspCas13d in vitro by purifying recombinant RspWYL1 for use in ssRNA cleavage biochemical assays. To enable high resolution of enhanced or decreased complex activity in the presence of WYL, we selected doses of Cas13d-crRNA complex resulting in approximately 50% cleavage of the target substrates based on a dose titration curve (FIGS. 24A-B). We pre-incubated Cas13d-crRNA with no RspWYL1, an equimolar ratio of RspWYL1 to Cas13d, or a molar excess of RspWYL1 over Cas13d, and the resulting samples were incubated with target and collateral ssRNA under the same conditions as in the target cleavage assays. We observed that RspWYL1 increases both the targeted and collateral ssRNA cleavage activity of RspCas13d in a dose-dependent manner, with a molar excess of RspWYL1 yielding the greatest increase in Cas13d activity (FIGS. 29A-C).

Given that Type VI-D CRISPR-Cas systems appear to have acquired WYL-domain containing accessory proteins on multiple, independent occasions (FIGS. 1, 6, 8, 9), we tested the specificity of RspWYL1 in modulating the cleavage activity of orthologous Cas13d effectors. We observed that RspWYL1 enhanced the targeted and collateral ssRNA nuclease activities of EsCas13d to a similar extent as observed for RspCas13d (FIG. 30A-B). Thus, the effects of WYL1 orthologs appear not to be limited to their native effectors, but instead reflect a modular regulatory mechanism for Cas13d effectors.

To test whether RspWYL1 could modulate the activity of a type VI-B Cas13b effector, in vitro ssRNA cleavage biochemical assays were performed using recombinant RspWYL1 and Bergeyella zoohelcum Cas13b (BzCas13b). As shown in FIG. 31, RspWYL1 enhanced the activity of BzCas13b, demonstrating that this accessory protein is also capable of enhancing the activity of Cas13b effectors.

### Example 5. Type VI-D CRISPR-Cas Systems can be Used with a Fluorescent Reporter for the Specific Detection of Nucleic Acid Species

The dual nuclease activities of Cas13 effectors (i.e., target-specific and non-specific collateral RNase activity) make these effectors promising candidates for use in the detection of nucleic acid species. Some of these methods have been previously described (see, e.g., East-Seletsky et al. (2016), Gootenberg et al. (2017), and Gootenberg et al. (2018) “Multiplexed and portable nucleic acid detection platform with Cas13, Cas12a, and Csm6” Science 15 Feb. 2018: eaaq0179), describing the general principle of RNA detection using Cas13a (East-Seletsky et al. (2016)), supplemented by amplification to increase the detection sensitivity and optimization of additional Cas13a enzymes (Gootenberg et al. (2017)), and most recently, the inclusion of additional RNA targets, orthologous and paralogous enzymes, and Csm6 activator to enable multiplexed detection of nucleic acids along with an increase in detection sensitivity (Gootenberg et al. (2018)). The addition of Cas13d to this toolkit not only provides an additional channel of orthogonal activity for nucleic acid detection, but the nuclease activity-enhancing effect of the WYL1 proteins across orthologous and paralogous effectors suggests that WYL1 proteins can play an activity-enhancing role.

We tested the ability of EsCas13d or RspCas13d to cleave RNaseAlert® v2 (Thermo Fisher) substrate under different buffer conditions. Using a buffer of 50 mM potassium acetate, 20 mM Tris-acetate, 10 mM magnesium acetate, 100 μg/ml BSA, pH 7.9 provided key improvements from the described cleavage or processing buffers in the following: 1) maximum differentiation of targeting vs. non-targeting, 2) total fluorescence signal intensity, and 3) sufficient stability to support enzyme activity for the duration of the measurement.

We next tested different short fluorescent-quencher RNA substrates for the fluorescent detection of the collateral effect. These included RNase alert v2, a poly-G, and a poly-U substrate. We performed this experiment using a final reaction concentration of 40 nM of the Cas13d effector, 20 nM of crRNA, 5 nM of the target or nontarget RNA, and 160 nM of the fluorescent-quencher substrate along with 0.5 μL of the murine RNase inhibitor (in 50 uL) in the optimized buffer condition as described above. The reaction was incubated for 3 hours at 37° C. and the fluorescence read out using a Lightcycler 480 II at one-minute intervals. This demonstrated that both RspCas13d and EsCas13d can differentiate between a targeting vs. a non-targeting RNA using a poly-U substrate (FIG. 32). Furthermore, the differences between the activity of the two Cas13d effectors on the different substrate identities suggests the possibility of having multiple channels for the reporter.

The methods described above can include additional improvements to increase detection sensitivity. For example, a pre-amplification step of a nucleic acid in the sample (e.g., a target nucleic acid of interest) may be performed. These pre-amplification step can be performed by any method known in the art including, but not limited to, enzymatic methods such as isothermal amplification and recombinase polymerase amplification (RPA), as well as physical enrichment using methods such as immunoprecipitation. Furthermore, for the detection of DNA species, samples including the DNA species may be transcribed to convert the substrate into a Cas13d compatible substrate (e.g., RNA) while amplifying the target. A number of existing methods for nucleic acid enrichment or background amplification suppression can also be performed to increase the sensitivity and specificity of detection.

### Example 6. Type VI-D CRISPR-Cas Systems can be Used to Provide Genotype-Gated Control of Cell Death or Dormancy

Hybridization of the Type VI-D CRISPR-Cas effector protein and crRNA with an RNA target complementary to the crRNA spacer forms an active complex that may exhibit nonspecific, “collateral” RNase activity. Such collateral RNAse activity can be used to provide genotype-gated control of cell death or dormancy. The dependence of such activity on the presence of a specific RNA target in a cell is valuable since it enables targeting of specific cell populations based on specific underlying transcriptional states or genotypes. Numerous applications exist in both eukaryotic and prokaryotic settings for such control of cell death or dormancy.

For prokaryotic applications, a Type VI-D CRISPR-Cas system (e.g., including a Type VI-D effector and a crRNA) can be delivered (e.g., in vitro or in vivo) in order to induce cell death or dormancy of specific prokaryote populations (e.g., bacterial populations) in a genotype and transcriptome-specific way. For instance, the Type VI-D CRISPR-Cas system can include one or more crRNAs that specifically target a particular prokaryotic genus, species, or strain. This specific targeting has many therapeutic benefits as it may be used to induce death or dormancy of undesireable bacteria (e.g., pathogenic bacteria such as Clostridium difficile). In addition, the Type VI-D systems provided herein may be used to target prokaryotic cells having specific genotypes or transcriptional states. Within the microbial diversity that colonizes humans, only a small number of bacterial strains can induce pathogenesis. Further, even within pathogenic strains such as Clostridium difficile, not all members of the bacterial population exist continuously in active, disease-causing states. Thus, using RNA-targeting to control the activity of an Type VI-D effector based on the genotype and transcriptional state of a prokaryotic cell allows for specific control of which cells are targeted without disrupting the entire microbiome.

Additionally, bacterial strains can be readily engineered with genetic circuits or environmentally-controlled expression elements to generate genetic kill switches that limit the growth, colonization, and/or shedding of the engineered bacterial strains. For example, the expression of a TypeVI-D effectors, specific crRNA, or specific target RNA, can be controlled using promoters derived from the regulatory regions of genes encoding proteins expressed in response to external stimuli, such as cold sensitive proteins (PcspA), heat shock proteins (Hsp), chemically inducible systems (Tet, Lac, AraC). The controlled expression of one or more elements of the Type VI-D system allows for the full functional system to be expressed only upon exposure to an environmental stimulus, which in turn activates the nonspecific RNase activity of the system and thereby induces cell death or dormancy. Kill switches including Cas13d effectors as those described herein may be advantageous over traditional kill switch designs such as toxin/antitoxin systems (e.g., CcdB/CcdA Type II toxin/antitoxin systems), since they are not dependent on relative protein expression ratios which may be affected by leaky expression from a promoter (e.g., an environmental-stimulus dependent promoter), and thus allow for more precise control of the kill-switch.

To assess the ability of Cas13d to directly induce the dormancy or death of bacteria cells upon recognition of a target RNA, a variation of the in vivo functional screening described in Example 2 was performed, in which the antibiotic tetracycline was removed from the culture plate. Removing tetracycline selection meant that the survival of the host E. coli was no longer dependent on the successful natural expression of the tetracycline resistance protein by pACYC184. However, the targeting library still contained crRNAs with spacers to the tetracycline resistance gene, Tc^{R}. When the dependence of E. coli survival on successful Tc^{R} expression is removed, one would expect that there would be no impact on E. coli survival if the Cas13d effector directly cleaved Tc^{R} mRNA, and thus no Tc^{R} targeting spacers should register as strong depletion event on the in vivo screen. Nevertheless, the screening data without tetracycline selection still showed strongly depleted spacers on the Tc^{R} gene (FIGS. 33A-B, 34A-B), suggesting that the effect of Cas13d targeting RNA alone can mediate a growth disadvantage or cell death, even without antibiotic selection.

For eukaryotic applications, many diseases result from specific genotypes or transcriptional states in the diseased cells that distinguish them from healthy cells. Disease related genotypes are often contained in regions of the genome that are expressed, generating transcripts that can be targeted by a Type VI-D effector using a crRNA that specifically targets the genotype. Such targeting can provide cell dormancy or cell death in a population of cells with a specific disease related mutations. An exemplary application is the targeted depletion of cancer cells containing specific mutations, such as driver mutations that occur spontaneously in the tumor microenvironment. In addition, the Type VI-D CRISPR-Cas systems described herein can be used as kill-switch mechanisms to induce the death or dormancy of recombinant eukaryotic cells, such as chimeric antigen receptor-expressing T-cells, to limit their activity in inappropriate environments or when no longer desired.

Additionally, in a therapeutic context, numerous disease processes often involve dysregulation of cellular pathways that result in transcriptional states that are different from the normal baseline. A Type VI-D CRISPR-Cas system can be used to specifically induce the death or dormancy of cells that have an altered transcriptome. For example, the system can be used to induce the death or dormancy of cells having a temporally altered transcriptome, such as cells involved in an anti-inflammatory response during an autoimmune disease flare that are differentiated from normal cells.

The expression of the Type VI-D CRISPR-Cas systems described herein can be controlled and expressed using synthetic biology to induced or trigger cell death or dormancy. For example, the expression of genes encoding each of the components of the Type VI-D CRISPR-Cas systems can be controlled using genetic elements including, but are not limited to, promoters that are regulated by environmental stimuli, such as hypoxia (hif), neuronal activity (fos, arc), heat-shock (HSF-1), or exogenous controls such as light (FixJ), steroids (LexA), alcohol (AlcA), tetracycline (Tet). These promoters can be used to control the expression of components of the Type VI-D CRISPR-Cas system and/or of a specific RNA target to activate the system, thereby inducing the death or dormancy of targeted cells in response to the particular environmental stimuli to which the promoters respond.

### Example 7. Adaptation of Type VI-D CRISPR Cas System Effectors for Eukaryotic and Mammalian Activity

Beyond the biochemical and diagnostic applications described herein, programmable RNA-modifying CRISPR-Cas systems such as Type VI-D, e.g., Cas13d, systems described herein have important applications in eukaryotic cells, ranging from therapeutic uses such as disease transcript correction, to research and development advances, such as for transcriptome engineering and RNA visualization.

To develop Type VI-D CRISPR Cas systems for eukaryotic applications, the constructs encoding the protein effectors are first codon-optimized for expression in mammalian cells, and specific localization tags are optionally appended to either or both the N-terminus or C-terminus of the effector protein. These localization tags can include sequences such as nuclear localization signal (NLS) sequences, which localize the effector to the nucleus for modification of nascent RNAs, as well as nuclear export signal (NES) sequences, which target the effector to the cytoplasm in order to modify mature RNAs.

These sequences are described above in the “Functional Mutations” section. Other accessory proteins, such as fluorescent proteins, may be further appended. It has been demonstrated that the addition of robust, “superfolding” proteins such as superfolding green fluorescent protein (GFP) can increase the activity of Cas13 enzymes in mammalian cells when appended to the effector (Abudayyeh et al. (2017) Nature 550(7675): 280-4, and Cox et al. (2017) Science 358(6366): 1019-27).

The codon-optimized sequence coding for the Cas13d effector and appended accessory proteins and localization signals is then cloned into a eukaryotic expression vector with the appropriate 5′ Kozak eukaryotic translation initiation sequence, eukaryotic promoters, and polyadenylation signals. In mammalian expression vectors, these promoters can include, e.g., general promoters such as CMV, EF1a, EFS, CAG, SV40, and cell-type specific RNA polymerase II promoters such as Syn and CamKIIa for neuronal expression, and thyroxine binding globulin (TBG) for hepatocyte expression to name a few. Similarly, useful polyadenylation signals include, but are not limited to, SV40, hGH, and BGH. For expression of the pre-crRNA or mature crRNA, RNA polymerase III promoters such as H1 or U6 can be used.

Depending on the application and mode of packaging, the eukaryotic expression vector can be a lentiviral plasmid backbone, adeno-associated viral (AAV) plasmid backbone, or similar plasmid backbone capable of use in recombinant viral vector production. Notably, the small size of Type VI-D CRISPR Cas effector proteins, e.g., Cas13d effector proteins, make them ideally suited for packaging along with its crRNA and appropriate control sequences into a single adeno-associated virus particle; the packaging size limit of 4.7 kb for AAV may preclude the use of larger Cas13 effectors.

After adapting the sequences, delivery vectors, and methods for eukaryotic and mammalian use, different Cas13d constructs as described herein are characterized for performance. For efficient testing of the mammalian activity levels of various constructs, we use a dual-luciferase reporter expressing both Gaussia luciferase (Gluc) and Cypridinia luciferase (Cluc) (Abudayyeh et al. (2017) Nature 550(7675): 280-4). Targeting the Gluc transcript and comparing the relative activity versus the internal control of the Cluc activity enables an estimation of Cas13d effectiveness in a mammalian context. This activity is corroborated on the reporter through knockdown of endogenous transcripts, such as from the well-characterized KRAS genetic locus. The dual-luciferase reporter construct along with plasmids expressing the type VI-D CRISPR-Cas system and cognate crRNA are delivered using transient transfection (e.g., Lipofectamine® 2000) into model cell lines such as HEK 293T cells.

In addition to testing various construct configurations and accessory sequences on individual targets, pooled library-based approaches are used to determine 1) any targeting dependency of specific Cas13d effector proteins in mammalian cells as well as 2) the effect of mismatch locations and combinations along the length of the targeting crRNA. Briefly, the pooled library includes a plasmid that expresses a target RNA containing different flanking sequences as well as mismatches to the guide or guides used in the screening experiment, such that the successful target recognition and cleavage results in depletion of the sequence from the library. Furthermore, mRNA sequencing can be used to determine the off-target RNA cleavage effects of the type VI-D CRISPR-Cas system.

Complementary to the possibilities of transcriptome modification using the RNA cleavage activity of Cas13d, we can also explore the applications of catalytically-inactive Cas13d effector proteins in which the conserved residues of the two HEPN domains are mutated from the arginine and histidine to alanine. Like other Cas13 enzymes, catalytically inactive Cas13d (known as dCas13d) likely will retain its programmable RNA binding activity, though it will no longer be able to cleave target or collateral RNA.

In addition to direct uses of dCas13d such as in RNA immunoprecipitation, transcript labeling (when dCas13d effector is fused with fluorescent protein), and translation modification through site-specific targeted disruption of native translational machinery, other domains can be appended onto the dCas13d protein to provide further functionality. Activities of these domains include, but are not limited to, RNA base modification (ADAR1, ADAR2, APOBEC), RNA methylation (m⁶A methyltransferases and demethylases), splicing modifiers (hnRNPA1), localization factors (KDEL retention sequence, mitochondrial targeting signal, peroxisomal targeting signal), translation modification factors (EIF4G translation initiation factor, GLD2 poly(A) polymerase, transcriptional repressors). Additionally, domains can be appended to provide additional control, such as light-gated control (cryptochromes) and chemically inducible components (FKBP-FRB chemically inducible dimerization).

Optimizing the activity of such fusion proteins requires a systematic way of comparing linkers that connect the dCas13d with the appended domain. These linkers may include, but are not limited to, flexible glycine-serine (GS) linkers in various combinations and lengths, rigid linkers such as the alpha-helix forming EAAAK (SEQ ID NO: 124) sequence, XTEN linker (Schellenberger V, et al. Nat. Biotechnol. 2009; 27:1186-1190), as well as different combinations thereof (see TABLE 10). The various designs are then assayed in parallel over the same crRNA target complex and functional readout to determine which one yields the desired properties.

For adapting Cas13d for use in targeted RNA base modification (see, e.g., Cox DBT et al., Science 2017 10.1126/science.aaq0180), we begin with the Cas13d ortholog and NES combination that yielded the highest endogenous mammalian RNA knockdown activity and mutate the conserved residues of the two HEPN domains to create a catalytically inactive enzyme. Next, a linker is used to create the fusion protein between Cas13d-NES and the base editing domain. Initially, this domain will consist of the ADAR2DD(E488Q/T375G) mutant engineered previously for hyperactivity and greater specificity when used with Cas13b in REPAIRv2, but alternate deaminases such as ADAR1 and APOBEC1, among others, can be engineered and assayed in parallel (TABLE 10). Given the likely structural differences between the smaller Cas13d versus the previously characterized Cas13 effectors, alternate linker designs and lengths may yield the optimal design of the base editing fusion protein.

To evaluate the activity of the dCas13d-derived base editors, the HEK 293T cells are transiently transfected with the dCas13d-ADAR construct, a plasmid expressing the crRNA, and optionally, a reporter plasmid if targeting the reporter and not an endogenous locus. The cells are harvested 48 hours after transient transfection, the the RNA is extracted and reverse-transcribed to yield a cDNA library that is prepared for next generation sequencing. Analysis of the base composition of loci of samples containing the targeting vs. negative control non-targeting crRNAs provide information about the editing efficiency, and analysis of broader changes to the transcriptome will yield information about the off-target activity.

One particular advantage of developing an RNA base editing system using Cas13d is that the small size, on average 20% smaller than the existing Cas13 effectors, enables more ready packaging in AAV of dCas13d-ADAR along with its crRNA and control elements without the need for protein truncations. This all-in-one AAV vector enables greater efficacy of in vivo base editing in tissues, which is particularly relevant as a path towards therapeutic applications of Cas13d. In base editing and other applications, the small size, the lack of a biochemical PFS, and robust activity of Cas13d effectors make it a valuable addition to the toolbox of programmable RNA modifying enzymes.

Multiplexing of Cas13d with multiple crRNAs targeting different sequences enables the manipulation of multiple RNA species for therapeutic applications requiring manipulation of multiple transcripts simultaneously.

TABLE 10
Amino Acid Sequences of Motifs and Functional
Domains in Engineered Variants of Type VI-D
CRISPR-Cas Effector Proteins

>LINKER_1
GS
>LINKER_2
GSGGGGS (SEQ ID NO: 125)
>LINKER_3
GGGGSGGGGSGGGGS (SEQ ID NO: 126)
>LINKER_4
GGSGGSGGSGGSGGSGGS (SEQ ID NO: 127)
[ADAR1, ADAR2: C-term fusion
(or optionally N-term)]
>ADAR1DD-WT
SLGTGNRCVKGDSLSLKGETVNDCHAEIISRRGFIRFLYSELMKYNSQTA
KDSIFEPAKGGEKLQIKKTVSFHLYISTAPCGDGALFDKSCSDRAMESTE
SRHYPVFENPKQGKLRTKVENGEGTIPVESSDIVPTWDGIRLGERLRTMS
CSDKILRWNVLGLQGALLTHFLQPIYLKSVTLGYLFSQGHLTRAICCRVT
RDGSAFEDGLRHPFIVNHPKVGRVSIYDSKRQSGKTKETSVNWCLADGYD
LEILDGTRGTVDGPRNELSRVSKKNIFLLFKKLCSFRYRRDLLRLSYGEA
KKAARDYETAKNYFKKGLKDMGYGNWISKPQEEKNF (SEQ ID NO:
128)
>ADAR1DD-E1008Q (Cox et al., 2017)
SLGTGNRCVKGDSLSLKGETVNDCHAEIISRRGFIRFLYSELMKYNSQTA
KDSIFEPAKGGEKLQIKKTVSFHLYISTAPCGDGALFDKSCSDRAMESTE
SRHYPVFENPKQGKLRTKVENGQGTIPVESSDIVPTWDGIRLGERLRTMS
CSDKILRWNVLGLQGALLTHFLQPIYLKSVTLGYLFSQGHLTRAICCRVT
RDGSAFEDGLRHPFIVNHPKVGRVSIYDSKRQSGKTKETSVNWCLADGYD
LEILDGTRGTVDGPRNELSRVSKKNIFLLFKKLCSFRYRRDLLRLSYGEA
KKAARDYETAKNYFKKGLKDMGYGNWISKPQEEKNF (SEQ ID NO:
129)
>ADAR2DD-WT
QLHLPQVLADAVSRLVLGKFGDLTDNFSSPHARRKVLAGVVMTTGTDVKD
AKVISVSTGTKCINGEYMSDRGLALNDCHAEIISRRSLLRFLYTQLELYL
NNKDDQKRSIFQKSERGGFRLKENVQFHLYISTSPCGDARIFSPHEPILE
EPADRHPNRKARGQLRTKIESGEGTIPVRSNASIQTWDGVLQGERLLTMS
CSDKIARWNVVGIQGSLLSIFVEPIYFSSIILGSLYHGDHLSRAMYQRIS
NIEDLPPLYTLNKPLLSGISNAEARQPGKAPNFSVNWTVGDSAIEVINAT
TGKDELGRASRLCKHALYCRWMRVHGKVPSHLLRSKITKPNVYHESKLAA
KEYQAAKARLFTAFIKAGLGAWVEKPTEQDQFSLT (SEQ ID NO:
130)
>ADAR2DD-E488Q (Cox et al., 2017)
QLHLPQVLADAVSRLVLGKFGDLTDNFSSPHARRKVLAGVVMTTGTDVKD
AKVISVSTGTKCINGEYMSDRGLALNDCHAEIISRRSLLRFLYTQLELYL
NNKDDQKRSIFQKSERGGFRLKENVQFHLYISTSPCGDARIFSPHEPILE
EPADRHPNRKARGQLRTKIESGQGTIPVRSNASIQTWDGVLQGERLLTMS
CSDKIARWNVVGIQGSLLSIFVEPIYFSSIILGSLYHGDHLSRAMYQRIS
NIEDLPPLYTLNKPLLSGISNAEARQPGKAPNFSVNWTVGDSAIEVINAT
TGKDELGRASRLCKHALYCRWMRVHGKVPSHLLRSKITKPNVYHESKLAA
KEYQAAKARLFTAFIKAGLGAWVEKPTEQDQFSLT (SEQ ID NO:
131)
[Cytidine deaminase, AID, APOBEC1: N-term
fusion (or optionally C-term)]
>AID-APOBEC1 (Dickerson et al., 2003,
Komor et al., 2017)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
(SEQ ID NO: 132)
>Lamprey_AID-APOBEC1 (Rogozin et al.,
2007, Komor et al., 2017)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV (SEQ ID NO: 133)
>APOBEC1_BE1 (Komor et al., 2016)
MSSETGPVAVDPTLRRRIEPHEFEVFFDPRELRKETCLLYEINWGGRHSI
RHTSQNTNKHVEVNFIEKFTTERYFCPNTRCSITWFLSWSPCGECSRAIT
EFLSRYPHVTLFIYIARLYHHADPRNRQGLRDLISSGVTIQIMTEQESGY
CWRNFVNYSPSNEAHWPRYPHLWVRLYVLELYCIILGLPPCLNILRRKQP
QLTFFTIALQSCHYQRLPPHILWATGLK (SEQ ID NO: 134)

### REFERENCES

Abudayyeh, O. O., Gootenberg, J. S., Essletzbichler, P., Han, S., Joung, J., Belanto, J. J., Verdine, V., Cox, D. B. T., Kellner, M. J., Regev, A., et al. (2017). RNA targeting with CRISPR-Cas13. Nature 550, 280-284.
Cox, D. B. T., Gootenberg, J. S., Abudayyeh, O. O., Franklin, B., Kellner, M. J., Joung, J., and Zhang, F. (2017). RNA editing with CRISPR-Cas13. Science 358, 1019-1027.
Schellenberger V., Wang C. W., Geething N. C., Spink, B. J., Campbell, A., To, W., Scholle, M. D., Yin, Y., Yao, Y., Bogin, O., et al. (2009). A recombinant polypeptide extends the in vivo half-life of peptides and proteins in a tunable manner. Nat Biotechnol 2009; 27:1186-1190.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered, non-naturally occurring Clustered Regularly Interspaced Short Palindromic Repeat (CRISPR)—associated (Cas) system comprising:
an RNA guide or a nucleic acid encoding the RNA guide, wherein the RNA guide comprises a direct repeat sequence and a spacer sequence capable of hybridizing to a target nucleic acid; and
a Type VI-D CRISPR-Cas effector protein or a nucleic acid encoding the effector protein, wherein the effector protein comprises an amino acid sequence having at least 85% sequence identity to an amino acid sequence provided in Table 2, wherein the effector protein is capable of binding to the RNA guide and of targeting the target nucleic acid sequence complementary to the RNA guide spacer sequence.

2. The system of claim 1, wherein the effector protein comprises an amino acid sequence provided in Table 2.

3. The system of claim 1, wherein the effector protein is RspCas13d (SEQ ID NO: 2) or EsCas13d (SEQ ID NO: 1).

4. The system of any one of claims 1-3, wherein the effector protein comprises at least two HEPN domains, wherein none, one, or two of the HEPN domains are catalytically deactivated.

5. An engineered, non-naturally occurring Clustered Regularly Interspaced Short Palindromic Repeat (CRISPR)—associated (Cas) system comprising:
an RNA guide or a nucleic acid encoding the RNA guide, wherein the RNA guide comprises a direct repeat sequence and a spacer sequence capable of hybridizing to a target nucleic acid;
a CRISPR-associated protein or a nucleic acid encoding the CRISPR-associated protein; and
an accessory protein or a nucleic acid encoding the accessory protein, wherein the accessory protein comprises:
i) at least one WYL domain, wherein the WYL domain comprises an amino acid sequence PXXX₁XXXXXXXXXYL (SEQ ID NO: 198), wherein X₁ is C, V, I, L, P, F, Y, M, or W, and wherein X is any amino acid; and
ii) at least one ribbon-ribbon-helix (RHH) fold or at least one helix-turn-helix (HTH) domain;
wherein the CRISPR-associated protein is capable of binding to the RNA guide and of targeting the target nucleic acid sequence complementary to the spacer sequence, and wherein the accessory protein modulates an activity of the CRISPR-associated protein.

6. An engineered, non-naturally occurring Clustered Regularly Interspaced Short Palindromic Repeat (CRISPR)—associated (Cas) system comprising:
an RNA guide or a nucleic acid encoding the RNA guide, wherein the RNA guide comprises a direct repeat sequence and a spacer sequence capable of hybridizing to a target nucleic acid;
a CRISPR-associated protein or a nucleic acid encoding the CRISPR-associated protein; and
an accessory protein or a nucleic acid encoding the accessory protein, wherein the accessory protein comprises at least one WYL domain, and wherein the accessory protein comprises an amino acid sequence having at least 85% sequence identity to an amino acid sequence provided in any one of Tables 4, 5, and 6;
wherein the CRISPR-associated protein is capable of binding to the RNA guide and of targeting the target nucleic acid sequence complementary to the spacer sequence, and wherein the accessory protein modulates an activity of the CRISPR-associated protein.

7. The system of claim 5 or claim 6, wherein the activity is a nuclease activity.

8. The system of claim 7, wherein the nuclease activity is a DNAse activity.

9. The system of claim 7, wherein the nuclease activity is a targeted RNAse activity or a collateral RNAse activity.

10. The system of any one of claims 5-9, wherein the accessory protein increases the activity of the CRISPR-associated protein.

11. The system of any one of claims 5-9, wherein the accessory protein decreases the activity of the CRISPR-associated protein.

12. The system of any one of claims 6-11, wherein the accessory protein comprises an amino acid sequence provided in any one of Tables 4, 5, and 6.

13. The system of claim 5 or claim 6, wherein the accessory protein is RspWYL1 (SEQ ID NO: 81).

14. The system of any one of claims 5-13, wherein the targeting of the target nucleic acid results in a modification of the target nucleic acid.

15. The system of any one of claims 5-14, wherein the CRISPR-associated protein is a Class 2 CRISPR-Cas system protein.

16. The system of any one of claims 5-15, wherein the CRISPR-associated protein comprises a RuvC domain.

17. The system of any one of claims 5-15, wherein the CRISPR-associated protein is selected from the group consisting of a Type VI Cas protein, a Type V Cas protein, and a Type II Cas protein.

18. The system of any one of claims 5-15, wherein the CRISPR-associated protein is a Cas13a protein, a Cas13b protein, a Cas13c protein, a Cas12a protein, or a Cas9 protein.

19. The system of any one of claims 5-15, wherein the CRISPR-associated protein is a Type VI-D CRISPR-Cas effector protein comprising at least two HEPN domains, wherein none, one, or two of the HEPN domains are catalytically deactivated.

20. The system of claim 19, wherein the effector protein comprises an amino acid sequence having at least 85% sequence identity to an amino acid sequence provided in Table 2.

21. The system of claim 19 or claim 20, wherein the effector protein comprises an amino acid sequence provided in Table 2.

22. The system of any one of claims 19-21, wherein the effector protein is RspCas13d (SEQ ID NO: 2) or EsCas13d (SEQ ID NO: 1).

23. The system of any one of claims 1-22, wherein the target nucleic acid is an RNA.

24. The system of any one of claims 1-22, wherein the target nucleic acid is a DNA.

25. The system of any one of claims 1-4 and 14, wherein the modification of the target nucleic acid is a cleavage event.

26. The system of any one of claims 1-4, 14, and 25, wherein the modification results in: (a) decreased transcription; (b) decreased translation; or (c) both (a) and (b), of the target nucleic acid.

27. The system of any one of claims 1-4, 14, and 25, wherein the modification results in (a) increased transcription; (b) increased translation; or (c) both (a) and (b), of the target nucleic acid.

28. The system of any one of claims 4 and 19-22, wherein the effector protein comprises one or more amino acid substitutions within at least one of the HEPN domains.

29. The system of claim 28, wherein the one or more one amino acid substitutions comprise an alanine substitution at an amino acid residue corresponding to R295, H300, R849, or H854 of SEQ ID NO: 1, or R288, H293, R820, or H825 of SEQ ID NO: 2.

30. The system of claim 28 or claim 29, wherein the one or more amino acid substitutions result in a reduction of a nuclease activity of the Type VI-D CRISPR-Cas effector protein, as compared to the nuclease activity of the Type VI-D CRISPR-Cas effector protein without the one or more acid substitutions.

31. The system of any one of claims 1-30, wherein the direct repeat sequence comprises a nucleotide sequence provided in Table 3.

32. The system of any one of claims 1-30, wherein the direct repeat sequence comprises 5′-X₁X₂X₃X₄TX₅TX₆AAAC-3′ (SEQ ID NO: 199) at the 3′ terminal end of the RNA guide, and wherein X₁ is A or C or G, X₂ is A or G or T, X₃ is A or G or T, X₄ is C or G or T, X₅ is C or T, and X₆ is A or G.

33. The system of any one of claims 1-30, wherein the direct repeat sequence comprises either 5′-CACCCGTGCAAAATTGCAGGGGTCTAAAAC-3′ (SEQ ID NO: 152) or 5′-CACTGGTGCAAATTTGCACTAGTCTAAAAC-3′ (SEQ ID NO: 153).

34. The system of any one of claims 1-33, wherein the spacer comprises from about 15 to about 42 nucleotides.

35. The system of any one of claims 1-34, wherein the RNA guide further comprises a trans-activating CRISPR RNA (tracrRNA).

36. The system of any one of claims 1-35, further comprising a single-stranded donor template or a double-stranded donor template.

37. The system of claim 36, wherein the donor template is a DNA or an RNA.

38. The system of any one of claims 1-37, further comprising a target RNA or a nucleic acid encoding the target RNA, wherein the target RNA comprises a sequence that is capable of hybridizing to the spacer sequence of the RNA guide.

39. The system of any one of claims 1-38, wherein the system is present in a delivery system.

40. The system of claim 39, wherein the delivery system comprises a delivery vehicle selected from the group consisting of a nanoparticle, a liposome, an adeno-associated virus, an exosome, a microvesicle, and a gene-gun.

41. A cell comprising the system of any one of claims 1-40.

42. The cell of claim 41, wherein the cell is a eukaryotic cell.

43. The cell of claim 42, wherein the eukaryotic cell is a mammalian cell or a plant cell.

44. The cell of claim 41, wherein the cell is a prokaryotic cell.

45. The cell of claim 44, wherein the prokaryotic cell is a bacterial cell.

46. An animal model or a plant model comprising the cell of any one of claims 41-45.

47. A method of cleaving a target nucleic acid, the method comprising contacting the target nucleic acid with a system of any one of claims 1-40;
wherein the spacer sequence is complementary to at least 15 nucleotides of the target nucleic acid;
wherein the CRISPR-associated protein or the Type VI-D CRISPR effector protein associates with the RNA guide to form a complex;
wherein the complex binds to a target nucleic acid sequence that is complementary to the at least 15 nucleotides of the spacer sequence; and
wherein upon binding of the complex to the target nucleic acid sequence the CRISPR-associated protein or the Type VI-D CRISPR effector protein cleaves the target nucleic acid.

48. The method of claim 47, wherein the target nucleic acid is within a cell.

49. A method of inducing dormancy or death of a cell, the method comprising contacting the cell with a system of any one of claims 1-40;
wherein the spacer sequence is complementary to at least 15 nucleotides of the target nucleic acid;
wherein the CRISPR-associated protein or the Type VI-D CRISPR effector protein associates with the RNA guide to form a complex;
wherein the complex binds to a target nucleic acid sequence that is complementary to the at least 15 nucleotides of the spacer sequence; and
wherein upon binding of the complex to the target nucleic acid sequence the CRISPR-associated protein or the Type VI-D CRISPR-Cas effector protein cleaves a non-target nucleic acid within the cell, thereby inducing dormancy or death of the cell.

50. The method of any one of claims 47-49, wherein the target nucleic acid is an RNA selected from the group consisting of an mRNA, a tRNA, a ribosomal RNA, a non-coding RNA, a lncRNA, or a nuclear RNA.

51. The method of claim 49, wherein the target nucleic acid is a DNA selected from the group consisting of chromosomal DNA, mitochondrial DNA, single-stranded DNA, or plasmid DNA.

52. The method of any one of claims 47-51, wherein upon binding of the complex to the target nucleic acid, the CRISPR-associated protein or the Type VI-D CRISPR-Cas effector protein exhibits collateral RNAse activity.

53. The method of any one of claims 49-52, wherein the death is via apoptosis, necrosis, necroptosis, or a combination thereof.

54. The method of any one of claims 48-53, wherein the cell is a cancer cell.

55. The method of claim 54, wherein the cancer cell is a tumor cell.

56. The method of any one of claims 48-53, wherein the cell is an infectious agent cell or a cell infected with an infectious agent.

57. The method of claim 48-53, wherein the cell is a bacterial cell, a cell infected with a virus, a cell infected with a prion, a fungal cell, a protozoan, or a parasite cell.

58. A method of treating a condition or disease in a subject in need thereof, the method comprising administering to the subject a system of any one of claims 1-40,
wherein the spacer sequence is complementary to at least 15 nucleotides of a target nucleic acid associated with the condition or disease;
wherein the CRISPR-associated protein or the Type VI-D CRISPR-Cas effector protein associates with the RNA guide to form a complex;
wherein the complex binds to a target nucleic acid sequence that is complementary to the at least 15 nucleotides of the spacer sequence; and
wherein upon binding of the complex to the target nucleic acid sequence the CRISPR-associated protein or the Type VI-D CRISPR-Cas effector protein cleaves the target nucleic acid, thereby treating the condition or disease in the subject.

59. The method of claim 58, wherein the condition or disease is a cancer or an infectious disease.

60. The method of claim 59, wherein the condition or disease is cancer, and wherein the cancer is selected from the group consisting of Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

61. A system according to any one of claims 1-40, for use in a method selected from the group consisting of RNA sequence specific interference; RNA sequence-specific gene regulation; screening of RNA, RNA products, lncRNA, non-coding RNA, nuclear RNA, or mRNA; mutagenesis; inhibition of RNA splicing; fluorescence in situ hybridization; breeding; induction of cell dormancy; induction of cell cycle arrest; reduction of cell growth and/or cell proliferation; induction of cell anergy; induction of cell apoptosis; induction of cell necrosis; induction of cell death; or induction of programmed cell death.

62. The system of claim 1, wherein the effector protein is fused to a base-editing domain, a RNA methyltransferase, a RNA demethylase, a splicing modifier, a localization factor, or a translation modification factor.

63. The system of claim 5 or claim 6, wherein the CRISPR-associated protein is fused to a base-editing domain, a RNA methyltransferase, a RNA demethylase, a splicing modifier, a localization factor, or a translation modification factor.

64. The system of claim 62 or claim 63, wherein the base editing domain is selected from the group consisting of Adenosine Deaminase Acting on RNA (ADAR) 1 (ADAR1), ADAR2, apolipoprotein B mRNA editing enzyme, catalytic polypeptide-like (APOBEC), and activation-induced cytidine deaminase (AID).

65. The system of any one of claims 1-40, further comprising an RNA-binding fusion polypeptide that comprises an RNA-binding domain and a base-editing domain.

66. The system of claim 65, wherein the base-editing domain is selected from the group consisting of ADAR1, ADAR2, APOBEC, and AID.

67. The system of claim 65 or claim 66, wherein the RNA-binding domain is MS2.

68. A method of modifying an RNA molecule, comprising contacting the RNA molecule with a system according to any one of claims 62-67.

69. A method of detecting a target RNA in a sample, the method comprising:
a) contacting the sample with:
(i) an RNA guide or a nucleic acid encoding the RNA guide, wherein the RNA guide comprises a direct repeat sequence and a spacer sequence capable of hybridizing to the target RNA;
(ii) a Type VI-D CRISPR-Cas effector protein or a nucleic acid encoding the effector protein; and
(iii) a labeled detector RNA;
wherein the effector protein associates with the RNA guide to form a complex;
wherein the RNA guide hybridizes to the target RNA; and wherein upon binding of the complex to the target RNA, the effector protein exhibits collateral RNAse activity and cleaves the labeled detector RNA; and
b) measuring a detectable signal produced by cleavage of the labeled detector RNA,
wherein said measuring provides for detection of the target RNA in the sample.

70. The method of claim 69, wherein the effector protein comprises an amino acid sequence having at least 85% sequence identity to an amino acid sequence provided in Table 2.

71. The method of claim 69 or claim 70, wherein the target RNA is single-stranded.

72. The method of any one of claims 69-71, wherein the target RNA was transcribed from a DNA molecule.

73. The method of any one of claims 69-72, further comprising contacting the sample with an accessory protein comprising at least one WYL domain.

74. The method of claim 73, wherein the accessory protein comprises an amino acid sequence having at least 85% sequence identity to an amino acid sequence provided in any one of Tables 4, 5, and 6.

75. The method of any one of claims 69-74, further comprising comparing the detectable signal with a reference signal and determining the amount of target RNA in the sample.

76. The method of any one of claims 69-75, wherein the measuring is performed using gold nanoparticle detection, fluorescence polarization, colloid phase transition/dispersion, electrochemical detection, and semiconductor based-sensing.

77. The method of any one of claims 69-76, wherein the labeled detector RNA comprises a fluorescence-emitting dye pair, a fluorescence resonance energy transfer (FRET) pair, or a quencher/fluor pair.

78. The method of any one of claims 69-77, wherein upon cleavage of the labeled detector RNA by the effector protein, an amount of detectable signal produced by the labeled detector RNA is decreased.

79. The method of any one of claims 69-78, wherein upon cleavage of the labeled detector RNA by the effector protein, an amount of detectable signal produced by the labeled detector RNA is increased.

80. The method of any one of claims 69-79, wherein the labeled detector RNA produces a first detectable signal prior to cleavage by the effector protein and a second detectable signal after cleavage by the effector protein.

81. The method of any one of claims 69-80, wherein a detectable signal is produced when the labeled detector RNA is cleaved by the effector protein.

82. The method of any one of claims 69-81, further comprising pre-amplify a nucleic acid in the sample prior to the contacting step.
